(19) United States
(12) Patent Application Publication
(10) Pub. No.: US 2026/0226549 A1
(43) Pub. Date: Aug. 6, 2026

(54) Method for identifying kidney allograft rejection genes in urine and utility of making those measurements

(71) Applicant: Natera, Inc., San Carlos, CA (US)

(72) Inventors: Alain MIR, Castro Valley, CA (US); Alvin CHON, Auburn, WA (US); Aaron BRUNS, San Mateo, CA (US); Swee Seong WONG, Carmel, IN (US)

(73) Assignee: Natera, Inc., San Carlos, CA (US)

(21) Appl. No.: 19/153,213

(22) PCT Filed: Feb. 7, 2024
(86) PCT No.: PCT/US2024/014805

§ 371 Date: Aug. 1, 2025

Related U.S. Application Data
(60) Provisional application No. 63/443,873, filed on Feb. 7, 2023.

(51) Int. Cl. C12Q 1/6883; C12Q 1/6806; G01N 33/68; G16B 40/00

(52) U.S. Cl. CPC C12Q 1/6883; C12Q 1/6806; G01N 33/6893; G16B 40/00; C12Q 2600/158; C12Q 2600/16; C12Q 2600/178; G01N 2800/245; G01N 2800/50

(57) ABSTRACT

The present disclosure provides methods for preparation and analysis of biological samples of kidney transplant recipients, wherein the methods comprise nucleic acids or protein derived from a urine sample of the kidney transplant recipient, and measuring amounts of nucleic acids or protein expressed from pre-selected target genes indicative of kidney disease states. These methods enable assessment of kidney transplant rejection and kidney transplant rejection states such as T-cell mediated rejection (TCMR), antibody-mediated rejection (ABMR), or a mixed TCMR and ABMR disease state.

### CROSS-REFERENCE TO RELATED APPLICATION

This application claims priority to and the benefit of U.S. Provisional Patent Application No. 63/443,873 filed Feb. 7, 2023, which is incorporated herein by reference in its entirety.

### BACKGROUND

Over 26 million people in the US are afflicted with chronic kidney disease (CKD), as defined by persistent albuminuria and/or decline in glomerular filtration rate. Given this prevalence, kidney transplant accounts for the highest number of transplants per year. It also remains the most favorable therapy for end-stage renal disease. Kidney transplant allograft rejection may lead to a high risk of graft dysfunction, increased probability of chronic failure and eventual graft loss.

As of December 2018, there were 229,887 patients in the United States with a functioning kidney transplant, or 678 recipients per million people, representing 40% growth since 2008. In 2019, a record 24,273 kidney transplants were performed in the United States. In 2020, the average kidney transplant cost was US$442,500. Charges for the transplant admission, which include the surgery itself, are the most expensive line item, accounting for 34% of the total cost with increased transplant rates predicted. Additionally, as of 2018, nearly 95,000 patients were waiting for a kidney transplant in the United States, and more than half of listed candidates die or are removed from the list before transplant. Therefore, preserving the functionality of transplanted organs is of utmost importance for the health of the recipients, constraining the cost of care, and protecting the limited supply of organs to serve the needs of a growing number of patients.

Graft failure mainly results due to causes other than acute rejection. El-Zoghby et al., Am J Transplant, 9(3):527-35 (2009) found that out of 1317 conventional kidney recipient 330 grafts were lost. Disease pathogenesis remains complicated to unravel. The abnormal presence of plasma-derived proteins and associated factors likely incites tubulointerstitial damage may amplify an inherent susceptibility of the kidney to dysfunction. Specific inflammatory cells, arising from both resident and recruited circulating inflammatory cells, may both engage in phagocytosis of damaged cells and matrix after injury. However, if the inflammatory response is not well “titrated” it can become unrestrained and populations of kidney macrophages become injurious, cither through secretion of inflammatory cytokines (e.g., IL-1β, IL-18, TNF-α and C—X—C chemokines), which can stimulate apoptosis and further inflammation and eventual rejection.

Allograft rejection can lead to severely impaired transplant function and worsening survival prognosis, and is generally categorized as either cellular i.e. T-cell mediated rejection: (TCMR) or humoral i.e. antibody mediated rejection (ABMR), although it is also possible for patients to present with a combination of both (mixed). Both rejection pathologies are triggered by recognition of alloantigens on the allograft by recipient T cells. Moreover, insufficient suppression of T cells due to non-adherence with medications or intentional reduction of immunosuppressive agents is an important cause of allograft rejection.

Regardless of a strong and unambiguous understanding of the initiating events in CKD, premature graft loss is common. As a consequence, there is a pressing need for early, sensitive and accurate determination of kidney rejection states to improve monitoring, management and treatment strategies for graft rejection before it is too late to reverse the rejection process. The present disclosure provides this need.

### SUMMARY

In one aspect the present disclosure relates to a method of preparing a composition of nucleic acids derived from a urine sample of a kidney transplant recipient useful for determination of kidney transplant rejection or risk of kidney transplant rejection comprising: (a) extracting nucleic acids from the urine sample of the kidney transplant recipient, wherein the extracted nucleic acids comprise one or more mRNAs of target genes and/or miRNAs associated with kidney transplant rejection; (b) preparing a composition of nucleic acids from the extracted nucleic acids in step (a) by isolating mRNAs and/or miRNAs and removing contaminating molecules, optionally wherein preparing the composition comprises reverse transcribing complementary DNA (cDNA) from the nucleic acids extracted in step (a); (c) measuring an amount of the one or more mRNAs and/or miRNAs, and generating one or more transplant rejection scores from the measured amount of the one or more mRNAs and/or miRNAs, wherein the one or more transplant rejection scores provide a quantitative value of kidney transplant rejection risk or the presence of kidney transplant rejection.

In some embodiments, the method comprises generating two or more transplant rejection scores, wherein each transplant rejection score is based on a different subset of mRNAs and/or miRNAs.

In some embodiments, the one or more transplant rejection scores are generated using a predictive model, a machine learning based method, and/or an artificial intelligence method.

In some embodiments, the one or more transplant rejection scores are generated using logistic regression (Log Reg), t-test, violin plots, random forest (RE), a neural network, decision tree machine learning analysis, decision trees classification techniques, analysis of variants (ANOVA); Bayesian networks; boosting and Ada-boosting; bootstrap aggregating (or bagging) algorithms, Classification and Regression Trees (CART), boosted CART, Recursive Partitioning Trees (RPART), Curds and Whey (CW); Curds and Whey-Lasso; principal component analysis (PCA), factor rotation or factor analysis; discriminant analysis, Linear Discriminant Analysis (LDA), Eigengene Linear Discriminant Analysis (ELDA), quadratic discriminant analysis; Discriminant Function Analysis (DFA); factor rotation or factor analysis; genetic algorithms; Hidden Markov Models; kernel based machine algorithms such as kernel density estimation, kernel partial least squares algorithms, kernel matching pursuit algorithms, kernel Fisher's discriminate analysis algorithms, kernel principal components analysis algorithms; linear regression and generalized linear models, Forward Linear Stepwise Regression, Lasso (or LASSO) shrinkage and selection method, Elastic Net regularization and selection method; glmnet (Lasso and Elastic Net-regularized generalized linear model); meta-learner algorithms; nearest neighbor methods for classification or regression, Kth-nearest neighbor (KNN); non-linear regression or classification algorithms; neural networks; partial least square; rules based classifiers; shrunken centroids (SC); sliced inverse regression; Standard for the Exchange of Product model data, Application Interpreted Constructs (StepAIC); super principal component (SPC) regression; Support Vector Machines (SVM) and Recursive Support Vector Machines (RSVM), and/or combinations thereof.

In some embodiments, the one or more transplant rejection scores are generated using logistic regression (Log Reg), random forest (RE), a neural network, or decision tree machine learning analysis.

In some embodiments, the one or more mRNAs and/or miRNAs are examined by using 8 separate machine learning classifier methods based on 6 determined kidney disease states.

In some embodiments, a performance of the determination of kidney transplant rejection or risk of kidney transplant rejection is measured and characterized by an AUC value from about 0.6 to about 0.99, from about 0.7 to about 0.99, from about 0.8 to about 0.99, from about 0.9 to about 0.99, from about 0.7 to about 0.79, from about 0.8 to about 0.89, from about 0.6 to about 0.89, or from about 0.6 to about 0.79.

In some embodiments, the AUC value is from about 0.8 to about 0.99.

In some embodiments, the one or more transplant rejection scores provide a quantitative value of kidney transplant rejection risk that is predictive of a clinical assessment of a kidney disease state and can distinguish between two or more kidney disease states.

In some embodiments, the kidney disease state comprises non-rejection, T-cell mediated rejection (TCMR), antibody-mediated rejection (ABMR), or a mixed TCMR and ABMR disease state.

In some embodiments, the TCMR further comprises molecularly defined TCMR (mTCMR) and/or possible TCMR (pTCMR).

In some embodiments, the ABMR further comprises molecularly defined ABMR (mABMR) and/or possible ABMR (pABMR).

In some embodiments, the one or more transplant rejection scores comprise a first transplant rejection score based on a set of mRNAs and/or miRNAs associated with TCMR, and a second transplant rejection score based on a set of mRNAs and/or miRNAs associated with ABMR.

In some embodiments, the one or more transplant rejection scores comprise a transplant rejection score based on a set of mRNAs and/or miRNAs associated with inflammation, a transplant rejection score based on a set of mRNAs and/or miRNAs associated with allograft rejection, a transplant rejection score based on a set of mRNAs and/or miRNAs associated with T cell activation and/or differentiation, a transplant rejection score based on a set of mRNAs and/or miRNAs associated with B cell activation and/or differentiation, a transplant rejection score based on a set of mRNAs and/or miRNAs associated with a cytokine response, and/or a transplant rejection score based on a set of mRNAs and/or miRNAs associated with a chemokine response.

In some embodiments, the method disclosed herein comprises collecting the urine sample from the transplant recipient at different times and generating the one or more transplant rejection scores from each urine sample.

In some embodiments, the urine sample is collected from the transplant recipient prior to transplantation, simultaneous with transplantation, and/or after transplantation.

In some embodiments, the risk of transplant rejection is based on two or more transplant rejection scores generated at different time points, and wherein a change in two or more transplant rejection scores indicates a change in kidney disease state.

In some embodiments, the kidney transplant recipient is administered a treatment for the determined kidney disease state or kidney transplant rejection.

In some embodiments, the treatment comprises an anti-rejection or an immunosuppressive agent.

In some embodiments, a change in one or more transplant rejection scores specifies a presence, absence, or extent of therapeutic response to the treatment.

In some embodiments, the treatment is determined based on the one or more transplant rejection scores or a change in one or more transplant rejection scores.

In some embodiments, the nucleic acids comprise cellular nucleic acids, extra-cellular nucleic acids, and/or nucleic acids obtained from extracellular vesicles.

In some embodiments, the method comprises isolating cells from the urine samples, and extracting nucleic acids from the cells.

In some embodiments, the method further comprises isolating extracellular vesicles, and extracting nucleic acids from the extracellular vesicles.

In some embodiments, the cDNA is amplified prior to measuring of the amount.

In some embodiments, the extracted nucleic acids comprise one or more mRNAs.

In some embodiments, wherein the extracted nucleic acids comprise one or more miRNAs.

In some embodiments, the extracted nucleic acids comprise one or more mRNAs and one or more miRNAs.

In some embodiments, the step of preparing the composition of the nucleic acids extracted in step (a) or fractions thereof comprises amplification of cDNA derived from the nucleic acids.

In some embodiments, the amplification comprises performing multiplex targeted amplification of the cDNA at 10-50,000 target loci in a single reaction volume.

In some embodiments, the amplification comprises universal amplification.

In some embodiments, the amount of one or more mRNAs and/or miRNAs is measured by using quantitative PCR, real-time PCR, digital PCR, or sequencing.

In some embodiments, the amount of one or more mRNAs and/or miRNAs is measured by using multiplex quantitative PCR, multiplex real-time PCR, and/or multiplex digital PCR.

In some embodiments, sequencing comprises next-generation whole genome sequencing.

In some embodiments, the amount of one or more mRNAs and/or miRNAs is measured by using microarray.

In some embodiments, the amount of one or more mRNAs and/or miRNAs is measured by using molecular barcodes and microscopic imaging (such as NanoString nCounter®).

In some embodiments, the amount of one or more mRNAs and/or miRNAs is determined by measuring an absolute copy number of the one or more mRNAs and/or miRNAs per amount of total nucleic acids in the urine sample.

In some embodiments, one or more mRNAs and/or miRNAs are associated with antibody mediated transplant rejection (AMTR), T-cell mediated transplant rejection (TMTR), apoptosis pathways, cytokine, antimicrobial responses, and/or inflammatory cellular responses.

In some embodiments, the one or more mRNAs and/or miRNAs are associated with the antimicrobial responses are CXC motif chemokine ligand (CXCL) type genes.

In some embodiments, the one or more mRNAs are expressed from a gene selected from the group consisting of:

| | | | | |
|---|---|---|---|---|
| ITM2A | SLAMF6 | IKZF3 | CCL5 | CXCL9 |
| IGHM | ITM2A | HIST1H2AJ | CXCL10 | CD3E |
| NKG7 | TRBC2 | HIST1H3I | TRGC2 | HIST1H1B |
| CTLA4 | PDCD1 | GZMA | IL2RB | IKZF3 |
| CD69 | SIT1 | CD96 | FAIM3 | ZAP70 |
| HIST1H1D | TRAC | RNASE6 | SLAMF6 | Z98744.2 |
| GZMB | IL18R1 | IL1RL1 | HIST1H4L | C1QB |
| CD8A | HIST1H2BM | HLA-DQB1 | LCK | ASB2 |
| HIST1H2AI | SLFN12L | CD28 | TRBV28 | RP4-620F22.2 |
| GIMAP1 | ICOS | ZNF831 | TRAT1 | GIMAP7 |
| P2RY10 | HCST | HIST1H2AK | GBP5 | EMB |
| CD2 | KLRC1 | CD6 | GPR174 | AL049822.1 |
| HLA-DQA1 | TIGIT | ITK | ST8SIA4 | GIMAP6 |
| GVINP1 | HLA-DQB1- | MRC1 | HLA-DPB1 | CCR2 |
| | AS1 | | | |
| CCND2-AS1 | AL009179.1 | EVI2B | FCGR3B | ETV7 |
| CTSW | HIST1H2AL | THEMIS | CH17- | BLM |
| | | | 373J23.1 | |
| PRF1 | DOK2 | CXCR6 | NGFR | KCNE1 |
| HIST1H3H | CTD- | HLA-DRA | CD8B | NCF1 |
| | 2313F11.1 | | | |
| GZMK | MLKL | HIST1H3B | CD3G | JAK3 |
| CD3D | C1QA | IFNG | CDCA7 | Z98744.3 |
| CXCL11 | RP11-284N8.3 | HLA-H | TOX | GIMAP4 |
| FAM78A | FAM65B | HCG4P7 | SMAP2 | EOMES |
| RASAL3 | CD163 | FCRL3 | IFIT2 | SH2D1A |
| HCG4P11 | RGS18 | MIR155HG | IFITM1 | HIST1H2BL |
| ITGA4 | RP11- | MATK | KCNJ2 | KLRB1 |
| | 463J10.3 | | | |
| HIST1H2AH | APOBEC3G | PSMB9 | PTPRC | RCSD1 |
| PYHIN1 | BTN3A3 | AC007278.2 | C11orf21 | RHOH |
| TMEM156 | HIST2H3D | NLRP3 | AF001548.5 | HIST1H2BI |
| CD74 | AGAP2 | CD52 | GBP1 | GMFG |
| LAX1 | SASH3 | GNLY | HIST2H2AB | MS4A6A |
| PDE3B | SLAMF8 | AGER | PREX1 | IRF8 |
| IKZF1 | TBC1D10C | FCGR3A | BCL11B | HLA-DRB1 |
| CD97 | CTB-186G2.1 | NLRC3 | TAP1 | CD247 |
| HIST1H3G | GBP4 | RP11- | TMC8 | SELPLG |
| | | 1049A21.2 | | |
| HIST1H2BO | CCND2 | CST7 | CCR5 | RP11-264B17.5 |
| GRAP2 | HLA-B | IKBIP | KRT1 | SERPINB12 |
| ALB | TGM3 | HS3ST6 | NRTN | FAM180A |
| FALEC | FGF22 | HCN2 | SLC24A3 | RP11-167H9.5 |
| C1QB | IL18R1 | KCNE1 | CCR2 | C1QA |
| CD163 | CST7 | SLC1A3 | RNASE6 | HIST1H1B |
| TRGC2 | MS4A6A | C1QC | RGS1 | MSR1 |
| HIST1H1D | HIST1H3I | CD28 | LSM11 | HIST1H3B |
| HIST1H2AJ | GIMAP6 | DAAM2 | CCL5 | CTLA4 |
| IGHG1 | C1orf162 | MIR155HG | ITM2A | GIMAP4 |
| HIST1H2BM | HIST1H3G | FCER1G | FKBP5 | IL1R2 |
| GZMA | CLEC12A | LINC01127 | Z98744.2 | GPR34 |
| HLA-DMB | HIST1H4L | RRM2 | TREM2 | GIMAP8 |
| CD3E | AL049822.1 | HLA-DPB1 | LMNB1 | CD300C |
| IFITM1 | BLM | SAMSN1 | IRF8 | CLSPN |
| IL1RL1 | SDS | RP11-274E7.2 | MS4A4A | PDCD1 |
| ICOS | RP11- | TNFSF13B | HIST1H3F | RP11-777B9.5 |
| | 848G14.5 | | | |
| GIMAP2 | IL18RAP | VSIG4 | GIMAP7 | SRGN |
| TRBC2 | LIPA | SERPINE1 | CENPK | IL10 |
| GZMK | DTL | GLDN | SMAP2 | HIST1H2BB |
| SIT1 | IFITM3 | CTD- | HLA-DRA | THBS1 |
| | | 2033D15.2 | | |
| ADORA3 | STAB1 | AIM2 | EVI2A | TFPI2 |
| EMB | SLA | CD8A | RCSD1 | GIMAP1 |
| FAM105A | FPR2 | CD180 | RP11- | RP4-737E23.5 |
| | | | 463J10.3 | |
| FCGR3A | HIST1H2BL | CD1D | UBE2C | PDK4 |
| MLKL | RP11-7F17.3 | TLR7 | PTPRC | KBTBD8 |
| CD96 | HIST1H2AI | FAIM3 | CD48 | IFI27L2 |
| HIST1H4F | CTD- | SLC16A6 | LRRC8C | NLRP6 |
| | 2033D15.3 | | | |
| TLR8 | IRAK3 | LY96 | LILRA5 | AL009179.1 |
| KIAA0101 | GCA | CCDC109B | CTSL | CCR5 |
| BTN3A2 | STAMBPL1 | HLA-DPA1 | CTD- | LAIR1 |
| | | | 2313F11.1 | |
| FES | RNASE1 | CXCL10 | TRIM22 | HIST1H3H |
| CD52 | MYBL2 | RP11- | RNU4-1 | KLRC1 |
| | | 403P17.6 | | |
| FEN1 | MIR181A1HG | HCG27 | RP11- | BIN2 |
| | | | 335I12.2 | |
| RP4-620F22.2 | GBP5 | FYB | PCED1B | AL031777.1 |
| MMD | DOK2 | RP11- | PPT1 | FAM65B |
| | | 404F10.2 | | |
| GVINP1 | SERPINF1 | CARD16 | CEP55 | HIST1H2AL |
| CD86 | CD2 | JAK3 | SELPLG | AC007278.3 |
| FAM111B | GYG1 | NLRP3 | ZWINT | ITGA4 |
| TRAC | SELL | GPR183 | TRG-AS1 | ST8SIA4 |
| NCKAP1L | STK17B | OLAH | APOC1 | PCED1B-AS1 |
| SLFN13 | TIGIT | ATHL1 | HIST1H2BI | HLA-DMA |
| ENTPD1 | SLCO2B1 | IFNG | CD300LF | F13A1 |
| NEIL3 | TMEM14E | ACSL4 | RN7SKP48 | GZMH |
| LYAR | GPNMB | IKZF1 | RP3-455J7.4 | SCIMP |
| AMIGO2 | TLR5 | EXOC6 | IFITM2 | HAVCR2 |
| TLR2 | CTB- | FGL2 | A2M | LY9 |
| | 111H14.1 | | | |
| KIAA0825 | WDR76 | LILRB5 | SLAMF6 | CTB-41I6.2 |
| FGR | HELLS | CD274 | HIST1H2AB | MELK |
| ASB2 | SIGLEC9 | TPK1 | MAP2K6 | RP11-347P5.1 |
| HIST1H2AH | EVI2B | CTA-373H7.7 | FAM26F | KLRB1 |
| TRPV2 | CHIT1 | HTATSF1P2 | TSC22D3 | TLDC2 |
| MGAM | RP11-69L16.4 | CXCR6 | STAT4 | TNFSF8 |
| MB21D1 | SLFN11 | RP11- | TYROBP | ITGB2 |
| | | 124N14.3 | | |
| LILRB4 | CDK1 | PSMB8 | HIST2H2AB | DNA2 |
| PDCD1LG2 | CCL18 | RP11-25K21.6 | HLA-DQA1 | CELF2 |
| AIF1 | LBR | AL021807.1 | GMFG | PTGER2 |
| VIM | CASP1 | HCG4P11 | RP4- | HLA-DQB1 |
| | | | 671O14.5 | |
| ZNF367 | VNN1 | RP11-278C7.5 | HCST | SLC46A3 |
| SLAMF8 | HLA-H | CARD8-AS1 | RP1- | CTD-2033D15.1 |
| | | | 68D18.2 | |
| NCAPG | SMARCD3 | MMP19 | ARHGAP15 | FABP5P7 |
| HLA-DQB1- | HIST1H2BO | FCGR2A | ITGAM | MCM6 |
| AS1 | | | | |
| VCAN | CD37 | BTLA | Z98744.3 | NMI |
| RP11- | MARCH1 | RP11- | VNN2 | HLA-DOA |
| 1049A21.2 | | 290C10.1 | | |
| CD300E | RGS2 | HIST1H3J | NCAPH | HIST1H2BE |
| HLA-B | CD4 | MSNP1 | CTSC | CXCL5 |
| AC008984.2 | HIST1H2AE | APMAP | CD84 | SHC4 |
| GLIPR2 | ZPLD1 | GPR171 | FAM101B | C5AR1 |
| UBASH3B | FAM78A | SLED1 | CRTAM | PMP22 |
| ZEB2-AS1 | CTC-301O7.4 | PFN1P1 | EPSTI1 | HK3 |
| C18orf54 | ATP8A1 | C11orf21 | LCP1 | FAM198B |
| RP11-326C3.2 | ETV7 | WAS | RP11- | CYBB |
| | | | 415J8.3 | |
| CDCA7L | CMSS1 | RBL1 | FLI1 | RNF175 |
| IL27RA | CSF1R | TMX1 | FAR2 | HAUS1 |
| AOAH | AQP9 | ARHGEF6 | AC007278.2 | CLEC2B |
| CSGALNACT2 | LILRB1 | ITK | LINC01093 | P2RY14 |
| FPR1 | PLK4 | RP11- | MDM1 | PREX1 |
| | | 488L18.10 | | |
| RP11- | RP11-256L6.3 | RP6-159A1.4 | CD226 | CD300LB |
| 186N15.3 | | | | |
| SNX10 | CEP85L | CLEC10A | LTB4R | MYC |
| CENPH | FXYD4 | TRAF5 | CIITA | SOAT1 |
| RP11-325F22.2 | AGAP2 | NAIP | PLP2 | PSMB8-AS1 |
| ETS1 | IRF4 | NCF1 | E2F8 | CD33 |
| BTNL8 | CLEC4E | POLR3GL | IKZF3 | IL2RA |
| GFI1 | HHEX | SNX20 | APBB1IP | RP11-15A1.7 |
| DCK | SNORA12 | EMR1 | APOBEC3G | ANXA2R |
| ANKRD22 | AC025048.1 | MS4A7 | MSN | FABP5 |
| PYHIN1 | GPR141 | RP11-455F5.5 | HCG4P7 | GAB3 |
| PARVG | C17orf53 | RP11-297C4.2 | SLC4A7 | FLT3 |
| TNFAIP6 | HJURP | PATL2 | TLR4 | LAPTM5 |
| RP11- | RAB37 | MOB3C | GTF2H2C | NGFR |
| 876N24.3 | | | | |
| KIF14 | IGSF6 | TTC27 | PPP2R2B | LGALS1 |
| RP5-1171I10.5 | IKBIP | XXbac- | AC025171.1 | CTSW |
| | | BPG299F13.14 | | |
| SLC11A1 | HGF | CNTRL | UGT1A6 | CD53 |
| RP11-701P16.2 | DOCK11 | VIM-AS1 | RP11- | RP11-488L18.4 |
| | | | 272L13.4 | |
| EMP3 | CCL2 | EXO5 | PLA2G7 | GPSM3 |
| LINC01272 | STAT1 | GNG2 | CR1 | SNRPF |
| TNNI2 | HCLS1 | HIST1H3C | RP11- | HENMT1 |
| | | | 214O1.2 | |
| WIPF1 | TNFSF4 | DOCK10 | ACSL1 | IL10RA |
| HIST1H2AK | CD97 | ADAMTS2 | MAD2L1 | SASH3 |
| SLC16A1 | RN7SKP203 | RAC2 | CD36 | JAM3 |
| BUB1B | RRN3 | RAB20 | CTB-41I6.1 | RNU4-2 |
| CERKL | TSPAN2 | HLA-F | CFP | ARL6IP6 |
| TNFRSF25 | CPVL | CD6 | MYADM | RP11-149I23.3 |
| HLA-DRB5 | RFC3 | FBN1 | NUSAP1 | PLXNC1 |
| NLRC4 | CTD- | GPR68 | MAP7D3 | ARG1 |
| | 2047H16.3 | | | |
| F2R | PIK3CD-AS1 | GSG2 | HS3ST3B1 | IQGAP2 |
| ALOX5AP | AC007620.3 | IDO1 | BCL2A1 | CALHM2 |
| KIF11 | TTK | LTB | FCGR1A | EEF1E1 |
| AC006460.2 | RNASE2 | KIF15 | P2RY8 | LPAR6 |
| BASP1 | BMP7 | CALHM6 | CD14 | CD46 |
| CXCL14 | ENG | FN1 | FOXP3 | IFNGR1 |
| IL18BP | IL32 | INPP5D | IRAK2 | ISG20 |
| KLRC4- | MAP4K1 | NAMPT | PRPF31 | PRPF32 |
| KLRK1 | | | | |
| PRPF33 | PSMB10 | PYCARD | RUNX3 | SERPINA1 |
| TBP | TGFB1 | | | |
| ADD1 | AIFM3 | ANKH | ANXA1 | APP |
| ATF3 | AVPR1A | BAX | BCAP31 | BCL10 |
| BCL2L1 | BCL2L10 | BCL2L11 | BCL2L2 | BGN |
| BID | BIK | BIRC3 | BMF | BMP2 |
| BNIP3L | BRCA1 | BTG2 | BTG3 | CASP1 |
| CASP2 | CASP3 | CASP4 | CASP6 | CASP7 |
| CASP8 | CASP9 | CAV1 | CCNA1 | CCND1 |
| CCND2 | CD14 | CD2 | CD38 | CD44 |
| CD69 | CDC25B | CDK2 | CDKN1A | CDKN1B |
| CFLAR | CLU | CREBBP | CTH | CTNNB1 |
| CYLD | DAP | DAP3 | DCN | DDIT3 |
| DFFA | DIABLO | DNAJA1 | DNAJC3 | DNM1L |
| DPYD | EBP | EGR3 | EMP1 | ENO2 |
| ERBB2 | ERBB3 | EREG | ETF1 | F2 |
| F2R | FAS | FASLG | FDXR | FEZ1 |
| GADD45A | GADD45B | GCH1 | GNA15 | GPX1 |
| GPX3 | GPX4 | GSN | GSR | GSTM1 |
| GUCY2D | H1F0 | HGF | HMGB2 | HMOX1 |
| HSPB1 | IER3 | IFITM3 | IFNB1 | IFNGR1 |
| IGF2R | IGFBP6 | IL18 | IL1A | IL1B |
| IL6 | IRF1 | ISG20 | JUN | KRT18 |
| LEF1 | LGALS3 | LMNA | LPPR4 | LUM |
| MADD | MCL1 | MGMT | MMP2 | NEDD9 |
| NEFH | PAK1 | PDCD4 | PDGFRB | PEA15 |
| PLAT | PLCB2 | PMAIP1 | PPP2R5B | PPP3R1 |
| PPT1 | PRF1 | PSEN1 | PSEN2 | PTK2 |
| RARA | RELA | RETSAT | RHOB | RHOT2 |
| RNASEL | ROCK1 | SAT1 | SATB1 | SC5DL |
| SLC20A1 | SMAD7 | SOD1 | SOD2 | SPTAN1 |
| SQSTM1 | TAP1 | TGFB2 | TGFBR3 | TIMP1 |
| TIMP2 | TIMP3 | TNF | TNFRSF12A | TNFSF10 |
| TOP2A | TSPO | TXNIP | VDAC2 | WEE1 |
| XIAP | | | | |
| ALB | BASP1 | BMP7 | C11orf21 | C1QB |
| C3 | CALHM6 | CD14 | CD3D | CD3E |
| CD46 | CD6 | CD74 | CDH1 | CXCL10 |
| CXCL11 | CXCL14 | CXCL9 | ENG | FN1 |
| FOXP3 | GNLY | GZMB | HAVCR2 | IFNGR1 |
| IL18BP | IL2RA | IL32 | INPP5D | IRAK2 |
| ISG20 | KLRC4- | LCK | MAP4K1 | MRC1 |
| | KLRK1 | | | |
| NAMPT | NKG7 | PRF1 | PRPF31 | PRPF32 |
| PRPF33 | PSMB10 | PSMB9 | PYCARD | RGS1 |
| RUNX3 | SERPINA1 | SERPINB12 | STAT1 | TAP1 |
| TBP | TGFB1 | TMEM156 | | |
| RBM6 | PSMB1 | ITIH4 | TP53BP1 | DFNB31 |
| LGALS1 | AP4S1 | BRF2 | RAD23B | HNRNPA2B1 |
| ZNF436 | NECAB3 | HCST | AHDC1 | ARPP19 |
| C3orf20 | ACTR3B | UBAC2 | SETDB2 | STX17 |
| RAB30 | RPLP1 | SNRPF | CMTM3 | USP21 |
| LRIG1 | GTF2H2 | MPP7 | MERTK | ANGPT1 |
| AK9 | ITLN2 | CACHD1 | SYVN1 | ELF3 |
| UCN | ZMYM6 | BDH2 | DACT1 | COPS6 |
| 2-Sep | ZNF354A | BTD | SIGLEC17P | TADA3 |
| CTSF | LINC00469 | DMAP1 | DCTPP1 | MFSD6L |
| ADAMTSL5 | SUPT5H | S100A5 | ARMCX4 | ZNF502 |
| ZNF138 | ZNF841 | FICD | COL5A2 | RP11-15J10.1 |
| GNRHR2 | TRDC | AC016747.3 | AGGF1P1 | CHCHD3P3 |
| PRPF38AP1 | RP11- | AC010894.3 | RPL4P6 | AP000936.1 |
| | 242O24.3 | | | |
| LINC01278 | LINC00298 | AC159540.14 | LINC00299 | AMY2B |
| RPL34P18 | RP11- | RPP21 | RP11- | RP11-392P7.6 |
| | 1008M1.1 | | 20O24.4 | |
| CDK11B | RP1-16A9.1 | RP11-585P4.5 | P2RX5- | RP11-644F5.11 |
| | | | TAX1BP3 | |
| SPESP1 | RP11- | RP11-412D9.4 | RPL23AP97 | RP11-529K1.2 |
| | 603B24.1 | | | |
| RP5- | RN7SL246P | PARD6G-AS1 | RP11- | RP11-840I19.5 |
| 1107A17.4 | | | 635N19.1 | |
| RP11-179B2.2 | RP11- | LA16c- | F8A1 | uc_338 |
| | 313P22.1 | 360A4.1 | | |
| RP11-43N22.1 | RP11- | | | |
| | 133M8.3 | | | |

, and combinations thereof.

In some embodiments, the one or more miRNAs are binding one or more expression products from a gene selected from the group consisting of:

| | | | | |
|---|---|---|---|---|
| ITM2A | SLAMF6 | IKZF3 | CCL5 | CXCL9 |
| IGHM | ITM2A | HIST1H2AJ | CXCL10 | CD3E |
| NKG7 | TRBC2 | HIST1H3I | TRGC2 | HIST1H1B |
| CTLA4 | PDCD1 | GZMA | IL2RB | IKZF3 |
| CD69 | SIT1 | CD96 | FAIM3 | ZAP70 |
| HIST1H1D | TRAC | RNASE6 | SLAMF6 | Z98744.2 |
| GZMB | IL18R1 | IL1RL1 | HIST1H4L | C1QB |
| CD8A | HIST1H2BM | HLA-DQB1 | LCK | ASB2 |
| HIST1H2AI | SLFN12L | CD28 | TRBV28 | RP4-620F22.2 |
| GIMAP1 | ICOS | ZNF831 | TRAT1 | GIMAP7 |
| P2RY10 | HCST | HIST1H2AK | GBP5 | EMB |
| CD2 | KLRC1 | CD6 | GPR174 | AL049822.1 |
| HLA-DQA1 | TIGIT | ITK | ST8SIA4 | GIMAP6 |
| GVINP1 | HLA-DQB1- | MRC1 | HLA-DPB1 | CCR2 |
| | AS1 | | | |
| CCND2-AS1 | AL009179.1 | EVI2B | FCGR3B | ETV7 |
| CTSW | HIST1H2AL | THEMIS | CH17- | BLM |
| | | | 373J23.1 | |
| PRF1 | DOK2 | CXCR6 | NGFR | KCNE1 |
| HIST1H3H | CTD- | HLA-DRA | CD8B | NCF1 |
| | 2313F11.1 | | | |
| GZMK | MLKL | HIST1H3B | CD3G | JAK3 |
| CD3D | C1QA | IFNG | CDCA7 | Z98744.3 |
| CXCL11 | RP11-284N8.3 | HLA-H | TOX | GIMAP4 |
| FAM78A | FAM65B | HCG4P7 | SMAP2 | EOMES |
| RASAL3 | CD163 | FCRL3 | IFIT2 | SH2D1A |
| HCG4P11 | RGS18 | MIR155HG | IFITM1 | HIST1H2BL |
| ITGA4 | RP11- | MATK | KCNJ2 | KLRB1 |
| | 463J10.3 | | | |
| HIST1H2AH | APOBEC3G | PSMB9 | PTPRC | RCSD1 |
| PYHIN1 | BTN3A3 | AC007278.2 | C11orf21 | RHOH |
| TMEM156 | HIST2H3D | NLRP3 | AF001548.5 | HIST1H2BI |
| CD74 | AGAP2 | CD52 | GBP1 | GMFG |
| LAX1 | SASH3 | GNLY | HIST2H2AB | MS4A6A |
| PDE3B | SLAMF8 | AGER | PREX1 | IRF8 |
| IKZF1 | TBC1D10C | FCGR3A | BCL11B | HLA-DRB1 |
| CD97 | CTB-186G2.1 | NLRC3 | TAP1 | CD247 |
| HIST1H3G | GBP4 | RP11- | TMC8 | SELPLG |
| | | 1049A21.2 | | |
| HIST1H2BO | CCND2 | CST7 | CCR5 | RP11-264B17.5 |
| GRAP2 | HLA-B | IKBIP | KRT1 | SERPINB12 |
| ALB | TGM3 | HS3ST6 | NRTN | FAM180A |
| FALEC | FGF22 | HCN2 | SLC24A3 | RP11-167H9.5 |
| C1QB | IL18R1 | KCNE1 | CCR2 | C1QA |
| CD163 | CST7 | SLC1A3 | RNASE6 | HIST1H1B |
| TRGC2 | MS4A6A | C1QC | RGS1 | MSR1 |
| HIST1H1D | HIST1H3I | CD28 | LSM11 | HIST1H3B |
| HIST1H2AJ | GIMAP6 | DAAM2 | CCL5 | CTLA4 |
| IGHG1 | C1orf162 | MIR155HG | ITM2A | GIMAP4 |
| HIST1H2BM | HIST1H3G | FCER1G | FKBP5 | IL1R2 |
| GZMA | CLEC12A | LINC01127 | Z98744.2 | GPR34 |
| HLA-DMB | HIST1H4L | RRM2 | TREM2 | GIMAP8 |
| CD3E | AL049822.1 | HLA-DPB1 | LMNB 1 | CD300C |
| IFITM1 | BLM | SAMSN1 | IRF8 | CLSPN |
| IL1RL1 | SDS | RP11-274E7.2 | MS4A4A | PDCD1 |
| ICOS | RP11- | TNFSF13B | HIST1H3F | RP11-777B9.5 |
| | 848G14.5 | | | |
| GIMAP2 | IL18RAP | VSIG4 | GIMAP7 | SRGN |
| TRBC2 | LIPA | SERPINE1 | CENPK | IL10 |
| GZMK | DTL | GLDN | SMAP2 | HIST1H2BB |
| SIT1 | IFITM3 | CTD- | HLA-DRA | THBS1 |
| | | 2033D15.2 | | |
| ADORA3 | STAB1 | AIM2 | EVI2A | TFPI2 |
| EMB | SLA | CD8A | RCSD1 | GIMAP1 |
| FAM105A | FPR2 | CD180 | RP11- | RP4-737E23.5 |
| | | | 463J10.3 | |
| FCGR3A | HIST1H2BL | CD1D | UBE2C | PDK4 |
| MLKL | RP11-7F17.3 | TLR7 | PTPRC | KBTBD8 |
| CD96 | HIST1H2AI | FAIM3 | CD48 | IFI27L2 |
| HIST1H4F | CTD- | SLC16A6 | LRRC8C | NLRP6 |
| | 2033D15.3 | | | |
| TLR8 | IRAK3 | LY96 | LILRA5 | AL009179.1 |
| KIAA0101 | GCA | CCDC109B | CTSL | CCR5 |
| BTN3A2 | STAMBPL1 | HLA-DPA1 | CTD- | LAIR1 |
| | | | 2313F11.1 | |
| FES | RNASE1 | CXCL10 | TRIM22 | HIST1H3H |
| CD52 | MYBL2 | RP11- | RNU4-1 | KLRC1 |
| | | 403P17.6 | | |
| FEN1 | MIR181A1HG | HCG27 | RP11- | BIN2 |
| | | | 335I12.2 | |
| RP4-620F22.2 | GBP5 | FYB | PCED1B | AL031777.1 |
| MMD | DOK2 | RP11- | PPT1 | FAM65B |
| | | 404F10.2 | | |
| GVINP1 | SERPINF1 | CARD16 | CEP55 | HIST1H2AL |
| CD86 | CD2 | JAK3 | SELPLG | AC007278.3 |
| FAM111B | GYG1 | NLRP3 | ZWINT | ITGA4 |
| TRAC | SELL | GPR183 | TRG-AS1 | ST8SIA4 |
| NCKAP1L | STK17B | OLAH | APOC1 | PCED1B-AS1 |
| SLFN13 | TIGIT | ATHL1 | HIST1H2BI | HLA-DMA |
| ENTPD1 | SLCO2B1 | IFNG | CD300LF | F13A1 |
| NEIL3 | TMEM14E | ACSL4 | RN7SKP48 | GZMH |
| LYAR | GPNMB | IKZF1 | RP3-455J7.4 | SCIMP |
| AMIGO2 | TLR5 | EXOC6 | IFITM2 | HAVCR2 |
| TLR2 | CTB- | FGL2 | A2M | LY9 |
| | 111H14.1 | | | |
| KIAA0825 | WDR76 | LILRB5 | SLAMF6 | CTB-41I6.2 |
| FGR | HELLS | CD274 | HIST1H2AB | MELK |
| ASB2 | SIGLEC9 | TPK1 | MAP2K6 | RP11-347P5.1 |
| HIST1H2AH | EVI2B | CTA-373H7.7 | FAM26F | KLRB1 |
| TRPV2 | CHIT1 | HTATSF1P2 | TSC22D3 | TLDC2 |
| MGAM | RP11-69L16.4 | CXCR6 | STAT4 | TNFSF8 |
| MB21D1 | SLFN11 | RP11- | TYROBP | ITGB2 |
| | | 124N14.3 | | |
| LILRB4 | CDK1 | PSMB8 | HIST2H2AB | DNA2 |
| PDCD1LG2 | CCL18 | RP11-25K21.6 | HLA-DQA1 | CELF2 |
| AIF1 | LBR | AL021807.1 | GMFG | PTGER2 |
| VIM | CASP1 | HCG4P11 | RP4- | HLA-DQB1 |
| | | | 671O14.5 | |
| ZNF367 | VNN1 | RP11-278C7.5 | HCST | SLC46A3 |
| SLAMF8 | HLA-H | CARD8-AS1 | RP1- | CTD-2033D15.1 |
| | | | 68D18.2 | |
| NCAPG | SMARCD3 | MMP19 | ARHGAP15 | FABP5P7 |
| HLA-DQB1- | HIST1H2BO | FCGR2A | ITGAM | MCM6 |
| AS1 | | | | |
| VCAN | CD37 | BTLA | Z98744.3 | NMI |
| RP11- | MARCH1 | RP11- | VNN2 | HLA-DOA |
| 1049A21.2 | | 290C10.1 | | |
| CD300E | RGS2 | HIST1H3J | NCAPH | HIST1H2BE |
| HLA-B | CD4 | MSNP1 | CTSC | CXCL5 |
| AC008984.2 | HIST1H2AE | APMAP | CD84 | SHC4 |
| GLIPR2 | ZPLD1 | GPR171 | FAM101B | C5AR1 |
| UBASH3B | FAM78A | SLED1 | CRTAM | PMP22 |
| ZEB2-AS1 | CTC-301O7.4 | PFN1P1 | EPSTI1 | HK3 |
| C18orf54 | ATP8A1 | C11orf21 | LCP1 | FAM198B |
| RP11-326C3.2 | ETV7 | WAS | RP11- | CYBB |
| | | | 415J8.3 | |
| CDCA7L | CMSS1 | RBL1 | FLI1 | RNF175 |
| IL27RA | CSF1R | TMX1 | FAR2 | HAUS1 |
| AOAH | AQP9 | ARHGEF6 | AC007278.2 | CLEC2B |
| CSGALNACT2 | LILRB 1 | ITK | LINC01093 | P2RY14 |
| FPR1 | PLK4 | RP11- | MDM1 | PREX1 |
| | | 488L18.10 | | |
| RP11- | RP11-256L6.3 | RP6-159A1.4 | CD226 | CD300LB |
| 186N15.3 | | | | |
| SNX10 | CEP85L | CLEC10A | LTB4R | MYC |
| CENPH | FXYD4 | TRAF5 | CIITA | SOAT1 |
| RP11-325F22.2 | AGAP2 | NAIP | PLP2 | PSMB8-AS1 |
| ETS1 | IRF4 | NCF1 | E2F8 | CD33 |
| BTNL8 | CLEC4E | POLR3GL | IKZF3 | IL2RA |
| GFI1 | HHEX | SNX20 | APBB1IP | RP11-15A1.7 |
| DCK | SNORA12 | EMR1 | APOBEC3G | ANXA2R |
| ANKRD22 | AC025048.1 | MS4A7 | MSN | FABP5 |
| PYHIN1 | GPR141 | RP11-455F5.5 | HCG4P7 | GAB3 |
| PARVG | C17orf53 | RP11-297C4.2 | SLC4A7 | FLT3 |
| TNFAIP6 | HJURP | PATL2 | TLR4 | LAPTM5 |
| RP11- | RAB37 | MOB3C | GTF2H2C | NGFR |
| 876N24.3 | | | | |
| KIF14 | IGSF6 | TTC27 | PPP2R2B | LGALS1 |
| RP5-1171110.5 | IKBIP | XXbac- | AC025171.1 | CTSW |
| | | BPG299F13.14 | | |
| SLC11A1 | HGF | CNTRL | UGT1A6 | CD53 |
| RP11-701P16.2 | DOCK11 | VIM-AS1 | RP11- | RP11-488L18.4 |
| | | | 272L13.4 | |
| EMP3 | CCL2 | EXO5 | PLA2G7 | GPSM3 |
| LINC01272 | STAT1 | GNG2 | CR1 | SNRPF |
| TNNI2 | HCLS1 | HIST1H3C | RP11- | HENMT1 |
| | | | 214O1.2 | |
| WIPF1 | TNFSF4 | DOCK10 | ACSL1 | IL10RA |
| HIST1H2AK | CD97 | ADAMTS2 | MAD2L1 | SASH3 |
| SLC16A1 | RN7SKP203 | RAC2 | CD36 | JAM3 |
| BUB1B | RRN3 | RAB20 | CTB-41I6.1 | RNU4-2 |
| CERKL | TSPAN2 | HLA-F | CFP | ARL6IP6 |
| TNFRSF25 | CPVL | CD6 | MYADM | RP11-149I23.3 |
| HLA-DRB5 | RFC3 | FBN1 | NUSAP1 | PLXNC1 |
| NLRC4 | CTD- | GPR68 | MAP7D3 | ARG1 |
| | 2047H16.3 | | | |
| F2R | PIK3CD-AS1 | GSG2 | HS3ST3B1 | IQGAP2 |
| ALOX5AP | AC007620.3 | IDO1 | BCL2A1 | CALHM2 |
| KIF11 | TTK | LTB | FCGR1A | EEF1E1 |
| AC006460.2 | RNASE2 | KIF15 | P2RY8 | LPAR6 |
| BASP1 | BMP7 | CALHM6 | CD14 | CD46 |
| CXCL14 | ENG | FN1 | FOXP3 | IFNGR1 |
| IL18BP | IL32 | INPP5D | IRAK2 | ISG20 |
| KLRC4- | MAP4K1 | NAMPT | PRPF31 | PRPF32 |
| KLRK1 | | | | |
| PRPF33 | PSMB10 | PYCARD | RUNX3 | SERPINA1 |
| TBP | TGFB1 | | | |
| ADD1 | AIFM3 | ANKH | ANXA1 | APP |
| ATF3 | AVPRIA | BAX | BCAP31 | BCL10 |
| BCL2L1 | BCL2L10 | BCL2L11 | BCL2L2 | BGN |
| BID | BIK | BIRC3 | BMF | BMP2 |
| BNIP3L | BRCA1 | BTG2 | BTG3 | CASP1 |
| CASP2 | CASP3 | CASP4 | CASP6 | CASP7 |
| CASP8 | CASP9 | CAV1 | CCNA1 | CCND1 |
| CCND2 | CD14 | CD2 | CD38 | CD44 |
| CD69 | CDC25B | CDK2 | CDKN1A | CDKN1B |
| CFLAR | CLU | CREBBP | CTH | CTNNB1 |
| CYLD | DAP | DAP3 | DCN | DDIT3 |
| DFFA | DIABLO | DNAJA1 | DNAJC3 | DNM1L |
| DPYD | EBP | EGR3 | EMP1 | ENO2 |
| ERBB2 | ERBB3 | EREG | ETF1 | F2 |
| F2R | FAS | FASLG | FDXR | FEZ1 |
| GADD45A | GADD45B | GCH1 | GNA15 | GPX1 |
| GPX3 | GPX4 | GSN | GSR | GSTM1 |
| GUCY2D | H1F0 | HGF | HMGB2 | HMOX1 |
| HSPB1 | IER3 | IFITM3 | IFNB1 | IFNGR1 |
| IGF2R | IGFBP6 | IL18 | IL1A | IL1B |
| IL6 | IRF1 | ISG20 | JUN | KRT18 |
| LEF1 | LGALS3 | LMNA | LPPR4 | LUM |
| MADD | MCL1 | MGMT | MMP2 | NEDD9 |
| NEFH | PAK1 | PDCD4 | PDGFRB | PEA15 |
| PLAT | PLCB2 | PMAIP1 | PPP2R5B | PPP3R1 |
| PPT1 | PRF1 | PSEN1 | PSEN2 | PTK2 |
| RARA | RELA | RETSAT | RHOB | RHOT2 |
| RNASEL | ROCK1 | SAT1 | SATB1 | SC5DL |
| SLC20A1 | SMAD7 | SOD1 | SOD2 | SPTAN1 |
| SQSTM1 | TAP1 | TGFB2 | TGFBR3 | TIMP1 |
| TIMP2 | TIMP3 | TNF | TNFRSF12A | TNFSF10 |
| TOP2A | TSPO | TXNIP | VDAC2 | WEE1 |
| XIAP | | | | |
| ALB | BASP1 | BMP7 | C11orf21 | C1QB |
| C3 | CALHM6 | CD14 | CD3D | CD3E |
| CD46 | CD6 | CD74 | CDH1 | CXCL10 |
| CXCL11 | CXCL14 | CXCL9 | ENG | FN1 |
| FOXP3 | GNLY | GZMB | HAVCR2 | IFNGR1 |
| IL18BP | IL2RA | IL32 | INPP5D | IRAK2 |
| ISG20 | KLRC4- | LCK | MAP4K1 | MRC1 |
| | KLRK1 | | | |
| NAMPT | NKG7 | PRF1 | PRPF31 | PRPF32 |
| PRPF33 | PSMB10 | PSMB9 | PYCARD | RGS1 |
| RUNX3 | SERPINA1 | SERPINB12 | STAT1 | TAP1 |
| TBP | TGFB1 | TMEM156 | | |
| RBM6 | PSMB1 | ITIH4 | TP53BP1 | DFNB31 |
| LGALS1 | AP4S1 | BRF2 | RAD23B | HNRNPA2B1 |
| ZNF436 | NECAB3 | HCST | AHDC1 | ARPP19 |
| C3orf20 | ACTR3B | UBAC2 | SETDB2 | STX17 |
| RAB30 | RPLP1 | SNRPF | CMTM3 | USP21 |
| LRIG1 | GTF2H2 | MPP7 | MERTK | ANGPT1 |
| AK9 | ITLN2 | CACHD1 | SYVN1 | ELF3 |
| UCN | ZMYM6 | BDH2 | DACT1 | COPS6 |
| 2-Sep | ZNF354A | BTD | SIGLEC17P | TADA3 |
| CTSF | LINC00469 | DMAP1 | DCTPP1 | MFSD6L |
| ADAMTSL5 | SUPT5H | S100A5 | ARMCX4 | ZNF502 |
| ZNF138 | ZNF841 | FICD | COL5A2 | RP11-15J10.1 |
| GNRHR2 | TRDC | AC016747.3 | AGGF1P1 | CHCHD3P3 |
| PRPF38AP1 | RP11- | AC010894.3 | RPL4P6 | AP000936.1 |
| | 242O24.3 | | | |
| LINC01278 | LINC00298 | AC159540.14 | LINC00299 | AMY2B |
| RPL34P18 | RP11- | RPP21 | RP11- | RP11-392P7.6 |
| | 1008M1.1 | | 20O24.4 | |
| CDK11B | RP1-16A9.1 | RP11-585P4.5 | P2RX5- | RP11-644F5.11 |
| | | | TAX1BP3 | |
| SPESP1 | RP11- | RP11-412D9.4 | RPL23AP97 | RP11-529K1.2 |
| | 603B24.1 | | | |
| RP5- | RN7SL246P | PARD6G-AS1 | RP11- | RP11-840I19.5 |
| 1107A17.4 | | | 635N19.1 | |
| RP11-179B2.2 | RP11- | LA16c- | F8A1 | uc_338 |
| | 313P22.1 | 360A4.1 | | |
| RP11-43N22.1 | RP11- | | | |
| | 133M8.3 | | | |

, and combinations thereof.

In some embodiments, the one or more mRNAs are expressed from a gene selected from the group consisting of:

| | | | | |
|---|---|---|---|---|
| ALB | BASP1 | BMP7 | C11orf21 | C1QB |
| C3 | CALHM6 | CD14 | CD3D | CD3E |
| CD46 | CD6 | CD74 | CDH1 | CXCL10 |
| CXCL11 | CXCL14 | CXCL9 | ENG | FN1 |
| FOXP3 | GNLY | GZMB | HAVCR2 | IFNGR1 |
| IL18BP | IL2RA | IL32 | INPP5D | IRAK2 |
| ISG20 | KLRC4-KLRK1 | LCK | MAP4K1 | MRC1 |
| NAMPT | NKG7 | PRF1 | PRPF31 | PRPF32 |
| PRPF33 | PSMB10 | PSMB9 | PYCARD | RGS1 |
| RUNX3 | SERPINA1 | SERPINB12 | STAT1 | TAP1 |
| TBP | TGFB1 | TMEM156 | | |

, and combinations thereof.

In some embodiments, the one or more miRNAs are binding one or more mRNAs expressed from a gene selected from the group consisting of:

| | | | | |
|---|---|---|---|---|
| ALB | BASP1 | BMP7 | C11orf21 | C1QB |
| C3 | CALHM6 | CD14 | CD3D | CD3E |
| CD46 | CD6 | CD74 | CDH1 | CXCL10 |
| CXCL11 | CXCL14 | CXCL9 | ENG | FN1 |
| FOXP3 | GNLY | GZMB | HAVCR2 | IFNGR1 |
| IL18BP | IL2RA | IL32 | INPP5D | IRAK2 |
| ISG20 | KLRC4-KLRK1 | LCK | MAP4K1 | MRC1 |
| NAMPT | NKG7 | PRF1 | PRPF31 | PRPF32 |
| PRPF33 | PSMB10 | PSMB9 | PYCARD | RGS1 |
| RUNX3 | SERPINA1 | SERPINB12 | STAT1 | TAP1 |
| TBP | TGFB1 | TMEM156 | | |

, and combinations thereof.

In some embodiments, the one or more mRNAs are expressed from a gene selected from the group consisting of:

| | | | | | | | | | |
|---|---|---|---|---|---|---|---|---|---|
| ADD1 | AIFM3 | ANKH | ANXA1 | APP | ATF3 | AVPR1A | BAX | BCAP31 | BCL10 |
| BCL2L1 | BCL2L10 | BCL2L11 | BCL2L2 | BGN | BID | BIK | BIRC3 | BMF | BMP2 |
| BNIP3L | BRCA1 | BTG2 | BTG3 | CASP1 | CASP2 | CASP3 | CASP4 | CASP6 | CASP7 |
| CASP8 | CASP9 | CAV1 | CCNA1 | CCND1 | CCND2 | CD14 | CD2 | CD38 | CD44 |
| CD69 | CDC25B | CDK2 | CDKN1A | CDKN1B | CFLAR | CLU | CREBBP | CTH | CTNNB1 |
| CYLD | DAP | DAP3 | DCN | DDIT3 | DFFA | DIABLO | DNAJA1 | DNAJC3 | DNM1L |
| DPYD | EBP | EGR3 | EMP1 | ENO2 | ERBB2 | ERBB3 | EREG | ETF1 | F2 |
| F2R | FAS | FASLG | FDXR | FEZ1 | GADD45A | GADD45B | GCH1 | GNA15 | GPX1 |
| GPX3 | GPX4 | GSN | GSR | GSTM1 | GUCY2D | H1F0 | HGF | HMGB2 | HMOX1 |
| HSPB1 | IER3 | IFITM3 | IFNB1 | IFNGR1 | IGF2R | IGFBP6 | IL18 | IL1A | IL1B |
| IL6 | IRF1 | ISG20 | JUN | KRT18 | LEF1 | LGALS3 | LMNA | LPPR4 | LUM |
| MADD | MCL1 | MGMT | MMP2 | NEDD9 | NEFH | PAK1 | PDCD4 | PDGFRB | PEA15 |
| PLAT | PLCB2 | PMAIP1 | PPP2R5B | PPP3R1 | PPT1 | PRF1 | PSEN1 | PSEN2 | PTK2 |
| RARA | RELA | RETSAT | RHOB | RHOT2 | RNASEL | ROCK1 | SAT1 | SATB1 | SC5DL |
| SLC20A1 | SMAD7 | SOD1 | SOD2 | SPTAN1 | SQSTM1 | TAP1 | TGFB2 | TGFBR3 | TIMP1 |
| TIMP2 | TIMP3 | TNF | TNFRSF12A | TNFSF10 | TOP2A | TSPO | TXNIP | VDAC2 | WEE1 |
| XIAP | | | | | | | | | |

, and combinations thereof.

In some embodiments, the one or more miRNAs are binding one or more mRNAs expressed from a gene selected from the group consisting of:

| | | | | | | | | | |
|---|---|---|---|---|---|---|---|---|---|
| ADD1 | AIFM3 | ANKH | ANXA1 | APP | ATF3 | AVPR1A | BAX | BCAP31 | BCL10 |
| BCL2L1 | BCL2L10 | BCL2L11 | BCL2L2 | BGN | BID | BIK | BIRC3 | BMF | BMP2 |
| BNIP3L | BRCA1 | BTG2 | BTG3 | CASP1 | CASP2 | CASP3 | CASP4 | CASP6 | CASP7 |
| CASP8 | CASP9 | CAV1 | CCNA1 | CCND1 | CCND2 | CD14 | CD2 | CD38 | CD44 |
| CD69 | CDC25B | CDK2 | CDKN1A | CDKN1B | CFLAR | CLU | CREBBP | CTH | CTNNB1 |
| CYLD | DAP | DAP3 | DCN | DDIT3 | DFFA | DIABLO | DNAJA1 | DNAJC3 | DNM1L |
| DPYD | EBP | EGR3 | EMP1 | ENO2 | ERBB2 | ERBB3 | EREG | ETF1 | F2 |
| F2R | FAS | FASLG | FDXR | FEZ1 | GADD45A | GADD45B | GCH1 | GNA15 | GPX1 |
| GPX3 | GPX4 | GSN | GSR | GSTM1 | GUCY2D | H1F0 | HGF | HMGB2 | HMOX1 |
| HSPB1 | IER3 | IFITM3 | IFNB1 | IFNGR1 | IGF2R | IGFBP6 | IL18 | IL1A | IL1B |
| IL6 | IRF1 | ISG20 | JUN | KRT18 | LEF1 | LGALS3 | LMNA | LPPR4 | LUM |
| MADD | MCL1 | MGMT | MMP2 | NEDD9 | NEFH | PAK1 | PDCD4 | PDGFRB | PEA15 |
| PLAT | PLCB2 | PMAIP1 | PPP2R5B | PPP3R1 | PPT1 | PRF1 | PSEN1 | PSEN2 | PTK2 |
| RARA | RELA | RETSAT | RHOB | RHOT2 | RNASEL | ROCK1 | SAT1 | SATB1 | SC5DL |
| SLC20A1 | SMAD7 | SOD1 | SOD2 | SPTAN1 | SQSTM1 | TAP1 | TGFB2 | TGFBR3 | TIMP1 |
| TIMP2 | TIMP3 | TNF | TNFRSF12A | TNFSF10 | TOP2A | TSPO | TXNIP | VDAC2 | WEE1 |
| XIAP | | | | | | | | | |

, and combinations thereof.

In some embodiments, the one or more mRNAs are expressed from a gene selected from the group consisting of: CXCL9, CD3ϵ, IP-10, LCK, C1QB, PSMB9, Tim-3, Foxp3, FAM26F, ALB, CTB-186G2.1, CXCL11, GNLY, MRC1, RGS1, RP11-1049A21.2, TMEM156, Z98744.3, SERPINB12, C11orf21, AF001548, 18s ribosomal, Basp1, Cd6, Cxcl10, Cxcl9, Inpp5d, Isg20, Lck, Nkg7, Runx3, Tap1, GZMB, PRF1, CD74, IL32, STAT1, CXCL14, SERPINA1, B2M, C3, PYCARD, BMP7, TBP, NAMPT, IFNGR1, IRAK2, IL18BP, and combinations thereof.

In some embodiments, the one or more mRNAs are expressed from a gene selected from the group consisting of: CXCL9, CD3ϵ, IP-10, LCK, C1QB, PSMB9, Tim-3, Foxp3, FAM26F, ALB, CTB-186G2.1, CXCL11, GNLY, MRC1, RGS1, RP11-1049A21.2, TMEM156, Z98744.3, SERPINB12, C11orf21, AF001548, 18s ribosomal, Basp1, Cd6, Cxcl10, Cxcl9, Inpp5d, Isg20, Lck, Nkg7, Runx3, Tap1, GZMB, PRF1, CD74, IL32, STAT1, CXCL14, SERPINA1, B2M, C3, PYCARD, BMP7, TBP, NAMPT, IFNGR1, IRAK2, Calhm6, Klrc4-Klrk1, Psmb10, CD3delta, Map4k1, IL2Ra, TGFB1, FN1, CDH1, PRPF31, HAVCR2, IL18BP, and combinations thereof.

In some embodiments, the one or more mRNAs are expressed from a gene selected from the group consisting of: CXCL9, CD3ϵ, IP-10, LCK, C1QB, PSMB9, Tim-3, Foxp3, FAM26F, ALB, CTB-186G2.1, CXCL11, GNLY, MRC1, RGS1, RP11-1049A21.2, TMEM156, Z98744.3, SERPINB12, C11orf21, AF001548, 18s ribosomal, Basp1, Cd6, Cxcl10, Cxcl9, Inpp5d, Isg20, Lck, Nkg7, Runx3, Tap1, and combinations thereof.

In some embodiments, the one or more mRNAs are expressed from a gene selected from the group consisting of: CXCL11, CXCL9, GNLY, PSMB9, TAP1, CXCL10, NKG7, ISG20, RUNX3, C11orf21, C1QB, LCK, INPP5D, CD6, CD3ε, MRC1, BASP1, TMEM156, ALB, FOXP3, RGS1, SERPINB12, and combinations thereof.

In some embodiments, the one or more mRNAs are expressed from a gene selected from the group consisting of: CXCL11, CXCL9, GNLY, PSMB9, TAP1, CXCL10, NKG7, ISG20, RUNX3, C11orf21, C1QB, LCK, INPP5D, CD6, CD3ε, MRC1, BASP1, TMEM156, ALB, FOXP3, RGS1, SERPINB12, PRF1, CALHM6, KLRC4-KLRK1, CD74, GZMB, PSMB10, B2M, STAT1, CD3delta, PYCARD, IL18BP, MAP4K1, IL32, IL2RA, C3, IFNGR1, IRAK2, TGFB1, FN1, NAMPT, SERPINA1, TBP, CDH1, PRPF31, BMP7, CXCL14, HAVCR2, and combinations thereof.

In some embodiments, wherein the one or more mRNAs are not expressed from a gene selected from the group consisting of: PDCD1, MARCHF8, DCAF12, IL1R2, FLT3, ITGA4, ITGAM, PF4, C6orf25, SEMA7A, RHOU, and combinations thereof.

In some embodiments, the one or more mRNAs are not expressed from a gene selected from the group consisting of: CCDC159, dcaf12, DECR1, ERCC5, EWSR1, FLT3, GABPB2, GPI, IL1R2, ITGA4, ITGAM, MAP3K3, MAPK9, MARCHF8, NONO, PDCD1, PF4, RHOU, SEMA7A, SRRM1, TBC1D10B, TOP2B, and combinations thereof.

In some embodiments, the one or more miRNAs are selected from the group consisting of miR-186-5p, miR-665, miR-873-5p.1, miR-543, miR-330-3p, miR-362-5p/500b-5p, miR-217, miR-140-5p, miR-193-3p, miR-382-5p, miR-140-3p.2, miR-653-5p, miR-455-3p.2, miR-145-5p, miR-491-5p, miR-23-3p, miR-375, miR-129-5p, miR-96-5p/1271-5p, miR-182-5p, miR-371-5p, miR-203a-3p.1, miR-494-3p, miR-146-5p, miR-140-3p.1, miR-125-5p, miR-346, miR-760, miR-185-5p, miR-325-3p, miR-15-5p/16-5p/195-5p/424-5p/497-5p, miR-503-5p, miR-423-5p, miR-496.1, miR-155-5p, miR-142-3p.2, miR-24-3p, miR-874-3p, miR-25-3p/32-5p/92-3p/363-3p/367-3p, miR-17-5p/20-5p/93-5p/106-5p/519-3p, miR-181-5p, miR-142-5p, miR-130-3p/301-3p/454-3p, miR-21-5p/590-5p, miR-103-3p/107, miR-137, miR-340-5p, miR-490-3p, miR-143-3p, miR-409-3p, miR-27-3p, miR-138-5p, miR-485-5p, miR-328-3p, miR-326, miR-148-3p/152-3p, miR-9-5p, miR-31-5p, miR-452-5p/892-3p, miR-202-5p, miR-29-3p, miR-338-3p, miR-26-5p, let-7-5p/98-5p, miR-196-5p, miR-30-5p, miR-142-3p.1, miR-19-3p, miR-411-3p, miR-493-5p, miR-218-5p, miR-203a-3p.2, miR-495-3p, miR-425-5p, miR-135-5p, miR-154-3p/487-3p, miR-223-3p, miR-219-5p, miR-670-3p, miR-216b-5p, miR-200bc-3p/429, miR-320, miR-216a-5p, miR-141-3p/200a-3p, miR-144-3p, miR-128-3p, miR-455-3p.1, miR-219a-2-3p, miR-873-5p.2, miR-448, miR-183-5p.2, miR-374-5p, miR-505-3p.1, miR-433-3p, miR-377-3p, miR-365-3p, miR-124-3p.1, miR-410-3p, miR-199-3p, miR-22-3p, miR-129-3p, miR-383-5p.1, miR-1-3p/206, miR-296-5p, miR-299-3p, miR-212-5p, miR-331-3p, miR-378-3p, miR-136-5p, miR-1193, miR-505-3p.2, miR-302c-3p.2/520-3p, miR-421, miR-499a-5p, miR-302-3p/372-3p/373-3p/520-3p, miR-124-3p.2/506-3p, miR-34-5p/449-5p, miR-376c-3p, miR-139-5p, miR-221-3p/222-3p, miR-504-5p.1, miR-335-5p, miR-101-3p.1, miR-431-5p, miR-489-3p, miR-369-3p, miR-330-3p.2, miR-18-5p, miR-28-5p/708-5p, miR-133a-3p.2/133b, miR-205-5p, miR-199-5p, miR-455-5p, miR-126-3p.2, miR-7-5p, miR-483-3p.2, miR-668-3p, miR-1306-5p, miR-150-5p, miR-296-3p, miR-204-5p/211-5p, miR-3064-5p, miR-532-5p, miR-876-5p, miR-501-3p/502-3p, miR-33-5p, miR-153-3p, miR-214-5p, miR-655-3p, miR-342-3p, miR-133a-3p.1, miR-411-5p.1, miR-496.2, miR-411-5p.2, miR-582-5p, miR-381-3p, miR-188-5p, miR-383-5p.2, miR-486-5p, miR-183-5p.1, miR-208-3p, miR-193a-5p, miR-101-3p.2, miR-542-3p, miR-190-5p, miR-299-5p, miR-154-5p, miR-802, miR-323-3p, miR-532-3p, miR-224-5p, miR-339-5p, miR-194-5p, miR-149-5p, miR-493-3p, miR-382-3p, miR-132-3p/212-3p, miR-1197, miR-99-5p/100-5p, miR-877-5p, miR-483-3p.1, miR-10-5p, miR-361-5p, miR-539-3p, miR-191-5p, miR-329-3p/362-3p, miR-122-5p, miR-379-5p, miR-376-3p, miR-1298-5p, miR-451, miR-210-3p, miR-1224-5p, miR-324-5p, miR-544a-5p, miR-488-3p, miR-758-3p, miR-151-3p, miR-875-5p, miR-134-5p, miR-192-5p/215-5p, and miR-127-3p.

In some embodiments, the one or more miRNAs are selected from the group consisting of miR-96-5p/1271-5p, miR-493-5p, miR-183-5p.2, miR-150-5p, miR-7-5p, miR-653-5p, miR-200bc-3p/429, miR-212-5p, miR-1298-5p, miR-137, miR-758-3p, miR-325-3p, miR-542-3p, miR-25-3p/32-5p/92-3p/363-3p/367-3p, miR-495-3p, miR-30-5p, miR-873-5p.1, miR-146-5p, miR-505-3p.1, miR-539-3p, miR-216a-5p, miR-216b-5p, miR-340-5p, miR-361-5p, miR-338-3p, miR-217, miR-9-5p, miR-219a-2-3p, miR-15-5p/16-5p/195-5p/424-5p/497-5p, miR-503-5p, miR-199-3p, miR-1-3p/206, miR-17-5p/20-5p/93-5p/106-5p/519-3p, miR-302-3p/372-3p/373-3p/520-3p, miR-142-5p, miR-302c-3p.2/520-3p, miR-326, miR-760, miR-138-5p, miR-27-3p, miR-145-5p, miR-142-3p.2, miR-101-3p.2, miR-182-5p, miR-203a-3p.2, miR-140-3p.1, miR-183-5p.1, miR-144-3p, miR-101-3p.1, miR-330-3p, miR-224-5p, miR-148-3p/152-3p, miR-485-5p, miR-122-5p, miR-155-5p, miR-320, miR-23-3p, miR-124-3p.2/506-3p, miR-135-5p, miR-381-3p, miR-26-5p, miR-1224-5p, miR-192-5p/215-5p, miR-1249-3p, miR-125-5p, miR-483-3p.2, miR-668-3p, miR-223-3p, miR-655-3p, miR-382-5p, miR-130-3p/301-3p/454-3p, miR-19-3p, miR-582-5p, miR-194-5p, miR-802, miR-483-3p.1, miR-382-3p, miR-129-5p, miR-3064-5p, miR-873-5p.2, miR-499a-5p, miR-128-3p, miR-532-5p, miR-296-5p, miR-744-5p, miR-425-5p, miR-218-5p, and miR-496.1.

In some embodiments, the method further comprises: measuring an amount of donor-derived cell-free DNA in a sample obtained from the transplant recipient, extracting cell-free DNA from the sample obtained from the transplant recipient, wherein the extracted cell-free DNA comprises donor-derived cell-free DNA and recipient-derived cell-free DNA; performing targeted amplification of the extracted DNA at 50-50,000 target loci in a single reaction volume; sequencing the amplified DNA by high-throughput sequencing to obtain sequencing reads and measuring the amount of donor-derived cell-free DNA based on the sequencing reads, generating a transplant rejection score indicating transplant rejection based on whether the measured amount of donor-derived cell-free DNA or a function thereof exceeds a cutoff threshold of cell-free DNA amount that indicates transplant rejection, wherein transplant rejection is determined based on both the one or more transplant rejection scores from the measured amount of one or more mRNAs and/or miRNAs and the transplant rejection score determined based on the measured amount of donor-derived cell-free DNA.

In some embodiments, measuring the amount of mRNAs and/or miRNAs comprises amplification of at least 2, at least 5, at least 10, at least 20, at least 30, at least 50, at least 100 target nucleic acid molecules, from 2-10, 200-100, 50-500, or 50-2000 target nucleic acid molecules, using at least at least 2, at least 5, at least 10, at least 20, at least 30, at least 50, at least 100 target RNA molecules, from 2-10, 200-100, 50-500, or 50-2000 pairs of forward and reverse PCR primers.

In some embodiments, the one or more mRNAs and/or miRNAs are determined by text mining databases.

In some embodiments, the amount of one or more mRNAs and/or miRNAs is measured relative to a housekeeping gene.

In another aspect the present disclosure relates to a method of preparing a composition of nucleic acids derived from a urine sample of a kidney transplant recipient useful for determination of kidney transplant rejection or risk of kidney transplant rejection comprising: (a) extracting nucleic acids from the urine sample of the kidney transplant recipient, wherein the extracted nucleic acids comprise one or more RNA molecules associated with a risk of kidney transplant rejection; (b) preparing the composition of nucleic acids from the extracted nucleic acids from step (a) by isolating RNA molecules and removing contaminating molecules; optionally wherein preparing the composition comprises performing reverse transcription of the RNA molecules to synthesize cDNA; (c) measuring an amount of RNA molecules associated with a risk of kidney transplant rejection in the composition of nucleic acids, and generating one or more transplant rejection scores from the measured amount of one or more RNA molecules, wherein the one or more transplant rejection scores provide a quantitative value of kidney transplant rejection risk or the presence of kidney transplant rejection.

In some embodiments, the one or more RNA molecules are mRNA or miRNA.

In some embodiments, the present disclosure relates to a method of preparing a composition of protein derived from a urine sample of a kidney transplant recipient useful for determination of kidney transplant rejection or risk of kidney transplant rejection comprising: (a) extracting protein from the urine sample of the kidney transplant recipient, wherein the extracted proteins are associated with a risk of kidney transplant rejection; (b) preparing the composition of protein from the protein extracted in step (a) by removing contaminating molecule; (c) measuring an amount of proteins in the composition, and generating one or more transplant rejection scores from the measured amount, wherein the one or more transplant rejection scores provide a quantitative value of kidney transplant rejection risk or the presence of kidney transplant rejection.

In some embodiments, the measuring step is based on two or more transplant rejection scores, wherein each transplant rejection score is based on a different subset of proteins.

In some embodiments, the one or more transplant rejection scores are generated using a predictive model, a machine learning based method, and/or an artificial intelligence method.

In some embodiments, the one or more transplant rejection scores provide a quantitative value of kidney transplant rejection risk that is predictive of a clinical assessment of a kidney disease state and can distinguish between two or more kidney disease states.

In some embodiments, the kidney disease state comprises non-rejection, T-cell mediated rejection (TCMR), antibody-mediated rejection (ABMR), or a mixed TCMR and ABMR disease state.

In some embodiments, the TCMR further comprises molecularly defined TCMR (mTCMR) and/or possible TCMR (pTCMR).

In some embodiments, the ABMR further comprises molecularly defined ABMR (mABMR) and/or possible ABMR (pABMR).

In some embodiments, the one or more transplant rejection scores comprise a first transplant rejection score based on a set of proteins associated with TCMR, and a second transplant rejection score based on a set of proteins associated with ABMR.

In some embodiments, the one or more transplant rejection scores comprise a transplant rejection score based on a set of proteins associated with inflammation, a transplant rejection score based on a set of proteins associated with allograft rejection, a transplant rejection score based on a set of proteins associated with T cell activation and/or differentiation, a transplant rejection score based on a set of proteins associated with B cell activation and/or differentiation, a transplant rejection score based on a set of proteins associated with a cytokine response, and/or a transplant rejection score based on a set of proteins associated with a chemokine response.

In some embodiments, the presently disclosed method comprises collecting the urine sample from the transplant recipient at different times and generating the one or more transplant rejection scores from each urine sample.

In some embodiments, the urine sample is collected prior to transplantation, simultaneous with transplantation, and/or after transplantation.

In some embodiments, the risk of transplant rejection is based on multiple transplant rejection scores generated at different time points, and wherein a change in one or more transplant rejection scores indicates a change in kidney disease state.

In some embodiments, the kidney transplant recipient is administered a treatment for the determined kidney disease state or kidney transplant rejection.

In some embodiments, a change in one or more transplant rejection scores specifies a presence, absence, or extent of therapeutic response to the treatment.

In some embodiments, the treatment is determined based on the one or more transplant rejection scores or a change in one or more transplant rejection scores.

In some embodiments, the treatment comprises an anti-rejection or an immunosuppressive agent.

In some embodiments, the presently disclosed method further comprises isolating cells from the urine samples, and extracting protein from the cells.

In some embodiments, the presently disclosed method further comprises isolating extracellular vesicles, and extracting protein from the extracellular vesicles.

In some embodiments, the one or more proteins are expressed from or regulated by a gene selected from the group consisting of:

| | | | | |
|---|---|---|---|---|
| ITM2A | SLAMF6 | IKZF3 | CCL5 | CXCL9 |
| IGHM | ITM2A | HIST1H2AJ | CXCL10 | CD3E |
| NKG7 | TRBC2 | HIST1H3I | TRGC2 | HIST1H1B |
| CTLA4 | PDCD1 | GZMA | IL2RB | IKZF3 |
| CD69 | SIT1 | CD96 | FAIM3 | ZAP70 |
| HIST1H1D | TRAC | RNASE6 | SLAMF6 | Z98744.2 |
| GZMB | IL18R1 | IL1RL1 | HIST1H4L | C1QB |
| CD8A | HIST1H2BM | HLA-DQB1 | LCK | ASB2 |
| HIST1H2AI | SLFN12L | CD28 | TRBV28 | RP4-620F22.2 |
| GIMAP1 | ICOS | ZNF831 | TRAT1 | GIMAP7 |
| P2RY10 | HCST | HIST1H2AK | GBP5 | EMB |
| CD2 | KLRC1 | CD6 | GPR174 | AL049822.1 |
| HLA-DQA1 | TIGIT | ITK | ST8SIA4 | GIMAP6 |
| GVINP1 | HLA-DQB1- | MRC1 | HLA-DPB1 | CCR2 |
| | AS1 | | | |
| CCND2-AS1 | AL009179.1 | EVI2B | FCGR3B | ETV7 |
| CTSW | HIST1H2AL | THEMIS | CH17- | BLM |
| | | | 373J23.1 | |
| PRF1 | DOK2 | CXCR6 | NGFR | KCNE1 |
| HIST1H3H | CTD- | HLA-DRA | CD8B | NCF1 |
| | 2313F11.1 | | | |
| GZMK | MLKL | HIST1H3B | CD3G | JAK3 |
| CD3D | C1QA | IFNG | CDCA7 | Z98744.3 |
| CXCL11 | RP11-284N8.3 | HLA-H | TOX | GIMAP4 |
| FAM78A | FAM65B | HCG4P7 | SMAP2 | EOMES |
| RASAL3 | CD163 | FCRL3 | IFIT2 | SH2D1A |
| HCG4P11 | RGS18 | MIR155HG | IFITM1 | HIST1H2BL |
| ITGA4 | RP11- | MATK | KCNJ2 | KLRB1 |
| | 463J10.3 | | | |
| HIST1H2AH | APOBEC3G | PSMB9 | PTPRC | RCSD1 |
| PYHIN1 | BTN3A3 | AC007278.2 | C11orf21 | RHOH |
| TMEM156 | HIST2H3D | NLRP3 | AF001548.5 | HIST1H2BI |
| CD74 | AGAP2 | CD52 | GBP1 | GMFG |
| LAX1 | SASH3 | GNLY | HIST2H2AB | MS4A6A |
| PDE3B | SLAMF8 | AGER | PREX1 | IRF8 |
| IKZF1 | TBC1D10C | FCGR3A | BCL11B | HLA-DRB1 |
| CD97 | CTB-186G2.1 | NLRC3 | TAP1 | CD247 |
| HIST1H3G | GBP4 | RP11- | TMC8 | SELPLG |
| | | 1049A21.2 | | |
| HIST1H2BO | CCND2 | CST7 | CCR5 | RP11-264B17.5 |
| GRAP2 | HLA-B | IKBIP | KRT1 | SERPINB12 |
| ALB | TGM3 | HS3ST6 | NRTN | FAM180A |
| FALEC | FGF22 | HCN2 | SLC24A3 | RP11-167H9.5 |
| C1QB | IL18R1 | KCNE1 | CCR2 | C1QA |
| CD163 | CST7 | SLC1A3 | RNASE6 | HIST1H1B |
| TRGC2 | MS4A6A | C1QC | RGS1 | MSR1 |
| HIST1H1D | HIST1H3I | CD28 | LSM11 | HIST1H3B |
| HIST1H2AJ | GIMAP6 | DAAM2 | CCL5 | CTLA4 |
| IGHG1 | C1orf162 | MIR155HG | ITM2A | GIMAP4 |
| HIST1H2BM | HIST1H3G | FCER1G | FKBP5 | IL1R2 |
| GZMA | CLEC12A | LINC01127 | Z98744.2 | GPR34 |
| HLA-DMB | HIST1H4L | RRM2 | TREM2 | GIMAP8 |
| CD3E | AL049822.1 | HLA-DPB1 | LMNB1 | CD300C |
| IFITM1 | BLM | SAMSN1 | IRF8 | CLSPN |
| IL1RL1 | SDS | RP11-274E7.2 | MS4A4A | PDCD1 |
| ICOS | RP11- | TNFSF13B | HIST1H3F | RP11-777B9.5 |
| | 848G14.5 | | | |
| GIMAP2 | IL18RAP | VSIG4 | GIMAP7 | SRGN |
| TRBC2 | LIPA | SERPINE1 | CENPK | IL10 |
| GZMK | DTL | GLDN | SMAP2 | HIST1H2BB |
| SIT1 | IFITM3 | CTD- | HLA-DRA | THBS1 |
| | | 2033D15.2 | | |
| ADORA3 | STAB1 | AIM2 | EVI2A | TFPI2 |
| EMB | SLA | CD8A | RCSD1 | GIMAP1 |
| FAM105A | FPR2 | CD180 | RP11- | RP4-737E23.5 |
| | | | 463J10.3 | |
| FCGR3A | HIST1H2BL | CD1D | UBE2C | PDK4 |
| MLKL | RP11-7F17.3 | TLR7 | PTPRC | KBTBD8 |
| CD96 | HIST1H2AI | FAIM3 | CD48 | IFI27L2 |
| HIST1H4F | CTD- | SLC16A6 | LRRC8C | NLRP6 |
| | 2033D15.3 | | | |
| TLR8 | IRAK3 | LY96 | LILRA5 | AL009179.1 |
| KIAA0101 | GCA | CCDC109B | CTSL | CCR5 |
| BTN3A2 | STAMBPL1 | HLA-DPA1 | CTD- | LAIR1 |
| | | | 2313F11.1 | |
| FES | RNASE1 | CXCL10 | TRIM22 | HIST1H3H |
| CD52 | MYBL2 | RP11- | RNU4-1 | KLRC1 |
| | | 403P17.6 | | |
| FEN1 | MIR181A1HG | HCG27 | RP11- | BIN2 |
| | | | 335I12.2 | |
| RP4-620F22.2 | GBP5 | FYB | PCED1B | AL031777.1 |
| MMD | DOK2 | RP11- | PPT1 | FAM65B |
| | | 404F10.2 | | |
| GVINP1 | SERPINF1 | CARD16 | CEP55 | HIST1H2AL |
| CD86 | CD2 | JAK3 | SELPLG | AC007278.3 |
| FAM111B | GYG1 | NLRP3 | ZWINT | ITGA4 |
| TRAC | SELL | GPR183 | TRG-AS1 | ST8SIA4 |
| NCKAP1L | STK17B | OLAH | APOC1 | PCED1B-AS1 |
| SLFN13 | TIGIT | ATHL1 | HIST1H2BI | HLA-DMA |
| ENTPD1 | SLCO2B1 | IFNG | CD300LF | F13A1 |
| NEIL3 | TMEM14E | ACSL4 | RN7SKP48 | GZMH |
| LYAR | GPNMB | IKZF1 | RP3-455J7.4 | SCIMP |
| AMIGO2 | TLR5 | EXOC6 | IFITM2 | HAVCR2 |
| TLR2 | CTB- | FGL2 | A2M | LY9 |
| | 111H14.1 | | | |
| KIAA0825 | WDR76 | LILRB5 | SLAMF6 | CTB-41I6.2 |
| FGR | HELLS | CD274 | HIST1H2AB | MELK |
| ASB2 | SIGLEC9 | TPK1 | MAP2K6 | RP11-347P5.1 |
| HIST1H2AH | EVI2B | CTA-373H7.7 | FAM26F | KLRB1 |
| TRPV2 | CHIT1 | HTATSF1P2 | TSC22D3 | TLDC2 |
| MGAM | RP11-69L16.4 | CXCR6 | STAT4 | TNFSF8 |
| MB21D1 | SLFN11 | RP11- | TYROBP | ITGB2 |
| | | 124N14.3 | | |
| LILRB4 | CDK1 | PSMB8 | HIST2H2AB | DNA2 |
| PDCD1LG2 | CCL18 | RP11-25K21.6 | HLA-DQA1 | CELF2 |
| AIF1 | LBR | AL021807.1 | GMFG | PTGER2 |
| VIM | CASP1 | HCG4P11 | RP4- | HLA-DQB1 |
| | | | 671O14.5 | |
| ZNF367 | VNN1 | RP11-278C7.5 | HCST | SLC46A3 |
| SLAMF8 | HLA-H | CARD8-AS1 | RP1- | CTD-2033D15.1 |
| | | | 68D18.2 | |
| NCAPG | SMARCD3 | MMP19 | ARHGAP15 | FABP5P7 |
| HLA-DQB1- | HIST1H2BO | FCGR2A | ITGAM | MCM6 |
| AS1 | | | | |
| VCAN | CD37 | BTLA | Z98744.3 | NMI |
| RP11- | MARCH1 | RP11- | VNN2 | HLA-DOA |
| 1049A21.2 | | 290C10.1 | | |
| CD300E | RGS2 | HIST1H3J | NCAPH | HIST1H2BE |
| HLA-B | CD4 | MSNP1 | CTSC | CXCL5 |
| AC008984.2 | HIST1H2AE | APMAP | CD84 | SHC4 |
| GLIPR2 | ZPLD1 | GPR171 | FAM101B | C5AR1 |
| UBASH3B | FAM78A | SLED1 | CRTAM | PMP22 |
| ZEB2-AS1 | CTC-301O7.4 | PFN1P1 | EPSTI1 | HK3 |
| C18orf54 | ATP8A1 | C11orf21 | LCP1 | FAM198B |
| RP11-326C3.2 | ETV7 | WAS | RP11- | CYBB |
| | | | 415J8.3 | |
| CDCA7L | CMSS1 | RBL1 | FLI1 | RNF175 |
| IL27RA | CSF1R | TMX1 | FAR2 | HAUS1 |
| AOAH | AQP9 | ARHGEF6 | AC007278.2 | CLEC2B |
| CSGALNACT2 | LILRB1 | ITK | LINC01093 | P2RY14 |
| FPR1 | PLK4 | RP11- | MDM1 | PREX1 |
| | | 488L18.10 | | |
| RP11- | RP11-256L6.3 | RP6-159A1.4 | CD226 | CD300LB |
| 186N15.3 | | | | |
| SNX10 | CEP85L | CLEC10A | LTB4R | MYC |
| CENPH | FXYD4 | TRAF5 | CIITA | SOAT1 |
| RP11-325F22.2 | AGAP2 | NAIP | PLP2 | PSMB8-AS1 |
| ETS1 | IRF4 | NCF1 | E2F8 | CD33 |
| BTNL8 | CLEC4E | POLR3GL | IKZF3 | IL2RA |
| GFI1 | HHEX | SNX20 | APBB1IP | RP11-15A1.7 |
| DCK | SNORA12 | EMR1 | APOBEC3G | ANXA2R |
| ANKRD22 | AC025048.1 | MS4A7 | MSN | FABP5 |
| PYHIN1 | GPR141 | RP11-455F5.5 | HCG4P7 | GAB3 |
| PARVG | C17orf53 | RP11-297C4.2 | SLC4A7 | FLT3 |
| TNFAIP6 | HJURP | PATL2 | TLR4 | LAPTM5 |
| RP11- | RAB37 | MOB3C | GTF2H2C | NGFR |
| 876N24.3 | | | | |
| KIF14 | IGSF6 | TTC27 | PPP2R2B | LGALS1 |
| RP5-1171I10.5 | IKBIP | XXbac- | AC025171.1 | CTSW |
| | | BPG299F13.14 | | |
| SLC11A1 | HGF | CNTRL | UGT1A6 | CD53 |
| RP11-701P16.2 | DOCK11 | VIM-AS1 | RP11- | RP11-488L18.4 |
| | | | 272L13.4 | |
| EMP3 | CCL2 | EXO5 | PLA2G7 | GPSM3 |
| LINC01272 | STAT1 | GNG2 | CR1 | SNRPF |
| TNNI2 | HCLS1 | HIST1H3C | RP11- | HENMT1 |
| | | | 214O1.2 | |
| WIPF1 | TNFSF4 | DOCK10 | ACSL1 | IL10RA |
| HIST1H2AK | CD97 | ADAMTS2 | MAD2L1 | SASH3 |
| SLC16A1 | RN7SKP203 | RAC2 | CD36 | JAM3 |
| BUB1B | RRN3 | RAB20 | CTB-41I6.1 | RNU4-2 |
| CERKL | TSPAN2 | HLA-F | CFP | ARL6IP6 |
| TNFRSF25 | CPVL | CD6 | MYADM | RP11-149123.3 |
| HLA-DRB5 | RFC3 | FBN1 | NUSAP1 | PLXNC1 |
| NLRC4 | CTD- | GPR68 | MAP7D3 | ARG1 |
| | 2047H16.3 | | | |
| F2R | PIK3CD-AS1 | GSG2 | HS3ST3B1 | IQGAP2 |
| ALOX5AP | AC007620.3 | IDO1 | BCL2A1 | CALHM2 |
| KIF11 | TTK | LTB | FCGR1A | EEF1E1 |
| AC006460.2 | RNASE2 | KIF15 | P2RY8 | LPAR6 |
| BASP1 | BMP7 | CALHM6 | CD14 | CD46 |
| CXCL14 | ENG | FN1 | FOXP3 | IFNGR1 |
| IL18BP | IL32 | INPP5D | IRAK2 | ISG20 |
| KLRC4- | MAP4K1 | NAMPT | PRPF31 | PRPF32 |
| KLRK1 | | | | |
| PRPF33 | PSMB10 | PYCARD | RUNX3 | SERPINA1 |
| TBP | TGFB1 | | | |
| ADD1 | AIFM3 | ANKH | ANXA1 | APP |
| ATF3 | AVPR1A | BAX | BCAP31 | BCL10 |
| BCL2L1 | BCL2L10 | BCL2L11 | BCL2L2 | BGN |
| BID | BIK | BIRC3 | BMF | BMP2 |
| BNIP3L | BRCA1 | BTG2 | BTG3 | CASP1 |
| CASP2 | CASP3 | CASP4 | CASP6 | CASP7 |
| CASP8 | CASP9 | CAV1 | CCNA1 | CCND1 |
| CCND2 | CD14 | CD2 | CD38 | CD44 |
| CD69 | CDC25B | CDK2 | CDKN1A | CDKN1B |
| CFLAR | CLU | CREBBP | CTH | CTNNB1 |
| CYLD | DAP | DAP3 | DCN | DDIT3 |
| DFFA | DIABLO | DNAJA1 | DNAJC3 | DNM1L |
| DPYD | EBP | EGR3 | EMP1 | ENO2 |
| ERBB2 | ERBB3 | EREG | ETF1 | F2 |
| F2R | FAS | FASLG | FDXR | FEZ1 |
| GADD45A | GADD45B | GCH1 | GNA15 | GPX1 |
| GPX3 | GPX4 | GSN | GSR | GSTM1 |
| GUCY2D | H1F0 | HGF | HMGB2 | HMOX1 |
| HSPB1 | IER3 | IFITM3 | IFNB 1 | IFNGR1 |
| IGF2R | IGFBP6 | IL18 | IL1A | IL1B |
| IL6 | IRF1 | ISG20 | JUN | KRT18 |
| LEF1 | LGALS3 | LMNA | LPPR4 | LUM |
| MADD | MCL1 | MGMT | MMP2 | NEDD9 |
| NEFH | PAK1 | PDCD4 | PDGFRB | PEA15 |
| PLAT | PLCB2 | PMAIP1 | PPP2R5B | PPP3R1 |
| PPT1 | PRF1 | PSEN1 | PSEN2 | PTK2 |
| RARA | RELA | RETSAT | RHOB | RHOT2 |
| RNASEL | ROCK1 | SAT1 | SATB1 | SC5DL |
| SLC20A1 | SMAD7 | SOD1 | SOD2 | SPTAN1 |
| SQSTM1 | TAP1 | TGFB2 | TGFBR3 | TIMP1 |
| TIMP2 | TIMP3 | TNF | TNFRSF12A | TNFSF10 |
| TOP2A | TSPO | TXNIP | VDAC2 | WEE1 |
| XIAP | | | | |
| ALB | BASP1 | BMP7 | C11orf21 | C1QB |
| C3 | CALHM6 | CD14 | CD3D | CD3E |
| CD46 | CD6 | CD74 | CDH1 | CXCL10 |
| CXCL11 | CXCL14 | CXCL9 | ENG | FN1 |
| FOXP3 | GNLY | GZMB | HAVCR2 | IFNGR1 |
| IL18BP | IL2RA | IL32 | INPP5D | IRAK2 |
| ISG20 | KLRC4- | LCK | MAP4K1 | MRC1 |
| | KLRK1 | | | |
| NAMPT | NKG7 | PRF1 | PRPF31 | PRPF32 |
| PRPF33 | PSMB10 | PSMB9 | PYCARD | RGS1 |
| RUNX3 | SERPINA1 | SERPINB12 | STAT1 | TAP1 |
| TBP | TGFB1 | TMEM156 | | |
| RBM6 | PSMB1 | ITIH4 | TP53BP1 | DFNB31 |
| LGALS1 | AP4S1 | BRF2 | RAD23B | HNRNPA2B1 |
| ZNF436 | NECAB3 | HCST | AHDC1 | ARPP19 |
| C3orf20 | ACTR3B | UBAC2 | SETDB2 | STX17 |
| RAB30 | RPLP1 | SNRPF | CMTM3 | USP21 |
| LRIG1 | GTF2H2 | MPP7 | MERTK | ANGPT1 |
| AK9 | ITLN2 | CACHD1 | SYVN1 | ELF3 |
| UCN | ZMYM6 | BDH2 | DACT1 | COPS6 |
| 2-Sep | ZNF354A | BTD | SIGLEC17P | TADA3 |
| CTSF | LINC00469 | DMAP1 | DCTPP1 | MFSD6L |
| ADAMTSL5 | SUPT5H | S100A5 | ARMCX4 | ZNF502 |
| ZNF138 | ZNF841 | FICD | COL5A2 | RP11-15J10.1 |
| GNRHR2 | TRDC | AC016747.3 | AGGF1P1 | CHCHD3P3 |
| PRPF38AP1 | RP11- | AC010894.3 | RPL4P6 | AP000936.1 |
| | 242O24.3 | | | |
| LINC01278 | LINC00298 | AC159540.14 | LINC00299 | AMY2B |
| RPL34P18 | RP11- | RPP21 | RP11- | RP11-392P7.6 |
| | 1008M1.1 | | 20O24.4 | |
| CDK11B | RP1-16A9.1 | RP11-585P4.5 | P2RX5- | RP11-644F5.11 |
| | | | TAX1BP3 | |
| SPESP1 | RP11- | RP11-412D9.4 | RPL23AP97 | RP11-529K1.2 |
| | 603B24.1 | | | |
| RP5- | RN7SL246P | PARD6G-AS1 | RP11- | RP11-840I19.5 |
| 1107A17.4 | | | 635N19.1 | |
| RP11-179B2.2 | RP11- | LA16c- | F8A1 | uc_338 |
| | 313P22.1 | 360A4.1 | | |
| RP11-43N22.1 | RP11- | | | |
| | 133M8.3 | | | |

, and combinations thereof.

In some embodiments, the one or more proteins are expressed from a gene selected from the group consisting of:

| | | | | |
|---|---|---|---|---|
| ALB | BASP1 | BMP7 | C11orf21 | C1QB |
| C3 | CALHM6 | CD14 | CD3D | CD3E |
| CD46 | CD6 | CD74 | CDH1 | CXCL10 |
| CXCL11 | CXCL14 | CXCL9 | ENG | FN1 |
| FOXP3 | GNLY | GZMB | HAVCR2 | IFNGR1 |
| IL18BP | IL2RA | IL32 | INPP5D | IRAK2 |
| ISG20 | KLRC4-KLRK1 | LCK | MAP4K1 | MRC1 |
| NAMPT | NKG7 | PRF1 | PRPF31 | PRPF32 |
| PRPF33 | PSMB10 | PSMB9 | PYCARD | RGS1 |
| RUNX3 | SERPINA1 | SERPINB12 | STAT1 | TAP1 |
| TBP | TGFB1 | TMEM156 | | |

, and combinations thereof.

In some embodiments, the one or more proteins are expressed from a gene selected from the group consisting of:

| | | | | | | | | | |
|---|---|---|---|---|---|---|---|---|---|
| ADD1 | AIFM3 | ANKH | ANXA1 | APP | ATF3 | AVPR1A | BAX | BCAP31 | BCL10 |
| BCL2L1 | BCL2L10 | BCL2L11 | BCL2L2 | BGN | BID | BIK | BIRC3 | BMF | BMP2 |
| BNIP3L | BRCA1 | BTG2 | BTG3 | CASP1 | CASP2 | CASP3 | CASP4 | CASP6 | CASP7 |
| CASP8 | CASP9 | CAV1 | CCNA1 | CCND1 | CCND2 | CD14 | CD2 | CD38 | CD44 |
| CD69 | CDC25B | CDK2 | CDKN1A | CDKN1B | CFLAR | CLU | CREBBP | CTH | CTNNB1 |
| CYLD | DAP | DAP3 | DCN | DDIT3 | DFFA | DIABLO | DNAJA1 | DNAJC3 | DNM1L |
| DPYD | EBP | EGR3 | EMP1 | ENO2 | ERBB2 | ERBB3 | EREG | ETF1 | F2 |
| F2R | FAS | FASLG | FDXR | FEZ1 | GADD45A | GADD45B | GCH1 | GNA15 | GPX1 |
| GPX3 | GPX4 | GSN | GSR | GSTM1 | GUCY2D | H1F0 | HGF | HMGB2 | HMOX1 |
| HSPB1 | IER3 | IFITM3 | IFNB1 | IFNGR1 | IGF2R | IGFBP6 | IL18 | IL1A | IL1B |
| IL6 | IRF1 | ISG20 | JUN | KRT18 | LEF1 | LGALS3 | LMNA | LPPR4 | LUM |
| MADD | MCL1 | MGMT | MMP2 | NEDD9 | NEFH | PAK1 | PDCD4 | PDGFRB | PEA15 |
| PLAT | PLCB2 | PMAIP1 | PPP2R5B | PPP3R1 | PPT1 | PRF1 | PSEN1 | PSEN2 | PTK2 |
| RARA | RELA | RETSAT | RHOB | RHOT2 | RNASEL | ROCK1 | SAT1 | SATB1 | SC5DL |
| SLC20A1 | SMAD7 | SOD1 | SOD2 | SPTAN1 | SQSTM1 | TAP1 | TGFB2 | TGFBR3 | TIMP1 |
| TIMP2 | TIMP3 | TNF | TNFRSF12A | TNFSF10 | TOP2A | TSPO | TXNIP | VDAC2 | WEE1 |
| XIAP | | | | | | | | | |

, and combinations thereof.

In some embodiments, the one or more proteins are expressed from a gene selected from the group consisting of: CXCL9, CD3ϵ, IP-10, LCK, C1QB, PSMB9, Tim-3, Foxp3, FAM26F, ALB, CTB-186G2.1, CXCL11, GNLY, MRC1, RGS1, RP11-1049A21.2, TMEM156, Z98744.3, SERPINB12, C11orf21, AF001548, 18s ribosomal, Basp1, Cd6, Cxcl10, Cxcl9, Inpp5d, Isg20, Lck, Nkg7, Runx3, Tap1, GZMB, PRF1, CD74, IL32, STAT1, CXCL14, SERPINA1, B2M, C3, PYCARD, BMP7, TBP, NAMPT, IFNGR1, IRAK2, IL18BP, and combinations thereof.

In some embodiments, the one or more proteins are expressed from a gene selected from the group consisting of: CXCL9, CD3ϵ, IP-10, LCK, C1QB, PSMB9, Tim-3, Foxp3, FAM26F, ALB, CTB-186G2.1, CXCL11, GNLY, MRC1, RGS1, RP11-1049A21.2, TMEM156, Z98744.3, SERPINB12, C11orf21, AF001548, 18s ribosomal, Basp1, Cd6, Cxcl10, Cxcl9, Inpp5d, Isg20, Lck, Nkg7, Runx3, Tap1, GZMB, PRF1, CD74, IL32, STAT1, CXCL14, SERPINA1, B2M, C3, PYCARD, BMP7, TBP, NAMPT, IFNGR1, IRAK2, Calhm6, Klrc4-Klrk1, Psmb10, CD3delta, Map4k1, IL2Ra, TGFB1, FN1, CDH1, PRPF31, HAVCR2, IL18BP, and combinations thereof.

In some embodiments, the one or more proteins are expressed from a gene selected from the group consisting of: CXCL9, CD3ϵ, IP-10, LCK, C1QB, PSMB9, Tim-3, Foxp3, FAM26F, ALB, CTB-186G2.1, CXCL11, GNLY, MRC1, RGS1, RP11-1049A21.2, TMEM156, Z98744.3, SERPINB12, C11orf21, AF001548, 18s ribosomal, Basp1, Cd6, Cxcl10, Cxcl9, Inpp5d, Isg20, Lck, Nkg7, Runx3, Tap1, and combinations thereof.

In some embodiments, the one or more proteins are expressed from a gene selected from the group consisting of: CXCL11, CXCL9, GNLY, PSMB9, TAP1, CXCL10, NKG7, ISG20, RUNX3, C11orf21, C1QB, LCK, INPP5D, CD6, CD3ε, MRC1, BASP1, TMEM156, ALB, FOXP3, RGS1, SERPINB12, and combinations thereof.

In some embodiments, the one or more proteins are expressed from a gene selected from the group consisting of: CXCL11, CXCL9, GNLY, PSMB9, TAP1, CXCL10, NKG7, ISG20, RUNX3, C11orf21, C1QB, LCK, INPP5D, CD6, CD3ε, MRC1, BASP1, TMEM156, ALB, FOXP3, RGS1, SERPINB12, PRF1, CALHM6, KLRC4-KLRK1, CD74, GZMB, PSMB10, B2M, STAT1, CD3delta, PYCARD, IL18BP, MAP4K1, IL32, IL2RA, C3, IFNGR1, IRAK2, TGFB1, FN1, NAMPT, SERPINA1, TBP, CDH1, PRPF31, BMP7, CXCL14, HAVCR2 and combinations thereof.

In some embodiments, the one or more proteins are not expressed from a gene selected from the group consisting of: PDCD1, MARCHF8, DCAF12, IL1R2, FLT3, ITGA4, ITGAM, PF4, C6orf25, SEMA7A, RHOU, and combinations thereof.

In some embodiments, the one or more proteins are not expressed from a gene selected from the group consisting of: CCDC159, dcaf12, DECR1, ERCC5, EWSR1, FLT3, GABPB2, GPI, IL1R2, ITGA4, ITGAM, MAP3K3, MAPK9, MARCHF8, NONO, PDCD1, PF4, RHOU, SEMA7A, SRRM1, TBC1D10B, TOP2B, and combinations thereof.

In some embodiments, the presently disclosed method further comprises: (a) measuring an amount of donor-derived cell-free DNA in a sample obtained from the transplant recipient, extracting cell-free DNA from the sample obtained from the transplant recipient, wherein the extracted cell-free DNA comprises donor-derived cell-free DNA and recipient-derived cell-free DNA; (b) performing targeted amplification of the extracted DNA at 50-50,000 target loci in a single reaction volume; (c) sequencing the amplified DNA by high-throughput sequencing to obtain sequencing reads and measuring the amount of donor-derived cell-free DNA based on the sequencing reads, generating a score indicating transplant rejection based on whether the measured amount of donor-derived cell-free DNA or a function thereof exceeds a cutoff threshold of cell-free DNA amount that indicates transplant rejection, wherein transplant rejection is determined based on both the one or more scores based on the measured amount of proteins and the score determined based on the measured amount of donor-derived cell-free DNA.

### BRIEF DESCRIPTION OF THE DRAWINGS

FIG. 1 is a graphic depiction of exemplary mRNAs identified from Urine rejection papers that effectively differentiate kidney rejection states.

FIG. 2 is a graphic depiction of exemplary mRNAs identified from Urine rejection papers that effectively differentiate kidney rejection states.

FIG. 3 is a graphic depiction of exemplary mRNAs identified from Urine rejection papers that effectively differentiate kidney rejection states.

FIG. 4 is a graphic depiction of exemplary mRNAs identified from Urine rejection papers that effectively differentiate kidney rejection states.

FIG. 5 is a graphic depiction of exemplary mRNAs identified from Urine rejection papers that effectively differentiate kidney rejection states.

FIG. 6 is a graphic depiction of exemplary mRNAs identified from Urine rejection papers that effectively differentiate kidney rejection states.

FIG. 7 is a graphic depiction of exemplary mRNAs identified from Urine rejection papers that effectively differentiate kidney rejection states.

FIG. 8 is a graphic depiction of exemplary mRNAs identified from Urine rejection papers that effectively differentiate kidney rejection states.

FIG. 9 is a graphic depiction of exemplary mRNAs identified from Urine rejection papers that effectively differentiate kidney rejection states.

FIG. 10 is a graphic depiction of exemplary mRNAs identified from Urine rejection papers that effectively differentiate kidney rejection states.

FIG. 11 is a graphic depiction of exemplary mRNAs from CXCL type genes (antimicrobial response genes) identified by text mining that are useful for determining kidney rejection states.

FIG. 12 is a graphic depiction of exemplary mRNAs from housekeeping genes that do not differentiate rejection states.

FIG. 13 is a graphic depiction of exemplary mRNAs from housekeeping genes that do not differentiate rejection states.

FIG. 14 is a graphic depiction of exemplary mRNAs identified from the PaxGene® RNA blood collection tubes, of which very few genes effectively differentiate rejection states.

FIG. 15 is a graphic depiction of exemplary mRNAs identified from the PaxGene® RNA blood collection tubes, of which very few genes effectively differentiate rejection states.

FIG. 16 is a graphic depiction of exemplary mRNAs identified from the PaxGene® RNA blood collection tubes, of which very few genes effectively differentiate rejection states.

FIG. 17 is a graphic depiction of KEGG gene ontology analysis of the genes published in Akalin versus the herein identified genes enriched in urine samples. FIG. 17A shows the GO analysis of the Akalin genes. FIG. 17 B shows the GO analysis of the genes herein identified in urine samples.

FIG. 18 is a graphic depiction of Biochemistry Process based gene ontology analysis of the genes published in Akalin versus the herein identified genes enriched in urine samples. FIG. 18A shows the GO analysis of the Akalin genes. FIG. 18 B shows the GO analysis of the genes herein identified in urine samples.

FIG. 19 shows microRNA markers found in Urine (urine miRNA jmp) from 207 patients GSE128348_MBITZ1-CTOT2-Urine-Biopsy-Associated.

### DETAILED DESCRIPTION

The present disclosure relates to methods of identifying kidney allograft rejection genes in urine and use of those measurements for accurate and specific detection of kidney rejection states. Methods for accurate and specific detection of different states of kidney rejection could yield novel preventive, diagnostic, and therapeutic approaches. Two main types of kidney allograft rejection have been recognized: T-cell mediated (TCMR), and antibody mediated (ABMR). In addition, the kidney rejection status can be determined to be possible TCMR (pTCMR), possible ABMR (pABMR), and mixed (both TCMR and ABMR). TCMR and ABMR differ in terms of pathogenesis, pathology, and prognosis, and require tailored treatment, and cannot be distinguished exclusively based on clinical data or histology. Hence, the methods herein provides methods for distinguishing kidney rejection states based on measuring amounts of specific RNAs or proteins in urine. Identification of mRNAs, proteins, or miRNAs differentially expressed in urine samples from transplant recipients in different kidney rejection stats allowed building molecular classifiers that can distinguish kidney rejection states as further described below.

In one aspect the present disclosure relates to a method of preparing a composition of nucleic acids derived from a urine sample of a kidney transplant recipient useful for determination of kidney transplant rejection or risk of kidney transplant rejection comprising: (a) extracting nucleic acids from the urine sample of the kidney transplant recipient, wherein the extracted nucleic acids comprise one or more mRNAs of target genes and/or miRNAs associated with kidney transplant rejection; (b) preparing a composition of nucleic acids from the extracted nucleic acids in step (a) by isolating mRNAs and/or miRNAs and removing contaminating molecules, optionally wherein preparing the composition comprises reverse transcribing complementary DNA (cDNA) from the nucleic acids extracted in step (a); (c) measuring an amount of the one or more mRNAs and/or miRNAs, and generating one or more transplant rejection scores from the measured amount of the one or more mRNAs and/or miRNAs, wherein the one or more transplant rejection scores provide a quantitative value of kidney transplant rejection risk or the presence of kidney transplant rejection.

In some embodiments, the method comprises generating two or more transplant rejection scores, wherein each transplant rejection score is based on a different subset of mRNAs and/or miRNAs.

In another aspect the present disclosure relates to a method of preparing a composition of nucleic acids derived from a urine sample of a kidney transplant recipient useful for determination of kidney transplant rejection or risk of kidney transplant rejection comprising: (a) extracting nucleic acids from the urine sample of the kidney transplant recipient, wherein the extracted nucleic acids comprise one or more RNA molecules associated with a risk of kidney transplant rejection; (b) preparing the composition of nucleic acids from the extracted nucleic acids from step (a) by isolating RNA molecules and removing contaminating molecules; optionally wherein preparing the composition comprises performing reverse transcription of the RNA molecules to synthesize cDNA; (c) measuring an amount of RNA molecules associated with a risk of kidney transplant rejection in the composition of nucleic acids, and generating one or more transplant rejection scores from the measured amount of one or more RNA molecules, wherein the one or more transplant rejection scores provide a quantitative value of kidney transplant rejection risk or the presence of kidney transplant rejection.

In some embodiments, the present disclosure relates to a method of preparing a composition of protein derived from a urine sample of a kidney transplant recipient useful for determination of kidney transplant rejection or risk of kidney transplant rejection comprising: (a) extracting protein from the urine sample of the kidney transplant recipient, wherein the extracted proteins are associated with a risk of kidney transplant rejection; (b) preparing the composition of protein from the protein extracted in step (a) by removing contaminating molecules; (c) measuring an amount of proteins in the composition, and generating one or more transplant rejection scores from the measured amount, wherein the one or more transplant rejection scores provide a quantitative value of kidney transplant rejection risk or the presence of kidney transplant rejection. In some embodiments, the measuring step is based on two or more transplant rejection scores, wherein each transplant rejection score is based on a different subset of proteins. In some embodiments, the one or more transplant rejection scores comprise a first transplant rejection score based on a set of proteins associated with TCMR, and a second transplant rejection score based on a set of proteins associated with ABMR.

In some embodiments, the RNA such as mRNA or microRNA (miRNA), cell-free DNA, or protein is isolated from urine samples of a kidney transplant recipient and expression products from a set of genes determined to be able to differentiated between different rejection states are measured. In some embodiments, the miRNAs binding the mRNA expressed from the set of genes are measured. The working examples presented herein illustrate that mRNA found in urine can differentiated between different kidney rejection states. In particular, mRNAs and miRNAs useful for detecting and distinguish kidney rejection states are listed in Table 1, or more preferably Table 6. Alternatively, it is contemplated herein that the apoptosis pathway genes expressing mRNA found in urine may also be useful for detecting and distinguish kidney rejection states. Illustrative examples of apoptosis pathway genes determined herein to be useful for detecting and distinguish kidney rejection states are listed in Table 9. It is also considered herein that protein expressed from the genes listed in Tables 1, 6, and 9 can also be useful for detecting and distinguish kidney rejection states.

Determination of target genes useful for detecting and distinguish kidney rejection states is described in greater detail in working example 1. Briefly, the target genes are determined by cross-referencing target genes from the MMDx® diagnostic system with genes show to be expressed in urine samples and associated with particular kidney rejection states. Machine learning approaches are used to test the performance of these genes in detecting kidney rejection states in silico by using the MMDx® diagnostic system. Methods of measuring RNA and determining rejection scores based on these measurements to determine kidney rejection are further described below.

The determination of different kidney rejection or disease states can inform clinical treatment of the kidney transplant recipient with for example an anti-rejection agent to treat the rejection state. In some embodiments, the kidney transplant recipient is administered a treatment for the determined kidney disease state or kidney transplant rejection. In some embodiments, the treatment comprises an anti-rejection or an immunosuppressive agent. In some embodiments, a change in one or more transplant rejection scores specifies a presence, absence, or extent of therapeutic response to the treatment. In some embodiments, the treatment is determined based on the one or more transplant rejection scores or a change in one or more transplant rejection scores.

An “anti-rejection agent” is any substance administered to a subject for the purpose of preventing or ameliorating a rejection state. Anti-rejection agents include, but are not limited to, azathioprine, cyclosporine, FK506, tacrolimus, mycophenolate mofetil, anti-CD25 antibody, antithymocyte globulin, rapamycin, ACE inhibitors, perillyl alcohol, anti-CTLA4 antibody, anti-CD40L antibody, anti-thrombin III, tissue plasminogen activator, antioxidants, anti-CD 154, anti-CD3 antibody, thymoglobin, OKT3, corticosteroid, or a combination thereof. “Baseline therapeutic regimen” is understood to include those anti-rejection agents being administered at a baseline time, subsequent to the transplant. The baseline therapeutic regimen may be modified by the temporary or long-term addition of other anti-rejection agents, or by a temporary or long-term increase or decrease in the dose of one, or all, of the baseline anti-rejection agents. For TCMR, the initial treatment conventionally includes pulse methylprednisolone at 250-500 mg daily for 3-5 days or T cell depletion. For ABMR, the conventional treatment may be plasma exchange and intravenous Ig, with or without rituximab, or more recently, treatment of ABMR includes removing antibody-producing B cells or plasma cells, removing antibodies (DSA), and/or inhibiting the subsequent complement-regulated graft damage.

Methods of Determining and Monitoring Kidney Rejection Based on Measuring RNA (or miRNA Binding the mRNA) in Urine Samples.

In one aspect, the disclosure herein relates to a method of preparing a composition of complimentary DNA (cDNA) from RNA extracted from a urine sample of a kidney transplant recipient useful for determination of kidney rejection. In some embodiments, no amplification or pre-amplification is performed on the extracted RNA prior to measuring the amounts by quantitative PCR, microarray or sequencing.

In some embodiments, when the RNA amounts are measured by sequencing, a sequencing library is prepared from the cDNA comprising preparing a sequencing library comprises attaching adapters to the cDNA for example by ligation. In some embodiments, the cDNA fragments are repaired and filled to generated blunt ends. In some embodiments, adapters are appended to the cDNA fragments by blunt end ligation. In some embodiments, the cDNA are appended with adapters by sticky end ligation to generate a sequencing library of cDNA. In some embodiments, measuring the amount of mRNAs and/or miRNAs comprises amplification of at least 2, at least 5, at least 10, at least 20, at least 30, at least 50, at least 100 target nucleic acid molecules, from 2-10, 200-100, 50-500, or 50-2000 target nucleic acid molecules, using at least at least 2, at least 5, at least 10, at least 20, at least 30, at least 50, at least 100 target RNA molecules, from 2-10, 200-100, 50-500, or 50-2000 pairs of forward and reverse PCR primers.

In one aspect, the disclosure herein relates to a method of preparing a composition of amplified complimentary DNA (cDNA) from RNA extracted from a urine sample of a kidney transplant recipient useful for determination of kidney transplant rejection, comprising: (a) extracting RNA from the urine sample of the kidney transplant recipient; (b) preparing a composition of amplified cDNA derived from the extracted RNA by performing multiplex targeted amplification of the cDNA at 10-50,000 target loci in a single reaction volume to detect and quantify the amount RNA expressed from a plurality of target genes; (c) determining whether the amount RNA target loci or a function thereof exceeds a cutoff threshold indicating kidney transplant rejection.

In some embodiments, the nucleic acids comprise cellular nucleic acids, extra-cellular nucleic acids, and/or nucleic acids obtained from extracellular vesicles. In some embodiments, the method comprises isolating cells from the urine samples, and extracting nucleic acids from the cells. In some embodiments, the method further comprises isolating extracellular vesicles, and extracting nucleic acids from the extracellular vesicles. In some embodiments, the RNA is derived from extracellular vesicles (EVs) isolated from urine samples of a kidney transplant recipient.

### Methods of Determining and Monitoring Kidney Transplant Rejection Based on Measuring Protein.

In one aspect the present disclosure relates to a method of preparing a composition of protein derived from a urine sample of a kidney transplant recipient useful for determination of kidney transplant rejection, comprising: (a) extracting protein from the urine sample of the kidney recipient; (b) detecting and quantifying an amount of protein expressed from a target gene; (c) determining whether the amount of protein expressed from the target gene or a function thereof exceeds a cutoff threshold indicating kidney transplant rejection.

In one aspect the present disclosure relates to a method of preparing a composition of protein derived from extracellular vesicles (EVs) isolated from a urine sample of a kidney transplant recipient useful for determination of kidney transplant rejection, comprising: (a) extracting protein from extracellular vesicles (EVs) isolated from the urine sample of the kidney transplant recipient; (b) detecting and quantifying an amount of protein expressed from a target gene; (c) determining whether the amount of protein or a function thereof exceeds a cutoff threshold indicating kidney transplant rejection.

In one aspect the present disclosure relates to a method of administrating immunosuppressive therapy in a kidney transplant recipient, comprising: (a) measuring an amount of a protein of a target gene; and (b) titrating the dosage of an immunosuppressive therapy according to the amount of the protein or a function thereof.

In some embodiments, the method herein further comprises repeating steps (a)-(b) longitudinally for the same kidney transplant recipient, and determining a longitudinal change in the amount donor-derived protein, donor-derived target proteins, or a function thereof, and a longitudinal change in the amount of donor-derived protein, target proteins, or a function thereof.

In some embodiments, the method herein further comprises titrating the dosage of the immunosuppressive therapy according to the longitudinal change in the donor-derived protein, donor-derived target proteins, or a function thereof.

Methods of measuring protein amounts include, but are not limited to various sandwich, competitive, or non-competitive assay formats, to generate a signal that is related to the presence or amount of a protein analyte of interest. One agent for detecting a protein of the invention is e.g. an antibody capable of binding to the protein, preferably an antibody with a detectable label. Antibodies can be polyclonal, or preferably, monoclonal. An intact antibody or a fragment thereof (e.g. Fab or F(ab′)₂ can be used. The term “labeled” is intended to encompass direct labelling of the antibody by coupling a detectable substance to antibody, as well as indirect labeling of the antibody by reactivity with another reagent that is directly labeled.

A variety of formats can be employed to determine whether a sample contains a protein that binds to a given antibody. Examples of such formats include e.g. enzyme immunoassay, radioimmunoassay, Western blot analysis, and ELISA. Numerous formats for antibody arrays have been described proposed employing antibodies. Such arrays typically include different antibodies having specificity for different proteins intended to be detected. For example, usually at least one hundred different antibodies are used to detect one hundred different protein targets, each antibody being specific for one target. In some embodiments, protein amounts are measure by using mass spectrometry based approaches. In a related aspect, the invention provides arrays which contain a support or supports bearing a plurality of ligands that specifically bind to a plurality of proteins. The plurality of proteins includes at least two, three, four or five proteins determined to be indicative of a kidney rejection state. In some embodiments, the plurality of proteins are fewer than 1000 or fewer than 100 in number, and more than 100 or more than 10, respectively. In some embodiments, the plurality of ligands are attached to a planar support or to beads. In some embodiments, the ligands are different antibodies, and the different antibodies bind to different proteins of the plurality of proteins.

In some embodiments, the target proteins are encoded by the RNA targets disclosed elsewhere herein.

### Samples Comprising Nucleic Acids and Methods for Obtaining Samples and Extracting Nucleic Acids

The method disclosed herein comprises extracting fragmented or intact RNA derived from a sample obtained from a kidney recipient. In some embodiments, the method disclosed herein comprises collecting the urine sample from the transplant recipient at different times and generating the one or more transplant rejection scores from each urine sample.

In some embodiments, the urine sample is collected from the transplant recipient prior to transplantation, simultaneous with transplantation, and/or after transplantation.

In some embodiments, the risk of transplant rejection is based on two or more transplant rejection scores generated at different time points, and wherein a change in two or more transplant rejection scores indicates a change in kidney disease state.

In some embodiments, the sample is obtained from the kidney recipient less than 18 months post-transplantation, less than 17 months post-transplantation, less than 16 months post-transplantation, less than 15 months post-transplantation, less than 14 months post-transplantation, less than 13 months post-transplantation, or less than 12 months post-transplantation. In some embodiments, the sample is obtained from the transplant recipient between 0 and 2 months post-transplantation, between 2 and 4 months post-transplantation, between 4 and 6 months post-transplantation, between 6 and 9 months post-transplantation, between 9 and 12 months post-transplantation, or between 12 and 18 months post-transplantation.

In some embodiments, the sample is obtained from the kidney recipient prior to transplantation such as 1 day, 2 days, 3 days, 4, days, 5, days, 6, days or 7 days prior to transplantation. In some embodiments, the urine sample is obtained at the same day as the transplantation.

In some embodiments, the methods disclosed herein further comprise measuring the amounts of cell-free DNA, RNA, or protein longitudinally for the same kidney recipient; determining a longitudinal change in the amount of cell-free DNA, RNA, or protein. In some embodiments, the amounts of cell-free DNA, RNA, or protein is the total amount of cell-free DNA, RNA, or protein derived from the donor organ. In some embodiments, the amount of RNA or protein expressed from a target gene is measured. In some embodiments, the amount of one or more mRNAs and/or miRNAs is measured relative to a housekeeping gene.

In some embodiments, the urine samples may be extracellular vehicles, cells, or free-floating RNA, DNA or protein obtained from the urine.

### Nucleic Acids and Methods of Extracting or Enriching Nucleic Acids

The methods disclosed herein comprises extracting nucleic acids from a sample derived from a subject. The nucleic acids may be cell-free DNA, cellular DNA, DNA extracted from exosomes, cell-free RNA, cellular RNA, or RNA extracted from exosomes. The term “RNA” refers herein to any type of RNA, including messenger RNA (mRNA) or small non-coding RNA (sncRNA) such as micro RNA (miRNA). In some embodiments, wherein the extracted nucleic acids comprise one or more miRNAs.

In some embodiments, the extracted nucleic acids comprise one or more mRNAs and one or more miRNAs. In some embodiments, the RNA may be cell-free, cellular, or exosome RNA. In some embodiments, the RNA comprises small non-coding RNA (sncRNA). In some embodiments, the sncRNA comprises micro RNA (miRNA), piwi-interacting RNA (piRNA), small nucleolar RNA (snoRNA), small nuclear RNA (snRNA), or miscellaneous RNA (miscRNA). In some embodiments, the cell-free sncRNA is derived from exosomes or microvesicles.

In some embodiments, the presently disclosed method further comprises isolating cells from the urine samples, and extracting protein from the cells.

In some embodiments, the presently disclosed method further comprises isolating extracellular vesicles, and extracting protein from the extracellular vesicles.

In some embodiments, nucleic acids are extracted by using size exclusion. In some embodiments, cell-free DNA or RNA is isolated from cellular DNA or RNA based on size. In some embodiments, nucleic acids are isolated by using affinity chromatography.

In some embodiments, nucleic acids are preferentially enriched. Nucleic acids may be preferentially enriched by using preferential enrichment at a locus or target site. Such preferential enrichment refers to any method that results in the percentage of molecules of nucleic acids in a post-enrichment nucleic acid mixture that correspond to the locus being higher than the percentage of molecules of nucleic acids in the pre-enrichment nucleic acid mixture that correspond to the locus. The method may involve selective amplification of nucleic acid molecules that correspond to a locus. The method may involve removing nucleic acid molecules that do not correspond to the locus. The method may involve a combination of methods. The degree of enrichment is defined as the percentage of molecules of nucleic acids in the post-enrichment mixture that correspond to the locus or target divided by the percentage of molecules of nucleic acids in the pre-enrichment mixture that correspond to the locus or target. Preferential enrichment may be carried out at a plurality of loci. In some embodiments of the present disclosure, the degree of enrichment is greater than 20. In some embodiments of the present disclosure, the degree of enrichment is greater than 200. In some embodiments of the present disclosure, the degree of enrichment is greater than 2,000. When preferential enrichment is carried out at a plurality of loci, the degree of enrichment may refer to the average degree of enrichment of all of the loci in the set of loci.

The preferential enrichment of nucleic acids rely on the ability of primers or oligos to be hybridized to target nucleic acids or nucleic acids randomly and extended in polymerase reactions. The term “hybridization” includes a reaction in which one or more nucleic acids or polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, primer extension reaction, or the enzymatic cleavage of a polynucleotide by a ribozyme.

As used herein, the terms “hybridize” and “hybridization” refer to the annealing of a complementary sequence to the target nucleic acid, i.e., the ability of two polymers of nucleic acid (polynucleotides) containing complementary sequences to anneal through base pairing. The terms “annealed” and “hybridized” are used interchangeably throughout, and are intended to encompass any specific and reproducible interaction between a complementary sequence and a target nucleic acid, including binding of regions having only partial complementarity. Certain bases not commonly found in natural nucleic acids may be included in the nucleic acids of the present invention and include, for example, inosine and 7-deazaguanine. Those skilled in the art of nucleic acid technology can determine duplex stability empirically considering a number of variables including, for example, the length of the complementary sequence, base composition and sequence of the oligonucleotide, ionic strength and incidence of mismatched base pairs. The stability of a nucleic acid duplex is measured by the melting temperature, or “Tm”. The Tm of a particular nucleic acid duplex under specified conditions is the temperature at which on average half of the base pairs have disassociated.

Hybridization reactions can be performed under conditions of different “stringency”. The stringency of a hybridization reaction includes the difficulty with which any two nucleic acid molecules will hybridize to one another. Under stringent conditions, nucleic acid molecules at least 60%, 65%, 70%, 75%, 80%, 85%, 90%, 95%, 99%, or 100% identical to each other remain hybridized to each other, whereas molecules with low percent identity cannot remain hybridized. When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide if hybridization can occur between one of the strands of the first polynucleotide and the second polynucleotide. “Complementarity” or “homology” is quantifiable in terms of the proportion of bases in opposing strands that are expected to hydrogen bond with each other, according to generally accepted base-pairing rules.

The term “stringency” is used in reference to the conditions of temperature, ionic strength, and the presence of other compounds, under which nucleic acid hybridizations are conducted. With “high stringency” conditions, nucleic acid base pairing will occur only between nucleic acid fragments that have a high frequency of complementary base sequences. Thus, conditions of “medium” or “low” stringency are often required when it is desired that nucleic acids which are not completely complementary to one another be hybridized or annealed together. The art knows well that numerous equivalent conditions can be employed to comprise medium or low stringency conditions.

Amplification refers to a method that increases the number of copies of nucleic acid molecules. Selective Amplification may refer to a method that increases the number of copies of a particular nucleic acid molecules, or nucleic acid molecules that correspond to a particular region of nucleic acid molecules. It may also refer to a method that increases the number of copies of a particular targeted molecule of nucleic acid molecules, or targeted region of nucleic acid molecules more than it increases non-targeted molecules or regions of nucleic acid molecules.

Selective amplification may be a method of preferential enrichment. Universal Priming Sequence refers to a DNA sequence that may be appended to a population of target DNA molecules, for example by ligation, PCR, or ligation mediated PCR. Once added to the population of target molecules, primers specific to the universal priming sequences can be used to amplify the target population using a single pair of amplification primers. Universal priming sequences are typically not related to the target sequences. Universal Adapters, or ‘ligation adaptors’ or ‘library tags’ are DNA molecules containing a universal priming sequence that can be covalently linked to the 5-prime and 3-prime end of a population of target double stranded DNA molecules. The addition of the adapters provides universal priming sequences to the 5-prime and 3-prime end of the target population from which PCR amplification can take place, amplifying all molecules from the target population, using a single pair of amplification primers. Targeting refers to a method used to selectively amplify or otherwise preferentially enrich those molecules of DNA that correspond to a set of loci, in a mixture of DNA.

Particular nucleic acids may also be enriched for by using hybrid capture. In some embodiments, preferentially enriching the RNA at the plurality of biomarkers comprises: obtaining a set of hybrid capture probes; hybridizing the hybrid capture probes to the RNA in the sample; and physically separating the hybridized RNA from the sample of RNA from the unhybridized RNA from the sample. In some embodiments, preferentially enriching the sncRNA such as miRNA at the plurality of biomarkers comprises: obtaining a set of hybrid capture probes; hybridizing the hybrid capture probes to the miRNA in the sample; and physically separating the hybridized miRNA from the sample of RNA from the unhybridized RNA from the sample. In some embodiments, preferentially enriching preselected mRNA comprises: obtaining a set of hybrid capture probes; hybridizing the hybrid capture probes to the mRNA in the sample; and physically separating the hybridized mRNA from the sample of RNA from the unhybridized RNA from the sample.

In some embodiments, the term target loci refer to a particular target gene or any nucleic acid structure of interest such as genetic aberrations. Specifically, genetic aberrations include, without limitation, over-expression of a gene (e.g., an oncogene) or a panel of genes, under-expression of a gene (e.g., a tumor suppressor gene such as p53 or RB) or a panel of genes, alternative production of splice variants of a gene or a panel of genes, gene copy number variants (CNV) (e.g., DNA double minutes), nucleic acid modifications (e.g., methylation, acetylation and phosphorylation), single nucleotide polymorphisms (SNPs), chromosomal rearrangements (e.g., inversions, deletions and duplications), and mutations (insertions, deletions, duplications, missense, nonsense, synonymous or any other nucleotide changes) of a gene or a panel of genes, which mutations, in many cases, ultimately affect the activity and function of the gene products, lead to alternative transcriptional splice variants and/or changes of gene expression level, or combinations of any of the foregoing.

In some embodiments, preferentially enriching the nucleic acids in the sample at the plurality of polymorphic loci includes obtaining a plurality of pre-circularized probes where each probe targets one of the polymorphic loci, and where the 3′ and 5′ end of the probes are designed to hybridize to a region of nucleic acid sequence that is separated from the polymorphic site of the locus by a small number of bases, where the small number is 1, 2, 3, 4, 5, 6, 7, 8, 9, 10, 11, 12, 13, 14, 15, 16, 17, 18, 19, 20, 21 to 25, 26 to 30, 31 to 60, or a combination thereof, hybridizing the pre-circularized probes to nucleic acids from the sample, filling the gap between the hybridized probe ends using DNA polymerase, circularizing the pre-circularized probe, and amplifying the circularized probe.

In some embodiments, preferentially enriching the nucleic acids at the plurality of polymorphic loci includes obtaining a plurality of ligation-mediated PCR probes where each PCR probe targets one of the polymorphic loci, and where the upstream and downstream PCR probes are designed to hybridize to a region of DNA, on one strand of DNA, that is separated from the polymorphic site of the locus by a small number of bases, where the small number is 1, 2, 3, 4, 5, 6, 7, 8, 9, 10, 11, 12, 13, 14, 15, 16, 17, 18, 19, 20, 21 to 25, 26 to 30, 31 to 60, or a combination thereof, hybridizing the ligation-mediated PCR probes to the nucleic acids from the first sample, filling the gap between the ligation-mediated PCR probe ends using nucleic acids polymerase, ligating the ligation-mediated PCR probes, and amplifying the ligated ligation-mediated PCR probes.

In some embodiments, preferentially enriching the nucleic acids at the plurality of polymorphic loci includes obtaining a plurality of hybrid capture probes that target the polymorphic loci, hybridizing the hybrid capture probes to the nucleic acids in the sample and physically removing some or all of the unhybridized nucleic acids from the first sample of nucleic acids.

In some embodiments, the hybrid capture probes are designed to hybridize to a region that is flanking but not overlapping the polymorphic site. In some embodiments, the hybrid capture probes are designed to hybridize to a region that is flanking but not overlapping the polymorphic site, and where the length of the flanking capture probe may be selected from the group consisting of less than about 120 bases, less than about 110 bases, less than about 100 bases, less than about 90 bases, less than about 80 bases, less than about 70 bases, less than about 60 bases, less than about 50 bases, less than about 40 bases, less than about 30 bases, and less than about 25 bases. In some embodiments, the hybrid capture probes are designed to hybridize to a region that overlaps the polymorphic site, and where the plurality of hybrid capture probes comprise at least two hybrid capture probes for each polymorphic loci, and where each hybrid capture probe is designed to be complementary to a different allele at that polymorphic locus.

In some embodiments, preferentially enriching the nucleic acids at a plurality of target or polymorphic loci includes obtaining a plurality of inner forward primers where each primer targets one of the target or polymorphic loci, and where the 3′ end of the inner forward primers are designed to hybridize to a region of DNA upstream from the nucleic acids or polymorphic site, and separated from the polymorphic site by a small number of bases, where the small number is selected from the group consisting of 1, 2, 3, 4, 5, 6 to 10, 11 to 15, 16 to 20, 21 to 25, 26 to 30, or 31 to 60 base pairs, optionally obtaining a plurality of inner reverse primers where each primer targets one of the target loci or polymorphic loci, and where the 3′ end of the inner reverse primers are designed to hybridize to a region of nucleic acids upstream from the target or polymorphic site, and separated from the target loci or polymorphic site by a small number of bases, where the small number is selected from the group consisting of 1, 2, 3, 4, 5, 6 to 10, 11 to 15, 16 to 20, 21 to 25, 26 to 30, or 31 to 60 base pairs, hybridizing the inner primers to the nucleic acids, and amplifying the nucleic acids using the polymerase chain reaction to form amplicons.

In some embodiments, the method also includes obtaining a plurality of outer forward primers where each primer targets one of the polymorphic loci, and where the outer forward primers are designed to hybridize to the region of nucleic acids upstream from the inner forward primer, optionally obtaining a plurality of outer reverse primers where each primer targets one of the polymorphic loci, and where the outer reverse primers are designed to hybridize to the region of nucleic acids immediately downstream from the inner reverse primer, hybridizing the first primers to the nucleic acids, and amplifying the nucleic acids using the polymerase chain reaction.

In some embodiments, the method also includes obtaining a plurality of outer reverse primers where each primer targets one of the polymorphic loci, and where the outer reverse primers are designed to hybridize to the region of nucleic acids immediately downstream from the inner reverse primer, optionally obtaining a plurality of outer forward primers where each primer targets one of the polymorphic loci, and where the outer forward primers are designed to hybridize to the region of nucleic acids upstream from the inner forward primer, hybridizing the first primers to the nucleic acids, and amplifying the DNA using the polymerase chain reaction.

In some embodiments, preparing the first sample further includes appending universal adapters to the nucleic acids in the first sample and amplifying the nucleic acids in the first sample using the polymerase chain reaction. In some embodiments, at least a fraction of the amplicons that are amplified are less than 100 bp, less than 90 bp, less than 80 bp, less than 70 bp, less than 65 bp, less than 60 bp, less than 55 bp, less than 50 bp, or less than 45 bp, and where the fraction is 10%, 20%, 30%, 40%, 50%, 60%, 70%, 80%, 90%, or 99%.

In some embodiments, amplifying the nucleic acids is done in one or a plurality of individual reaction volumes, and where each individual reaction volume contains more than 100 different forward and reverse primer pairs, more than 200 different forward and reverse primer pairs, more than 500 different forward and reverse primer pairs, more than 1,000 different forward and reverse primer pairs, more than 2,000 different forward and reverse primer pairs, more than 5,000 different forward and reverse primer pairs, more than 10,000 different forward and reverse primer pairs, more than 20,000 different forward and reverse primer pairs, more than 50,000 different forward and reverse primer pairs, or more than 100,000 different forward and reverse primer pairs.

In some embodiments, preparing the sample further comprises dividing the sample into a plurality of portions, and where the nucleic acids in each portion is preferentially enriched at a subset of the plurality of polymorphic loci. In some embodiments, the inner primers are selected by identifying primer pairs likely to form undesired primer duplexes and removing from the plurality of primers at least one of the pair of primers identified as being likely to form undesired primer duplexes. In some embodiments, the inner primers contain a region that is designed to hybridize either upstream or downstream of the targeted polymorphic locus, and optionally contain a universal priming sequence designed to allow PCR amplification. In some embodiments, at least some of the primers additionally contain a random region that differs for each individual primer molecule. In some embodiments, at least some of the primers additionally contain a molecular barcode.

In some embodiments, the method comprises: (a) performing multiplex polymerase chain reaction (PCR) on a nucleic acid sample comprising target loci to simultaneously amplify at least 1,000 distinct target loci using either (i) at least 1,000 different primer pairs, or (ii) at least 1,000 target-specific primers and a universal or tag-specific primer, in a single reaction volume to produce amplified products comprising target amplicons; and (b) sequencing the amplified products. In some embodiments, the method does not comprise using a microarray.

In some embodiments, the method comprises (a) performing multiplex polymerase chain reaction (PCR) on the cell free DNA sample comprising target loci to simultaneously amplify at least 1,000 distinct target loci using either (i) at least 1,000 different primer pairs, or (ii) at least 1,000 target-specific primers and a universal or tag-specific primer, in a single reaction volume to produce amplified products comprising target amplicons; and b) sequencing the amplified products. In some embodiments, the method does not comprise using a microarray.

In some embodiments, mRNA is isolated by using probes that hybridize to the poly-A tail of the mRNA molecules.

### Target Genes and Loci and Protein Targets

The nucleic acids may comprise target loci or target genes indicative of an immune response, or various diseases or conditions as described elsewhere herein. In some embodiments, the target loci comprise one or more different sets of target loci. In some embodiments, the target loci comprises a set of target genes associated with a kidney rejection state such as non-rejection, T-cell mediated rejection (TCMR), antibody-mediated rejection (ABMR), or a mixed TCMR and ABMR disease state. In some embodiments, the TCMR further comprises molecularly defined TCMR (mTCMR) and/or possible TCMR (pTCMR). In some embodiments, the ABMR further comprises molecularly defined ABMR (mABMR) and/or possible ABMR (pABMR).

In some embodiments the target genes, or sets of target genes (including mRNA, miRNA or protein expressed by or associated with said target genes) are associated with inflammation, allograft rejection, T cell activation and/or differentiation, B cell activation and/or differentiation, a cytokine response, and/or a chemokine response. In some embodiments the target genes, or sets of target genes (including mRNA, miRNA or protein expressed by or associated with said target genes) are associated with apoptosis.

In some embodiments, one or more mRNAs and/or miRNAs are associated with antibody mediated transplant rejection (AMTR), T-cell mediated transplant rejection (TMTR), apoptosis pathways, cytokine, antimicrobial responses, and/or inflammatory cellular responses.

In some embodiments, the one or more mRNAs and/or miRNAs are associated with the antimicrobial responses are CXC motif chemokine ligand (CXCL) type genes.

In some embodiments, the one or more mRNAs are expressed from a gene selected from the group consisting of:

| | | | | |
|---|---|---|---|---|
| ITM2A | SLAMF6 | IKZF3 | CCL5 | CXCL9 |
| IGHM | ITM2A | HIST1H2AJ | CXCL10 | CD3E |
| NKG7 | TRBC2 | HIST1H3I | TRGC2 | HIST1H1B |
| CTLA4 | PDCD1 | GZMA | IL2RB | IKZF3 |
| CD69 | SIT1 | CD96 | FAIM3 | ZAP70 |
| HIST1H1D | TRAC | RNASE6 | SLAMF6 | Z98744.2 |
| GZMB | IL18R1 | IL1RL1 | HIST1H4L | C1QB |
| CD8A | HIST1H2BM | HLA-DQB1 | LCK | ASB2 |
| HIST1H2AI | SLFN12L | CD28 | TRBV28 | RP4-620F22.2 |
| GIMAP1 | ICOS | ZNF831 | TRAT1 | GIMAP7 |
| P2RY10 | HCST | HIST1H2AK | GBP5 | EMB |
| CD2 | KLRC1 | CD6 | GPR174 | AL049822.1 |
| HLA-DQA1 | TIGIT | ITK | ST8SIA4 | GIMAP6 |
| GVINP1 | HLA-DQB1- | MRC1 | HLA-DPB1 | CCR2 |
| | AS1 | | | |
| CCND2-AS1 | AL009179.1 | EVI2B | FCGR3B | ETV7 |
| CTSW | HIST1H2AL | THEMIS | CH17- | BLM |
| | | | 373J23.1 | |
| PRF1 | DOK2 | CXCR6 | NGFR | KCNE1 |
| HIST1H3H | CTD- | HLA-DRA | CD8B | NCF1 |
| | 2313F11.1 | | | |
| GZMK | MLKL | HIST1H3B | CD3G | JAK3 |
| CD3D | C1QA | IFNG | CDCA7 | Z98744.3 |
| CXCL11 | RP11- | HLA-H | TOX | GIMAP4 |
| | 284N8.3 | | | |
| FAM78A | FAM65B | HCG4P7 | SMAP2 | EOMES |
| RASAL3 | CD163 | FCRL3 | IFIT2 | SH2D1A |
| HCG4P11 | RGS18 | MIR155HG | IFITM1 | HIST1H2BL |
| ITGA4 | RP11- | MATK | KCNJ2 | KLRB1 |
| | 463J10.3 | | | |
| HIST1H2AH | APOBEC3G | PSMB9 | PTPRC | RCSD1 |
| PYHIN1 | BTN3A3 | AC007278.2 | C11orf21 | RHOH |
| TMEM156 | HIST2H3D | NLRP3 | AF001548.5 | HIST1H2BI |
| CD74 | AGAP2 | CD52 | GBP1 | GMFG |
| LAX1 | SASH3 | GNLY | HIST2H2AB | MS4A6A |
| PDE3B | SLAMF8 | AGER | PREX1 | IRF8 |
| IKZF1 | TBC1D10C | FCGR3A | BCL11B | HLA-DRB1 |
| CD97 | CTB-186G2.1 | NLRC3 | TAP1 | CD247 |
| HIST1H3G | GBP4 | RP11-1049A21.2 | TMC8 | SELPLG |
| HIST1H2BO | CCND2 | CST7 | CCR5 | RP11- |
| | | | | 264B17.5 |
| GRAP2 | HLA-B | IKBIP | KRT1 | SERPINB12 |
| ALB | TGM3 | HS3ST6 | NRTN | FAM180A |
| FALEC | FGF22 | HCN2 | SLC24A3 | RP11- |
| | | | | 167H9.5 |
| C1QB | IL18R1 | KCNE1 | CCR2 | C1QA |
| CD163 | CST7 | SLC1A3 | RNASE6 | HIST1H1B |
| TRGC2 | MS4A6A | C1QC | RGS1 | MSR1 |
| HIST1H1D | HIST1H3I | CD28 | LSM11 | HIST1H3B |
| HIST1H2AJ | GIMAP6 | DAAM2 | CCL5 | CTLA4 |
| IGHG1 | C1orf162 | MIR155HG | ITM2A | GIMAP4 |
| HIST1H2BM | HIST1H3G | FCER1G | FKBP5 | IL1R2 |
| GZMA | CLEC12A | LINC01127 | Z98744.2 | GPR34 |
| HLA-DMB | HIST1H4L | RRM2 | TREM2 | GIMAP8 |
| CD3E | AL049822.1 | HLA-DPB1 | LMNB1 | CD300C |
| IFITM1 | BLM | SAMSN1 | IRF8 | CLSPN |
| IL1RL1 | SDS | RP11-274E7.2 | MS4A4A | PDCD1 |
| ICOS | RP11- | TNFSF13B | HIST1H3F | RP11- |
| | 848G14.5 | | | 777B9.5 |
| GIMAP2 | IL18RAP | VSIG4 | GIMAP7 | SRGN |
| TRBC2 | LIPA | SERPINE1 | CENPK | IL10 |
| GZMK | DTL | GLDN | SMAP2 | HIST1H2BB |
| SIT1 | IFITM3 | CTD-2033D15.2 | HLA-DRA | THBS1 |
| ADORA3 | STAB1 | AIM2 | EVI2A | TFPI2 |
| EMB | SLA | CD8A | RCSD1 | GIMAP1 |
| FAM105A | FPR2 | CD180 | RP11- | RP4- |
| | | | 463J10.3 | 737E23.5 |
| FCGR3A | HIST1H2BL | CD1D | UBE2C | PDK4 |
| MLKL | RP11-7F17.3 | TLR7 | PTPRC | KBTBD8 |
| CD96 | HIST1H2AI | FAIM3 | CD48 | IFI27L2 |
| HIST1H4F | CTD- | SLC16A6 | LRRC8C | NLRP6 |
| | 2033D15.3 | | | |
| TLR8 | IRAK3 | LY96 | LILRA5 | AL009179.1 |
| KIAA0101 | GCA | CCDC109B | CTSL | CCR5 |
| BTN3A2 | STAMBPL1 | HLA-DPA1 | CTD- | LAIR1 |
| | | | 2313F11.1 | |
| FES | RNASE1 | CXCL10 | TRIM22 | HIST1H3H |
| CD52 | MYBL2 | RP11-403P17.6 | RNU4-1 | KLRC1 |
| FEN1 | MIR181A1HG | HCG27 | RP11- | BIN2 |
| | | | 335I12.2 | |
| RP4- | GBP5 | FYB | PCED1B | AL031777.1 |
| 620F22.2 | | | | |
| MMD | DOK2 | RP11-404F10.2 | PPT1 | FAM65B |
| GVINP1 | SERPINF1 | CARD16 | CEP55 | HIST1H2AL |
| CD86 | CD2 | JAK3 | SELPLG | AC007278.3 |
| FAM111B | GYG1 | NLRP3 | ZWINT | ITGA4 |
| TRAC | SELL | GPR183 | TRG-AS1 | ST8SIA4 |
| NCKAP1L | STK17B | OLAH | APOC1 | PCED1B-AS1 |
| SLFN13 | TIGIT | ATHL1 | HIST1H2BI | HLA-DMA |
| ENTPD1 | SLCO2B1 | IFNG | CD300LF | F13A1 |
| NEIL3 | TMEM14E | ACSL4 | RN7SKP48 | GZMH |
| LYAR | GPNMB | IKZF1 | RP3-455J7.4 | SCIMP |
| AMIGO2 | TLR5 | EXOC6 | IFITM2 | HAVCR2 |
| TLR2 | CTB- | FGL2 | A2M | LY9 |
| | 111H14.1 | | | |
| KIAA0825 | WDR76 | LILRB5 | SLAMF6 | CTB-41I6.2 |
| FGR | HELLS | CD274 | HIST1H2AB | MELK |
| ASB2 | SIGLEC9 | TPK1 | MAP2K6 | RP11- |
| | | | | 347P5.1 |
| HIST1H2AH | EVI2B | CTA-373H7.7 | FAM26F | KLRB1 |
| TRPV2 | CHIT1 | HTATSF1P2 | TSC22D3 | TLDC2 |
| MGAM | RP11-69L16.4 | CXCR6 | STAT4 | TNFSF8 |
| MB21D1 | SLFN11 | RP11-124N14.3 | TYROBP | ITGB2 |
| LILRB4 | CDK1 | PSMB8 | HIST2H2AB | DNA2 |
| PDCD1LG2 | CCL18 | RP11-25K21.6 | HLA-DQA1 | CELF2 |
| AIF1 | LBR | AL021807.1 | GMFG | PTGER2 |
| VIM | CASP1 | HCG4P11 | RP4- | HLA-DQB1 |
| | | | 671O14.5 | |
| ZNF367 | VNN1 | RP11-278C7.5 | HCST | SLC46A3 |
| SLAMF8 | HLA-H | CARD8-AS1 | RP1-68D18.2 | CTD- |
| | | | | 2033D15.1 |
| NCAPG | SMARCD3 | MMP19 | ARHGAP15 | FABP5P7 |
| HLA-DQB1- | HIST1H2BO | FCGR2A | ITGAM | MCM6 |
| AS1 | | | | |
| VCAN | CD37 | BTLA | Z98744.3 | NMI |
| RP11- | MARCH1 | RP11-290C10.1 | VNN2 | HLA-DOA |
| 1049A21.2 | | | | |
| CD300E | RGS2 | HIST1H3J | NCAPH | HIST1H2BE |
| HLA-B | CD4 | MSNP1 | CTSC | CXCL5 |
| AC008984.2 | HIST1H2AE | APMAP | CD84 | SHC4 |
| GLIPR2 | ZPLD1 | GPR171 | FAM101B | C5AR1 |
| UBASH3B | FAM78A | SLED1 | CRTAM | PMP22 |
| ZEB2-AS1 | CTC-301O7.4 | PFN1P1 | EPSTI1 | HK3 |
| C18orf54 | ATP8A1 | C11orf21 | LCP1 | FAM198B |
| RP11- | ETV7 | WAS | RP11-415J8.3 | CYBB |
| 326C3.2 | | | | |
| CDCA7L | CMSS1 | RBL1 | FLI1 | RNF175 |
| IL27RA | CSF1R | TMX1 | FAR2 | HAUS1 |
| AOAH | AQP9 | ARHGEF6 | AC007278.2 | CLEC2B |
| CSGALNACT2 | LILRB1 | ITK | LINC01093 | P2RY14 |
| FPR1 | PLK4 | RP11-488L18.10 | MDM1 | PREX1 |
| RP11- | RP11-256L6.3 | RP6-159A1.4 | CD226 | CD300LB |
| 186N15.3 | | | | |
| SNX10 | CEP85L | CLEC10A | LTB4R | MYC |
| CENPH | FXYD4 | TRAF5 | CIITA | SOAT1 |
| RP11- | AGAP2 | NAIP | PLP2 | PSMB8-AS1 |
| 325F22.2 | | | | |
| ETS1 | IRF4 | NCF1 | E2F8 | CD33 |
| BTNL8 | CLEC4E | POLR3GL | IKZF3 | IL2RA |
| GFI1 | HHEX | SNX20 | APBB1IP | RP11-15A1.7 |
| DCK | SNORA12 | EMR1 | APOBEC3G | ANXA2R |
| ANKRD22 | AC025048.1 | MS4A7 | MSN | FABP5 |
| PYHIN1 | GPR141 | RP11-455F5.5 | HCG4P7 | GAB3 |
| PARVG | C17orf53 | RP11-297C4.2 | SLC4A7 | FLT3 |
| TNFAIP6 | HJURP | PATL2 | TLR4 | LAPTM5 |
| RP11- | RAB37 | MOB3C | GTF2H2C | NGFR |
| 876N24.3 | | | | |
| KIF14 | IGSF6 | TTC27 | PPP2R2B | LGALS1 |
| RP5- | IKBIP | XXbac- | AC025171.1 | CTSW |
| 1171I10.5 | | BPG299F13.14 | | |
| SLC11A1 | HGF | CNTRL | UGT1A6 | CD53 |
| RP11- | DOCK11 | VIM-AS1 | RP11- | RP11- |
| 701P16.2 | | | 272L13.4 | 488L18.4 |
| EMP3 | CCL2 | EXO5 | PLA2G7 | GPSM3 |
| LINC01272 | STAT1 | GNG2 | CR1 | SNRPF |
| TNNI2 | HCLS1 | HIST1H3C | RP11- | HENMT1 |
| | | | 214O1.2 | |
| WIPF1 | TNFSF4 | DOCK10 | ACSL1 | IL10RA |
| HIST1H2AK | CD97 | ADAMTS2 | MAD2L1 | SASH3 |
| SLC16A1 | RN7SKP203 | RAC2 | CD36 | JAM3 |
| BUB1B | RRN3 | RAB20 | CTB-41I6.1 | RNU4-2 |
| CERKL | TSPAN2 | HLA-F | CFP | ARL6IP6 |
| TNFRSF25 | CPVL | CD6 | MYADM | RP11- |
| | | | | 149I23.3 |
| HLA-DRB5 | RFC3 | FBN1 | NUSAP1 | PLXNC1 |
| NLRC4 | CTD- | GPR68 | MAP7D3 | ARG1 |
| | 2047H16.3 | | | |
| F2R | PIK3CD-AS1 | GSG2 | HS3ST3B1 | IQGAP2 |
| ALOX5AP | AC007620.3 | IDO1 | BCL2A1 | CALHM2 |
| KIF11 | TTK | LTB | FCGR1A | EEF1E1 |
| AC006460.2 | RNASE2 | KIF15 | P2RY8 | LPAR6 |
| BASP1 | BMP7 | CALHM6 | CD14 | CD46 |
| CXCL14 | ENG | FN1 | FOXP3 | IFNGR1 |
| IL18BP | IL32 | INPP5D | IRAK2 | ISG20 |
| KLRC4- | MAP4K1 | NAMPT | PRPF31 | PRPF32 |
| KLRK1 | | | | |
| PRPF33 | PSMB10 | PYCARD | RUNX3 | SERPINA1 |
| TBP | TGFB1 | | | |
| ADD1 | AIFM3 | ANKH | ANXA1 | APP |
| ATF3 | AVPR1A | BAX | BCAP31 | BCL10 |
| BCL2L1 | BCL2L10 | BCL2L11 | BCL2L2 | BGN |
| BID | BIK | BIRC3 | BMF | BMP2 |
| BNIP3L | BRCA1 | BTG2 | BTG3 | CASP1 |
| CASP2 | CASP3 | CASP4 | CASP6 | CASP7 |
| CASP8 | CASP9 | CAV1 | CCNA1 | CCND1 |
| CCND2 | CD14 | CD2 | CD38 | CD44 |
| CD69 | CDC25B | CDK2 | CDKN1A | CDKN1B |
| CFLAR | CLU | CREBBP | CTH | CTNNB1 |
| CYLD | DAP | DAP3 | DCN | DDIT3 |
| DFFA | DIABLO | DNAJA1 | DNAJC3 | DNM1L |
| DPYD | EBP | EGR3 | EMP1 | ENO2 |
| ERBB2 | ERBB3 | EREG | ETF1 | F2 |
| F2R | FAS | FASLG | FDXR | FEZ1 |
| GADD45A | GADD45B | GCH1 | GNA15 | GPX1 |
| GPX3 | GPX4 | GSN | GSR | GSTM1 |
| GUCY2D | H1F0 | HGF | HMGB2 | HMOX1 |
| HSPB1 | IER3 | IFITM3 | IFNB1 | IFNGR1 |
| IGF2R | IGFBP6 | IL18 | IL1A | IL1B |
| IL6 | IRF1 | ISG20 | JUN | KRT18 |
| LEF1 | LGALS3 | LMNA | LPPR4 | LUM |
| MADD | MCL1 | MGMT | MMP2 | NEDD9 |
| NEFH | PAK1 | PDCD4 | PDGFRB | PEA15 |
| PLAT | PLCB2 | PMAIP1 | PPP2R5B | PPP3R1 |
| PPT1 | PRF1 | PSEN1 | PSEN2 | PTK2 |
| RARA | RELA | RETSAT | RHOB | RHOT2 |
| RNASEL | ROCK1 | SAT1 | SATB1 | SC5DL |
| SLC20A1 | SMAD7 | SOD1 | SOD2 | SPTAN1 |
| SQSTM1 | TAP1 | TGFB2 | TGFBR3 | TIMP1 |
| TIMP2 | TIMP3 | TNF | TNFRSF12A | TNFSF10 |
| TOP2A | TSPO | TXNIP | VDAC2 | WEE1 |
| XIAP | | | | |
| ALB | BASP1 | BMP7 | C11orf21 | C1QB |
| C3 | CALHM6 | CD14 | CD3D | CD3E |
| CD46 | CD6 | CD74 | CDH1 | CXCL10 |
| CXCL11 | CXCL14 | CXCL9 | ENG | FN1 |
| FOXP3 | GNLY | GZMB | HAVCR2 | IFNGR1 |
| IL18BP | IL2RA | IL32 | INPP5D | IRAK2 |
| ISG20 | KLRC4- | LCK | MAP4K1 | MRC1 |
| | KLRK1 | | | |
| NAMPT | NKG7 | PRF1 | PRPF31 | PRPF32 |
| PRPF33 | PSMB10 | PSMB9 | PYCARD | RGS1 |
| RUNX3 | SERPINA1 | SERPINB12 | STAT1 | TAP1 |
| TBP | TGFB1 | TMEM156 | | |
| RBM6 | PSMB1 | ITIH4 | TP53BP1 | DFNB31 |
| LGALS1 | AP4S1 | BRF2 | RAD23B | HNRNPA2B1 |
| ZNF436 | NECAB3 | HCST | AHDC1 | ARPP19 |
| C3orf20 | ACTR3B | UBAC2 | SETDB2 | STX17 |
| RAB30 | RPLP1 | SNRPF | CMTM3 | USP21 |
| LRIG1 | GTF2H2 | MPP7 | MERTK | ANGPT1 |
| AK9 | ITLN2 | CACHD1 | SYVN1 | ELF3 |
| UCN | ZMYM6 | BDH2 | DACT1 | COPS6 |
| 2-Sep | ZNF354A | BTD | SIGLEC17P | TADA3 |
| CTSF | LINC00469 | DMAP1 | DCTPP1 | MFSD6L |
| ADAMTSL5 | SUPT5H | S100A5 | ARMCX4 | ZNF502 |
| ZNF138 | ZNF841 | FICD | COL5A2 | RP11-15J10.1 |
| GNRHR2 | TRDC | AC016747.3 | AGGF1P1 | CHCHD3P3 |
| PRPF38AP1 | RP11- | AC010894.3 | RPL4P6 | AP000936.1 |
| | 242O24.3 | | | |
| LINC01278 | LINC00298 | AC159540.14 | LINC00299 | AMY2B |
| RPL34P18 | RP11- | RPP21 | RP11- | RP11- |
| | 1008M1.1 | | 20O24.4 | 392P7.6 |
| CDK11B | RP1-16A9.1 | RP11-585P4.5 | P2RX5- | RP11- |
| | | | TAX1BP3 | 644F5.11 |
| SPESP1 | RP11- | RP11-412D9.4 | RPL23AP97 | RP11- |
| | 603B24.1 | | | 529K1.2 |
| RP5- | RN7SL246P | PARD6G-AS1 | RP11- | RP11- |
| 1107A17.4 | | | 635N19.1 | 840I19.5 |
| RP11- | RP11- | LA16c-360A4.1 | F8A1 | uc_338 |
| 179B2.2 | 313P22.1 | | | |
| RP11- | RP11- | | | |
| 43N22.1 | 133M8.3 | | | |

, and combinations thereof.

In some embodiments, the one or more miRNAs are binding one or more expression products from a gene selected from the group consisting of:

| | | | | |
|---|---|---|---|---|
| ITM2A | SLAMF6 | IKZF3 | CCL5 | CXCL9 |
| IGHM | ITM2A | HIST1H2AJ | CXCL10 | CD3E |
| NKG7 | TRBC2 | HIST1H3I | TRGC2 | HIST1H1B |
| CTLA4 | PDCD1 | GZMA | IL2RB | IKZF3 |
| CD69 | SIT1 | CD96 | FAIM3 | ZAP70 |
| HIST1H1D | TRAC | RNASE6 | SLAMF6 | Z98744.2 |
| GZMB | IL18R1 | IL1RL1 | HIST1H4L | C1QB |
| CD8A | HIST1H2BM | HLA-DQB1 | LCK | ASB2 |
| HIST1H2AI | SLFN12L | CD28 | TRBV28 | RP4-620F22.2 |
| GIMAP1 | ICOS | ZNF831 | TRAT1 | GIMAP7 |
| P2RY10 | HCST | HIST1H2AK | GBP5 | EMB |
| CD2 | KLRC1 | CD6 | GPR174 | AL049822.1 |
| HLA-DQA1 | TIGIT | ITK | ST8SIA4 | GIMAP6 |
| GVINP1 | HLA-DQB1- | MRC1 | HLA-DPB1 | CCR2 |
| | AS1 | | | |
| CCND2-AS1 | AL009179.1 | EVI2B | FCGR3B | ETV7 |
| CTSW | HIST1H2AL | THEMIS | CH17- | BLM |
| | | | 373J23.1 | |
| PRF1 | DOK2 | CXCR6 | NGFR | KCNE1 |
| HIST1H3H | CTD- | HLA-DRA | CD8B | NCF1 |
| | 2313F11.1 | | | |
| GZMK | MLKL | HIST1H3B | CD3G | JAK3 |
| CD3D | C1QA | IFNG | CDCA7 | Z98744.3 |
| CXCL11 | RP11- | HLA-H | TOX | GIMAP4 |
| | 284N8.3 | | | |
| FAM78A | FAM65B | HCG4P7 | SMAP2 | EOMES |
| RASAL3 | CD163 | FCRL3 | IFIT2 | SH2D1A |
| HCG4P11 | RGS18 | MIR155HG | IFITM1 | HIST1H2BL |
| ITGA4 | RP11- | MATK | KCNJ2 | KLRB1 |
| | 463J10.3 | | | |
| HIST1H2AH | APOBEC3G | PSMB9 | PTPRC | RCSD1 |
| PYHIN1 | BTN3A3 | AC007278.2 | C11orf21 | RHOH |
| TMEM156 | HIST2H3D | NLRP3 | AF001548.5 | HIST1H2BI |
| CD74 | AGAP2 | CD52 | GBP1 | GMFG |
| LAX1 | SASH3 | GNLY | HIST2H2AB | MS4A6A |
| PDE3B | SLAMF8 | AGER | PREX1 | IRF8 |
| IKZF1 | TBC1D10C | FCGR3A | BCL11B | HLA-DRB1 |
| CD97 | CTB-186G2.1 | NLRC3 | TAP1 | CD247 |
| HIST1H3G | GBP4 | RP11-1049A21.2 | TMC8 | SELPLG |
| HIST1H2BO | CCND2 | CST7 | CCR5 | RP11- |
| | | | | 264B17.5 |
| GRAP2 | HLA-B | IKBIP | KRT1 | SERPINB12 |
| ALB | TGM3 | HS3ST6 | NRTN | FAM180A |
| FALEC | FGF22 | HCN2 | SLC24A3 | RP11- |
| | | | | 167H9.5 |
| C1QB | IL18R1 | KCNE1 | CCR2 | C1QA |
| CD163 | CST7 | SLC1A3 | RNASE6 | HIST1H1B |
| TRGC2 | MS4A6A | C1QC | RGS1 | MSR1 |
| HIST1H1D | HIST1H3I | CD28 | LSM11 | HIST1H3B |
| HIST1H2AJ | GIMAP6 | DAAM2 | CCL5 | CTLA4 |
| IGHG1 | C1orf162 | MIR155HG | ITM2A | GIMAP4 |
| HIST1H2BM | HIST1H3G | FCER1G | FKBP5 | IL1R2 |
| GZMA | CLEC12A | LINC01127 | Z98744.2 | GPR34 |
| HLA-DMB | HIST1H4L | RRM2 | TREM2 | GIMAP8 |
| CD3E | AL049822.1 | HLA-DPB1 | LMNB1 | CD300C |
| IFITM1 | BLM | SAMSN1 | IRF8 | CLSPN |
| IL1RL1 | SDS | RP11-274E7.2 | MS4A4A | PDCD1 |
| ICOS | RP11- | TNFSF13B | HIST1H3F | RP11- |
| | 848G14.5 | | | 777B9.5 |
| GIMAP2 | IL18RAP | VSIG4 | GIMAP7 | SRGN |
| TRBC2 | LIPA | SERPINE1 | CENPK | IL10 |
| GZMK | DTL | GLDN | SMAP2 | HIST1H2BB |
| SIT1 | IFITM3 | CTD-2033D15.2 | HLA-DRA | THBS1 |
| ADORA3 | STAB1 | AIM2 | EVI2A | TFPI2 |
| EMB | SLA | CD8A | RCSD1 | GIMAP1 |
| FAM105A | FPR2 | CD180 | RP11- | RP4- |
| | | | 463J10.3 | 737E23.5 |
| FCGR3A | HIST1H2BL | CD1D | UBE2C | PDK4 |
| MLKL | RP11-7F17.3 | TLR7 | PTPRC | KBTBD8 |
| CD96 | HIST1H2AI | FAIM3 | CD48 | IFI27L2 |
| HIST1H4F | CTD- | SLC16A6 | LRRC8C | NLRP6 |
| | 2033D15.3 | | | |
| TLR8 | IRAK3 | LY96 | LILRA5 | AL009179.1 |
| KIAA0101 | GCA | CCDC109B | CTSL | CCR5 |
| BTN3A2 | STAMBPL1 | HLA-DPA1 | CTD- | LAIR1 |
| | | | 2313F11.1 | |
| FES | RNASE1 | CXCL10 | TRIM22 | HIST1H3H |
| CD52 | MYBL2 | RP11-403P17.6 | RNU4-1 | KLRC1 |
| FEN1 | MIR181A1HG | HCG27 | RP11- | BIN2 |
| | | | 335I12.2 | |
| RP4- | GBP5 | FYB | PCED1B | AL031777.1 |
| 620F22.2 | | | | |
| MMD | DOK2 | RP11-404F10.2 | PPT1 | FAM65B |
| GVINP1 | SERPINF1 | CARD16 | CEP55 | HIST1H2AL |
| CD86 | CD2 | JAK3 | SELPLG | AC007278.3 |
| FAM111B | GYG1 | NLRP3 | ZWINT | ITGA4 |
| TRAC | SELL | GPR183 | TRG-AS1 | ST8SIA4 |
| NCKAP1L | STK17B | OLAH | APOC1 | PCED1B-AS1 |
| SLFN13 | TIGIT | ATHL1 | HIST1H2BI | HLA-DMA |
| ENTPD1 | SLCO2B1 | IFNG | CD300LF | F13A1 |
| NEIL3 | TMEM14E | ACSL4 | RN7SKP48 | GZMH |
| LYAR | GPNMB | IKZF1 | RP3-455J7.4 | SCIMP |
| AMIGO2 | TLR5 | EXOC6 | IFITM2 | HAVCR2 |
| TLR2 | CTB- | FGL2 | A2M | LY9 |
| | 111H14.1 | | | |
| KIAA0825 | WDR76 | LILRB5 | SLAMF6 | CTB-41I6.2 |
| FGR | HELLS | CD274 | HIST1H2AB | MELK |
| ASB2 | SIGLEC9 | TPK1 | MAP2K6 | RP11- |
| | | | | 347P5.1 |
| HIST1H2AH | EVI2B | CTA-373H7.7 | FAM26F | KLRB1 |
| TRPV2 | CHIT1 | HTATSF1P2 | TSC22D3 | TLDC2 |
| MGAM | RP11-69L16.4 | CXCR6 | STAT4 | TNFSF8 |
| MB21D1 | SLFN11 | RP11-124N14.3 | TYROBP | ITGB2 |
| LILRB4 | CDK1 | PSMB8 | HIST2H2AB | DNA2 |
| PDCD1LG2 | CCL18 | RP11-25K21.6 | HLA-DQA1 | CELF2 |
| AIF1 | LBR | AL021807.1 | GMFG | PTGER2 |
| VIM | CASP1 | HCG4P11 | RP4- | HLA-DQB1 |
| | | | 671O14.5 | |
| ZNF367 | VNN1 | RP11-278C7.5 | HCST | SLC46A3 |
| SLAMF8 | HLA-H | CARD8-AS1 | RP1-68D18.2 | CTD- |
| | | | | 2033D15.1 |
| NCAPG | SMARCD3 | MMP19 | ARHGAP15 | FABP5P7 |
| HLA-DQB1- | HIST1H2BO | FCGR2A | ITGAM | MCM6 |
| AS1 | | | | |
| VCAN | CD37 | BTLA | Z98744.3 | NMI |
| RP11- | MARCH1 | RP11-290C10.1 | VNN2 | HLA-DOA |
| 1049A21.2 | | | | |
| CD300E | RGS2 | HIST1H3J | NCAPH | HIST1H2BE |
| HLA-B | CD4 | MSNP1 | CTSC | CXCL5 |
| AC008984.2 | HIST1H2AE | APMAP | CD84 | SHC4 |
| GLIPR2 | ZPLD1 | GPR171 | FAM101B | C5AR1 |
| UBASH3B | FAM78A | SLED1 | CRTAM | PMP22 |
| ZEB2-AS1 | CTC-301O7.4 | PFN1P1 | EPSTI1 | HK3 |
| C18orf54 | ATP8A1 | C11orf21 | LCP1 | FAM198B |
| RP11- | ETV7 | WAS | RP11-415J8.3 | CYBB |
| 326C3.2 | | | | |
| CDCA7L | CMSS1 | RBL1 | FLI1 | RNF175 |
| IL27RA | CSF1R | TMX1 | FAR2 | HAUS1 |
| AOAH | AQP9 | ARHGEF6 | AC007278.2 | CLEC2B |
| CSGALNACT2 | LILRB1 | ITK | LINC01093 | P2RY14 |
| FPR1 | PLK4 | RP11-488L18.10 | MDM1 | PREX1 |
| RP11- | RP11-256L6.3 | RP6-159A1.4 | CD226 | CD300LB |
| 186N15.3 | | | | |
| SNX10 | CEP85L | CLEC10A | LTB4R | MYC |
| CENPH | FXYD4 | TRAF5 | CIITA | SOAT1 |
| RP11- | AGAP2 | NAIP | PLP2 | PSMB8-AS1 |
| 325F22.2 | | | | |
| ETS1 | IRF4 | NCF1 | E2F8 | CD33 |
| BTNL8 | CLEC4E | POLR3GL | IKZF3 | IL2RA |
| GFI1 | HHEX | SNX20 | APBB1IP | RP11-15A1.7 |
| DCK | SNORA12 | EMR1 | APOBEC3G | ANXA2R |
| ANKRD22 | AC025048.1 | MS4A7 | MSN | FABP5 |
| PYHIN1 | GPR141 | RP11-455F5.5 | HCG4P7 | GAB3 |
| PARVG | C17orf53 | RP11-297C4.2 | SLC4A7 | FLT3 |
| TNFAIP6 | HJURP | PATL2 | TLR4 | LAPTM5 |
| RP11- | RAB37 | MOB3C | GTF2H2C | NGFR |
| 876N24.3 | | | | |
| KIF14 | IGSF6 | TTC27 | PPP2R2B | LGALS1 |
| RP5- | IKBIP | XXbac- | AC025171.1 | CTSW |
| 1171I10.5 | | BPG299F13.14 | | |
| SLC11A1 | HGF | CNTRL | UGT1A6 | CD53 |
| RP11- | DOCK11 | VIM-AS1 | RP11- | RP11- |
| 701P16.2 | | | 272L13.4 | 488L18.4 |
| EMP3 | CCL2 | EXO5 | PLA2G7 | GPSM3 |
| LINC01272 | STAT1 | GNG2 | CR1 | SNRPF |
| TNNI2 | HCLS1 | HIST1H3C | RP11- | HENMT1 |
| | | | 214O1.2 | |
| WIPF1 | TNFSF4 | DOCK10 | ACSL1 | IL10RA |
| HIST1H2AK | CD97 | ADAMTS2 | MAD2L1 | SASH3 |
| SLC16A1 | RN7SKP203 | RAC2 | CD36 | JAM3 |
| BUB1B | RRN3 | RAB20 | CTB-41I6.1 | RNU4-2 |
| CERKL | TSPAN2 | HLA-F | CFP | ARL6IP6 |
| TNFRSF25 | CPVL | CD6 | MYADM | RP11- |
| | | | | 149I23.3 |
| HLA-DRB5 | RFC3 | FBN1 | NUSAP1 | PLXNC1 |
| NLRC4 | CTD- | GPR68 | MAP7D3 | ARG1 |
| | 2047H16.3 | | | |
| F2R | PIK3CD-AS1 | GSG2 | HS3ST3B1 | IQGAP2 |
| ALOX5AP | AC007620.3 | IDO1 | BCL2A1 | CALHM2 |
| KIF11 | TTK | LTB | FCGR1A | EEF1E1 |
| AC006460.2 | RNASE2 | KIF15 | P2RY8 | LPAR6 |
| BASP1 | BMP7 | CALHM6 | CD14 | CD46 |
| CXCL14 | ENG | FN1 | FOXP3 | IFNGR1 |
| IL18BP | IL32 | INPP5D | IRAK2 | ISG20 |
| KLRC4- | MAP4K1 | NAMPT | PRPF31 | PRPF32 |
| KLRK1 | | | | |
| PRPF33 | PSMB10 | PYCARD | RUNX3 | SERPINA1 |
| TBP | TGFB1 | | | |
| ADD1 | AIFM3 | ANKH | ANXA1 | APP |
| ATF3 | AVPR1A | BAX | BCAP31 | BCL10 |
| BCL2L1 | BCL2L10 | BCL2L11 | BCL2L2 | BGN |
| BID | BIK | BIRC3 | BMF | BMP2 |
| BNIP3L | BRCA1 | BTG2 | BTG3 | CASP1 |
| CASP2 | CASP3 | CASP4 | CASP6 | CASP7 |
| CASP8 | CASP9 | CAV1 | CCNA1 | CCND1 |
| CCND2 | CD14 | CD2 | CD38 | CD44 |
| CD69 | CDC25B | CDK2 | CDKN1A | CDKN1B |
| CFLAR | CLU | CREBBP | CTH | CTNNB1 |
| CYLD | DAP | DAP3 | DCN | DDIT3 |
| DFFA | DIABLO | DNAJA1 | DNAJC3 | DNM1L |
| DPYD | EBP | EGR3 | EMP1 | ENO2 |
| ERBB2 | ERBB3 | EREG | ETF1 | F2 |
| F2R | FAS | FASLG | FDXR | FEZ1 |
| GADD45A | GADD45B | GCH1 | GNA15 | GPX1 |
| GPX3 | GPX4 | GSN | GSR | GSTM1 |
| GUCY2D | H1F0 | HGF | HMGB2 | HMOX1 |
| HSPB1 | IER3 | IFITM3 | IFNB1 | IFNGR1 |
| IGF2R | IGFBP6 | IL18 | IL1A | IL1B |
| IL6 | IRF1 | ISG20 | JUN | KRT18 |
| LEF1 | LGALS3 | LMNA | LPPR4 | LUM |
| MADD | MCL1 | MGMT | MMP2 | NEDD9 |
| NEFH | PAK1 | PDCD4 | PDGFRB | PEA15 |
| PLAT | PLCB2 | PMAIP1 | PPP2R5B | PPP3R1 |
| PPT1 | PRF1 | PSEN1 | PSEN2 | PTK2 |
| RARA | RELA | RETSAT | RHOB | RHOT2 |
| RNASEL | ROCK1 | SAT1 | SATB1 | SC5DL |
| SLC20A1 | SMAD7 | SOD1 | SOD2 | SPTAN1 |
| SQSTM1 | TAP1 | TGFB2 | TGFBR3 | TIMP1 |
| TIMP2 | TIMP3 | TNF | TNFRSF12A | TNFSF10 |
| TOP2A | TSPO | TXNIP | VDAC2 | WEE1 |
| XIAP | | | | |
| ALB | BASP1 | BMP7 | C11orf21 | C1QB |
| C3 | CALHM6 | CD14 | CD3D | CD3E |
| CD46 | CD6 | CD74 | CDH1 | CXCL10 |
| CXCL11 | CXCL14 | CXCL9 | ENG | FN1 |
| FOXP3 | GNLY | GZMB | HAVCR2 | IFNGR1 |
| IL18BP | IL2RA | IL32 | INPP5D | IRAK2 |
| ISG20 | KLRC4- | LCK | MAP4K1 | MRC1 |
| | KLRK1 | | | |
| NAMPT | NKG7 | PRF1 | PRPF31 | PRPF32 |
| PRPF33 | PSMB10 | PSMB9 | PYCARD | RGS1 |
| RUNX3 | SERPINA1 | SERPINB12 | STAT1 | TAP1 |
| TBP | TGFB1 | TMEM156 | | |
| RBM6 | PSMB1 | ITIH4 | TP53BP1 | DFNB31 |
| LGALS1 | AP4S1 | BRF2 | RAD23B | HNRNPA2B1 |
| ZNF436 | NECAB3 | HCST | AHDC1 | ARPP19 |
| C3orf20 | ACTR3B | UBAC2 | SETDB2 | STX17 |
| RAB30 | RPLP1 | SNRPF | CMTM3 | USP21 |
| LRIG1 | GTF2H2 | MPP7 | MERTK | ANGPT1 |
| AK9 | ITLN2 | CACHD1 | SYVN1 | ELF3 |
| UCN | ZMYM6 | BDH2 | DACT1 | COPS6 |
| 2-Sep | ZNF354A | BTD | SIGLEC17P | TADA3 |
| CTSF | LINC00469 | DMAP1 | DCTPP1 | MFSD6L |
| ADAMTSL5 | SUPT5H | S100A5 | ARMCX4 | ZNF502 |
| ZNF138 | ZNF841 | FICD | COL5A2 | RP11-15J10.1 |
| GNRHR2 | TRDC | AC016747.3 | AGGF1P1 | CHCHD3P3 |
| PRPF38AP1 | RP11- | AC010894.3 | RPL4P6 | AP000936.1 |
| | 242O24.3 | | | |
| LINC01278 | LINC00298 | AC159540.14 | LINC00299 | AMY2B |
| RPL34P18 | RP11- | RPP21 | RP11- | RP11- |
| | 1008M1.1 | | 20O24.4 | 392P7.6 |
| CDK11B | RP1-16A9.1 | RP11-585P4.5 | P2RX5- | RP11- |
| | | | TAX1BP3 | 644F5.11 |
| SPESP1 | RP11- | RP11-412D9.4 | RPL23AP97 | RP11- |
| | 603B24.1 | | | 529K1.2 |
| RP5- | RN7SL246P | PARD6G-AS1 | RP11- | RP11- |
| 1107A17.4 | | | 635N19.1 | 840I19.5 |
| RP11- | RP11- | LA16c-360A4.1 | F8A1 | uc_338 |
| 179B2.2 | 313P22.1 | | | |
| RP11- | RP11- | | | |
| 43N22.1 | 133M8.3 | | | |

, and combinations thereof.

In some embodiments, the one or more mRNAs are expressed from a gene selected from the group consisting of:

| | | | | |
|---|---|---|---|---|
| ALB | BASP1 | BMP7 | C11orf21 | C1QB |
| C3 | CALHM6 | CD14 | CD3D | CD3E |
| CD46 | CD6 | CD74 | CDH1 | CXCL10 |
| CXCL11 | CXCL14 | CXCL9 | ENG | FN1 |
| FOXP3 | GNLY | GZMB | HAVCR2 | IFNGR1 |
| IL18BP | IL2RA | IL32 | INPP5D | IRAK2 |
| ISG20 | KLRC4-KLRK1 | LCK | MAP4K1 | MRC1 |
| NAMPT | NKG7 | PRF1 | PRPF31 | PRPF32 |
| PRPF33 | PSMB10 | PSMB9 | PYCARD | RGS1 |
| RUNX3 | SERPINA1 | SERPINB12 | STAT1 | TAP1 |
| TBP | TGFB1 | TMEM156 | | |

, and combinations thereof.

In some embodiments, the one or more miRNAs are binding one or more mRNAs expressed from a gene selected from the group consisting of:

| | | | | |
|---|---|---|---|---|
| ALB | BASP1 | BMP7 | C11orf21 | C1QB |
| C3 | CALHM6 | CD14 | CD3D | CD3E |
| CD46 | CD6 | CD74 | CDH1 | CXCL10 |
| CXCL11 | CXCL14 | CXCL9 | ENG | FN1 |
| FOXP3 | GNLY | GZMB | HAVCR2 | IFNGR1 |
| IL18BP | IL2RA | IL32 | INPP5D | IRAK2 |
| ISG20 | KLRC4-KLRK1 | LCK | MAP4K1 | MRC1 |
| NAMPT | NKG7 | PRF1 | PRPF31 | PRPF32 |
| PRPF33 | PSMB10 | PSMB9 | PYCARD | RGS1 |
| RUNX3 | SERPINA1 | SERPINB12 | STAT1 | TAP1 |
| TBP | TGFB1 | TMEM156 | | |

, and combinations thereof.

In some embodiments, the one or more mRNAs are expressed from a gene selected from the group consisting of:

| | | | | | | | | | |
|---|---|---|---|---|---|---|---|---|---|
| AD | AIF | AN | ANXA | APP | ATF | AVP | BA | BC | BC |
| BCL | BC | BC | BCL2 | BG | BID | BIK | BIR | BM | BM |
| BNI | BR | BT | BTG3 | CA | CAS | CAS | CA | CA | CA |
| CA | CA | CA | CCNA | CC | CCN | CD1 | CD | CD | CD |
| CD | CD | CD | CDKN | CD | CFL | CLU | CR | CT | CT |
| CYL | DA | DA | DCN | DDI | DFF | DIA | DN | DN | DN |
| DP | EB | EG | EMP1 | EN | ERB | ERB | ER | ETF | F2 |
| F2R | FA | FA | FDXR | FEZ | GAD | GAD | GC | GN | GP |
| GP | GP | GS | GSR | GS | GUC | H1F | HG | HM | HM |
| HS | IER | IFIT | IFNB1 | IFN | IGF2 | IGF | IL1 | IL1 | IL1 |
| IL6 | IRF | ISG | JUN | KRT | LEF | LGA | LM | LPP | LU |
| MA | MC | MG | MMP | NE | NEF | PAK | PD | PD | PE |
| PLA | PL | PM | PPP2 | PPP | PPT | PRF | PS | PS | PT |
| RA | RE | RE | RHO | RH | RNA | ROC | SA | SA | SC |
| SLC | SM | SO | SOD2 | SPT | SQS | TAP | TG | TG | TIM |
| TIM | TIM | TN | TNFR | TNF | TOP | TSP | TX | VD | WE |
| XIA | | | | | | | | | |

, and combinations thereof.

In some embodiments, the one or more miRNAs are binding one or more mRNAs expressed from a gene selected from the group consisting of:

| | | | | | | | | | |
|---|---|---|---|---|---|---|---|---|---|
| AD | AIF | AN | ANXA | APP | ATF | AVP | BA | BC | BC |
| BCL | BCL | BCL | BCL2 | BG | BID | BIK | BIR | BM | BM |
| BNI | BR | BT | BTG3 | CAS | CAS | CAS | CA | CA | CA |
| CAS | CA | CA | CCNA | CC | CCN | CD1 | CD | CD | CD |
| CD6 | CD | CD | CDKN | CD | CFL | CLU | CR | CT | CT |
| CYL | DA | DA | DCN | DDI | DFF | DIA | DN | DN | DN |
| DPY | EB | EG | EMP1 | EN | ERB | ERB | ER | ETF | F2 |
| F2R | FAS | FAS | FDXR | FEZ | GAD | GAD | GC | GN | GP |
| GP | GP | GS | GSR | GST | GUC | H1F | HG | HM | HM |
| HSP | IER | IFIT | IFNB1 | IFN | IGF2 | IGF | IL18 | IL1 | IL1 |
| IL6 | IRF | ISG | JUN | KRT | LEF | LGA | LM | LPP | LU |
| MA | MC | MG | MMP2 | NE | NEF | PAK | PD | PD | PE |
| PLA | PLC | PM | PPP2 | PPP | PPT | PRF | PS | PS | PT |
| RA | REL | RE | RHOB | RH | RNA | ROC | SAT | SAT | SC |
| SLC | SM | SO | SOD2 | SPT | SQS | TAP | TG | TG | TIM |
| TIM | TIM | TNF | TNFR | TNF | TOP | TSP | TX | VD | WE |
| XIA | | | | | | | | | |

, and combinations thereof.

In some embodiments, the one or more mRNAs are expressed from a gene selected from the group consisting of: CXCL9, CD3ϵ, IP-10, LCK, C1QB, PSMB9, Tim-3, Foxp3, FAM26F, ALB, CTB-186G2.1, CXCL11, GNLY, MRC1, RGS1, RP11-1049A21.2, TMEM156, Z98744.3, SERPINB12, C11orf21, AF001548, 18s ribosomal, Basp1, Cd6, Cxcl10, Cxcl9, Inpp5d, Isg20, Lck, Nkg7, Runx3, Tap1, GZMB, PRF1, CD74, IL32, STAT1, CXCL14, SERPINA1, B2M, C3, PYCARD, BMP7, TBP, NAMPT, IFNGR1, IRAK2, IL18BP, and combinations thereof.

In some embodiments, the one or more mRNAs are expressed from a gene selected from the group consisting of: CXCL9, CD3ϵ, IP-10, LCK, C1QB, PSMB9, Tim-3, Foxp3, FAM26F, ALB, CTB-186G2.1, CXCL11, GNLY, MRC1, RGS1, RP11-1049A21.2, TMEM156, Z98744.3, SERPINB12, C11orf21, AF001548, 18s ribosomal, Basp1, Cd6, Cxcl10, Cxcl9, Inpp5d, Isg20, Lck, Nkg7, Runx3, Tap1, GZMB, PRF1, CD74, IL32, STAT1, CXCL14, SERPINA1, B2M, C3, PYCARD, BMP7, TBP, NAMPT, IFNGR1, IRAK2, Calhm6, Klrc4-Klrk1, Psmb10, CD3delta, Map4k1, IL2Ra, TGFB1, FN1, CDH1, PRPF31, HAVCR2, IL18BP, and combinations thereof.

In some embodiments, the one or more mRNAs are expressed from a gene selected from the group consisting of: CXCL9, CD3ϵ, IP-10, LCK, C1QB, PSMB9, Tim-3, Foxp3, FAM26F, ALB, CTB-186G2.1, CXCL11, GNLY, MRC1, RGS1, RP11-1049A21.2, TMEM156, Z98744.3, SERPINB12, C11orf21, AF001548, 18s ribosomal, Basp1, Cd6, Cxcl10, Cxcl9, Inpp5d, Isg20, Lck, Nkg7, Runx3, Tap1, and combinations thereof.

In some embodiments, the one or more mRNAs are expressed from a gene selected from the group consisting of: CXCL11, CXCL9, GNLY, PSMB9, TAP1, CXCL10, NKG7, ISG20, RUNX3, C11orf21, C1QB, LCK, INPP5D, CD6, CD3ε, MRC1, BASP1, TMEM156, ALB, FOXP3, RGS1, SERPINB12, and combinations thereof.

In some embodiments, the one or more mRNAs are expressed from a gene selected from the group consisting of: CXCL11, CXCL9, GNLY, PSMB9, TAP1, CXCL10, NKG7, ISG20, RUNX3, C11orf21, C1QB, LCK, INPP5D, CD6, CD3ε, MRC1, BASP1, TMEM156, ALB, FOXP3, RGS1, SERPINB12, PRF1, CALHM6, KLRC4-KLRK1, CD74, GZMB, PSMB10, B2M, STAT1, CD3delta, PYCARD, IL18BP, MAP4K1, IL32, IL2RA, C3, IFNGR1, IRAK2, TGFB1, FN1, NAMPT, SERPINA1, TBP, CDH1, PRPF31, BMP7, CXCL14, HAVCR2, and combinations thereof.

In some embodiments, wherein the one or more mRNAs are not expressed from a gene selected from the group consisting of: PDCD1, MARCHF8, DCAF12, IL1R2, FLT3, ITGA4, ITGAM, PF4, C6orf25, SEMA7A, RHOU, and combinations thereof.

In some embodiments, the one or more mRNAs are not expressed from a gene selected from the group consisting of: CCDC159, dcaf12, DECR1, ERCC5, EWSR1, FLT3, GABPB2, GPI, IL1R2, ITGA4, ITGAM, MAP3K3, MAPK9, MARCHF8, NONO, PDCD1, PF4, RHOU, SEMA7A, SRRM1, TBC1D10B, TOP2B, and combinations thereof.

In some embodiments, the one or more miRNAs are selected from the group consisting of miR-186-5p, miR-665, miR-873-5p.1, miR-543, miR-330-3p, miR-362-5p/500b-5p, miR-217, miR-140-5p, miR-193-3p, miR-382-5p, miR-140-3p.2, miR-653-5p, miR-455-3p.2, miR-145-5p, miR-491-5p, miR-23-3p, miR-375, miR-129-5p, miR-96-5p/1271-5p, miR-182-5p, miR-371-5p, miR-203a-3p.1, miR-494-3p, miR-146-5p, miR-140-3p.1, miR-125-5p, miR-346, miR-760, miR-185-5p, miR-325-3p, miR-15-5p/16-5p/195-5p/424-5p/497-5p, miR-503-5p, miR-423-5p, miR-496.1, miR-155-5p, miR-142-3p.2, miR-24-3p, miR-874-3p, miR-25-3p/32-5p/92-3p/363-3p/367-3p, miR-17-5p/20-5p/93-5p/106-5p/519-3p, miR-181-5p, miR-142-5p, miR-130-3p/301-3p/454-3p, miR-21-5p/590-5p, miR-103-3p/107, miR-137, miR-340-5p, miR-490-3p, miR-143-3p, miR-409-3p, miR-27-3p, miR-138-5p, miR-485-5p, miR-328-3p, miR-326, miR-148-3p/152-3p, miR-9-5p, miR-31-5p, miR-452-5p/892-3p, miR-202-5p, miR-29-3p, miR-338-3p, miR-26-5p, let-7-5p/98-5p, miR-196-5p, miR-30-5p, miR-142-3p.1, miR-19-3p, miR-411-3p, miR-493-5p, miR-218-5p, miR-203a-3p.2, miR-495-3p, miR-425-5p, miR-135-5p, miR-154-3p/487-3p, miR-223-3p, miR-219-5p, miR-670-3p, miR-216b-5p, miR-200bc-3p/429, miR-320, miR-216a-5p, miR-141-3p/200a-3p, miR-144-3p, miR-128-3p, miR-455-3p.1, miR-219a-2-3p, miR-873-5p.2, miR-448, miR-183-5p.2, miR-374-5p, miR-505-3p.1, miR-433-3p, miR-377-3p, miR-365-3p, miR-124-3p.1, miR-410-3p, miR-199-3p, miR-22-3p, miR-129-3p, miR-383-5p.1, miR-1-3p/206, miR-296-5p, miR-299-3p, miR-212-5p, miR-331-3p, miR-378-3p, miR-136-5p, miR-1193, miR-505-3p.2, miR-302c-3p.2/520-3p, miR-421, miR-499a-5p, miR-302-3p/372-3p/373-3p/520-3p, miR-124-3p.2/506-3p, miR-34-5p/449-5p, miR-376c-3p, miR-139-5p, miR-221-3p/222-3p, miR-504-5p.1, miR-335-5p, miR-101-3p.1, miR-431-5p, miR-489-3p, miR-369-3p, miR-330-3p.2, miR-18-5p, miR-28-5p/708-5p, miR-133a-3p.2/133b, miR-205-5p, miR-199-5p, miR-455-5p, miR-126-3p.2, miR-7-5p, miR-483-3p.2, miR-668-3p, miR-1306-5p, miR-150-5p, miR-296-3p, miR-204-5p/211-5p, miR-3064-5p, miR-532-5p, miR-876-5p, miR-501-3p/502-3p, miR-33-5p, miR-153-3p, miR-214-5p, miR-655-3p, miR-342-3p, miR-133a-3p.1, miR-411-5p.1, miR-496.2, miR-411-5p.2, miR-582-5p, miR-381-3p, miR-188-5p, miR-383-5p.2, miR-486-5p, miR-183-5p.1, miR-208-3p, miR-193a-5p, miR-101-3p.2, miR-542-3p, miR-190-5p, miR-299-5p, miR-154-5p, miR-802, miR-323-3p, miR-532-3p, miR-224-5p, miR-339-5p, miR-194-5p, miR-149-5p, miR-493-3p, miR-382-3p, miR-132-3p/212-3p, miR-1197, miR-99-5p/100-5p, miR-877-5p, miR-483-3p.1, miR-10-5p, miR-361-5p, miR-539-3p, miR-191-5p, miR-329-3p/362-3p, miR-122-5p, miR-379-5p, miR-376-3p, miR-1298-5p, miR-451, miR-210-3p, miR-1224-5p, miR-324-5p, miR-544a-5p, miR-488-3p, miR-758-3p, miR-151-3p, miR-875-5p, miR-134-5p, miR-192-5p/215-5p, and miR-127-3p.

In some embodiments, the one or more miRNAs are selected from the group consisting of miR-96-5p/1271-5p, miR-493-5p, miR-183-5p.2, miR-150-5p, miR-7-5p, miR-653-5p, miR-200bc-3p/429, miR-212-5p, miR-1298-5p, miR-137, miR-758-3p, miR-325-3p, miR-542-3p, miR-25-3p/32-5p/92-3p/363-3p/367-3p, miR-495-3p, miR-30-5p, miR-873-5p.1, miR-146-5p, miR-505-3p.1, miR-539-3p, miR-216a-5p, miR-216b-5p, miR-340-5p, miR-361-5p, miR-338-3p, miR-217, miR-9-5p, miR-219a-2-3p, miR-15-5p/16-5p/195-5p/424-5p/497-5p, miR-503-5p, miR-199-3p, miR-1-3p/206, miR-17-5p/20-5p/93-5p/106-5p/519-3p, miR-302-3p/372-3p/373-3p/520-3p, miR-142-5p, miR-302c-3p.2/520-3p, miR-326, miR-760, miR-138-5p, miR-27-3p, miR-145-5p, miR-142-3p.2, miR-101-3p.2, miR-182-5p, miR-203a-3p.2, miR-140-3p.1, miR-183-5p.1, miR-144-3p, miR-101-3p.1, miR-330-3p, miR-224-5p, miR-148-3p/152-3p, miR-485-5p, miR-122-5p, miR-155-5p, miR-320, miR-23-3p, miR-124-3p.2/506-3p, miR-135-5p, miR-381-3p, miR-26-5p, miR-1224-5p, miR-192-5p/215-5p, miR-1249-3p, miR-125-5p, miR-483-3p.2, miR-668-3p, miR-223-3p, miR-655-3p, miR-382-5p, miR-130-3p/301-3p/454-3p, miR-19-3p, miR-582-5p, miR-194-5p, miR-802, miR-483-3p.1, miR-382-3p, miR-129-5p, miR-3064-5p, miR-873-5p.2, miR-499a-5p, miR-128-3p, miR-532-5p, miR-296-5p, miR-744-5p, miR-425-5p, miR-218-5p, and miR-496.1.

In some embodiments, the one or more proteins are expressed from or regulated by a gene selected from the group consisting of:

| | | | | |
|---|---|---|---|---|
| ITM2A | SLAMF6 | IKZF3 | CCL5 | CXCL9 |
| IGHM | ITM2A | HIST1H2AJ | CXCL10 | CD3E |
| NKG7 | TRBC2 | HIST1H3I | TRGC2 | HIST1H1B |
| CTLA4 | PDCD1 | GZMA | IL2RB | IKZF3 |
| CD69 | SIT1 | CD96 | FAIM3 | ZAP70 |
| HIST1H1D | TRAC | RNASE6 | SLAMF6 | Z98744.2 |
| GZMB | IL18R1 | IL1RL1 | HIST1H4L | C1QB |
| CD8A | HIST1H2BM | HLA-DQB1 | LCK | ASB2 |
| HIST1H2AI | SLFN12L | CD28 | TRBV28 | RP4-620F22.2 |
| GIMAP1 | ICOS | ZNF831 | TRAT1 | GIMAP7 |
| P2RY10 | HCST | HIST1H2AK | GBP5 | EMB |
| CD2 | KLRC1 | CD6 | GPR174 | AL049822.1 |
| HLA-DQA1 | TIGIT | ITK | ST8SIA4 | GIMAP6 |
| GVINP1 | HLA-DQB1- | MRC1 | HLA-DPB1 | CCR2 |
| | AS1 | | | |
| CCND2-AS1 | AL009179.1 | EVI2B | FCGR3B | ETV7 |
| CTSW | HIST1H2AL | THEMIS | CH17- | BLM |
| | | | 373J23.1 | |
| PRF1 | DOK2 | CXCR6 | NGFR | KCNE1 |
| HIST1H3H | CTD- | HLA-DRA | CD8B | NCF1 |
| | 2313F11.1 | | | |
| GZMK | MLKL | HIST1H3B | CD3G | JAK3 |
| CD3D | C1QA | IFNG | CDCA7 | Z98744.3 |
| CXCL11 | RP11- | HLA-H | TOX | GIMAP4 |
| | 284N8.3 | | | |
| FAM78A | FAM65B | HCG4P7 | SMAP2 | EOMES |
| RASAL3 | CD163 | FCRL3 | IFIT2 | SH2D1A |
| HCG4P11 | RGS18 | MIR155HG | IFITM1 | HIST1H2BL |
| ITGA4 | RP11- | MATK | KCNJ2 | KLRB1 |
| | 463J10.3 | | | |
| HIST1H2AH | APOBEC3G | PSMB9 | PTPRC | RCSD1 |
| PYHIN1 | BTN3A3 | AC007278.2 | C11orf21 | RHOH |
| TMEM156 | HIST2H3D | NLRP3 | AF001548.5 | HIST1H2BI |
| CD74 | AGAP2 | CD52 | GBP1 | GMFG |
| LAX1 | SASH3 | GNLY | HIST2H2AB | MS4A6A |
| PDE3B | SLAMF8 | AGER | PREX1 | IRF8 |
| IKZF1 | TBC1D10C | FCGR3A | BCL11B | HLA-DRB1 |
| CD97 | CTB-186G2.1 | NLRC3 | TAP1 | CD247 |
| HIST1H3G | GBP4 | RP11-1049A21.2 | TMC8 | SELPLG |
| HIST1H2BO | CCND2 | CST7 | CCR5 | RP11- |
| | | | | 264B17.5 |
| GRAP2 | HLA-B | IKBIP | KRT1 | SERPINB12 |
| ALB | TGM3 | HS3ST6 | NRTN | FAM180A |
| FALEC | FGF22 | HCN2 | SLC24A3 | RP11- |
| | | | | 167H9.5 |
| C1QB | IL18R1 | KCNE1 | CCR2 | C1QA |
| CD163 | CST7 | SLC1A3 | RNASE6 | HIST1H1B |
| TRGC2 | MS4A6A | C1QC | RGS1 | MSR1 |
| HIST1H1D | HIST1H3I | CD28 | LSM11 | HIST1H3B |
| HIST1H2AJ | GIMAP6 | DAAM2 | CCL5 | CTLA4 |
| IGHG1 | C1orf162 | MIR155HG | ITM2A | GIMAP4 |
| HIST1H2BM | HIST1H3G | FCER1G | FKBP5 | IL1R2 |
| GZMA | CLEC12A | LINC01127 | Z98744.2 | GPR34 |
| HLA-DMB | HIST1H4L | RRM2 | TREM2 | GIMAP8 |
| CD3E | AL049822.1 | HLA-DPB1 | LMNB1 | CD300C |
| IFITM1 | BLM | SAMSN1 | IRF8 | CLSPN |
| IL1RL1 | SDS | RP11-274E7.2 | MS4A4A | PDCD1 |
| ICOS | RP11- | TNFSF13B | HIST1H3F | RP11- |
| | 848G14.5 | | | 777B9.5 |
| GIMAP2 | IL18RAP | VSIG4 | GIMAP7 | SRGN |
| TRBC2 | LIPA | SERPINE1 | CENPK | IL10 |
| GZMK | DTL | GLDN | SMAP2 | HIST1H2BB |
| SIT1 | IFITM3 | CTD-2033D15.2 | HLA-DRA | THBS1 |
| ADORA3 | STAB1 | AIM2 | EVI2A | TFPI2 |
| EMB | SLA | CD8A | RCSD1 | GIMAP1 |
| FAM105A | FPR2 | CD180 | RP11- | RP4- |
| | | | 463J10.3 | 737E23.5 |
| FCGR3A | HIST1H2BL | CD1D | UBE2C | PDK4 |
| MLKL | RP11-7F17.3 | TLR7 | PTPRC | KBTBD8 |
| CD96 | HIST1H2AI | FAIM3 | CD48 | IFI27L2 |
| HIST1H4F | CTD- | SLC16A6 | LRRC8C | NLRP6 |
| | 2033D15.3 | | | |
| TLR8 | IRAK3 | LY96 | LILRA5 | AL009179.1 |
| KIAA0101 | GCA | CCDC109B | CTSL | CCR5 |
| BTN3A2 | STAMBPL1 | HLA-DPA1 | CTD- | LAIR1 |
| | | | 2313F11.1 | |
| FES | RNASE1 | CXCL10 | TRIM22 | HIST1H3H |
| CD52 | MYBL2 | RP11-403P17.6 | RNU4-1 | KLRC1 |
| FEN1 | MIR181A1HG | HCG27 | RP11- | BIN2 |
| | | | 335I12.2 | |
| RP4- | GBP5 | FYB | PCED1B | AL031777.1 |
| 620F22.2 | | | | |
| MMD | DOK2 | RP11-404F10.2 | PPT1 | FAM65B |
| GVINP1 | SERPINF1 | CARD16 | CEP55 | HIST1H2AL |
| CD86 | CD2 | JAK3 | SELPLG | AC007278.3 |
| FAM111B | GYG1 | NLRP3 | ZWINT | ITGA4 |
| TRAC | SELL | GPR183 | TRG-AS1 | ST8SIA4 |
| NCKAP1L | STK17B | OLAH | APOC1 | PCED1B-AS1 |
| SLFN13 | TIGIT | ATHL1 | HIST1H2BI | HLA-DMA |
| ENTPD1 | SLCO2B1 | IFNG | CD300LF | F13A1 |
| NEIL3 | TMEM14E | ACSL4 | RN7SKP48 | GZMH |
| LYAR | GPNMB | IKZF1 | RP3-455J7.4 | SCIMP |
| AMIGO2 | TLR5 | EXOC6 | IFITM2 | HAVCR2 |
| TLR2 | CTB- | FGL2 | A2M | LY9 |
| | 111H14.1 | | | |
| KIAA0825 | WDR76 | LILRB5 | SLAMF6 | CTB-41I6.2 |
| FGR | HELLS | CD274 | HIST1H2AB | MELK |
| ASB2 | SIGLEC9 | TPK1 | MAP2K6 | RP11- |
| | | | | 347P5.1 |
| HIST1H2AH | EVI2B | CTA-373H7.7 | FAM26F | KLRB1 |
| TRPV2 | CHIT1 | HTATSF1P2 | TSC22D3 | TLDC2 |
| MGAM | RP11-69L16.4 | CXCR6 | STAT4 | TNFSF8 |
| MB21D1 | SLFN11 | RP11-124N14.3 | TYROBP | ITGB2 |
| LILRB4 | CDK1 | PSMB8 | HIST2H2AB | DNA2 |
| PDCD1LG2 | CCL18 | RP11-25K21.6 | HLA-DQA1 | CELF2 |
| AIF1 | LBR | AL021807.1 | GMFG | PTGER2 |
| VIM | CASP1 | HCG4P11 | RP4- | HLA-DQB1 |
| | | | 671O14.5 | |
| ZNF367 | VNN1 | RP11-278C7.5 | HCST | SLC46A3 |
| SLAMF8 | HLA-H | CARD8-AS1 | RP1-68D18.2 | CTD- |
| | | | | 2033D15.1 |
| NCAPG | SMARCD3 | MMP19 | ARHGAP15 | FABP5P7 |
| HLA-DQB1- | HIST1H2BO | FCGR2A | ITGAM | MCM6 |
| AS1 | | | | |
| VCAN | CD37 | BTLA | Z98744.3 | NMI |
| RP11- | MARCH1 | RP11-290C10.1 | VNN2 | HLA-DOA |
| 1049A21.2 | | | | |
| CD300E | RGS2 | HIST1H3J | NCAPH | HIST1H2BE |
| HLA-B | CD4 | MSNP1 | CTSC | CXCL5 |
| AC008984.2 | HIST1H2AE | APMAP | CD84 | SHC4 |
| GLIPR2 | ZPLD1 | GPR171 | FAM101B | C5AR1 |
| UBASH3B | FAM78A | SLED1 | CRTAM | PMP22 |
| ZEB2-AS1 | CTC-301O7.4 | PFN1P1 | EPSTI1 | HK3 |
| C18orf54 | ATP8A1 | C11orf21 | LCP1 | FAM198B |
| RP11- | ETV7 | WAS | RP11-415J8.3 | CYBB |
| 326C3.2 | | | | |
| CDCA7L | CMSS1 | RBL1 | FLI1 | RNF175 |
| IL27RA | CSF1R | TMX1 | FAR2 | HAUS1 |
| AOAH | AQP9 | ARHGEF6 | AC007278.2 | CLEC2B |
| CSGALNACT2 | LILRB1 | ITK | LINC01093 | P2RY14 |
| FPR1 | PLK4 | RP11-488L18.10 | MDM1 | PREX1 |
| RP11- | RP11-256L6.3 | RP6-159A1.4 | CD226 | CD300LB |
| 186N15.3 | | | | |
| SNX10 | CEP85L | CLEC10A | LTB4R | MYC |
| CENPH | FXYD4 | TRAF5 | CIITA | SOAT1 |
| RP11- | AGAP2 | NAIP | PLP2 | PSMB8-AS1 |
| 325F22.2 | | | | |
| ETS1 | IRF4 | NCF1 | E2F8 | CD33 |
| BTNL8 | CLEC4E | POLR3GL | IKZF3 | IL2RA |
| GFI1 | HHEX | SNX20 | APBB1IP | RP11-15A1.7 |
| DCK | SNORA12 | EMR1 | APOBEC3G | ANXA2R |
| ANKRD22 | AC025048.1 | MS4A7 | MSN | FABP5 |
| PYHIN1 | GPR141 | RP11-455F5.5 | HCG4P7 | GAB3 |
| PARVG | C17orf53 | RP11-297C4.2 | SLC4A7 | FLT3 |
| TNFAIP6 | HJURP | PATL2 | TLR4 | LAPTM5 |
| RP11- | RAB37 | MOB3C | GTF2H2C | NGFR |
| 876N24.3 | | | | |
| KIF14 | IGSF6 | TTC27 | PPP2R2B | LGALS1 |
| RP5- | IKBIP | XXbac- | AC025171.1 | CTSW |
| 1171I10.5 | | BPG299F13.14 | | |
| SLC11A1 | HGF | CNTRL | UGT1A6 | CD53 |
| RP11- | DOCK11 | VIM-AS1 | RP11- | RP11- |
| 701P16.2 | | | 272L13.4 | 488L18.4 |
| EMP3 | CCL2 | EXO5 | PLA2G7 | GPSM3 |
| LINC01272 | STAT1 | GNG2 | CR1 | SNRPF |
| TNNI2 | HCLS1 | HIST1H3C | RP11- | HENMT1 |
| | | | 214O1.2 | |
| WIPF1 | TNFSF4 | DOCK10 | ACSL1 | IL10RA |
| HIST1H2AK | CD97 | ADAMTS2 | MAD2L1 | SASH3 |
| SLC16A1 | RN7SKP203 | RAC2 | CD36 | JAM3 |
| BUB1B | RRN3 | RAB20 | CTB-41I6.1 | RNU4-2 |
| CERKL | TSPAN2 | HLA-F | CFP | ARL6IP6 |
| TNFRSF25 | CPVL | CD6 | MYADM | RP11- |
| | | | | 149I23.3 |
| HLA-DRB5 | RFC3 | FBN1 | NUSAP1 | PLXNC1 |
| NLRC4 | CTD- | GPR68 | MAP7D3 | ARG1 |
| | 2047H16.3 | | | |
| F2R | PIK3CD-AS1 | GSG2 | HS3ST3B1 | IQGAP2 |
| ALOX5AP | AC007620.3 | IDO1 | BCL2A1 | CALHM2 |
| KIF11 | TTK | LTB | FCGR1A | EEF1E1 |
| AC006460.2 | RNASE2 | KIF15 | P2RY8 | LPAR6 |
| BASP1 | BMP7 | CALHM6 | CD14 | CD46 |
| CXCL14 | ENG | FN1 | FOXP3 | IFNGR1 |
| IL18BP | IL32 | INPP5D | IRAK2 | ISG20 |
| KLRC4- | MAP4K1 | NAMPT | PRPF31 | PRPF32 |
| KLRK1 | | | | |
| PRPF33 | PSMB10 | PYCARD | RUNX3 | SERPINA1 |
| TBP | TGFB1 | | | |
| ADD1 | AIFM3 | ANKH | ANXA1 | APP |
| ATF3 | AVPR1A | BAX | BCAP31 | BCL10 |
| BCL2L1 | BCL2L10 | BCL2L11 | BCL2L2 | BGN |
| BID | BIK | BIRC3 | BMF | BMP2 |
| BNIP3L | BRCA1 | BTG2 | BTG3 | CASP1 |
| CASP2 | CASP3 | CASP4 | CASP6 | CASP7 |
| CASP8 | CASP9 | CAV1 | CCNA1 | CCND1 |
| CCND2 | CD14 | CD2 | CD38 | CD44 |
| CD69 | CDC25B | CDK2 | CDKN1A | CDKN1B |
| CFLAR | CLU | CREBBP | CTH | CTNNB1 |
| CYLD | DAP | DAP3 | DCN | DDIT3 |
| DFFA | DIABLO | DNAJA1 | DNAJC3 | DNM1L |
| DPYD | EBP | EGR3 | EMP1 | ENO2 |
| ERBB2 | ERBB3 | EREG | ETF1 | F2 |
| F2R | FAS | FASLG | FDXR | FEZ1 |
| GADD45A | GADD45B | GCH1 | GNA15 | GPX1 |
| GPX3 | GPX4 | GSN | GSR | GSTM1 |
| GUCY2D | H1F0 | HGF | HMGB2 | HMOX1 |
| HSPB1 | IER3 | IFITM3 | IFNB1 | IFNGR1 |
| IGF2R | IGFBP6 | IL18 | IL1A | IL1B |
| IL6 | IRF1 | ISG20 | JUN | KRT18 |
| LEF1 | LGALS3 | LMNA | LPPR4 | LUM |
| MADD | MCL1 | MGMT | MMP2 | NEDD9 |
| NEFH | PAK1 | PDCD4 | PDGFRB | PEA15 |
| PLAT | PLCB2 | PMAIP1 | PPP2R5B | PPP3R1 |
| PPT1 | PRF1 | PSEN1 | PSEN2 | PTK2 |
| RARA | RELA | RETSAT | RHOB | RHOT2 |
| RNASEL | ROCK1 | SAT1 | SATB1 | SC5DL |
| SLC20A1 | SMAD7 | SOD1 | SOD2 | SPTAN1 |
| SQSTM1 | TAP1 | TGFB2 | TGFBR3 | TIMP1 |
| TIMP2 | TIMP3 | TNF | TNFRSF12A | TNFSF10 |
| TOP2A | TSPO | TXNIP | VDAC2 | WEE1 |
| XIAP | | | | |
| ALB | BASP1 | BMP7 | C11orf21 | C1QB |
| C3 | CALHM6 | CD14 | CD3D | CD3E |
| CD46 | CD6 | CD74 | CDH1 | CXCL10 |
| CXCL11 | CXCL14 | CXCL9 | ENG | FN1 |
| FOXP3 | GNLY | GZMB | HAVCR2 | IFNGR1 |
| IL18BP | IL2RA | IL32 | INPP5D | IRAK2 |
| ISG20 | KLRC4- | LCK | MAP4K1 | MRC1 |
| | KLRK1 | | | |
| NAMPT | NKG7 | PRF1 | PRPF31 | PRPF32 |
| PRPF33 | PSMB10 | PSMB9 | PYCARD | RGS1 |
| RUNX3 | SERPINA1 | SERPINB12 | STAT1 | TAP1 |
| TBP | TGFB1 | TMEM156 | | |
| RBM6 | PSMB1 | ITIH4 | TP53BP1 | DFNB31 |
| LGALS1 | AP4S1 | BRF2 | RAD23B | HNRNPA2B1 |
| ZNF436 | NECAB3 | HCST | AHDC1 | ARPP19 |
| C3orf20 | ACTR3B | UBAC2 | SETDB2 | STX17 |
| RAB30 | RPLP1 | SNRPF | CMTM3 | USP21 |
| LRIG1 | GTF2H2 | MPP7 | MERTK | ANGPT1 |
| AK9 | ITLN2 | CACHD1 | SYVN1 | ELF3 |
| UCN | ZMYM6 | BDH2 | DACT1 | COPS6 |
| 2-Sep | ZNF354A | BTD | SIGLEC17P | TADA3 |
| CTSF | LINC00469 | DMAP1 | DCTPP1 | MFSD6L |
| ADAMTSL5 | SUPT5H | S100A5 | ARMCX4 | ZNF502 |
| ZNF138 | ZNF841 | FICD | COL5A2 | RP11-15J10.1 |
| GNRHR2 | TRDC | AC016747.3 | AGGF1P1 | CHCHD3P3 |
| PRPF38AP1 | RP11- | AC010894.3 | RPL4P6 | AP000936.1 |
| | 242O24.3 | | | |
| LINC01278 | LINC00298 | AC159540.14 | LINC00299 | AMY2B |
| RPL34P18 | RP11- | RPP21 | RP11- | RP11- |
| | 1008M1.1 | | 20O24.4 | 392P7.6 |
| CDK11B | RP1-16A9.1 | RP11-585P4.5 | P2RX5- | RP11- |
| | | | TAX1BP3 | 644F5.11 |
| SPESP1 | RP11- | RP11-412D9.4 | RPL23AP97 | RP11- |
| | 603B24.1 | | | 529K1.2 |
| RP5- | RN7SL246P | PARD6G-AS1 | RP11- | RP11- |
| 1107A17.4 | | | 635N19.1 | 840I19.5 |
| RP11- | RP11- | LA16c-360A4.1 | F8A1 | uc_338 |
| 179B2.2 | 313P22.1 | | | |
| RP11- | RP11- | | | |
| 43N22.1 | 133M8.3 | | | |

, and combinations thereof.

In some embodiments, the one or more proteins are expressed from a gene selected from the group consisting of:

| | | | | |
|---|---|---|---|---|
| ALB | BASP1 | BMP7 | C11orf21 | C1QB |
| C3 | CALHM6 | CD14 | CD3D | CD3E |
| CD46 | CD6 | CD74 | CDH1 | CXCL10 |
| CXCL11 | CXCL14 | CXCL9 | ENG | FN1 |
| FOXP3 | GNLY | GZMB | HAVCR2 | IFNGR1 |
| IL18BP | IL2RA | IL32 | INPP5D | IRAK2 |
| ISG20 | KLRC4-KLRK1 | LCK | MAP4K1 | MRC1 |
| NAMPT | NKG7 | PRF1 | PRPF31 | PRPF32 |
| PRPF33 | PSMB10 | PSMB9 | PYCARD | RGS1 |
| RUNX3 | SERPINA1 | SERPINB12 | STAT1 | TAP1 |
| TBP | TGFB1 | TMEM156 | | |

, and combinations thereof.

In some embodiments, the one or more proteins are expressed from a gene selected from the group consisting of:

| | | | | | | | | | |
|---|---|---|---|---|---|---|---|---|---|
| AD | AIF | AN | ANXA | APP | ATF | AVP | BA | BC | BC |
| BCL | BCL | BCL | BCL2 | BG | BID | BIK | BIR | BM | BM |
| BNI | BR | BT | BTG3 | CAS | CAS | CAS | CA | CA | CA |
| CAS | CA | CA | CCNA | CC | CCN | CD1 | CD | CD | CD |
| CD6 | CD | CD | CDKN | CD | CFL | CLU | CR | CT | CT |
| CYL | DA | DA | DCN | DDI | DFF | DIA | DN | DN | DN |
| DPY | EB | EG | EMP1 | EN | ERB | ERB | ER | ETF | F2 |
| F2R | FAS | FAS | FDXR | FEZ | GAD | GAD | GC | GN | GP |
| GP | GP | GS | GSR | GST | GUC | H1F | HG | HM | HM |
| HSP | IER | IFIT | IFNB1 | IFN | IGF2 | IGF | IL18 | IL1 | IL1 |
| IL6 | IRF | ISG | JUN | KRT | LEF | LGA | LM | LPP | LU |
| MA | MC | MG | MMP2 | NE | NEF | PAK | PD | PD | PE |
| PLA | PLC | PM | PPP2 | PPP | PPT | PRF | PS | PS | PT |
| RA | REL | RE | RHOB | RH | RNA | ROC | SAT | SAT | SC |
| SLC | SM | SO | SOD2 | SPT | SQS | TAP | TG | TG | TIM |
| TIM | TIM | TNF | TNFR | TNF | TOP | TSP | TX | VD | WE |
| XIA | | | | | | | | | |

, and combinations thereof.

In some embodiments, the one or more proteins are expressed from a gene selected from the group consisting of: CXCL9, CD3ϵ, IP-10, LCK, C1QB, PSMB9, Tim-3, Foxp3, FAM26F, ALB, CTB-186G2.1, CXCL11, GNLY, MRC1, RGS1, RP11-1049A21.2, TMEM156, Z98744.3, SERPINB12, C11orf21, AF001548, 18s ribosomal, Basp1, Cd6, Cxcl10, Cxcl9, Inpp5d, Isg20, Lck, Nkg7, Runx3, Tap1, GZMB, PRF1, CD74, IL32, STAT1, CXCL14, SERPINA1, B2M, C3, PYCARD, BMP7, TBP, NAMPT, IFNGR1, IRAK2, IL18BP, and combinations thereof.

In some embodiments, the one or more proteins are expressed from a gene selected from the group consisting of: CXCL9, CD3ϵ, IP-10, LCK, C1QB, PSMB9, Tim-3, Foxp3, FAM26F, ALB, CTB-186G2.1, CXCL11, GNLY, MRC1, RGS1, RP11-1049A21.2, TMEM156, Z98744.3, SERPINB12, C11orf21, AF001548, 18s ribosomal, Basp1, Cd6, Cxcl10, Cxcl9, Inpp5d, Isg20, Lck, Nkg7, Runx3, Tap1, GZMB, PRF1, CD74, IL32, STAT1, CXCL14, SERPINA1, B2M, C3, PYCARD, BMP7, TBP, NAMPT, IFNGR1, IRAK2, Calhm6, Klrc4-Klrk1, Psmb10, CD3delta, Map4k1, IL2Ra, TGFB1, FN1, CDH1, PRPF31, HAVCR2, IL18BP, and combinations thereof.

In some embodiments, the one or more proteins are expressed from a gene selected from the group consisting of: CXCL9, CD3ϵ, IP-10, LCK, C1QB, PSMB9, Tim-3, Foxp3, FAM26F, ALB, CTB-186G2.1, CXCL11, GNLY, MRC1, RGS1, RP11-1049A21.2, TMEM156, Z98744.3, SERPINB12, C11orf21, AF001548, 18s ribosomal, Basp1, Cd6, Cxcl10, Cxcl9, Inpp5d, Isg20, Lck, Nkg7, Runx3, Tap1, and combinations thereof.

In some embodiments, the one or more proteins are expressed from a gene selected from the group consisting of: CXCL11, CXCL9, GNLY, PSMB9, TAP1, CXCL10, NKG7, ISG20, RUNX3, C11orf21, C1QB, LCK, INPP5D, CD6, CD3ε, MRC1, BASP1, TMEM156, ALB, FOXP3, RGS1, SERPINB12, and combinations thereof.

In some embodiments, the one or more proteins are expressed from a gene selected from the group consisting of: CXCL11, CXCL9, GNLY, PSMB9, TAP1, CXCL10, NKG7, ISG20, RUNX3, C11orf21, C1QB, LCK, INPP5D, CD6, CD3ε, MRC1, BASP1, TMEM156, ALB, FOXP3, RGS1, SERPINB12, PRF1, CALHM6, KLRC4-KLRK1, CD74, GZMB, PSMB10, B2M, STAT1, CD3delta, PYCARD, IL18BP, MAP4K1, IL32, IL2RA, C3, IFNGR1, IRAK2, TGFB1, FN1, NAMPT, SERPINA1, TBP, CDH1, PRPF31, BMP7, CXCL14, HAVCR2 and combinations thereof.

In some embodiments, the one or more proteins are not expressed from a gene selected from the group consisting of: PDCD1, MARCHF8, DCAF12, IL1R2, FLT3, ITGA4, ITGAM, PF4, C6orf25, SEMA7A, RHOU, and combinations thereof.

In some embodiments, the one or more proteins are not expressed from a gene selected from the group consisting of: CCDC159, dcaf12, DECR1, ERCC5, EWSR1, FLT3, GABPB2, GPI, IL1R2, ITGA4, ITGAM, MAP3K3, MAPK9, MARCHF8, NONO, PDCD1, PF4, RHOU, SEMA7A, SRRM1, TBC1D10B, TOP2B, and combinations thereof.

Samples and Methods for Isolating Nucleic Acids from the Samples

In some embodiments, the nucleic acid sample includes fragmented or digested nucleic acids. In some embodiments, the nucleic acid sample includes DNA, such as genomic DNA, cDNA, cell-free DNA (cfDNA), cell-free mitochondrial DNA (cf mDNA), cell-free DNA that originated from nuclear DNA (cf nDNA), cellular DNA, or mitochondrial DNA.

In some embodiments, nucleic acid sample includes RNA, such as cfRNA, cellular RNA, cytoplasmic RNA, coding cytoplasmic RNA, non-coding cytoplasmic RNA, mRNA, miRNA, mitochondrial RNA, rRNA, or tRNA. In some embodiments, the nucleic acid sample includes DNA from a single cell, 2 cells, 3 cells, 4 cells, 5 cells, 6 cells, 7 cells, 8 cells, 9 cell, 10 cells, or more than 10 cells. In some embodiments, the nucleic acid sample is a urine sample that is substantially free of cells. In some embodiments, the target loci are segments of human nucleic acids found in the human genome. In some embodiments, the target loci comprise or consist of single nucleotide polymorphisms (SNPs).

In some embodiments, the method includes isolating or purifying the DNA and/or RNA. There are a number of standard procedures known in the art to accomplish such an end. In some embodiments, the sample may be centrifuged to separate various layers. In some embodiments, the DNA or RNA may be isolated using filtration. In some embodiments, the preparation of the DNA or RNA may involve amplification, separation, purification by chromatography, liquid separation, isolation, preferential enrichment, preferential amplification, targeted amplification, or any of a number of other techniques either known in the art or described herein. In some embodiments for the isolation of DNA, RNase is used to degrade RNA. In some embodiments for the isolation of RNA, DNase (such as DNase I from Invitrogen, Carlsbad, Calif., USA) is used to degrade DNA. In some embodiments, an RNeasy™ mini kit (Qiagen), is used to isolate RNA according to the manufacturer's protocol. In some embodiments, small RNA molecules are isolated using the mirVana™ PARIS kit (Ambion, Austin, Tex., USA) according to the manufacturer's protocol (Gu et al., J. Neurochem. 122:641-649, 2012, which is hereby incorporated by reference in its entirety). The concentration and purity of RNA may optionally be determined using Nanovue (GE Healthcare, Piscataway, N.J., USA), and RNA integrity may optionally be measured by use of the 2100 Bioanalyzer (Agilent Technologies, Santa Clara, Calif., USA) (Gu et al., J. Neurochem. 122:641-649, 2012, which is hereby incorporated by reference in its entirety). In some embodiments, TRIZOL or RNAlater™ (Ambion) is used to stabilize RNA during storage.

In some embodiments, adaptors are added to make a sequencing library. Prior to ligation, sample DNA may be blunt ended, and then a single adenosine base is added to the 3-prime end. In some embodiments, ligation of adaptors to nucleic acids is a sticky end ligation. Prior to ligation the DNA may be cleaved using a restriction enzyme or some other cleavage method. During ligation the 3-prime adenosine of the sample fragments and the complementary 3-prime tyrosine overhang of adaptor can enhance ligation efficiency. In some embodiments, adaptor ligation is performed using the ligation kit found in the AGILENT SURESELECT™ kit.

In some embodiments, the library is amplified using universal primers. In an embodiment, the amplified library is fractionated by size separation or by using products such as AGENCOURT AMPURE™ beads or other similar methods. In some embodiments, PCR amplification is used to amplify target loci. In some embodiments, the amplified DNA is sequenced (such as sequencing using an ILLUMINA IIGAX™ or HiSeq sequencer). In some embodiments, the amplified DNA is sequenced from each end of the amplified DNA to reduce sequencing errors. If there is a sequence error in a particular base when sequencing from one end of the amplified DNA, there is less likely to be a sequence error in the complementary base when sequencing from the other side of the amplified DNA (compared to sequencing multiple times from the same end of the amplified DNA).

In some embodiments, miRNA can be separated from fragments of RNA caused by degradation because degraded RNA has lost phosphorylation groups at the ends. The miRNA retains the phosphorylation groups at the ends. An adapter can be ligated to phosphorylated miRNA ends, but the adaptor will not ligate to unphosphorylated RNA species such as degraded mRNA. The adaptor can contain sequences that allow for primer binding to aid reverse transcription to produce complementary DNA (cDNA) selectively from the RNA molecules produced by a target gene of interest.

As nonlimiting examples, a locus can be a single nucleotide polymorphism, an intron, or an exon. In some embodiments, a locus can include an insertion, deletion, or transposition.

In some embodiments, free floating DNA or RNA is isolated. Free floating or cell-free DNA is typically present in fragments about 160 nucleotides in length. In some embodiments, the free-floating DNA is isolated using an EDTA-2Na tube after removal of cellular debris and platelets by centrifugation. The plasma samples can be stored at −80.degree. C. until the DNA is extracted using, for example, QIAamp™ DNA Mini Kit (Qiagen, Hilden, Germany), (e.g. Hamakawa et al., Br J Cancer. 2015; 112:352-356). However, the sample can be derived from other sources and nucleic acid molecules from any organism can be used for this method. In some embodiments, DNA derived from bacteria and/or viruses can be used to analyze true sequence variants within a mixed population, especially in environmental and biodiversity sampling.

Many kits and methods are known in the art for generating libraries of nucleic acid molecules for subsequent sequencing. Kits especially adapted for preparing libraries from small nucleic acid fragments, especially circulating cell-free DNA, can be useful for practicing methods provided herein. For example, the NEXTflex™ Cell Free kits (Bioo Scientific, Austin, Tex.) or the Natera Library Prep Kit (Natera, San Carlos, Calif.). Such kits would typically be modified to include adaptors that are customized for the amplification and sequencing steps of the methods provided herein. Adaptor ligation can also be performed using commercially available kits such as the ligation kit found in the Agilent SureSelect™ kit (Agilent, Santa Clara, Calif.).

Sample nucleic acid molecules are composed of naturally occurring or non-naturally occurring ribonucleotides or deoxyribonucleotides linked through phosphodiester linkages. Furthermore, sample nucleic acid molecules are composed of a nucleic acid segment that is targeted for sequencing. Sample nucleic acid molecules can be or can include nucleic acid segments that are at least 20, 25, 50, 75, 100, 125, 150, 200, 250, 300, 400, 500, 600, 700, 800, 900, or 1,000 nucleotides in length. In any of the embodiments disclosed herein the sample nucleic acid molecules or nucleic acid segments can be between 1, 2, 3, 4, 5, 6, 7, 8, 9, 10, 11, 12, 13, 14, 15, 16, 17, 18, 19, 20, 25, 50, 75, 100, 125, 150, 200, 250, 300, 400, and 500 nucleotides in length on the low end of the range and 10, 11, 12, 13, 14, 15, 16, 17, 18, 19, 20, 25, 50, 75, 100, 125, 150, 200, 250, 300, 400, 500, 1,000, 2,000, 3,000, 4,000, 5,000, 6,000, 7,000, 8,000, 9,000, and 10,000 nucleotides in length on the high end of the range. In some embodiments, the nucleic acid molecules can be fragments of genomic DNA and can be between 1, 2, 3, 4, 5, 6, 7, 8, 9, 10, 11, 12, 13, 14, 15, 16, 17, 18, 19, 20, 25, 50, 75, 100, 125, 150, 200, 250, 300, 400, and 500 nucleotides in length on the low end of the range and 10, 11, 12, 13, 14, 15, 16, 17, 18, 19, 20, 25, 50, 75, 100, 125, 150, 200, 250, 300, 400, 500, 1,000, 2,000, 3,000, 4,000, 5,000, 6,000, 7,000, 8,000, 9,000, and 10,000 nucleotides in length on the high end of the range. For the sake of clarity, nucleic acids initially isolated from a living tissue, fluid, or cultured cells, can be much longer than sample nucleic acid molecules processed using methods herein. As discussed herein, for example, such initially isolated nucleic acid molecules can be fragmented to generate nucleic acid segments, before being used in the methods herein. In some embodiments, the nucleic acid molecules and nucleic acid segments can be identical. The sample nucleic acid molecule or sample nucleic acid segment can include a target locus that contains the nucleotide or nucleotides that are being queried, especially a single nucleotide polymorphism or single nucleotide variant. In any of the disclosed embodiments, the target loci can be at least 1, 2, 3, 4, 5, 6, 7, 8, 9, 10, 11, 12, 13, 14, 15, 16, 17, 18, 19, 20, 25, 50, 75, 100, 125, 150, 200, 250, 300, 400, 500, 600, 700, 800, 900, or 1,000 nucleotides in length and include a portion of or the entirety of the sample nucleic acid molecule and/or the sample nucleic acid segment. In other embodiments, the target loci can be between 1, 2, 3, 4, 5, 6, 7, 8, 9, 10, 11, 12, 13, 14, 15, 16, 17, 18, 19, 20, 25, 50, 75, 100, 125, 150, 200, 250, 300, 400, and 500 nucleotides in length on the low end of the range and 10, 11, 12, 13, 14, 15, 16, 17, 18, 19, 20, 25, 50, 75, 100, 125, 150, 200, 250, 300, 400, 500, 1,000, 2,000, 3,000, 4,000, 5,000, 6,000, 7,000, 8,000, 9,000, and 10,000 nucleotides in length on the high end of the range. In some embodiments, the target loci on different sample nucleic acid molecules can be at least 50%, 60%, 70%, 80%, 90% 95%, 96%, 97%, 98%, 99%, 99.9%, or 100% identical. In some embodiments, the target loci on different sample nucleic acid molecules can share at least 50%, 60%, 70%, 80%, 90% 95%, 96%, 97%, 98%, 99%, 99.9%, or 100% sequence identity.

In some embodiments, the entire sample nucleic acid molecule is a sample nucleic acid segment. For example, in certain embodiments where adaptors are ligated directly to the ends of sample nucleic acid molecules, or ligated to a nucleic acid(s) ligated to the ends of sample nucleic acid molecules, or ligated as part of primers that bind to sequences at the termini of sample nucleic acid segments, or adapters, such as universal adapters added thereto, as discussed further herein, the entire nucleic acid molecule can be a sample nucleic acid segment. In other embodiments, for example certain embodiments where adaptors are attached to sample nucleic acid molecules as part of primers that target binding sites internal to the termini of sample nucleic acid molecules, a portion of the sample nucleic acid molecule can be the sample nucleic acid segment that is targeted for downstream sequencing. For example, at least 50%, 60%, 70%, 75%, 80%, 85%, 90%, 95%, 96%, 97%, 98%, 99%, or 100% of a sample nucleic acid molecule can be a nucleic acid segment.

In some embodiments, sample nucleic acid molecules are a mixture of nucleic acids isolated from a natural source, some sample nucleic acid molecules having identical sequences, some having sequences sharing at least 50%, 60%, 70%, 80%, 90%, 95%, 98%, or 99% sequence identity, and some with less than 50%, 40%, 30%, 20%, 10%, or 5% sequence identity over between 20, 25, 50, 75, 100, 125, 150, 200, 250 nucleotides on the low end of the range, and 50, 75, 100, 125, 150, 200, 250, 300, 400, or 500 nucleotides on the high end of the range. Such sample nucleic acid molecules can be nucleic acid samples isolated from tissues or fluids of a mammal, such as a human, without enriching one sequence over another. In other embodiments, target sequences, for example, those from a gene of interest, can be enriched prior to performing methods provided herein.

### Methods of Identification of Target Genes to Build Molecular Classifiers of Kidney Rejection States.

Identification of one or more mRNAs from target genes and/or miRNAs associated with kidney transplant rejection and found to be present in urine samples of subjects with known kidney rejection states can be achieved by text mining databases. Artificial intelligence may be used for text mining and predicting target genes of known interest for kidney transplant rejection and kidney health.

In some embodiments, the one or more transplant rejection scores are generated using a predictive model, a machine learning based method, and/or an artificial intelligence method.

In some embodiments, the one or more transplant rejection scores are generated using logistic regression (Log Reg), t-test, violin plots, random forest (RE), a neural network, decision tree machine learning analysis, decision trees classification techniques, analysis of variants (ANOVA); Bayesian networks; boosting and Ada-boosting; bootstrap aggregating (or bagging) algorithms, Classification and Regression Trees (CART), boosted CART, Recursive Partitioning Trees (RPART), Curds and Whey (CW); Curds and Whey-Lasso; principal component analysis (PCA), factor rotation or factor analysis; discriminant analysis, Linear Discriminant Analysis (LDA), Eigengene Linear Discriminant Analysis (ELDA), quadratic discriminant analysis; Discriminant Function Analysis (DFA); factor rotation or factor analysis; genetic algorithms; Hidden Markov Models; kernel based machine algorithms such as kernel density estimation, kernel partial least squares algorithms, kernel matching pursuit algorithms, kernel Fisher's discriminate analysis algorithms, kernel principal components analysis algorithms; linear regression and generalized linear models, Forward Linear Stepwise Regression, Lasso (or LASSO) shrinkage and selection method, Elastic Net regularization and selection method; glmnet (Lasso and Elastic Net-regularized generalized linear model); meta-learner algorithms; nearest neighbor methods for classification or regression, Kth-nearest neighbor (KNN); non-linear regression or classification algorithms; neural networks; partial least square; rules based classifiers; shrunken centroids (SC); sliced inverse regression; Standard for the Exchange of Product model data, Application Interpreted Constructs (StepAIC); super principal component (SPC) regression; Support Vector Machines (SVM) and Recursive Support Vector Machines (RSVM), and/or combinations thereof.

In some embodiments, the one or more transplant rejection scores are generated using logistic regression (Log Reg), random forest (RE), a neural network, or decision tree machine learning analysis.

In some embodiments, the one or more mRNAs and/or miRNAs are examined by using 8 separate machine learning classifier methods based on 6 determined kidney disease states. In some embodiments, the one or more transplant rejection scores comprise a first transplant rejection score based on a set of mRNAs and/or miRNAs associated with TCMR, and a second transplant rejection score based on a set of mRNAs and/or miRNAs associated with ABMR. In some embodiments, the one or more transplant rejection scores comprise a transplant rejection score based on a set of mRNAs and/or miRNAs associated with inflammation, a transplant rejection score based on a set of mRNAs and/or miRNAs associated with allograft rejection, a transplant rejection score based on a set of mRNAs and/or miRNAs associated with T cell activation and/or differentiation, a transplant rejection score based on a set of mRNAs and/or miRNAs associated with B cell activation and/or differentiation, a transplant rejection score based on a set of mRNAs and/or miRNAs associated with a cytokine response, and/or a transplant rejection score based on a set of mRNAs and/or miRNAs associated with a chemokine response. In some embodiments, the one or more transplant rejection scores comprise a transplant rejection score based on a set of proteins associated with inflammation, a transplant rejection score based on a set of proteins associated with allograft rejection, a transplant rejection score based on a set of proteins associated with T cell activation and/or differentiation, a transplant rejection score based on a set of proteins associated with B cell activation and/or differentiation, a transplant rejection score based on a set of proteins associated with a cytokine response, and/or a transplant rejection score based on a set of proteins associated with a chemokine response.

In some embodiments, a performance of the determination of kidney transplant rejection or risk of kidney transplant rejection is measured and characterized by an AUC value from about 0.6 to about 0.99, from about 0.7 to about 0.99, from about 0.8 to about 0.99, from about 0.9 to about 0.99, from about 0.7 to about 0.79, from about 0.8 to about 0.89, from about 0.6 to about 0.89, or from about 0.6 to about 0.79.

In some embodiments, the AUC value is from about 0.8 to about 0.99.

In some embodiments, the one or more transplant rejection scores provide a quantitative value of kidney transplant rejection risk that is predictive of a clinical assessment of a kidney disease state and can distinguish between two or more kidney disease states.

Determination of target gene sets useful for determining a transplant rejection scores provide a quantitative value of kidney transplant rejection risk or the presence of kidney transplant rejection.

The sets of target genes found to be differentially expressed in urine over different kidney rejection states may be used for building machine learning classifiers for distinguishing the different kidney rejection or disease states. In particular, classifiers are built to distinguish T-cell mediated (TCMR) or antibody mediated kidney rejection (ABMR). Furthermore, classifiers can determine possible TCMR (pTCMR) or possible ABMR (pABMR), or a mixed state of TCMR and ABMR (referred to as “mixed”).

It is also possible to use classifiers that determine kidney rejection states characterized by apoptosis, cytotoxic T-cell infiltration, cytokine response, chemokine response, parenchymal detoriation, atrophy, fibrosis, or combinations thereof.

For example, a molecular classifier for ABMR may be built by selecting a plurality of differentially expressed genes ABMR and other kidney rejection states. A molecular classifier for TCMR may be built by selecting a plurality of differentially expressed genes TCMR and other kidney rejection states.

The performance of the classifiers may be determined by calculating area under the curve (AUC) of the receiver operating characteristic curve (ROC) that plots true positive rate (sensitivity) and the false positive rate (specificity). In some embodiments, a performance of the determination of kidney transplant rejection or risk of kidney transplant rejection is measured and characterized by an AUC value from about 0.6 to about 0.99, from about 0.7 to about 0.99, from about 0.8 to about 0.99, from about 0.9 to about 0.99, from about 0.7 to about 0.79, from about 0.8 to about 0.89, from about 0.6 to about 0.89, or from about 0.6 to about 0.79

Working example 1 provides further details of this process and an illustrative example thereof.

Combining Measuring Cell-Free DNA with Measuring RNA to Determine and/or Monitoring Transplant Rejection

In some embodiments, the method herein further comprises: (i) measuring the amount of donor-derived cell-free DNA in a sample obtained from the kidney transplant recipient, extracting cell-free DNA from the sample obtained from the kidney transplant recipient, wherein the extracted cell-free DNA comprises donor-derived cell-free DNA and recipient-derived cell-free DNA; (ii) performing targeted amplification of the extracted DNA at 50-50,000 target loci in a single reaction volume; (iii) sequencing the amplified DNA by high-throughput sequencing to obtain sequencing reads and quantifying the amount of donor-derived cell-free DNA based on the sequencing reads, determining kidney transplant rejection based on whether the amount of donor-derived cell-free DNA or a function thereof exceeds a cutoff threshold of cell-free DNA amount that indicates kidney transplant rejection, wherein kidney transplant rejection is determined based on whether (a) the amount of donor-derived cell-free DNA or function thereof exceeds a cutoff threshold that indicates kidney transplant rejection, and (b) the one or more transplant rejection scores that provide a quantitative value of kidney transplant rejection risk or the presence of kidney transplant rejection determined from measuring mRNAs and/or miRNAs in a urine sample from the kidney transplant recipient as shown elsewhere herein. In some embodiments, the amount of one or more mRNAs and/or miRNAs is measured relative to a housekeeping gene.

In some embodiment, the combination of an amount of mRNA targets selected from a group of preselected targets in combination with an amount of cfDNA in the samples indicates transplant rejection or a kidney transplant state. In another aspect, a rejection risk for the transplant recipient can be determined based on the amount of miRNA that provide a quantitative value of kidney transplant rejection risk or state, and an amount of cell-free DNA that is indicated of kidney transplant rejection. In some embodiments, the amount of one or more mRNAs and/or miRNAs is measured relative to a housekeeping gene.

### Determination of Rejection Risk for the Transplant Recipient

In some embodiments, wherein the rejection risk or kidney disease state for the kidney transplant recipient is determined using logistic regression, random forest, or decision tree machine learning analysis. In some embodiments, the machine learning analysis incorporates the amount of RNA (mRNA or miRNA from a target gene) in the sample of the transplant recipient or a function thereof as a parameter. In some embodiments, the machine learning analysis incorporates the number of reads of RNA/DNA or a function thereof as a parameter. In some embodiments, the machine learning analysis incorporates the estimated percentage of donor-derived RNA out of total RNA as a parameter. In some embodiments, the machine learning analysis incorporates the amount of cell-free DNA, the number of reads of cell-free DNA, or the estimated percentage of cell-free DNA out of total cell-free DNA in the sample of the transplant recipient as a parameter. In some embodiments, the machine learning analysis incorporates the amount of total amount of a plurality of proteins derived from the kidney transplant. In some embodiments, the machine learning analysis further incorporates the amount of total cell-free DNA in the sample of the transplant recipient or a function thereof as a parameter. In some embodiments, the machine learning analysis further incorporates the number of reads of total cell-free DNA or a function thereof as a parameter.

Machine learning is disclosed in WO2020/018522, titled “Methods and Systems for calling Ploidy States using a Neural Network” and filed on Jul. 16, 2019 as PCT/US2019/041981, which is incorporated herein by reference in its entirety.

In some embodiments, the cutoff threshold value or rejection scores referred to herein take into account the body mass, BMI, or blood volume of the patient. In some embodiments, the cutoff threshold or rejection scores take into account one or more of the following: donor genome copies per volume of plasma, cell-free DNA yield per volume of plasma, donor height, donor weight, donor age, donor gender, donor ethnicity, donor organ mass, donor organ, live vs deceased donor, the donor's familial relationship to the recipient (or lack thereof), recipient height, recipient weight, recipient age, recipient gender, recipient ethnicity, creatinine, eGFR (estimated glomerular filtration rate), cfDNA methylation, DSA (donor-specific antibodies), KDPI (kidney donor profile index), medications (immunosuppression, steroids, blood thinners, etc.), infections (BKV, EBV, CMV, UTI), recipient and/or donor HLA alleles or epitope mismatches, Banff classification of renal allograft pathology, and for-cause vs surveillance or protocol biopsy.

The methods disclosed herein may have a sensitivity of at least 50% in determining a kidney rejection or disease state and a confidence interval of 95%. The methods disclosed herein may have a sensitivity of at least 50% in determining a kidney rejection or disease state and a confidence interval of 95%. The methods disclosed herein may have a sensitivity of at least 60% in determining a kidney rejection or disease state and a confidence interval of 95%. The methods disclosed herein may have a sensitivity of at least 70% in determining a kidney rejection or disease state and a confidence interval of 95%. The methods disclosed herein may have a sensitivity of at least 80% in determining a kidney rejection or disease state and a confidence interval of 95%. The methods disclosed herein may have a sensitivity of at least 90% in determining a kidney rejection or disease state and a confidence interval of 95%. The methods disclosed herein may have a sensitivity of at least 99% in determining a kidney rejection or disease state and a confidence interval of 95%. The methods disclosed herein may have a sensitivity of between 70 to 99% in determining a kidney rejection or disease state and a confidence interval of 95%. The methods disclosed herein may have a sensitivity of between 80 to 99% in determining a kidney rejection or disease state and a confidence interval of 95%. The methods disclosed herein may have a sensitivity of between 70 to 89% in determining a kidney rejection or disease state and a confidence interval of 95%.

Some embodiments use either a fixed threshold of donor nucleic acids per urine volume or one that is not fixed, such as adjusted or scaled as noted herein. The way that this is determined can be based on using a training data set to build an algorithm to maximize performance. It may also take into account other data such as patient weight, age, or other clinical factors.

In some embodiments, the method further comprises determining the occurrence or likely occurrence of transplant rejection using the amount of donor-derived cell-free DNA in a urine sample. In some embodiments, the amount of donor-derived cell-free DNA is compared to a cutoff threshold value to determine the occurrence or likely occurrence of transplant rejection, wherein the cutoff threshold value is adjusted or scaled according to the amount of total cell-free DNA. In some embodiments, the cutoff threshold value is a function of the number of reads of the donor-derived cell-free DNA.

In some embodiments, the method comprises applying a scaled or dynamic threshold metric that takes into account the amount of total cfDNA in the samples to more accurately assess transplant rejection. In some embodiments, the method further comprises flagging the sample if the amount of total cell-free DNA is above a pre-determined value. In some embodiments, the method further comprises flagging the sample if the amount of total cell-free DNA is below a pre-determined value.

The RNA, DNA, or protein may be extracted from a sample from the transplant recipient, wherein the sample comprises urine.

In some embodiments, the machine learning analysis further incorporates time post-transplantation as a parameter. In some embodiments, the machine learning analysis further incorporates the age of transplant recipient and/or transplant donor as a parameter. In some embodiments, the machine learning analysis further incorporates the gender of transplant recipient and/or transplant donor as a parameter.

In some embodiments, the rejection risk for the transplant recipient is determined with a sensitivity of at least 0.81, or at least 0.82, or at least 0.83, or at least 0.84, or at least 0.85, or at least 0.86, or at least 0.87, or at least 0.88, or at least 0.89, or at least 0.90. In some embodiments, the rejection risk for the transplant recipient is determined with a specificity of at least 0.81, or at least 0.82, or at least 0.83, or at least 0.84, or at least 0.85, or at least 0.86, or at least 0.87, or at least 0.88, or at least 0.89, or at least 0.90. In some embodiments, the rejection risk for the transplant recipient is determined with an area under the curve (AUC) of at least at least 0.86, or at least 0.87, or at least 0.88, or at least 0.89, or at least 0.90, or at least 0.91 or at least 0.92, or at least 0.93, or at least 0.94, or at least 0.95.

In some embodiments, the rejection state for the transplant recipient is determined with a sensitivity of at least 0.81, or at least 0.82, or at least 0.83, or at least 0.84, or at least 0.85, or at least 0.86, or at least 0.87, or at least 0.88, or at least 0.89, or at least 0.90. In some embodiments, the rejection state for the transplant recipient is determined with a specificity of at least 0.81, or at least 0.82, or at least 0.83, or at least 0.84, or at least 0.85, or at least 0.86, or at least 0.87, or at least 0.88, or at least 0.89, or at least 0.90. In some embodiments, the rejection state for the transplant recipient is determined with an area under the curve (AUC) of at least at least 0.86, or at least 0.87, or at least 0.88, or at least 0.89, or at least 0.90, or at least 0.91 or at least 0.92, or at least 0.93, or at least 0.94, or at least 0.95.

### Methods for Measuring the Amount of Nucleic Acids

In some embodiments, the amount of RNA is measured by quantitative PCR. In some embodiments, the amount of RNA is measured by real-time PCR. In some embodiments, the amount of RNA is measured by digital PCR. In some embodiments, the amount of RNA is measured by sequencing such as high-throughput sequencing, next-generation sequence, or sequencing-by-synthesis.

In some embodiments, the amount of donor-derived nucleic acids (e.g. RNA and/or DNA) is determined by using ratiometric and/or machine learning-artificial intelligence comparisons at a single or a plurality of time points. In some embodiments, the amount of donor-derived mRNA is determined by using ratiometric and/or machine learning-artificial intelligence comparisons at a single or a plurality of time points. In some embodiments, the amount of donor-derived miRNA is determined by using ratiometric and/or machine learning-artificial intelligence comparisons at a single or a plurality of time points.

In some embodiments, the amount of RNA or cell-free DNA is measured by a quantitative PCR method. In some embodiments, the amount of mRNA is measured by a quantitative PCR method. In some embodiments, the amount of miRNA is measured by a quantitative PCR method. In some embodiments, the quantitative PCR method comprises real-time PCR or digital PCR.

In some embodiments, the amount of mRNA or cell-free DNA is measured by massively multiplex PCR (mmPCR) to obtain amplicons comprising biomarkers, and sequencing of the amplicons.

In some embodiments, the amount of nucleic acids (e.g. mRNA, miRNA, or cell-free DNA) is measured by using microarray.

In some embodiments, the amount of nucleic acids (e.g. mRNA, miRNA, or cell-free DNA) is measured by using molecular barcodes and microscopic imaging (such as NanoString nCounter®).

In some embodiments, the amplification of RNA comprises performing reverse transcriptase to obtain complementary DNA (cDNA). In some embodiments, the step of preparing the composition of the nucleic acids extracted in step (a) or fractions thereof comprises amplification of cDNA derived from the nucleic acids.

In some embodiments, the amplification comprises performing multiplex targeted amplification of the cDNA at 10-50,000 target loci in a single reaction volume.

In some embodiments, the amplification comprises universal amplification.

In some embodiments, the amount of one or more mRNAs and/or miRNAs is measured by using quantitative PCR, real-time PCR, digital PCR, or sequencing.

In some embodiments, the amount of one or more mRNAs and/or miRNAs is measured by using multiplex quantitative PCR, multiplex real-time PCR, and/or multiplex digital PCR.

In some embodiments, sequencing comprises next-generation whole genome sequencing.

In some embodiments, the amount of one or more mRNAs and/or miRNAs is measured by using microarray.

In some embodiments, the amount of one or more mRNAs and/or miRNAs is measured by using molecular barcodes and microscopic imaging (such as NanoString nCounter®).

In some embodiments, the amount of one or more mRNAs and/or miRNAs is determined by measuring an absolute copy number of the one or more mRNAs and/or miRNAs per amount of total nucleic acids in the urine sample.

In some embodiments, the amount of nucleic acids is measured by targeted amplification. In some embodiments, the amount of a particular mRNA target is measured by targeted amplification. In some embodiments, the targeted amplification comprises PCR. In some embodiments, the primers for the targeted amplification include 10-50,000, 100-50,000, 200-50,000, 500-20,000, or 1,000-10,000, 200-500, 500-1,000, 1,000-2,000, 2,000-5,000, 5,000-10,000, 10,000-20,000, or 20,000-50,000 pairs of forward and reverse PCR primers. In some embodiments, the targeted amplification comprises performing amplification at 100-20,000, 500-20,000, 1,000-10,000, 200-500, 500-1,000, 1,000-2,000, 2,000-5,000, 5,000-10,000, 10,000-20,000, 20,000-50,000 target loci in a single reaction volume using 500-20,000, 1,000-10,000, 200-500, 500-1,000, 1,000-2,000, 2,000-5,000, 5,000-10,000, 10,000-20,000, or 20,000-50,000 primer pairs to obtain amplification products.

In some embodiments, the targeted amplification comprises nested PCR. In some embodiments, the primers for the targeted amplification include a first universal primer and 10-50,000, 100-50,000, 200-50,000, 500-20,000, or 1,000-10,000, 200-500, 500-1,000, 1,000-2,000, 2,000-5,000, 5,000-10,000, 10,000-20,000, or 20,000-50,000 target-specific primers, and a second universal primer and 10-50,000, 100-50,000, 200-50,000, 500-20,000, or 1,000-10,000, 200-500, 500-1,000, 1,000-2,000, 2,000-5,000, 5,000-10,000, 10,000-20,000, or 20,000-50,000 inner target-specific primers. In some embodiments, the targeted amplification comprises performing amplification at 10-50,000, 100-50,000, 200-50,000, 500-20,000, or 1,000-10,000, 200-500, 500-1,000, 1,000-2,000, 2,000-5,000, 5,000-10,000, 10,000-20,000, or 20,000-50,000 target loci in a single reaction volume using a first universal primer and 10-50,000, 100-50,000, 200-50,000, 500-20,000, or 1,000-10,000, 200-500, 500-1,000, 1,000-2,000, 2,000-5,000, 5,000-10,000, 10,000-20,000, or 20,000-50,000 target-specific primers to obtain amplification products. In some embodiments, the targeted amplification comprises performing amplification at 10-50,000, 100-50,000, 200-50,000, 500-20,000, or 1,000-10,000, 200-500, 500-1,000, 1,000-2,000, 2,000-5,000, 5,000-10,000, 10,000-20,000, or 20,000-50,000 target loci in a single reaction volume using a second universal primer and 10-50,000, 100-50,000, 200-50,000, 500-20,000, or 1,000-10,000, 200-500, 500-1,000, 1,000-2,000, 2,000-5,000, 5,000-10,000, 10,000-20,000, or 20,000-50,000 inner target-specific primers to obtain amplification products. In some embodiments, the methods disclosed herein comprise PCR amplification of at least 10, at least 100, at least 500, at least 1000, at least 2000 biomarkers, from 10-1000, 100-10000, 200-50000, or 500-20000 RNA biomarkers, using at least 10, at least 100, at least 500, at least 1000, at least 2000, from 10-1000, 100-10000, 200-50000, 500-20000 pairs of forward and reverse PCR primers. In some embodiments, step (b) comprises amplification of at least 2, at least 5, at least 10, at least 20, at least 30, at least 50, at least 100 target RNA molecules, from 2-10, 200-100, 50-500, or 50-2000 target RNA molecules, using at least at least 2, at least 5, at least 10, at least 20, at least 30, at least 50, at least 100 target RNA molecules, from 2-10, 200-100, 50-500, or 50-2000 pairs of forward and reverse PCR primers.

In some embodiments, the method further comprises attaching tags to the amplification products prior to performing high-throughput sequencing, wherein the tags comprise sequencing-compatible adaptors. In some embodiments, the method further comprises attaching tags to the extracted RNA prior to performing targeted amplification, wherein the tags comprise adaptors for amplification. In some embodiment, the tags comprise sample-specific barcodes, and wherein the method further comprises pooling the amplification products from a plurality of samples prior to high-throughput sequencing and sequencing the pool of amplification products together in a single run during the high-throughput sequencing.

In some embodiments, the amount of nucleic acids is determined by using for example, tracer nucleic acids, or internal calibration nucleic acids. The terms “tracer nucleic acids,” or “internal calibration nucleic acids” are used interchangeably and refer to a composition of nucleic acids for which one or more of the following is known advance-length, sequence, nucleotide composition, quantity, or biological origin. The tracer can be added to a biological sample derived from a human subject to help estimate the amount of total RNA or cfDNA in said sample. It can also be added to reaction mixtures other than the biological sample itself.

Performance of the Methods for Determining Transplant Rejection or Rejection States when Combining the Measurements of Specific Urine Target Genes and Amounts of Donor RNA or DNA.

In one aspect, the herein described methods of determining a kidney transplant rejection state can be combined with measuring transplant rejection risk based on determining the amount of donor-derived RNA (dd-RNA) or cell-free DNA (dd-cfDNA)) in a biological sample from the kidney transplant recipient.

In some embodiments, the method has a sensitivity of at least 80%, or at least 85%, or at least 90%, or at least 95%, or at least 98% in identifying acute rejection (AR) over non-AR with a cutoff threshold of 1% dd-RNA or dd-cfDNA and a confidence interval of 95%.

In some embodiments, the method has a specificity of at least 60%, or at least 65%, or at least 70%, or at least 75%, or at least 80%, or at least 85%, or at least 90% in identifying AR over non-AR with a cutoff threshold of 1% dd-RNA or dd-cfDNA and a confidence interval of 95%.

In some embodiments, the method has an area under the curve (AUC) of at least 0.8, or 0.85, or at least 0.9, or at least 0.95 in identifying AR over non-AR with a cutoff threshold of 1% dd-RNA or dd-cfDNA and a confidence interval of 95%.

In some embodiments, the method has a sensitivity of at least 80%, or at least 85%, or at least 90%, or at least 95%, or at least 98% in identifying AR over normal, stable allografts (STA) with a cutoff threshold of 1% dd-RNA or dd-cfDNA and a confidence interval of 95%.

In some embodiments, the method has a specificity of at least 80%, or at least 85%, or at least 90%, or at least 95%, or at least 98% in identifying AR over STA with a cutoff threshold of 1% dd-RNA or dd-cfDNA and a confidence interval of 95%.

In some embodiments, the method has an AUC of at least 0.8, or 0.85, or at least 0.9, or at least 0.95, or at least 0.98, or at least 0.99 in identifying AR over STA with a cutoff threshold of 1% dd-RNA or dd-cfDNA and a confidence interval of 95%.

In some embodiments, the method has a sensitivity as determined by a limit of blank (LoB) of 0.5% or less, and a limit of detection (LoD) of 0.5% or less. In some embodiments, LoB is 0.23% or less and LoD is 0.29% or less. In some embodiments, the sensitivity is further determined by a limit of quantitation (LoQ). In some embodiments, LoQ is 10 times greater than the LoD; LoQ may be 5 times greater than the LoD; LoQ may be 1.5 times greater than the LoD; LoQ may be 1.2 times greater than the LoD; LoQ may be 1.1 times greater than the LoD; or LoQ may be equal to or greater than the LoD. In some embodiments, LoB is equal to or less than 0.04%, LoD is equal to or less than 0.05%, and/or LoQ is equal to the LoD.

In some embodiments, the method has an accuracy as determined by evaluating a linearity value obtained from linear regression analysis of measured donor fractions as a function of the corresponding attempted spike levels, wherein the linearity value is a R² value, wherein the R² value is from about 0.98 to about 1.0. In some embodiments, the R² value is 0.999. In some embodiments, the method has an accuracy as determined by using linear regression on measured donor fractions as a function of the corresponding attempted spike levels to calculate a slope value and an intercept value, wherein the slope value is from about 0.9 to about 1.2 and the intercept value is from about-0.0001 to about 0.01. In some embodiments, the slope value is approximately 1, and the intercept value is approximately 0.

In some embodiments, the method has a precision as determined by calculating a coefficient of variation (CV), wherein the CV is less than about 10.0%. CV is less than about 6%. In some embodiments, the CV is less than about 4%. In some embodiments, the CV is less than about 2%. In some embodiments, the CV is less than about 1%.

In some embodiments, the AR is antibody-mediated rejection (ABMR). In some embodiments, the AR is T-cell-mediated rejection (TCMR).

In some embodiments, the cutoff threshold is an estimate percentage of RNA targets out of total RNA or a function thereof. In some embodiments, the cutoff threshold is 1.0%, 1.1%, 1.2%, 1.3%, 1.4%, 1.5%, 1.6%, 1.7%, 1.8%, 1.9%, or 2.0% RNA (e.g. mRNA or miRNA). In some embodiments, the cutoff threshold is 1.0%, 1.1%, 1.2%, 1.3%, 1.4%, 1.5%, 1.6%, 1.7%, 1.8%, 1.9%, or 2.0% cell-free DNA or combination of cell-free DNA and RNA. In some embodiments, the cutoff threshold is adjusted according to the type of organs transplanted. In some embodiments, the cutoff threshold is adjusted according to the number of organs transplanted.

In some embodiments, the cutoff threshold is an estimate percentage of donor-derived RNA out of total RNA or a function thereof. In some embodiments, the cutoff threshold is 1.0%, 1.1%, 1.2%, 1.3%, 1.4%, 1.5%, 1.6%, 1.7%, 1.8%, 1.9%, or 2.0% RNA. In some embodiments, the cutoff threshold is 1.0%, 1.1%, 1.2%, 1.3%, 1.4%, 1.5%, 1.6%, 1.7%, 1.8%, 1.9%, or 2.0% cell-free DNA or combination of cell-free DNA and RNA.

In some embodiments, the cutoff threshold is an estimate percentage of amounts of pre-selected mRNA out of total mRNA or a function thereof. In some embodiments, the cutoff threshold is 1.0%, 1.1%, 1.2%, 1.3%, 1.4%, 1.5%, 1.6%, 1.7%, 1.8%, 1.9%, or 2.0% RNA. In some embodiments, the cutoff threshold is 1.0%, 1.1%, 1.2%, 1.3%, 1.4%, 1.5%, 1.6%, 1.7%, 1.8%, 1.9%, or 2.0% cell-free DNA or combination of cell-free DNA and mRNA In some embodiments, the cutoff threshold is adjusted according to the type of organs transplanted. In some embodiments, the cutoff threshold is adjusted according to the number of organs transplanted.

In some embodiments, the cutoff threshold is an estimate percentage of donor-derived preselected mRNA targets out of total mRNA or a function thereof. In some embodiments, the cutoff threshold is 1.0%, 1.1%, 1.2%, 1.3%, 1.4%, 1.5%, 1.6%, 1.7%, 1.8%, 1.9%, or 2.0% RNA.

In some embodiments, the cutoff threshold is an estimate percentage of amount of preselected protein out of total protein or a function thereof. In some embodiments, the cutoff threshold is 1.0%, 1.1%, 1.2%, 1.3%, 1.4%, 1.5%, 1.6%, 1.7%, 1.8%, 1.9%, or 2.0% protein.

In some embodiments, the cutoff threshold is proportional to an absolute donor-derived RNA concentration. In some embodiments, the cutoff threshold is a copy number of donor-derived RNA or a function thereof. In some embodiments, the cutoff threshold is expressed as quantity or absolute quantity of RNA. In some embodiments, the cutoff threshold is expressed as quantity or absolute quantity of RNA per volume unit of the blood sample. In some embodiments, the cutoff threshold is expressed as quantity or absolute quantity of RNA per volume unit of the blood sample multiplied by body mass, BMI, or blood volume of the transplant recipient.

In some embodiments, the cutoff threshold is proportional to an absolute donor-derived RNA concentration. In some embodiments, the cutoff threshold is a copy number of donor-derived RNA or a function thereof. In some embodiments, the cutoff threshold is expressed as quantity or absolute quantity of RNA. In some embodiments, the cutoff threshold is expressed as quantity or absolute quantity of RNA per volume unit of the blood sample. In some embodiments, the cutoff threshold is expressed as quantity or absolute quantity of RNA per volume unit of the sample multiplied by body mass, BMI.

In some embodiments, the cutoff threshold is proportional to an absolute donor-derived protein concentration. In some embodiments, the cutoff threshold is expressed as quantity or absolute quantity of protein. In some embodiments, the cutoff threshold is expressed as quantity or absolute quantity of protein per volume unit of the blood sample. In some embodiments, the cutoff threshold is expressed as quantity or absolute quantity of protein per volume unit of the blood sample multiplied by body mass, BMI, or blood volume of the transplant recipient.

### Definitions

As used herein the term “single nucleotide polymorphism (SNP)” refers to a single nucleotide that may differ between the genomes of two members of the same species. The usage of the term does not imply any limit on the frequency with which each variant occurs.

In some embodiments, for example, sequence refers to a DNA or RNA sequence or a genetic sequence. It may refer to the primary, physical structure of the DNA or RNA molecule or strand in an individual. It may refer to the sequence of nucleotides found in that DNA or RNA molecule, or the complementary strand to the DNA or RNA molecule. It may refer to the information contained in the DNA or RNA molecule as its representation in silico.

“Baseline level of gene expression level” includes the particular gene expression level of a healthy subject or a subject with a well-functioning transplant. The baseline level of gene expression includes the gene expression level of a subject without acute rejection. The baseline level of gene expression can be a number on paper or the baseline level of gene expression from a control sample of a healthy subject or a subject with a well-functioning transplant.

A “gene product” includes a peptide, polypeptide, or structural RNA generated when a gene is transcribed and/or translated. While an mRNA encoding a peptide or polypeptide can be translated to generate the peptide or polypeptide, a structural RNA (e.g., an rRNA) is not translated.

The term “level of gene expression” as used herein refers to quantifying gene expression. In some embodiments, to accurately assess whether increased mRNA or rRNA is significant, it is preferable to “normalize” gene expression to accurately compare levels of expression between samples, i.e., it is a baseline level against which gene expression is compared. Quantification of gene expression can be accomplished by methods known in the art, such as, for example, reverse transcription polymerase chain reaction (RT-PCR), TAQMAN® assays or the like. Gene expression can also be quantified by detecting a protein, peptide or structural RNA gene product directly, in a variety of assay formats known to those of ordinary skill in the art. For example, proteins and peptides can be detected by an assay such as an enzyme linked immunosorbent assay (ELISA), radioimmunoassay (RIA), immunofluorimetry, immunoprecipitation, equilibrium dialysis, immunodiffusion, immunoblotting, mass spectrometry and other techniques. See, e.g., Harlow and Lane, Antibodies: A Laboratory Manual, Cold Spring Harbor Laboratory, 1 88; Weir, D. M., Handbook of Experimental Immunology, 1986, Blackwell Scientific, Boston.

In some embodiments, for example, locus refers to a particular region of interest on the DNA or RNA of an individual and includes without limitation one or more SNPs, the site of a possible insertion or deletion, or the site of some other relevant genetic variation. Disease-linked SNPs may also refer to disease-linked loci.

In some embodiments, for example, polymorphic allele, also “polymorphic locus,” refers to an allele or locus where the genotype varies between individuals within a given species. Some examples of polymorphic alleles include single nucleotide polymorphisms (SNPs), short tandem repcats, deletions, duplications, and inversions.

In some embodiments, for example, allele refers to the nucleotides or nucleotide sequence occupying a particular locus.

In some embodiments, for example, genetic data also “genotypic data” refers to the data describing aspects of the genome of one or more individuals. It may refer to one or a set of loci, partial or entire sequences, partial or entire chromosomes, or the entire genome. It may refer to the identity of one or a plurality of nucleotides; it may refer to a set of sequential nucleotides, or nucleotides from different locations in the genome, or a combination thereof. Genotypic data is typically in silico, however, it is also possible to consider physical nucleotides in a sequence as chemically encoded genetic data. Genotypic Data may be said to be “on,” “of,” “at,” “from” or “on” the individual(s). Genotypic Data may refer to output measurements from a genotyping platform where those measurements are made on genetic material.

In some embodiments, for example, genetic material also “genetic sample” refers to physical matter, such as tissue or urine, from one or more individuals comprising nucleic acids (e.g., comprising DNA or RNA).

As used herein, the term “transplantation” refers to the process of taking a cell, tissue, or organ, called a “transplant” or “graft” from one individual and placing it or them into a (usually) different individual. The individual who provides the transplant is called the “donor” and the individual who received the transplant is called the “recipient” (or “host”). An organ, or graft, transplanted between two genetically different individuals of the same species is called an “allograft.” A graft transplanted between individuals of different species is called a “xenograft.”

As used herein, “transplant rejection” refers to a functional and structural deterioration of the organ due to an active immune response expressed by the recipient, and independent of non-immunologic causes of organ dysfunction. Acute transplant rejection can result from the activation of recipient's T cells and/or B cells; the rejection primarily due to T cells is classified as T cell mediated acute rejection (TCMR) and the rejection in which B cells are primarily responsible is classified as antibody mediated rejection (AMR). In some embodiments, the methods and compositions provided can detect and/or predict acute cellular rejection. In some embodiments, the methods can distinguish between different states of kidney rejection such as TCMR or AMR.

In some embodiments, for example, allelic data refers to a set of genotypic data concerning a set of one or more alleles. It may refer to the phased, haplotypic data. It may refer to SNP identities, and it may refer to the sequence data of the nucleic acid, including insertions, deletions, repeats and mutations.

As used herein, “subject” means a mammal and includes a “transplant recipient.” “Mammals” means any member of the class. Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; or the like. The term “subject” does not denote a particular age or sex. Preferably the subject is a human patient. In some embodiments, the subject is a human who has received an organ transplant; i.e. a transplant recipient.

The term “up-regulation,” “up-regulated,” “increased expression,” and “higher expression” are used interchangeably herein and refer to the increase or elevation in the amount of a target mRNA or a target protein. In some embodiments, up-regulation,” “up-regulated,” “increased expression,” and “higher expression” includes increases above a baseline (e.g., a control, or reference) level of 10%, 15%, 20%, 25%, 30%, 35%, 40%, 45%, 50%, 55%, 60%, 65%, 70%, 75%, 80%, 85%, 90%, 95%, 100% or higher.

In some embodiments, for example, allelic state refers to the actual state of the genes in a set of one or more alleles. It may refer to the actual state of the genes described by the allelic data.

In some embodiments, for example, allelic ratio or allele ratio, refers to the ratio between the amount of each allele at a locus that is present in a sample or in an individual. When the sample was measured by sequencing, the allelic ratio may refer to the ratio of sequence reads that map to each allele at the locus. When the sample was measured by an intensity based measurement method, the allele ratio may refer to the ratio of the amounts of each allele present at that locus as estimated by the measurement method.

In some embodiments, for example, allele count refers to the number of sequences that map to a particular locus, and if that locus is polymorphic, it refers to the number of sequences that map to each of the alleles. If each allele is counted in a binary fashion, then the allele count will be whole number. If the alleles are counted probabilistically, then the allele count can be a fractional number.

In some embodiments, for example, primer, also “PCR probe” refers to a single DNA molecule (a DNA oligomer) or a collection of DNA molecules (DNA oligomers) where the DNA molecules are identical, or nearly so, and where the primer contains a region that is designed to hybridize to a targeted polymorphic locus, and contain a priming sequence designed to allow amplification such as PCR amplification. A primer may also contain a molecular barcode. A primer may contain a random region that differs for each individual molecule.

In some embodiments, for example, hybrid capture probe refers to any nucleic acid sequence, possibly modified, that is generated by various methods such as PCR or direct synthesis and intended to be complementary to one strand of a specific target DNA or RNA sequence in a sample. The exogenous hybrid capture probes may be added to a prepared sample and hybridized through a denaturation-reannealing process to form duplexes of exogenous-endogenous fragments. These duplexes may then be physically separated from the sample by various means.

In some embodiments, for example, sequence read refers to data representing a sequence of nucleotide bases that were measured using a clonal sequencing method. Clonal sequencing may produce sequence data representing single, or clones, or clusters of one original DNA or RNA molecule. A sequence read may also have associated quality score at each base position of the sequence indicating the probability that nucleotide has been called correctly.

In some embodiments, for example, mapping a sequence read is the process of determining a sequence read's location of origin in the genome sequence of a particular organism. The location of origin of sequence reads is based on similarity of nucleotide sequence of the read and the genome sequence.

In some embodiments, for example, DNA or RNA of donor origin refers to DNA or RNA that was originally part of a cell whose genotype was essentially equivalent to that of the transplant donor. The donor can be a human or a non-human mammalian (e.g., pig).

In some embodiments, for example, DNA or RNA of recipient origin refers to DNA or RNA that was originally part of a cell whose genotype was essentially equivalent to that of the transplant recipient.

In some embodiments, RNA may refer to messenger RNA (mRNA), small non-coding RNA (sncRNA), transfer RNA (tRNA), or a non-protein coding RNA from cells. In some embodiments, sncRNA comprises micro RNA (miRNA), piwi-interacting RNA (piRNA), small nucleolar RNA (snoRNA), small nuclear RNA (snRNA), or miscellaneous RNA (miscRNA). In some embodiments, the RNA is cell-free RNA. In some embodiments, the cell-free RNA is derived from exosomes or microvesicles.

In some embodiments, amplification of RNA comprises reverse transcription of RNA to produce complementary DNA (cDNA) followed by amplification of cDNA by amplification methods disclosed elsewhere herein.

In some embodiments, for example, preferential enrichment of DNA or RNA that corresponds to a locus, or preferential enrichment of DNA or RNA at a locus, refers to any technique that results in the percentage of molecules of DNA or RNA in a post-enrichment DNA or RNA mixture that correspond to the locus being higher than the percentage of molecules of DNA or RNA in the pre-enrichment DNA or RNA mixture that correspond to the locus. The technique may involve selective amplification of DNA or RNA molecules that correspond to a locus. The technique may involve removing DNA or RNA molecules that do not correspond to the locus. The technique may involve a combination of methods. The degree of enrichment is defined as the percentage of molecules of DNA or RNA in the post-enrichment mixture that correspond to the locus divided by the percentage of molecules of DNA or RNA in the pre-enrichment mixture that correspond to the locus. Preferential enrichment may be carried out at a plurality of loci. In some embodiments of the present disclosure, the degree of enrichment is greater than 20. In some embodiments of the present disclosure, the degree of enrichment is greater than 200. In some embodiments of the present disclosure, the degree of enrichment is greater than 2,000. When preferential enrichment is carried out at a plurality of loci, the degree of enrichment may refer to the average degree of enrichment of all of the loci in the set of loci.

In some embodiments, for example, amplification refers to a technique that increases the number of copies of a molecule of RNA and/or DNA.

In some embodiments, for example, selective amplification may refer to a technique that increases the number of copies of a particular molecule of RNA and/or DNA, or molecules of RNA and/or DNA that correspond to a particular region of RNA and/or DNA. It may also refer to a technique that increases the number of copies of a particular targeted molecule of RNA and/or DNA, or targeted region of RNA and/or DNA more than it increases non-targeted molecules or regions of RNA and/or DNA. Selective amplification may be a method of preferential enrichment.

In some embodiments, for example, universal priming sequence refers to a DNA sequence that may be appended to a population of target nucleic acid molecules, for example by ligation, PCR, or ligation mediated PCR. Once added to the population of target molecules, primers specific to the universal priming sequences can be used to amplify the target population using a single pair of amplification primers. Universal priming sequences need not be related to the target sequences.

In some embodiments, for example, universal adapters, or ‘ligation adaptors’ or ‘library tags’ are DNA molecules containing a universal priming sequence that can be covalently linked to the 5-prime and 3-prime end of a population of target double stranded DNA molecules. The addition of the adapters provides universal priming sequences to the 5-prime and 3-prime end of the target population from which PCR amplification can take place, amplifying all molecules from the target population, using a single pair of amplification primers.

In some embodiments, for example, targeting refers to a method used to selectively amplify or otherwise preferentially enrich those molecules of DNA or RNA that correspond to a set of loci in a mixture of DNA or RNA.

“Acute rejection or AR” is the rejection by the immune system of a tissue transplant recipient when the transplanted tissue is immunologically foreign. Acute rejection is characterized by infiltration of the transplanted tissue by immune cells of the recipient, which carry out their effector function and destroy the transplanted tissue. The onset of acute rejection is rapid and generally occurs in humans within a few weeks after transplant surgery. Generally, acute rejection can be inhibited or suppressed with immunosuppressive drugs such as rapamycin, cyclosporin A, anti-CD40L monoclonal antibody and the like.

“Chronic transplant rejection or injury” or “CAI” generally occurs in humans within several months to years after engraftment, even in the presence of successful immunosuppression of acute rejection. Fibrosis is a common factor in chronic rejection of all types of organ transplants. Chronic rejection can typically be described by a range of specific disorders that are characteristic of the particular organ. In kidney transplants, such disorders include, obstructive nephropathy, nephrosclerosis, tubulointerstitial nephropathy. Chronic rejection can also be characterized by ischemic insult, denervation of the transplanted tissue, hyperlipidemia and hypertension associated with immunosuppressive drugs.

The term “transplant injury” refers to all manners of graft dysfunction, irrespective of pathological diagnosis. The term “organ injury” refers to target loci that track with poor function of the organ, irrespective of the organ being native or a transplant, and irrespective of the etiology.

### Multiplex Amplification

In some embodiments, the method comprises performing a multiplex amplification reaction to amplify a plurality of target loci in one reaction mixture before determining the sequences of the selectively enriched RNA or DNA.

In certain illustrative embodiments, the nucleic acid sequence data is generated by performing high throughput RNA sequencing of a plurality of copies of a series of amplicons generated using a multiplex amplification reaction, wherein each amplicon of the series of amplicons spans at least one polymorphic locus of the set of polymorphic loci and wherein each of the polymeric loci of the set is amplified. In certain illustrative embodiments, the nucleic acid sequence data is generated by performing high throughput DNA sequencing of a plurality of copies of a series of amplicons generated using a multiplex amplification reaction, wherein each amplicon of the series of amplicons spans at least one polymorphic locus of the set of polymorphic loci and wherein each of the polymeric loci of the set is amplified. For example, in these embodiments a multiplex PCR to amplify amplicons across at least 100; 200; 500; 1,000; 2,000; 5,000; 10,000; 20,000; 50,000; or 100,000 polymorphic loci (e.g., SNP loci) may be performed. This multiplex reaction can be set up as a single reaction or as pools of different subset multiplex reactions. The multiplex reaction methods provided herein, such as the massive multiplex PCR disclosed herein provide an exemplary process for carrying out the amplification reaction to help attain improved multiplexing and therefore, sensitivity levels.

In some embodiments, amplification is performed using direct multiplexed PCR, sequential PCR, nested PCR, doubly nested PCR, one-and-a-half sided nested PCR, fully nested PCR, one sided fully nested PCR, one-sided nested PCR, hemi-nested PCR, hemi-nested PCR, triply hemi-nested PCR, semi-nested PCR, one sided semi-nested PCR, reverse semi-nested PCR method, or one-sided PCR, which are described in U.S. application Ser. No. 13/683,604, filed Nov. 21, 2012, U.S. Publication No. 2013/0123120, U.S. application Ser. No. 13/300,235, filed Nov. 18, 2011, U.S. Publication No 2012/0270212, and U.S. Ser. No. 61/994,791, filed May 16, 2014, all of which are hereby incorporated by reference in their entirety.

In some embodiments, multiplex PCR is used. In some embodiments, the method of amplifying target loci in a nucleic acid sample involves (i) contacting the nucleic acid sample with a library of primers that simultaneously hybridize to at least 100; 200; 500; 1,000; 2,000; 5,000; 10,000; 20,000; 50,000; or 100,000 different target loci to produce a single reaction mixture; and (ii) subjecting the reaction mixture to primer extension reaction conditions (such as PCR conditions) to produce amplified products that include target amplicons. In some embodiments, at least 50, 60, 70, 80, 90, 95, 96, 97, 98, 99, or 99.5% of the targeted loci are amplified. In various embodiments, less than 60, 50, 40, 30, 20, 10, 5, 4, 3, 2, 1, 0.5, 0.25, 0.1, or 0.05% of the amplified products are primer dimers. In some embodiments, the primers are in solution (such as being dissolved in the liquid phase rather than in a solid phase). In some embodiments, the primers are in solution and are not immobilized on a solid support. In some embodiments, the primers are not part of a microarray.

In certain embodiments, the multiplex amplification reaction is performed under limiting primer conditions for at least ½ of the reactions. In some embodiments, limiting primer concentrations are used in 1/10, 1/5, 1/4, 1/3, 1/2, or all of the reactions of the multiplex reaction. Provided herein are factors to consider in achieving limiting primer conditions in an amplification reaction such as PCR.

In certain embodiments, the multiplex amplification reaction can include, for example, between 2,500 and 50,000 multiplex reactions. In certain embodiments, the following ranges of multiplex reactions are performed: between 100, 200, 250, 500, 1000, 2500, 5000, 10,000, 20,000, 25000, 50000 on the low end of the range and between 200, 250, 500, 1000, 2500, 5000, 10,000, 20,000, 25000, 50000, and 100,000 on the high end of the range.

In an embodiment, a multiplex PCR assay is designed to amplify potentially heterozygous SNP or other polymorphic or non-polymorphic loci on one or more chromosomes and these assays are used in a single reaction to amplify DNA. The number of PCR assays may be between 50 and 200 PCR assays, between 200 and 1,000 PCR assays, between 1,000 and 5,000 PCR assays, or between 5,000 and 20,000 PCR assays (50 to 200-plex, 200 to 1,000-plex, 1,000 to 5,000-plex, 5,000 to 20,000-plex, more than 20,000-plex respectively). In an embodiment, a multiplex pool of at least 10,000 PCR assays (10,000-plex) are designed to amplify potentially heterozygous SNP loci a single reaction to amplify RNA or cfDNA obtained from a urine sample. The SNP frequencies of each locus may be determined by clonal or some other method of sequencing of the amplicons. In another embodiment the original cfDNA samples is split into two samples and parallel 5,000-plex assays are performed. In another embodiment the original cfDNA samples is split into n samples and parallel (~10,000/n)-plex assays are performed where n is between 2 and 12, or between 12 and 24, or between 24 and 48, or between 48 and 96.

In an embodiment, a method disclosed herein uses highly efficient highly multiplexed targeted PCR to amplify DNA followed by high throughput sequencing to determine the allele frequencies at each target locus. One technique that allows highly multiplexed targeted PCR to perform in a highly efficient manner involves designing primers that are unlikely to hybridize with one another. The PCR probes, typically referred to as primers, are selected by creating a thermodynamic model of potentially adverse interactions between at least 100, at least 200, at least 500, at least 1,000, at least 2,000, at least 5,000, at least 10,000, at least 20,000, or at least 50,000 potential primer pairs, or unintended interactions between primers and sample DNA, and then using the model to eliminate designs that are incompatible with other the designs in the pool. Another technique that allows highly multiplexed targeted PCR to perform in a highly efficient manner is using a partial or full nesting approach to the targeted PCR. Using one or a combination of these approaches allows multiplexing of at least 100, at least 200, at least 500, at least 1,000, at least 2,000, at least 5,000, at least 10,000, at least 20,000, or at least 50,000 primers in a single pool with the resulting amplified DNA comprising a majority of DNA molecules that, when sequenced, will map to targeted loci. Using one or a combination of these approaches allows multiplexing of a large number of primers in a single pool with the resulting amplified DNA comprising greater than 50%, greater than 80%, greater than 90%, greater than 95%, greater than 98%, or greater than 99% DNA molecules that map to targeted loci.

Bioinformatics methods are used to analyze the genetic data obtained from multiplex PCR. The bioinformatics methods useful and relevant to the methods disclosed herein can be found in U.S. Patent Publication No. 2018/0025109, incorporated by reference herein.

### High-Throughput Sequencing

In some embodiments, the sequences of the amplicons are determined by performing high-throughput sequencing.

The genetic data of the transplant recipient and/or of the transplant donor can be transformed from a molecular state to an electronic state by measuring the appropriate genetic material using tools and or techniques taken from a group including, but not limited to: genotyping microarrays, and high throughput sequencing. Some high throughput sequencing methods include Sanger DNA sequencing, pyrosequencing, the ILLUMINA SOLEXA platform, ILLUMINA's GENOME ANALYZER, or APPLIED BIOSYSTEM's 454 sequencing platform, HELICOS's TRUE SINGLE MOLECULE SEQUENCING platform, HALCYON MOLECULAR's electron microscope sequencing method, PacBio®, Oxford Nanopore®, or any other sequencing method. In some embodiments, the high throughput sequencing is performed on Illumina NextSeq®. All of these methods physically transform the genetic data stored in a sample of DNA into a set of genetic data that is typically stored in a memory device en route to being processed.

In some embodiments, the sequences of the selectively enriched DNA are determined by performing microarray analysis. In an embodiment, the microarray may be an ILLUMINA SNP microarray, or an AFFYMETRIX SNP microarray.

In some embodiments, the sequences of the selectively enriched DNA are determined by performing quantitative PCR (qPCR) or digital droplet PCR (ddPCR) analysis. qPCR measures the intensity of fluorescence at specific times (generally after every amplification cycle) to determine the relative amount of target molecule (DNA). ddPCR measures the actual number of molecules (target DNA) as each molecule is in one droplet, thus making it a discrete “digital” measurement. It provides absolute quantification because ddPCR measures the positive fraction of samples, which is the number of droplets that are fluorescing due to proper amplification. This positive fraction accurately indicates the initial amount of template nucleic acid.

The present description is further illustrated by the following examples, which should not be construed as limiting in any way.

### WORKING EXAMPLES

### Example 1: Identify mRNA Targets in Urine that can Determine Kidney Rejection

This example is illustrative only, and a skilled artisan will appreciate that the invention disclosed herein can be practiced in a variety of other ways.

The purpose of this Example is to illustrate identification of mRNA targets in urine samples that can effectively differentiate rejection over different kidney disease states.

To identify mRNA targets that can effectively differentiate rejection over different kidney disease states, Affymetrix® mRNA expression data of 1745 samples were sourced from the MMDx® diagnostic system. MMDx® Kidney & Heart are biopsy-based Laboratory Developed Tests that measure gene expression profiling and provide risk assessment for rejection and injury in transplant organs. The mRNA targets associated with rejection from the MMDx® diagnostic system were correlated with mRNA targets found in literature to be associated with kidney rejection in urine samples.

Table 1 (see Tables section herein) shows mRNAs found expressed in urine samples and corresponding miRNAs.

In particular, 96% of the MMDx® samples (1,679) were histologically examined and classified by machine learning techniques. mRNAs found independently in literature were examined via 8 separate machine learning (ML) classifier methods to 6 MMDx® determined kidney disease states, which includes: Antibody mediated rejection (ABMR), T-cell mediated rejection (TCMR), possible ABMR (pABMR), possible TCMR (pTCMR), Mixed (both ABMR and TCMR), and no rejection. Among these 1679 samples, 509 samples were ABMR, 52 were pABMR, 123 were TCMR, 21 were pTMCR, 69 were mixed (ABMR and TCMR), and 905 were non-rejection (NR).

AUC (area under the curve) based on sensitivity (true positive results) and specificity (false positive results) curves was determined by using 8 classifiers to evaluate the performance of the mRNA targets over the 6 MMDx® determined urine kidney disease states as shown in Table 2 and 10 in the Tables section below. In particular, AUCs calculated via 8 classifiers ranged between 0.82 and 0.99 for the indicated disease state comparisons.

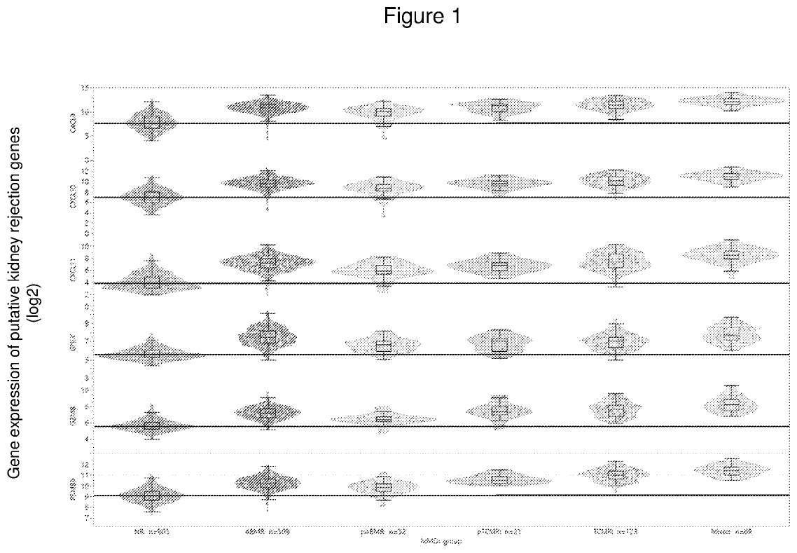

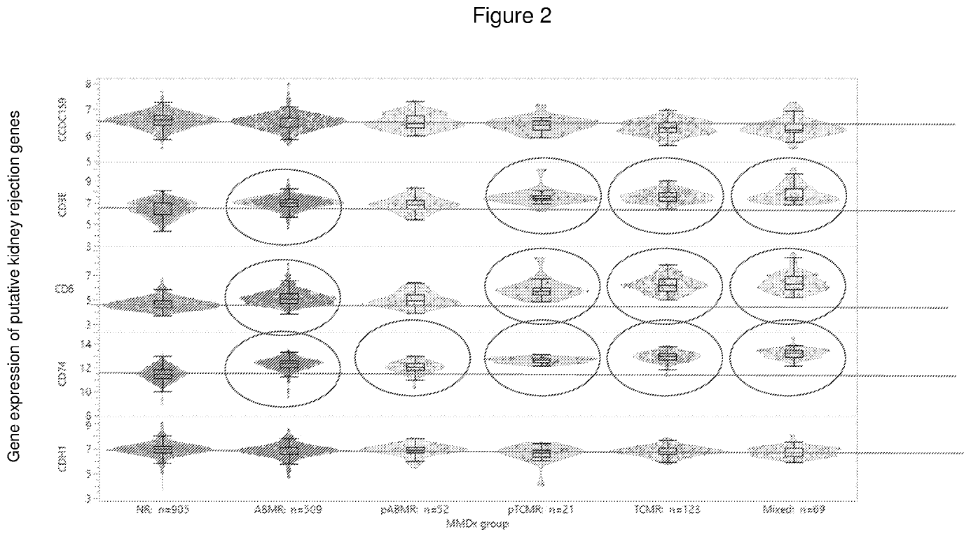

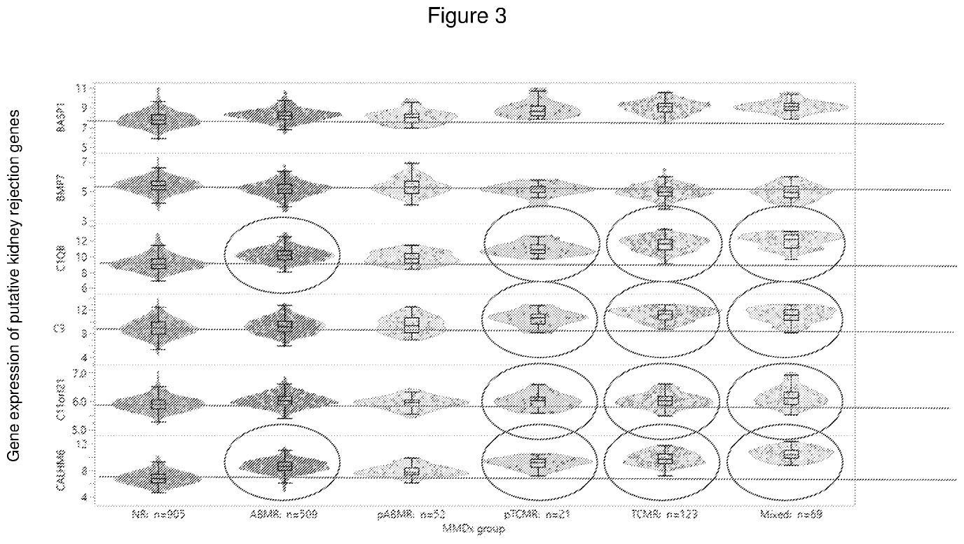

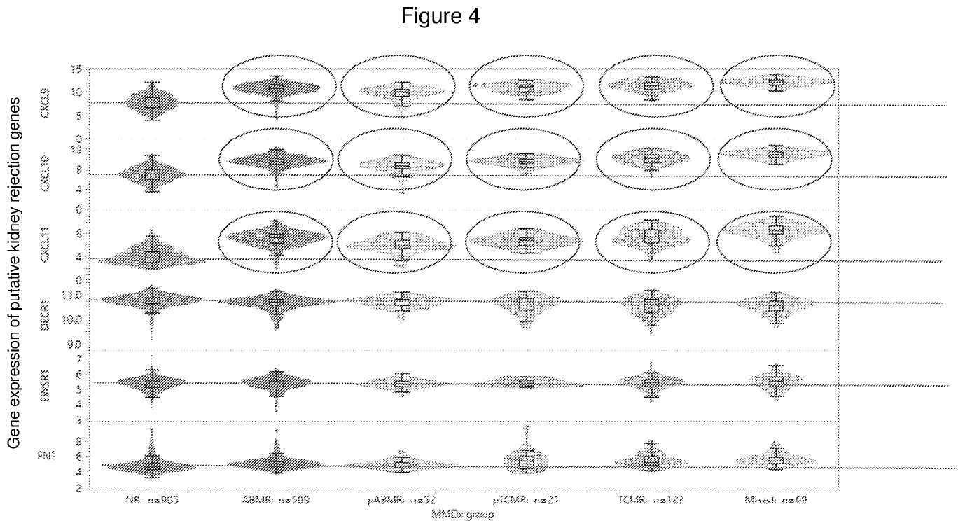

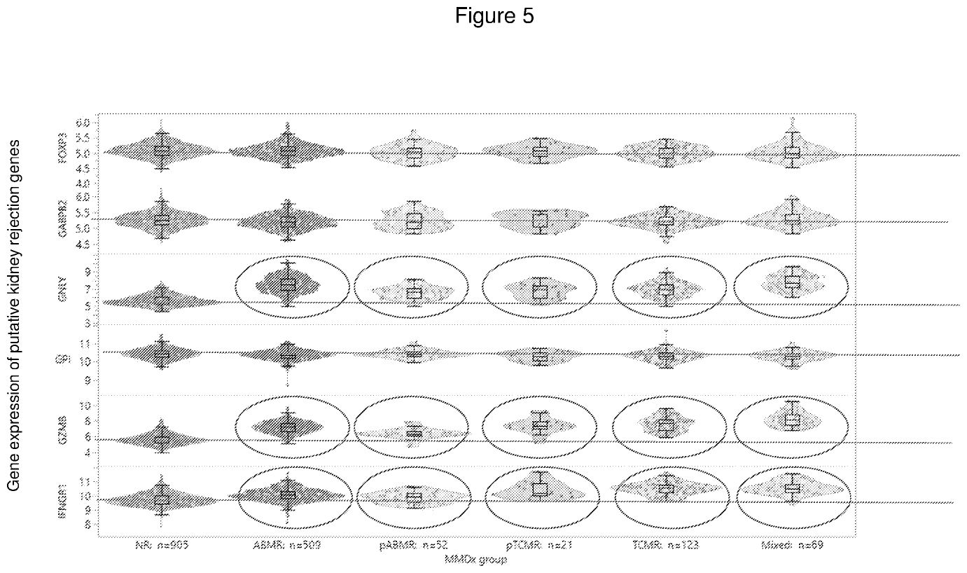

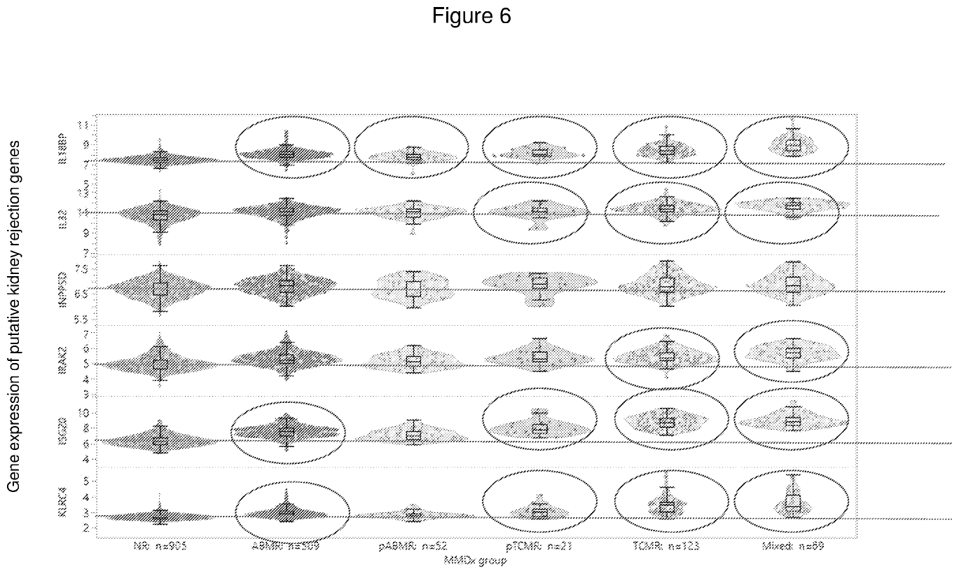

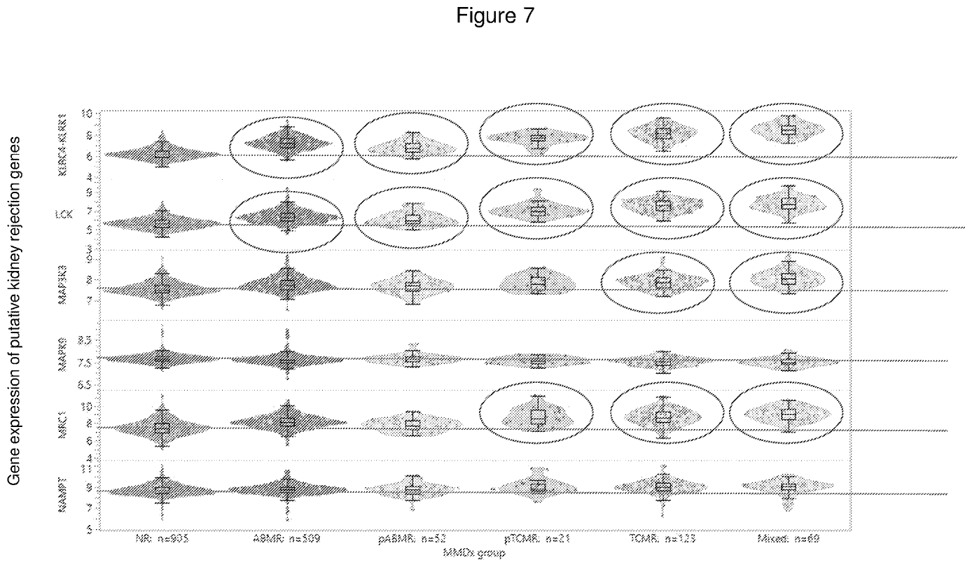

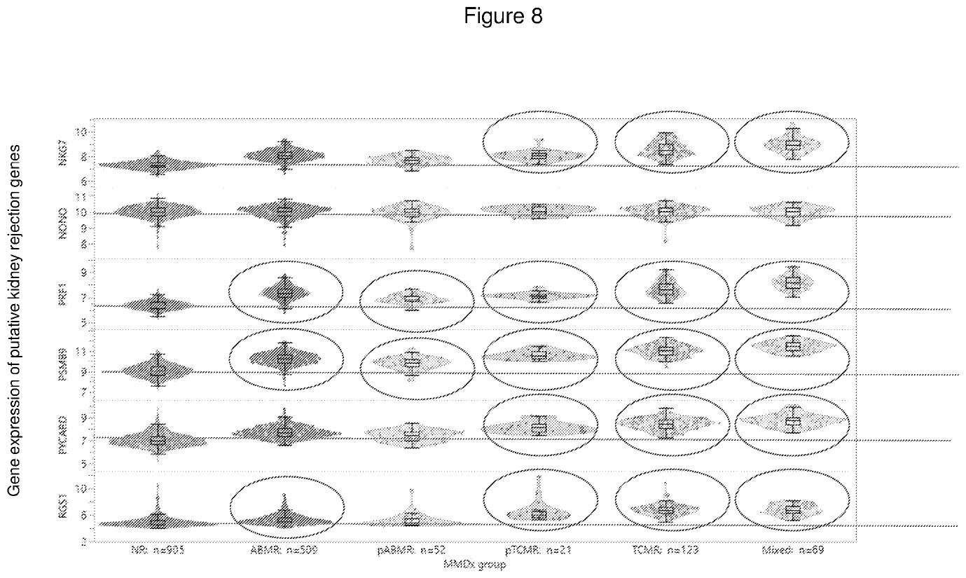

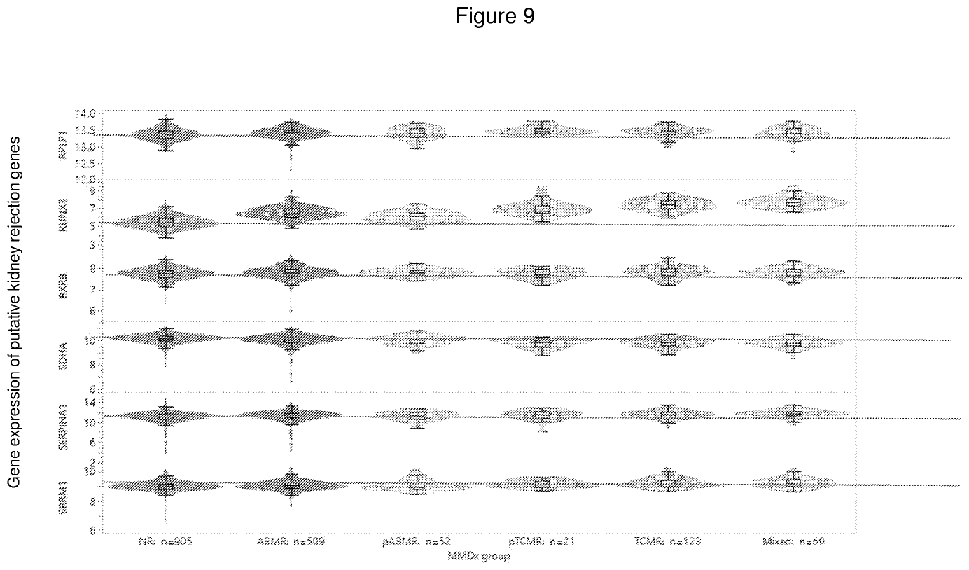

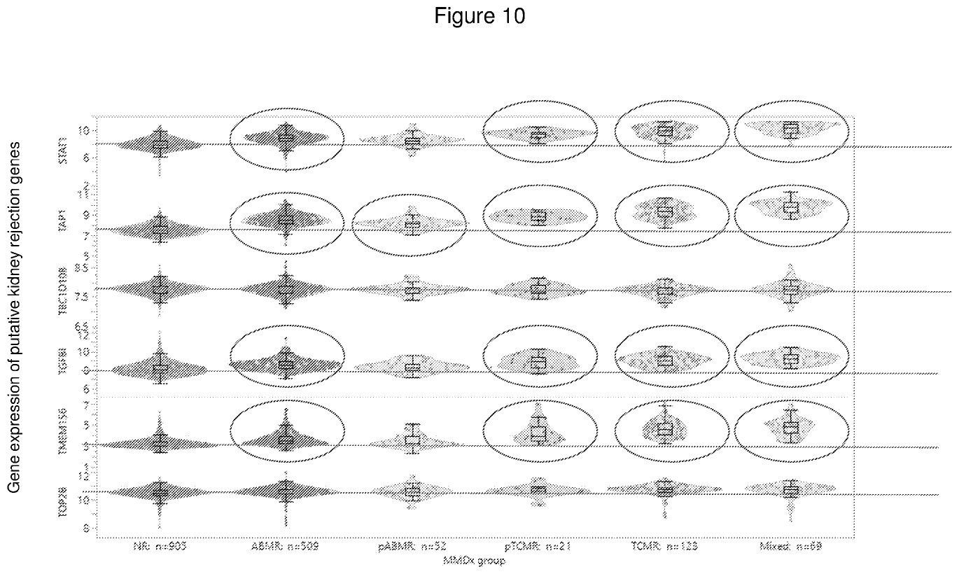

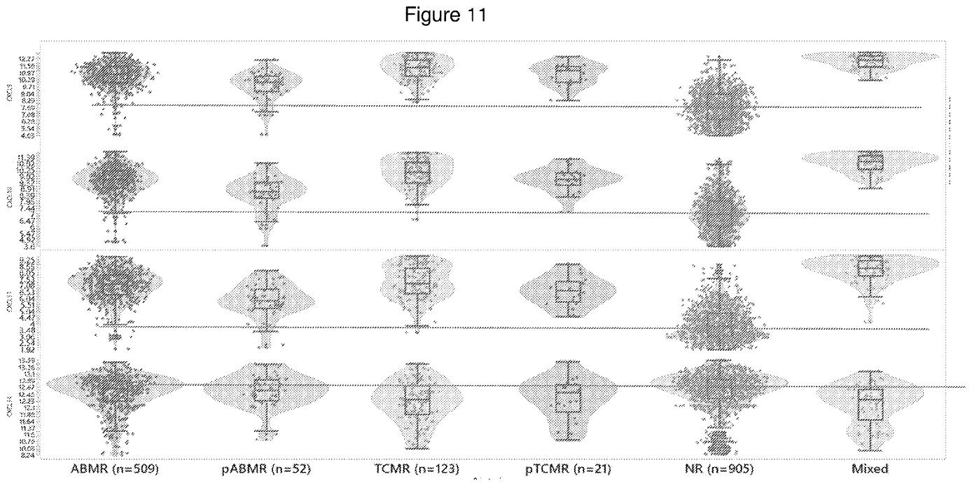

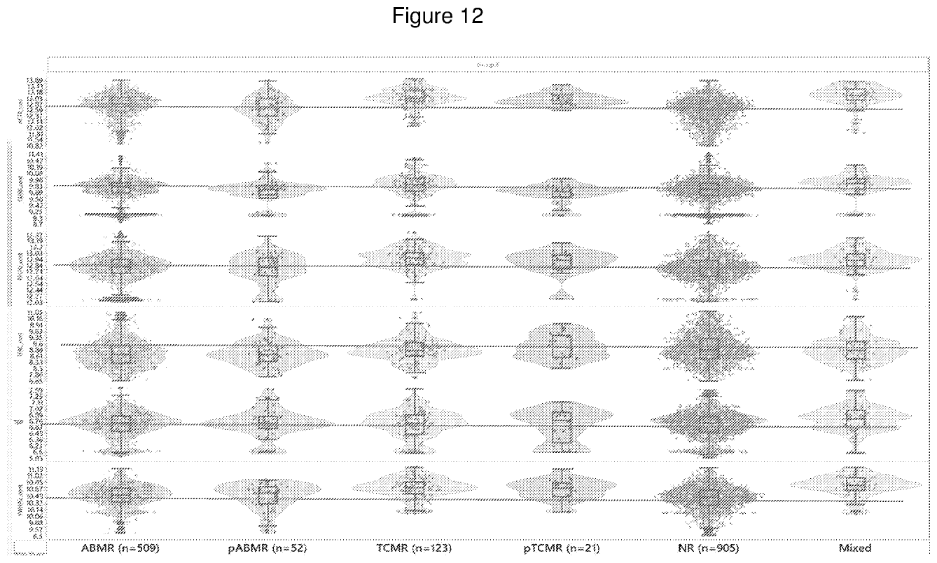

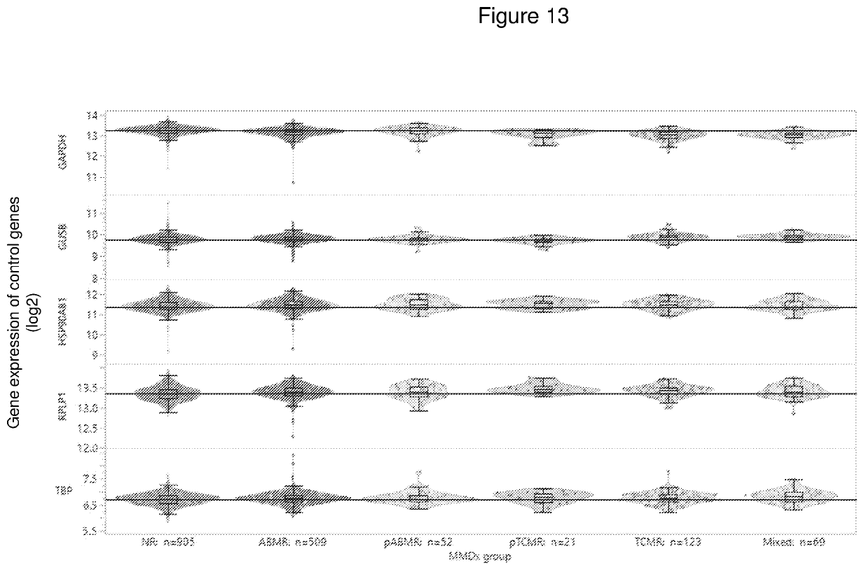

A t-Test was used to evaluate the performance of mRNAs expressed from target genes to differentiate between the different kidney rejection states as shown in Table 3 (see Tables section below). 49 target genes that can effectively differentiate between the different kidney rejection states were identified herein by correlating MMDx® genes with genes expressed in urine samples from subjects representing different rejection states and evaluating the performance with a t-Test. FIGS. 1-11 show the expression of some of these target gene (PSMB9, GZMB, GNLY, CXCL11, and CXCL9) over the different kidney rejection states. Control genes did not show any variation over the different kidney rejection states as shown in FIGS. 12-13.

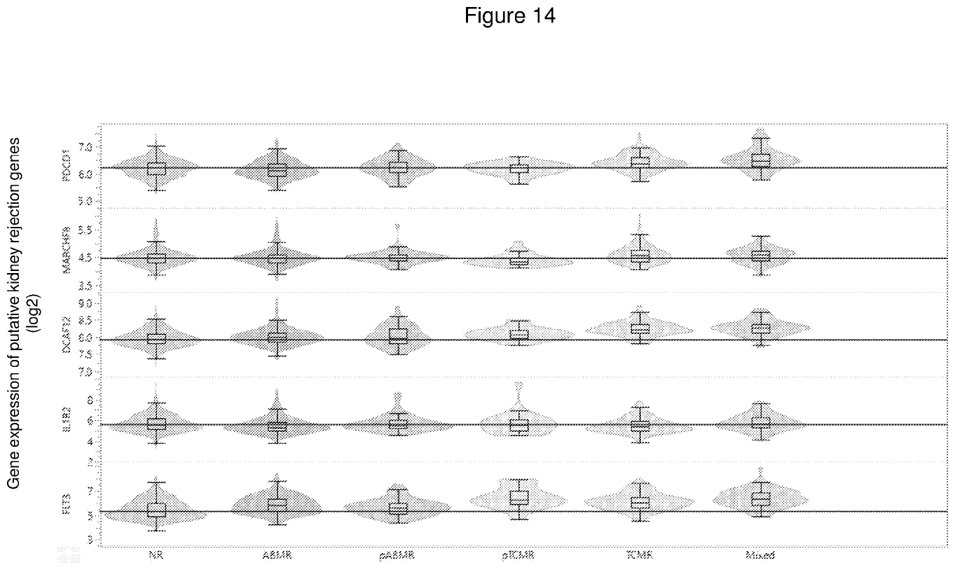

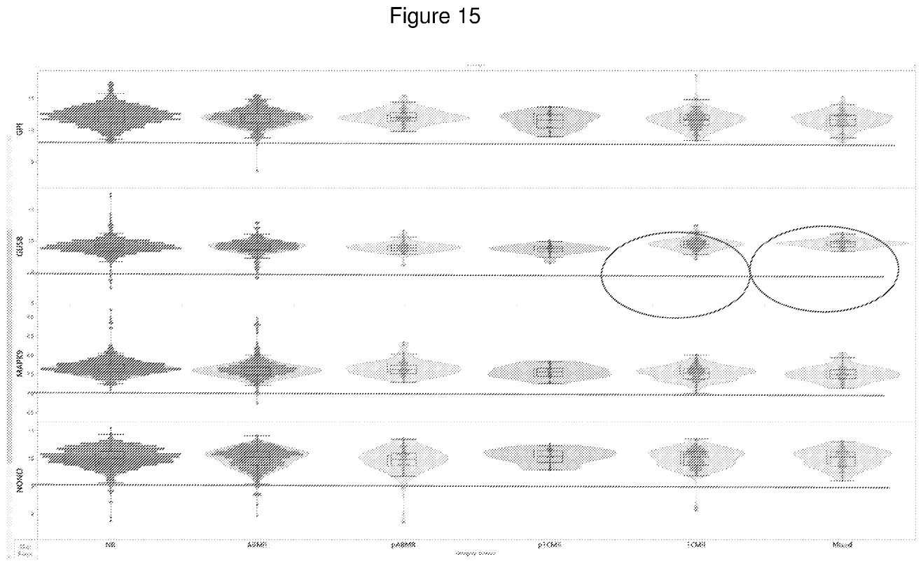

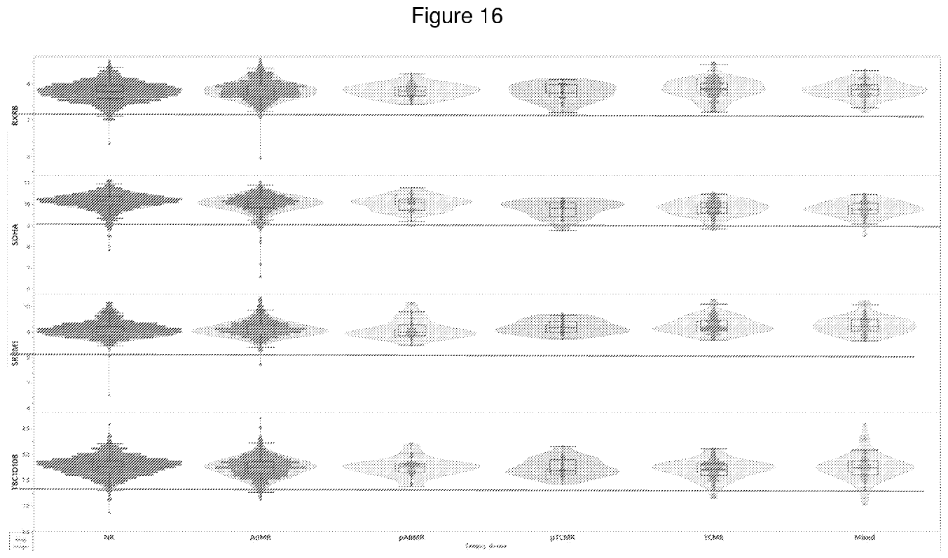

As shown in Table 3 and FIGS. 14-16, only a few of the genes identified from blood samples from the PaxGene® RNA study reported in Akalin et al., Kidney 360, 2021, 2 (12) 1998-2009 (hereinafter referred to as “Akalin”) could be used to differentiate between the different kidney rejection states. The following genes identified from blood sample as reported in Akalin were found to be not particular effective at differentiating between the different kidney rejection stages: PDCD1, MARCHF8, DCAF12, IL1R2, FLT3, ITGA4, ITGAM, PF4, C6orf25, SEMA7A, and RHOU.

The 49 target genes identified by cross-referencing MMDx® and urine expression data described in Table 3 are CXCL9, CD3ϵ, IP-10, LCK, C1QB, PSMB9, Tim-3, Foxp3, FAM26F, ALB, CTB-186G2.1, CXCL11, GNLY, MRC1, RGS1, RP11-1049A21.2, TMEM156, Z98744.3, SERPINB12, C11orf21, AF001548, 18s ribosomal, Basp1, Cd6, Cxcl10, Cxcl9, Inpp5d, Isg20, Lck, Nkg7, Runx3, Tap1, GZMB, PRF1, CD74, IL32, STAT1, CXCL14, SERPINA1, B2M, C3, PYCARD, BMP7, TBP, NAMPT, IFNGR1, IRAK2, Calhm6, Klrc4-Klrk1, Psmb10, CD3delta, Map4k1, IL2Ra, TGFB1, FN1, CDH1, PRPF31, HAVCR2, and IL18BP.

Further analysis of the performance of target genes identified by cross-referencing the MMDx® data with publications of genes differentially expressed in urine samples from subjects having different rejection states was performed by using logistic regression analysis and determination of AUC values as shown in Table 4. The urine63p23g gene set is CXCL11, CXCL9, GNLY, PSMB9, TAP1, CXCL10, NKG7, ISG20, RUNX3, C11orf21, C1QB, LCK, INPP5D, CD6, CD3ε, MRC1, BASP1, TMEM156, ALB, FOXP3, RGS1, and SERPINB12. The urine139p49g gene set is: CXCL9, CD3ϵ, IP-10, LCK, C1QB, PSMB9, Tim-3, Foxp3, FAM26F, ALB, CTB-186G2.1, CXCL11, GNLY, MRC1, RGS1, RP11-1049A21.2, TMEM156, Z98744.3, SERPINB12, C11orf21, AF001548, 18s ribosomal, Basp1, Cd6, Cxcl10, Cxcl9, Inpp5d, Isg20, Lck, Nkg7, Runx3, and Tap1. These gene sets were compared with MMDx® 30p39g gene set, which is the 39 top MMDx® probe sets by t-tests from the paper (20 ABMR vs rest, 20 TCMR vs rest, 1 not found therefore 39 probe sets from 26 genes). See also Table 11 in Tables section below. In addition, the urine gene sets were compared with the Akalin gene sets Akalin10p5g, Akalin30p11g, and Akalinp14g, which are established gene signatures reported in Akalin. In particular, Akalin10p5g is a set of 5 target genes reported to be predictive of kidney transplant rejection in Akalin. Akalin30p11g includes the five target genes from Akalin10p5g and adds genes predictive of heart transplant rejection. Akalinp14g includes 14 genes that selected as reference genes, and these genes should not be predictive of kidney rejection states. As expected, the Akalinp14g had a poor performance and the gave lower AUC values than the target gene sets. Akalin30p11g performed better than Akalin10p5g, but none of the Akalin gene sets performed at the level of Urine 63p23g or Urine 139p49g.

The performance of the target gene sets was also evaluated by using the Banff® data set containing 1208 samples, including 215 ABMR samples, 87 TCMR samples, and 274 NR samples as shown in Table 5. Consistent with the results from the MMDx® system, the performance of the Urine 63p23g or Urine 139p49g was better than the performance of Akalin gene sets also in the Banff® system.

A total of 53 genes as shown in Table 6 were found herein to be particularly effective at differentiate between the different kidney rejection states. Detection of micro-RNAs (miRNAs) binding the mRNAs produced by these genes can also be used to distinguish between different kidney rejection genes. Table 6 also lists miRNAs predicted to bind the target gene products.

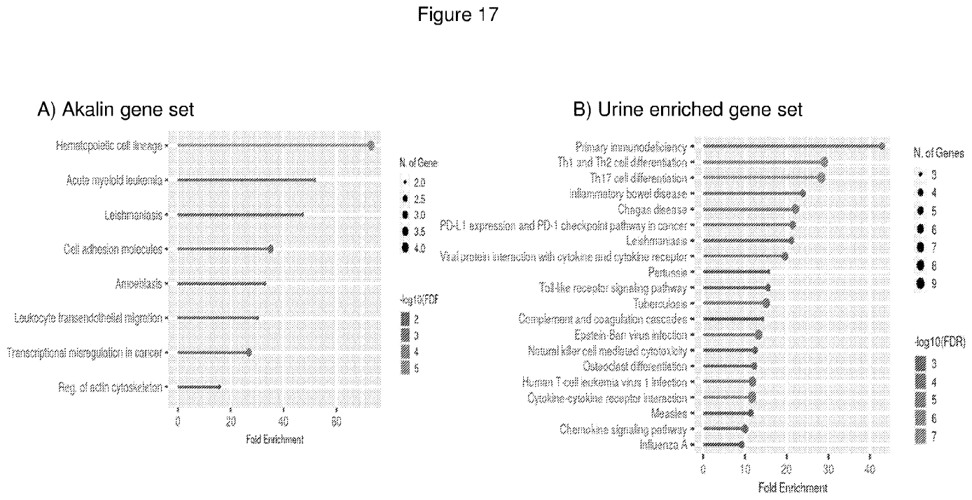

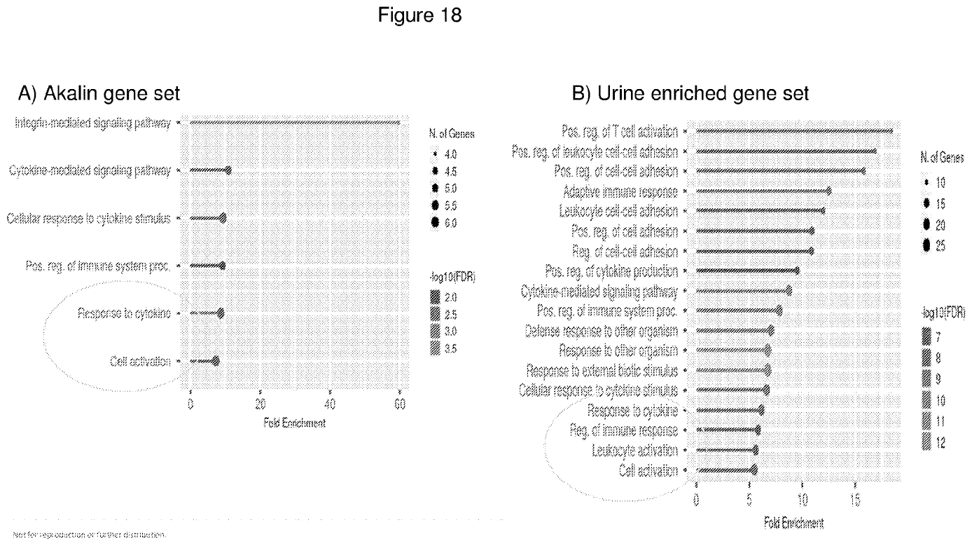

Gene ontology (GO) enrichment analysis demonstrated that the Urine 63p23g or Urine 139p49g gene sets were enriched for genes related to cytokine response/stimulus and immune activation to foreign agents as shown in Table 7. The GO analysis was performed using the following gene sets from the Human Molecular Signatures Database (MSigDB) for Gene Set Enrichment Analysis (GSEA): the hallmark allograft rejection gene set (GSEA-MSigDB: 200 genes), the hallmark inflammatory response gene set (GSEA-MSigDB: 200 genes), the KEGG allograft rejection gene set (GSEA-MSigDB: 38 genes), and the KEGG T-cell receptor gene set (GSEA-MSigDB: 108 genes). Although the Akalin gene set Akalin30p11g was enriched for cytokine response pathways, the Urine 63p23g or Urine 139p49g gene sets showed a much higher degree of immune action genes. See Table 7. In addition, the Urine 63p23g or Urine 139p49g gene sets were enriched for allograft and inflammatory genes, whereas the Akalin gene sets were not as readily matched with GSEA-MSigDB gene sets as shown in Table 8 and FIGS. 17-18.

The mRNAs expressed in urine appeared to be highly enriched in MMDx® disease classification data as determined by t-tests, one way anova, and AUC classifications.

It is also contemplated herein that target genes in the apoptosis pathway are useful for differentiating between kidney transplant rejection states such as the genes listed in Table 9 from the hallmark apoptosis gene set.

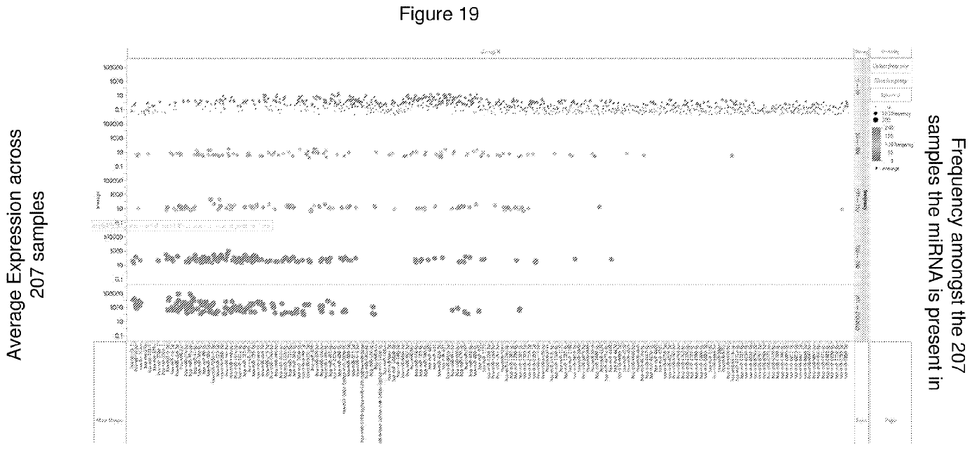

miRNAs can also be found in urine samples as shown in FIG. 19. Distinguishing between different kidney rejection states may also be performed by using miRNAs such as miRNAs shown in FIG. 19 or the miRNAs binding the mRNAs expressed from the target genes listed in Tables 1 and 6. In some embodiments, the one or more miRNAs useful for determining kidney rejection states are selected from the group consisting of miR-186-5p, miR-665, miR-873-5p.1, miR-543, miR-330-3p, miR-362-5p/500b-5p, miR-217, miR-140-5p, miR-193-3p, miR-382-5p, miR-140-3p.2, miR-653-5p, miR-455-3p.2, miR-145-5p, miR-491-5p, miR-23-3p, miR-375, miR-129-5p, miR-96-5p/1271-5p, miR-182-5p, miR-371-5p, miR-203a-3p.1, miR-494-3p, miR-146-5p, miR-140-3p.1, miR-125-5p, miR-346, miR-760, miR-185-5p, miR-325-3p, miR-15-5p/16-5p/195-5p/424-5p/497-5p, miR-503-5p, miR-423-5p, miR-496.1, miR-155-5p, miR-142-3p.2, miR-24-3p, miR-874-3p, miR-25-3p/32-5p/92-3p/363-3p/367-3p, miR-17-5p/20-5p/93-5p/106-5p/519-3p, miR-181-5p, miR-142-5p, miR-130-3p/301-3p/454-3p, miR-21-5p/590-5p, miR-103-3p/107, miR-137, miR-340-5p, miR-490-3p, miR-143-3p, miR-409-3p, miR-27-3p, miR-138-5p, miR-485-5p, miR-328-3p, miR-326, miR-148-3p/152-3p, miR-9-5p, miR-31-5p, miR-452-5p/892-3p, miR-202-5p, miR-29-3p, miR-338-3p, miR-26-5p, let-7-5p/98-5p, miR-196-5p, miR-30-5p, miR-142-3p.1, miR-19-3p, miR-411-3p, miR-493-5p, miR-218-5p, miR-203a-3p.2, miR-495-3p, miR-425-5p, miR-135-5p, miR-154-3p/487-3p, miR-223-3p, miR-219-5p, miR-670-3p, miR-216b-5p, miR-200bc-3p/429, miR-320, miR-216a-5p, miR-141-3p/200a-3p, miR-144-3p, miR-128-3p, miR-455-3p.1, miR-219a-2-3p, miR-873-5p.2, miR-448, miR-183-5p.2, miR-374-5p, miR-505-3p.1, miR-433-3p, miR-377-3p, miR-365-3p, miR-124-3p.1, miR-410-3p, miR-199-3p, miR-22-3p, miR-129-3p, miR-383-5p.1, miR-1-3p/206, miR-296-5p, miR-299-3p, miR-212-5p, miR-331-3p, miR-378-3p, miR-136-5p, miR-1193, miR-505-3p.2, miR-302c-3p.2/520-3p, miR-421, miR-499a-5p, miR-302-3p/372-3p/373-3p/520-3p, miR-124-3p.2/506-3p, miR-34-5p/449-5p, miR-376c-3p, miR-139-5p, miR-221-3p/222-3p, miR-504-5p.1, miR-335-5p, miR-101-3p.1, miR-431-5p, miR-489-3p, miR-369-3p, miR-330-3p.2, miR-18-5p, miR-28-5p/708-5p, miR-133a-3p.2/133b, miR-205-5p, miR-199-5p, miR-455-5p, miR-126-3p.2, miR-7-5p, miR-483-3p.2, miR-668-3p, miR-1306-5p, miR-150-5p, miR-296-3p, miR-204-5p/211-5p, miR-3064-5p, miR-532-5p, miR-876-5p, miR-501-3p/502-3p, miR-33-5p, miR-153-3p, miR-214-5p, miR-655-3p, miR-342-3p, miR-133a-3p.1, miR-411-5p.1, miR-496.2, miR-411-5p.2, miR-582-5p, miR-381-3p, miR-188-5p, miR-383-5p.2, miR-486-5p, miR-183-5p.1, miR-208-3p, miR-193a-5p, miR-101-3p.2, miR-542-3p, miR-190-5p, miR-299-5p, miR-154-5p, miR-802, miR-323-3p, miR-532-3p, miR-224-5p, miR-339-5p, miR-194-5p, miR-149-5p, miR-493-3p, miR-382-3p, miR-132-3p/212-3p, miR-1197, miR-99-5p/100-5p, miR-877-5p, miR-483-3p.1, miR-10-5p, miR-361-5p, miR-539-3p, miR-191-5p, miR-329-3p/362-3p, miR-122-5p, miR-379-5p, miR-376-3p, miR-1298-5p, miR-451, miR-210-3p, miR-1224-5p, miR-324-5p, miR-544a-5p, miR-488-3p, miR-758-3p, miR-151-3p, miR-875-5p, miR-134-5p, miR-192-5p/215-5p, and miR-127-3p. Preferably, the one or more miRNAs useful for determining kidney rejection states are selected from the group consisting of miR-96-5p/1271-5p, miR-493-5p, miR-183-5p.2, miR-150-5p, miR-7-5p, miR-653-5p, miR-200bc-3p/429, miR-212-5p, miR-1298-5p, miR-137, miR-758-3p, miR-325-3p, miR-542-3p, miR-25-3p/32-5p/92-3p/363-3p/367-3p, miR-495-3p, miR-30-5p, miR-873-5p.1, miR-146-5p, miR-505-3p.1, miR-539-3p, miR-216a-5p, miR-216b-5p, miR-340-5p, miR-361-5p, miR-338-3p, miR-217, miR-9-5p, miR-219a-2-3p, miR-15-5p/16-5p/195-5p/424-5p/497-5p, miR-503-5p, miR-199-3p, miR-1-3p/206, miR-17-5p/20-5p/93-5p/106-5p/519-3p, miR-302-3p/372-3p/373-3p/520-3p, miR-142-5p, miR-302c-3p.2/520-3p, miR-326, miR-760, miR-138-5p, miR-27-3p, miR-145-5p, miR-142-3p.2, miR-101-3p.2, miR-182-5p, miR-203a-3p.2, miR-140-3p.1, miR-183-5p.1, miR-144-3p, miR-101-3p.1, miR-330-3p, miR-224-5p, miR-148-3p/152-3p, miR-485-5p, miR-122-5p, miR-155-5p, miR-320, miR-23-3p, miR-124-3p.2/506-3p, miR-135-5p, miR-381-3p, miR-26-5p, miR-1224-5p, miR-192-5p/215-5p, miR-1249-3p, miR-125-5p, miR-483-3p.2, miR-668-3p, miR-223-3p, miR-655-3p, miR-382-5p, miR-130-3p/301-3p/454-3p, miR-19-3p, miR-582-5p, miR-194-5p, miR-802, miR-483-3p.1, miR-382-3p, miR-129-5p, miR-3064-5p, miR-873-5p.2, miR-499a-5p, miR-128-3p, miR-532-5p, miR-296-5p, miR-744-5p, miR-425-5p, miR-218-5p, and miR-496.1.

### Example 2: Obtaining Urine Samples and Measuring RNA

### Urine Samples

Urine samples are collected from patients receiving a donor kidney. In particular, urine samples (about 50 ml) from the enrolled kidney transplant recipients may be collected longitudinally with the pre-specified schedule for collection being pre-transplantation, post-transplant days 3, 7, 15 and 30 and months 2, 3, 4, 5, 6, 9 and 12, and at the time of any renal allograft biopsy and prior to treatment and 2-weeks after biopsy. Urine cell pellets are prepared using a standard protocol for urine cell sedimentation, and pellets are stored at −80° C. Time points of obtaining patient urine samples are prior to, at the time, and at various time intervals following transplantation surgery. Samples may be biopsy-matched and urine samples are obtained at the time of clinical dysfunction and biopsy or at the time of protocol biopsy (at which time most patients do not have clinical dysfunction). In addition, urine sample may be serially obtained post transplantation.

### Isolation of Nucleic Acids in Urine Samples

Nucleic acids such as RNA or DNA, and in particular cell-free DNA, mRNA, and microRNA is extracted from urine samples. Total RNA are isolated from urinary cell pellets using commercially available kits for isolating RNA. In general, the urinary cell pellets are lysed by adding one volume of a lysis buffer and vortexing. Following the addition of an equal volume of 100% ethanol, the samples are transferred to an RNA spin cartridge. The cartridge is then washed 3 to 4 times with the wash buffers provided in the kit and the total RNA is eluted from the cartridge with 30 μl of RNase-free water.

Circulating nucleic acids may be obtained by using the QIAamp™ Circulating Nucleic Acid Kit (Qiagen). Cellular nucleic acids are obtained by isolating cells from the urine samples by centrifugation. LabChip™ NGS 5k kit (Perkin Elmer, Waltham, MA, USA) is used for quantification.

The quantity (absorbance at 260 nm) and purity (ratio of the absorbance at 260 and 280 nm) of the RNA isolated from the urine cell pellet are measured using for example the NanoDrop® ND-1000 UV-Vis spectrophotometer (Thermo Scientific).

Measurement of Target mRNAs from Urine Sample

The total RNA is reverse transcribed (RT) to cDNA using for example the TaqMan® reverse transcription kit (Cat. N808-0234, Applied Biosystems) on the same day the total RNA is isolated. The RT is performed by combining 1.0 μg of total RNA in 100 μl volume, and the final concentration of 1×TaqMan RT buffer, 5.5 mM of Magnesium Chloride, 500 μM each of 4 dNTPs, 2.5 μM of Random Hexamer, 0.4 Unit/μl of RNase inhibitor, and 1.25 Unit/μl of MultiScribe® Reverse Transcriptase. The sample was incubated at 25° C. for 10 min, 48° C. for 30 min, and 95° C. for 5 min.

Multiplex Real-time PCR reactions are performed on the cDNA using for example Amplifluor® Universal Detection system (Intergen) and iCycler® (BioRad). The qPCR assays may be run on an Applied Biosystems 7500 Real Time PCR instrument and/or the Biorad CFX, but useful instrument platforms are not limited thereto. The qPCR assays of the present invention may be adapted to work on most Real-Time PCR instruments. The following PCR conditions may be used, but they can be modified as necessary: 10 min 95° C. denaturation cycle, followed by 32 cycles of 2-step qPCR (15 s at 95° C. and 2 min at 60° C. (annealing) and 10 minutes at 72° C. (extension time)). Additional PCR parameters (i.e. cycle number, denaturation and annealing/extension times and temperatures) are investigated to obtain a robust, sensitive qPCR multiplex as described elsewhere herein.

The quantitation of RNA molecules is performed by using standard methods known in the art such as the comparative CT method (2-AA CT Method).

In some embodiment, the quantitative real-time PCR is used to determine the quantity of 6, 12, 24, 48, or 96 target loci in one reaction. In some embodiments, single gene specific oligonucleotide pairs and TaqMan® probes are used to measure RNA molecules. The real time PCR can be performed with any list of RNA molecules provided in herein or combinations thereof. In particular, the quantitative PCR is performed to measure the quantity of the mRNAs expressed by the target genes CXCL11, CXCL9, GNLY, PSMB9, TAP1, CXCL10, NKG7, ISG20, RUNX3, C11orf21, C1QB, LCK, INPP5D, CD6, CD3ϵ, MRC1, BASP1, TMEM156, ALB, FOXP3, RGS1, and SERPINB12. The urine139p49g gene set is: CXCL9, CD3€, IP-10, LCK, C1QB, PSMB9, Tim-3, Foxp3, FAM26F, ALB, CTB-186G2.1, CXCL11, GNLY, MRC1, RGS1, RP11-1049A21.2, TMEM156, Z98744.3, SERPINB12, C11orf21, AF001548, 18s ribosomal, Basp1, Cd6, Cxcl10, Cxcl9, Inpp5d, Isg20, Lck, Nkg7, Runx3, Tap1, and combinations thereof.

Alternatively, RNA expression may be quantified by using microarray. For example, the Human Genome U219 Array comprised of more than 530,000 probes covering more than 36,000 transcripts and variants, which represent more than 20,000 genes mapped through UniGene or via RefSeq annotation. The EST and mRNA sequences used in the design were clustered and assembled to create consensus sequences that represent alternative splice forms. Each assembly was then analyzed for orientation and alternative 3′ end evidence. Content was chosen to cover all well-annotated genes and transcripts from RefSeq v36 and by leveraging all available EST and mRNA evidence that fall into the same clusters, to rigorously detect alternate 3′ ends of those well-annotated genes. More than 1,000 probe sets represent transcripts that have no official gene symbol in UniGene, but are based on predicted RefSeq sequences and UniGene clusters with good evidence of actual transcription

The Prime View Human Genome 96-array Plate and Trays enables high-throughput expression profiling of 96 samples at a time using probe sets with an emphasis on established, well-annotated content. Sequences used in the design of the array were selected from the UniGene database, RefSeq version 36, and full-length human mRNAs from GenBank.

Matched (urine collected minus 3 days to plus 1 day of biopsy) and quality control passed urine samples may be included for those who underwent a biopsy, whereas all quality control passed urine samples collected longitudinally may be included for the no biopsy group.

### Example 3: Prediction of Rejection States from the RNA Measurements

In each sample, RNA is measured and correlated with rejection status. Where applicable, all statistical tests are two sided. Significance is set at p<0.05. Data may be analyzed using a Kruskal-Wallis rank sum test followed by Dunn multiple comparison tests with Holm correction.

The data was evaluated by the area under the ROC curve (AUC) of a fitted model in addition to sensitivity and specificity for diagnosing rejection. An AUC of 1.0 indicates perfect concordance; e.g., every rejection state has a higher score on the diagnostic signature than every non-rejection state. An AUC of 0.50 would indicate that the ability of the diagnostic signature to differentiate an acute cellular rejection biopsy from a biopsy without acute cellular rejection is no better than chance.

### Example 4: Combining RNA Measurements with cfDNA Measurements

The determination of rejection status as shown in Examples 1-3 may be combined with predictions of rejection status based on measuring cell-free DNA (cfDNA) as described elsewhere.

### TABLES

TABLE 1
List of 727 mRNAs expressed in urine samples and miRNAs predicted to bind the mRNAs

| Urine all 727 | | |
|---|---|---|
| Dooley genes | | |
| (Dooley et al. | | |
| Transplant | | |
| Direct. 2020 | | How |
| August; 6(8): | | many |
| e588.) | miRNAs predicted to bind | found |
| ITM2A | | 0 |
| SLAMF6 | miR-186-5p, miR-665 | 2 |
| IKZF3 | miR-873-5p.1, miR-543, miR-330-3p, miR-362-5p/500b-5p, miR-217, miR-140-5p, | 31 |
| | miR-193-3p, miR-382-5p, miR-140-3p.2, miR-653-5p, miR-455-3p.2, miR-145-5p, | |
| | miR-491-5p, miR-23-3p, miR-23-3p, miR-375, miR-129-5p, miR-96-5p/1271-5p, miR- | |
| | 182-5p, miR-182-5p, miR-96-5p/1271-5p, miR-371-5p, miR-203a-3p.1, miR-494-3p, | |
| | miR-146-5p, miR-140-3p.1, miR-125-5p, miR-346, miR-346, miR-760, miR-185-5p | |
| CCL5 | | 0 |
| CXCL9 | miR-325-3p | 1 |
| IGHM | | 0 |
| ITM2A | | 0 |
| HIST1H2AJ | miR-760 | 1 |
| CXCL10 | miR-15-5p/16-5p/195-5p/424-5p/497-5p, miR-503-5p | 2 |
| CD3E | | 0 |
| NKG7 | | 0 |
| TRBC2 | | 0 |
| HIST1H3I | miR-760, miR-423-5p | 2 |
| TRGC2 | | 0 |
| HIST1H1B | miR-760 | 1 |
| CTLA4 | miR-496.1, miR-155-5p, miR-142-3p.2, miR-142-3p.2 | 4 |
| PDCD1 | miR-15-5p/16-5p/195-5p/424-5p/497-5p, miR-15-5p/16-5p/195-5p/424-5p/497-5p, | 7 |
| | miR-15-5p/16-5p/195-5p/424-5p/497-5p, miR-15-5p/16-5p/195-5p/424-5p/497-5p, | |
| | miR-15-5p/16-5p/195-5p/424-5p/497-5p, miR-15-5p/16-5p/195-5p/424-5p/497-5p, | |
| | miR-15-5p/16-5p/195-5p/424-5p/497-5p | |
| GZMA | | 0 |
| IL2RB | miR-24-3p, miR-15-5p/16-5p/195-5p/424-5p/497-5p, miR-874-3p | 3 |
| IKZF3 | miR-873-5p.1, miR-543, miR-330-3p, miR-362-5p/500b-5p, miR-217, miR-140-5p, | 31 |
| | miR-193-3p, miR-382-5p, miR-140-3p.2, miR-653-5p, miR-455-3p.2, miR-145-5p, | |
| | miR-491-5p, miR-23-3p, miR-23-3p, miR-375, miR-129-5p, miR-96-5p/1271-5p, miR- | |
| | 182-5p, miR-182-5p, miR-96-5p/1271-5p, miR-371-5p, miR-203a-3p.1, miR-494-3p, | |
| | miR-146-5p, miR-140-3p.1, miR-125-5p, miR-346, miR-346, miR-760, miR-185-5p | |
| CD69 | miR-25-3p/32-5p/92-3p/363-3p/367-3p, miR-25-3p/32-5p/92-3p/363-3p/367-3p, miR- | 13 |
| | 25-3p/32-5p/92-3p/363-3p/367-3p, miR-17-5p/20-5p/93-5p/106-5p/519-3p, miR-181- | |
| | 5p, miR-15-5p/16-5p/195-5p/424-5p/497-5p, miR-142-5p, miR-130-3p/301-3p/454-3p, | |
| | miR-21-5p/590-5p, miR-103-3p/107, miR-137, miR-325-3p, miR-340-5p | |
| SIT1 | miR-491-5p, miR-125-5p, miR-15-5p/16-5p/195-5p/424-5p/497-5p, miR-15-5p/16- | 7 |
| | 5p/195-5p/424-5p/497-5p, miR-760, miR-490-3p, miR-143-3p | |
| CD96 | miR-409-3p, miR-182-5p | 2 |
| FAIM3 | miR-27-3p, miR-138-5p, miR-96-5p/1271-5p | 3 |
| ZAP70 | | 0 |
| HIST1H1D | miR-760 | 1 |
| TRAC | | 0 |
| RNASE6 | miR-330-3p, miR-137, miR-325-3p | 3 |
| SLAMF6 | miR-186-5p, miR-665 | 2 |
| Z98744.2 | | 0 |
| GZMB | | 0 |
| IL18R1 | miR-485-5p | 1 |
| IL1RL1 | | 0 |
| HIST1H4L | miR-328-3p | 1 |
| C1QB | | 0 |
| CD8A | miR-326 | 1 |
| HIST1H2BM | miR-760 | 1 |
| HLA-DQB1 | miR-148-3p/152-3p | 1 |
| LCK | miR-325-3p | 1 |
| ASB2 | miR-874-3p | 1 |
| HIST1H2AI | miR-760, miR-9-5p | 2 |
| SLFN12L | miR-23-3p | 1 |
| CD28 | miR-31-5p, miR-24-3p, miR-452-5p/892-3p, miR-15-5p/16-5p/195-5p/424-5p/497-5p, | 8 |
| | miR-503-5p, miR-145-5p, miR-27-3p, miR-202-5p | |
| TRBV28 | | 0 |
| RP4-620F22.2 | | 0 |
| GIMAP1 | | 0 |
| ICOS | miR-29-3p, miR-338-3p, miR-26-5p, miR-27-3p, let-7-5p/98-5p, miR-196-5p | 5 |
| ZNF831 | miR-30-5p, miR-142-3p.1, miR-19-3p, miR-19-3p, miR-411-3p, miR-485-5p, miR-493- | 23 |
| | 5p, miR-218-5p, miR-325-3p, miR-203a-3p.2, miR-203a-3p.2, miR-142-3p.2, miR-340- | |
| | 5p, miR-15-5p/16-5p/195-5p/424-5p/497-5p, miR-15-5p/16-5p/195-5p/424-5p/497-5p, | |
| | miR-495-3p, miR-186-5p, miR-425-5p, miR-135-5p, miR-362-5p/500b-5p, miR-142- | |
| | 5p, miR-142-5p, miR-154-3p/487-3p | |
| RGS1 | miR-223-3p, miR-655-3p, miR-27-3p | 3 |
| TRAT1 | miR-325-3p, miR-203a-3p.1, miR-203a-3p.2 | 3 |
| GIMAP7 | | 0 |
| P2RY10 | | 0 |
| HCST | | 0 |
| HIST1H2AK | miR-760 | 1 |
| GBP5 | miR-219-5p | 1 |
| EMB | let-7-5p/98-5p, miR-142-3p.2, miR-670-3p, miR-29-3p, miR-216b-5p, miR-200bc- | 8 |
| | 3p/429, miR-320, miR-9-5p, miR-330-3p | |
| CD2 | | 0 |
| KLRC1 | | 0 |
| CD6 | miR-216a-5p, miR-216b-5p | 2 |
| GPR174 | miR-15-5p/16-5p/195-5p/424-5p/497-5p, miR-503-5p, miR-141-3p/200a-3p, miR-144- | 8 |
| | 3p, miR-219-5p, miR-27-3p, miR-130-3p/301-3p/454-3p, miR-128-3p | |
| AL049822.1 | | 0 |
| HLA-DQA1 | miR-137, miR-325-3p | 2 |
| TIGIT | miR-455-3p.1, miR-23-3p, miR-219a-2-3p | 3 |
| ITK | miR-873-5p.2, miR-155-5p, miR-140-3p.2 | 3 |
| ST8SIA4 | miR-19-3p, miR-203a-3p.2, miR-448, miR-218-5p, miR-183-5p.2, miR-103-3p/107, | 30 |
| | miR-30-5p, miR-374-5p, miR-505-3p.1, miR-30-5p, miR-27-3p, miR-433-3p, miR-26- | |
| | 5p, miR-377-3p, miR-653-5p, miR-365-3p, miR-181-5p, miR-125-5p, miR-124-3p.1, | |
| | miR-181-5p, miR-142-5p, miR-410-3p, miR-199-3p, miR-22-3p, miR-129-3p, miR- | |
| | 143-3p, miR-203a-3p.1, miR-9-5p, miR-128-3p, miR-383-5p.1 | |
| GIMAP6 | | 0 |
| GVINP1 | | 0 |
| HLA-DQB1- | | 0 |
| AS1 | | |
| MRC1 | miR-23-3p | 1 |
| HLA-DPB1 | | 0 |
| CCR2 | | 0 |
| CCND2-AS1 | | 0 |
| AL009179.1 | | 0 |
| EVI2B | miR-203a-3p.2, miR-325-3p, miR-340-5p, miR-203a-3p.1 | 4 |
| FCGR3B | | 0 |
| ETV7 | | 0 |
| CTSW | miR-140-3p.1 | 1 |
| HIST1H2AL | miR-760 | 1 |
| THEMIS | miR-186-5p | 1 |
| CH17-373J23.1 | | 0 |
| BLM | | 0 |
| PRF1 | | 0 |
| DOK2 | | 0 |
| CXCR6 | | 0 |
| NGFR | miR-760, miR-1-3p/206, miR-423-5p, miR-296-5p, miR-27-3p, miR-299-3p, miR-128- | 10 |
| | 3p, miR-212-5p, miR-326, miR-326 | |
| KCNE1 | miR-22-3p, miR-193-3p, miR-218-5p, miR-331-3p | 4 |
| HIST1H3H | miR-760 | 1 |
| CTD- | | 0 |
| 2313F11.1 | | |
| HLA-DRA | miR-325-3p | 1 |
| CD8B | | 0 |
| NCF1 | miR-325-3p, miR-19-3p | 2 |
| GZMK | | 0 |
| MLKL | | 0 |
| HIST1H3B | miR-760 | 1 |
| CD3G | miR-653-5p, miR-494-3p, miR-378-3p, miR-136-5p | 4 |
| JAK3 | miR-1193 | 1 |
| CD3D | miR-146-5p, miR-325-3p, miR-505-3p.1 | 3 |
| C1QA | | 0 |
| IFNG | miR-340-5p, miR-29-3p, miR-24-3p, miR-125-5p | 4 |
| CDCA7 | miR-505-3p.2, miR-30-5p, miR-302c-3p.2/520-3p, miR-302c-3p.2/520-3p, miR-421, | 11 |
| | miR-499a-5p, miR-124-3p.1, miR-495-3p, miR-17-5p/20-5p/93-5p/106-5p/519-3p, | |
| | miR-302-3p/372-3p/373-3p/520-3p, miR-302-3p/372-3p/373-3p/520-3p | |
| Z98744.3 | | 0 |
| CXCL11 | miR-199-3p, miR-9-5p, miR-1-3p/206 | 3 |
| RP11-284N8.3 | | 0 |
| HLA-H | | 0 |
| TOX | miR-124-3p.2/506-3p, miR-27-3p, miR-34-5p/449-5p, miR-30-5p, miR-96-5p/1271-5p, | 20 |
| | miR-129-5p, miR-23-3p, miR-182-5p, miR-543, miR-144-3p, miR-376c-3p, miR-302- | |
| | 3p/372-3p/373-3p/520-3p, miR-124-3p.1, miR-139-5p, miR-139-5p, miR-221-3p/222- | |
| | 3p, miR-142-5p, miR-181-5p, miR-302c-3p.2/520-3p, miR-504-5p.1 | |
| GIMAP4 | | 0 |
| FAM78A | miR-124-3p.1, miR-27-3p, miR-137, miR-218-5p, miR-96-5p/1271-5p, miR-182-5p, | 19 |
| | miR-182-5p, miR-302-3p/372-3p/373-3p/520-3p, miR-128-3p, miR-128-3p, miR-130- | |
| | 3p/301-3p/454-3p, miR-335-5p, miR-330-3p, miR-125-5p, miR-101-3p.1, miR-124- | |
| | 3p.2/506-3p, miR-124-3p.2/506-3p, miR-31-5p, miR-24-3p | |
| FAM65B | miR-340-5p, miR-431-5p, miR-146-5p, miR-218-5p, miR-489-3p, miR-27-3p, miR-15- | 14 |
| | 5p/16-5p/195-5p/424-5p/497-5p, miR-369-3p, miR-128-3p, miR-410-3p, miR-29-3p, | |
| | miR-125-5p, miR-330-3p.2, miR-374-5p | |
| HCG4P7 | | 0 |
| SMAP2 | miR-18-5p, miR-320, miR-9-5p, miR-138-5p | 4 |
| EOMES | miR-96-5p/1271-5p, miR-330-3p.2, miR-25-3p/32-5p/92-3p/363-3p/367-3p, miR-330- | 12 |
| | 3p, miR-29-3p, miR-28-5p/708-5p, miR-182-5p, miR-182-5p, miR-23-3p, miR-183- | |
| | 5p.2, miR-493-5p, miR-96-5p/1271-5p | |
| RASAL3 | | 0 |
| CD163 | miR-136-5p, miR-23-3p, miR-181-5p, miR-15-5p/16-5p/195-5p/424-5p/497-5p | 4 |
| FCRL3 | | 0 |
| IFIT2 | miR-133a-3p.2/133b | 1 |
| SH2D1A | miR-31-5p | 1 |
| HCG4P11 | | 0 |
| RGS18 | miR-205-5p | 1 |
| MIR155HG | | 0 |
| IFITM1 | miR-325-3p | 1 |
| HIST1H2BL | miR-760 | 1 |
| ITGA4 | miR-130-3p/301-3p/454-3p, miR-17-5p/20-5p/93-5p/106-5p/519-3p, miR-142-3p.2, | 5 |
| | miR-30-5p, miR-199-5p | |
| RP11-463J10.3 | | 0 |
| MATK | | 0 |
| KCNJ2 | miR-15-5p/16-5p/195-5p/424-5p/497-5p, miR-15-5p/16-5p/195-5p/424-5p/497-5p, | 24 |
| | miR-196-5p, miR-455-5p, miR-411-3p, miR-302c-3p.2/520-3p, miR-124-3p.1, miR- | |
| | 141-3p/200a-3p, miR-18-5p, miR-193-3p, miR-365-3p, miR-9-5p, miR-126-3p.2, miR- | |
| | 19-3p, miR-19-3p, miR-124-3p.2/506-3p, miR-26-5p, miR-26-5p, miR-505-3p.2, miR- | |
| | 219-5p, miR-9-5p, miR-1-3p/206, miR-24-3p, miR-7-5p | |
| KLRB1 | | 0 |
| HIST1H2AH | miR-760 | 1 |
| APOBEC3G | | 0 |
| PSMB9 | miR-125-5p, miR-483-3p.2, miR-668-3p | 3 |
| PTPRC | | 0 |
| RCSD1 | miR-26-5p, miR-330-3p, miR-146-5p, miR-1306-5p, miR-150-5p, let-7-5p/98-5p | 5 |
| PYHIN1 | | 0 |
| BTN3A3 | | 0 |
| AC007278.2 | | 0 |
| C11orf21 | | 0 |
| RHOH | miR-505-3p.1 | 1 |
| TMEM156 | miR-218-5p, miR-135-5p, miR-496.1 | 3 |
| HIST2H3D | miR-760 | 1 |
| NLRP3 | miR-223-3p | 1 |
| AF001548.5 | | 0 |
| HIST1H2BI | miR-760 | 1 |
| CD74 | | 0 |
| HAVCR2 | | 0 |
| AGAP2 | miR-330-3p, miR-665, miR-302-3p/372-3p/373-3p/520-3p, miR-296-3p, miR-34- | 9 |
| | 5p/449-5p, miR-423-5p, miR-204-5p/211-5p, miR-136-5p, miR-3064-5p | |
| CD52 | miR-325-3p | 1 |
| GBP1 | miR-532-5p, miR-199-5p, miR-23-3p | 3 |
| GMFG | miR-876-5p | 1 |
| LAX1 | | 0 |
| SASH3 | miR-325-3p, miR-137 | 2 |
| GNLY | | 0 |
| HIST2H2AB | | 0 |
| MS4A6A | | 0 |
| PDE3B | miR-369-3p, miR-425-5p, miR-1193, miR-130-3p/301-3p/454-3p, miR-501-3p/502-3p, | 35 |
| | miR-135-5p, miR-25-3p/32-5p/92-3p/363-3p/367-3p, miR-203a-3p.2, miR-23-3p, miR- | |
| | 330-3p, miR-17-5p/20-5p/93-5p/106-5p/519-3p, miR-320, miR-186-5p, miR-33-5p, | |
| | miR-153-3p, miR-144-3p, miR-15-5p/16-5p/195-5p/424-5p/497-5p, miR-223-3p, miR- | |
| | 19-3p, miR-668-3p, miR-103-3p/107, miR-214-5p, miR-876-5p, miR-499a-5p, miR-27- | |
| | 3p, miR-26-5p, miR-124-3p.2/506-3p, miR-655-3p, miR-455-3p.1, miR-128-3p, miR- | |
| | 205-5p, miR-9-5p, miR-143-3p, miR-342-3p, miR-205-5p | |
| SLAMF8 | miR-199-5p | 1 |
| AGER | | 0 |
| PREX1 | miR-493-5p, miR-133a-3p.2/133b, miR-17-5p/20-5p/93-5p/106-5p/519-3p, miR-133a- | 16 |
| | 3p.1, miR-504-5p.1, miR-3064-5p, miR-142-5p, miR-411-5p.1, miR-410-3p, miR-148- | |
| | 3p/152-3p, miR-1-3p/206, miR-377-3p, miR-340-5p, miR-433-3p, miR-496.2, miR- | |
| | 411-5p.2 | |
| IRF8 | miR-186-5p | 1 |
| IKZF1 | miR-218-5p, miR-153-3p, miR-27-3p, miR-27-3p, miR-448, miR-448, miR-182-5p, | 16 |
| | miR-218-5p, miR-493-5p, miR-34-5p/449-5p, miR-19-3p, miR-582-5p, miR-137, miR- | |
| | 320, miR-128-3p, miR-31-5p | |
| TBC1D10C | miR-431-5p, miR-17-5p/20-5p/93-5p/106-5p/519-3p, miR-665 | 3 |
| FCGR3A | miR-326 | 1 |
| BCL11B | miR-381-3p, miR-188-5p, miR-1-3p/206, miR-29-3p, miR-383-5p.2, miR-21-5p/590- | 55 |
| | 5p, miR-15-5p/16-5p/195-5p/424-5p/497-5p, miR-486-5p, miR-1306-5p, miR-203a- | |
| | 3p.1, miR-203a-3p.1, miR-219a-2-3p, miR-221-3p/222-3p, miR-203a-3p.1, miR-183- | |
| | 5p.1, miR-183-5p.1, miR-330-3p, miR-25-3p/32-5p/92-3p/363-3p/367-3p, miR-124- | |
| | 3p.1, miR-124-3p.1, miR-302-3p/372-3p/373-3p/520-3p, miR-17-5p/20-5p/93-5p/106- | |
| | 5p/519-3p, miR-186-5p, miR-17-5p/20-5p/93-5p/106-5p/519-3p, miR-17-5p/20-5p/93- | |
| | 5p/106-5p/519-3p, miR-101-3p.1, miR-365-3p, miR-217, miR-144-3p, miR-200bc- | |
| | 3p/429, miR-200bc-3p/429, miR-216a-5p, miR-203a-3p.2, miR-203a-3p.2, miR-204- | |
| | 5p/211-5p, miR-374-5p, miR-137, miR-505-3p.2, miR-182-5p, miR-129-5p, miR-33- | |
| | 5p, miR-218-5p, miR-218-5p, miR-30-5p, miR-30-5p, miR-208-3p, miR-34-5p/449-5p, | |
| | miR-34-5p/449-5p, miR-193a-5p, miR-101-3p.2, miR-499a-5p, miR-101-3p.2, miR- | |
| | 582-5p, miR-582-5p, miR-582-5p | |
| HLA-DRB1 | | 0 |
| CD97 | miR-505-3p.1, miR-21-5p/590-5p, miR-126-3p.2, miR-320 | 4 |
| CTB-186G2.1 | | 0 |
| NLRC3 | miR-199-5p | 1 |
| TAP1 | miR-532-5p, miR-340-5p, miR-101-3p.1 | 3 |
| CD247 | miR-326 | 1 |
| HIST1H3G | | 0 |
| GBP4 | | 0 |
| RP11- | | 0 |
| 1049A21.2 | | |
| TMC8 | | 0 |
| SELPLG | miR-325-3p | 1 |
| HIST1H2BO | miR-760, miR-542-3p | 2 |
| CCND2 | miR-653-5p, miR-153-3p, miR-26-5p, miR-145-5p, miR-448, miR-325-3p, miR-96- | 52 |
| | 5p/1271-5p, miR-96-5p/1271-5p, miR-375, miR-204-5p/211-5p, miR-204-5p/211-5p, | |
| | miR-182-5p, miR-182-5p, let-7-5p/98-5p, let-7-5p/98-5p, let-7-5p/98-5p, miR-494-3p, | |
| | miR-190-5p, miR-29-3p, miR-185-5p, miR-299-5p, miR-383-5p.1, miR-1-3p/206, miR- | |
| | 1-3p/206, miR-1-3p/206, miR-150-5p, miR-302c-3p.2/520-3p, miR-154-5p, miR-183- | |
| | 5p.1, miR-135-5p, miR-139-5p, miR-802, miR-381-3p, miR-1193, miR-15-5p/16- | |
| | 5p/195-5p/424-5p/497-5p, miR-15-5p/16-5p/195-5p/424-5p/497-5p, miR-15-5p/16- | |
| | 5p/195-5p/424-5p/497-5p, miR-196-5p, miR-503-5p, miR-503-5p, miR-503-5p, miR- | |
| | 330-3p.2, miR-186-5p, miR-320, miR-323-3p, miR-340-5p, miR-124-3p.1, miR-340-5p, | |
| | miR-124-3p.2/506-3p, miR-19-3p, miR-409-3p, miR-141-3p/200a-3p, miR-17-5p/20- | |
| | 5p/93-5p/106-5p/519-3p, miR-302-3p/372-3p/373-3p/520-3p, miR-18-5p | |
| CST7 | | 0 |
| CCR5 | | 0 |
| RP11-264B17.5 | | 0 |
| GRAP2 | miR-326, miR-532-3p, miR-29-3p, miR-330-3p.2, miR-224-5p, miR-200bc-3p/429, | 10 |
| | miR-23-3p, miR-26-5p, miR-383-5p.1, miR-34-5p/449-5p | |
| HLA-B | | 0 |
| IKBIP | | 0 |
| KRT1 | miR-25-3p/32-5p/92-3p/363-3p/367-3p, miR-325-3p, miR-203a-3p.2, miR-203a-3p.1, | 6 |
| | miR-3064-5p, miR-137 | |
| SERPINB12 | | 0 |
| ALB | | 0 |
| TGM3 | miR-19-3p, miR-103-3p/107 | 2 |
| HS3ST6 | | 0 |
| NRTN | | 0 |
| FAM180A | miR-1193, miR-199-3p, miR-133a-3p.1 | 3 |
| FALEC | | 0 |
| SERPINB12 | | 0 |
| FGF22 | | 0 |
| HCN2 | miR-137, miR-9-5p, miR-137, miR-325-3p, miR-135-5p, miR-135-5p, miR-25-3p/32- | 8 |
| | 5p/92-3p/363-3p/367-3p, miR-181-5p | |
| SLC24A3 | miR-339-5p, miR-199-5p, miR-199-5p, miR-31-5p, miR-19-3p, miR-19-3p, miR-137, | 11 |
| | miR-130-3p/301-3p/454-3p, miR-15-5p/16-5p/195-5p/424-5p/497-5p, miR-302c- | |
| | 3p.2/520-3p, miR-25-3p/32-5p/92-3p/363-3p/367-3p | |
| RP11-167H9.5 | | 0 |
| C1QB | | 0 |
| IL18R1 | miR-485-5p | 1 |
| KCNE1 | miR-22-3p, miR-193-3p, miR-218-5p, miR-331-3p | 4 |
| CCR2 | | 0 |
| C1QA | | 0 |
| CD163 | miR-136-5p, miR-23-3p, miR-181-5p, miR-15-5p/16-5p/195-5p/424-5p/497-5p | 4 |
| CST7 | | 0 |
| SLC1A3 | miR-142-3p.1, miR-142-3p.2, miR-455-3p.1, miR-194-5p, miR-490-3p, miR-23-3p, | 7 |
| | miR-325-3p | |
| RNASE6 | miR-330-3p, miR-137, miR-325-3p | 3 |
| HIST1H1B | miR-760 | 1 |
| TRGC2 | | 0 |
| MS4A6A | | 0 |
| C1QC | | 0 |
| RGS1 | miR-223-3p, miR-655-3p, miR-27-3p | 3 |
| MSR1 | | 0 |
| HIST1H1D | miR-760 | 1 |
| HIST1H3I | miR-760, miR-423-5p | 2 |
| CD28 | miR-31-5p, miR-24-3p, miR-452-5p/892-3p, miR-15-5p/16-5p/195-5p/424-5p/497-5p, | 8 |
| | miR-503-5p, miR-145-5p, miR-27-3p, miR-202-5p | |
| LSM11 | miR-146-5p, miR-149-5p, miR-9-5p, miR-493-3p, let-7-5p/98-5p, miR-26-5p, miR- | 19 |
| | 433-3p, miR-542-3p, miR-582-5p, miR-183-5p.2, miR-382-3p, miR-15-5p/16-5p/195- | |
| | 5p/424-5p/497-5p, miR-15-5p/16-5p/195-5p/424-5p/497-5p, miR-132-3p/212-3p, miR- | |
| | 29-3p, miR-29-3p, miR-873-5p.1, miR-140-3p.2, miR-183-5p.1, miR-503-5p | |
| HIST1H3B | miR-760 | 1 |
| HIST1H2AJ | miR-760 | 1 |
| GIMAP6 | | 0 |
| DAAM2 | miR-1197, miR-1193, miR-18-5p, miR-29-3p, miR-193-3p, miR-873-5p.1, miR-496.2, | 15 |
| | miR-411-5p.2, miR-340-5p, miR-133a-3p.1, miR-125-5p, miR-339-5p, miR-423-5p, | |
| | miR-138-5p, miR-124-3p.1 | |
| CCL5 | | 0 |
| CTLA4 | miR-496.1, miR-155-5p, miR-142-3p.2, miR-142-3p.2 | 4 |
| IGHG1 | | 0 |
| C1orf162 | | 0 |
| MIR155HG | | 0 |
| ITM2A | | 0 |
| GIMAP4 | | 0 |
| HIST1H2BM | miR-760 | 1 |
| HIST1H3G | | 0 |
| FCER1G | miR-325-3p | 1 |
| GZMB | | 0 |
| FKBP5 | miR-143-3p, miR-203a-3p.1, miR-3064-5p, miR-149-5p, miR-320, miR-423-5p, miR- | 28 |
| | 330-3p.2, miR-122-5p, miR-122-5p, miR-99-5p/100-5p, miR-15-5p/16-5p/195-5p/424- | |
| | 5p/497-5p, miR-376c-3p, miR-495-3p, miR-141-3p/200a-3p, miR-203a-3p.2, miR-23- | |
| | 3p, miR-874-3p, miR-378-3p, miR-129-5p, miR-375, miR-133a-3p.1, miR-199-5p, | |
| | miR-411-3p, miR-381-3p, miR-877-5p, miR-499a-5p, miR-208-3p, miR-760 | |
| IL1R2 | | 0 |
| GZMA | | 0 |
| CLEC12A | | 0 |
| LINC01127 | | 0 |
| Z98744.2 | | 0 |
| GPR34 | miR-582-5p, miR-381-3p | 2 |
| HLA-DMB | | 0 |
| HIST1H4L | miR-328-3p | 1 |
| RRM2 | miR-204-5p/211-5p, miR-17-5p/20-5p/93-5p/106-5p/519-3p, miR-485-5p, let-7-5p/98- | 3 |
| | 5p | |
| TREM2 | | 0 |
| GIMAP8 | | 0 |
| CD3E | | 0 |
| AL049822.1 | | 0 |
| HLA-DPB1 | | 0 |
| LMNB1 | miR-140-5p, miR-128-3p, miR-23-3p, miR-101-3p.1, miR-218-5p, miR-124-3p.2/506- | 6 |
| | 3p | |
| CD300C | | 0 |
| IFITM1 | miR-325-3p | 1 |
| BLM | | 0 |
| SAMSN1 | | 0 |
| IRF8 | miR-186-5p | 1 |
| CLSPN | miR-381-3p, miR-15-5p/16-5p/195-5p/424-5p/497-5p, miR-325-3p, miR-496.1, miR- | 10 |
| | 371-5p, miR-330-3p, miR-330-3p, miR-103-3p/107, miR-302c-3p.2/520-3p, miR-24-3p | |
| IL1RL1 | | 0 |
| SDS | | 0 |
| RP11-274E7.2 | | 0 |
| MS4A4A | | 0 |
| PDCD1 | miR-15-5p/16-5p/195-5p/424-5p/497-5p, miR-15-5p/16-5p/195-5p/424-5p/497-5p, | 7 |
| | miR-15-5p/16-5p/195-5p/424-5p/497-5p, miR-15-5p/16-5p/195-5p/424-5p/497-5p, | |
| | miR-15-5p/16-5p/195-5p/424-5p/497-5p, miR-15-5p/16-5p/195-5p/424-5p/497-5p, | |
| | miR-15-5p/16-5p/195-5p/424-5p/497-5p | |
| ICOS | miR-29-3p, miR-338-3p, miR-26-5p, miR-27-3p, let-7-5p/98-5p, miR-196-5p | 5 |
| RP11-848G14.5 | | 0 |
| TNFSF13B | miR-653-5p, miR-452-5p/892-3p, miR-15-5p/16-5p/195-5p/424-5p/497-5p, miR-425- | 8 |
| | 5p, miR-23-3p, miR-15-5p/16-5p/195-5p/424-5p/497-5p, miR-503-5p, miR-503-5p | |
| HIST1H3F | miR-760 | 1 |
| RP11-777B9.5 | | 0 |
| GIMAP2 | | 0 |
| IL18RAP | | 0 |
| VSIG4 | miR-325-3p, miR-483-3p.1 | 2 |
| GIMAP7 | | 0 |
| SRGN | | 0 |
| TRBC2 | | 0 |
| LIPA | miR-148-3p/152-3p, miR-125-5p, miR-140-3p.1, miR-325-3p, miR-124-3p.2/506-3p | 5 |
| SERPINE1 | miR-224-5p, miR-34-5p/449-5p, miR-143-3p, miR-342-3p, miR-199-5p, miR-425-5p, | 11 |
| | miR-181-5p, miR-10-5p, miR-148-3p/152-3p, miR-145-5p, miR-30-5p | |
| CENPK | miR-186-5p, miR-362-5p/500b-5p | 2 |
| IL10 | miR-802, miR-27-3p, miR-543, miR-369-3p, let-7-5p/98-5p, miR-374-5p, miR-340-5p, | 7 |
| | miR-361-5p | |
| GZMK | | 0 |
| DTL | miR-330-3p, miR-96-5p/1271-5p, miR-142-5p | 3 |
| GLDN | miR-383-5p.1, miR-489-3p, miR-22-3p | 3 |
| SMAP2 | miR-18-5p, miR-320, miR-9-5p, miR-138-5p | 4 |
| HIST1H2BB | miR-760 | 1 |
| SIT1 | miR-491-5p, miR-125-5p, miR-15-5p/16-5p/195-5p/424-5p/497-5p, miR-15-5p/16- | 7 |
| | 5p/195-5p/424-5p/497-5p, miR-760, miR-490-3p, miR-143-3p | |
| MRC1 | miR-23-3p | 1 |
| IFITM3 | miR-325-3p | 1 |
| CTD- | | 0 |
| 2033D15.2 | | |
| HLA-DRA | miR-325-3p | 1 |
| THBS1 | miR-493-5p, miR-325-3p, miR-377-3p, miR-539-3p, miR-182-5p, miR-221-3p/222-3p, | 31 |
| | miR-1-3p/206, miR-221-3p/222-3p, miR-19-3p, miR-194-5p, miR-340-5p, miR-582-5p, | |
| | miR-101-3p.2, miR-101-3p.2, miR-144-3p, miR-144-3p, miR-381-3p, miR-132-3p/212- | |
| | 3p, miR-330-3p.2, miR-101-3p.1, miR-18-5p, miR-330-3p.2, miR-143-3p, miR-493-3p, | |
| | let-7-5p/98-5p, miR-331-3p, miR-338-3p, miR-139-5p, miR-139-5p, miR-205-5p, miR- | |
| | 543, miR-411-3p | |
| ADORA3 | miR-505-3p.2, miR-325-3p | 2 |
| STAB1 | | 0 |
| AIM2 | | 0 |
| EVI2A | miR-371-5p, miR-411-5p.2, miR-181-5p | 3 |
| TFPI2 | miR-23-3p | 1 |
| EMB | let-7-5p/98-5p, miR-142-3p.2, miR-670-3p, miR-29-3p, miR-216b-5p, miR-200bc- | 8 |
| | 3p/429, miR-320, miR-9-5p, miR-330-3p | |
| SLA | miR-383-5p.1, miR-155-5p, miR-142-3p.2, miR-191-5p, miR-181-5p, miR-873-5p.1, | 7 |
| | miR-339-5p | |
| CD8A | miR-326 | 1 |
| RCSD1 | miR-26-5p, miR-330-3p, miR-146-5p, miR-1306-5p, miR-150-5p, let-7-5p/98-5p | 5 |
| GIMAP1 | | 0 |
| FAM105A | miR-140-5p, miR-155-5p, miR-186-5p, miR-129-5p | 4 |
| FPR2 | | 0 |
| CD180 | miR-203a-3p.2, miR-452-5p/892-3p, miR-15-5p/16-5p/195-5p/424-5p/497-5p, miR- | 11 |
| | 329-3p/362-3p, miR-383-5p.2, miR-28-5p/708-5p, miR-204-5p/211-5p, miR-330-3p.2, | |
| | miR-203a-3p.1, miR-342-3p, miR-214-5p | |
| RP11-463J10.3 | | 0 |
| RP4-737E23.5 | | 0 |
| FCGR3A | miR-326 | 1 |
| HIST1H2BL | miR-760 | 1 |
| CD1D | | 0 |
| UBE2C | | 0 |
| PDK4 | miR-15-5p/16-5p/195-5p/424-5p/497-5p, miR-15-5p/16-5p/195-5p/424-5p/497-5p, | 14 |
| | miR-148-3p/152-3p, miR-29-3p, miR-361-5p, miR-493-5p, miR-122-5p, miR-181-5p, | |
| | miR-340-5p, miR-9-5p, miR-182-5p, miR-103-3p/107, miR-23-3p, miR-27-3p | |
| MLKL | | 0 |
| RP11-7F17.3 | | 0 |
| TLR7 | | 0 |
| PTPRC | | 0 |
| KBTBD8 | miR-99-5p/100-5p, miR-144-3p, miR-144-3p, miR-101-3p.1, miR-200bc-3p/429, miR- | 19 |
| | 153-3p, miR-539-3p, miR-139-5p, miR-219-5p, miR-23-3p, miR-101-3p.2, miR-19-3p, | |
| | miR-26-5p, miR-27-3p, miR-130-3p/301-3p/454-3p, miR-25-3p/32-5p/92-3p/363- | |
| | 3p/367-3p, miR-128-3p, miR-29-3p, miR-221-3p/222-3p | |
| CD96 | miR-409-3p, miR-182-5p | 2 |
| HIST1H2AI | miR-760, miR-9-5p | 2 |
| FAIM3 | miR-27-3p, miR-138-5p, miR-96-5p/1271-5p | 3 |
| CD48 | | 0 |
| IFI27L2 | | 0 |
| HIST1H4F | | 0 |
| CTD- | | 0 |
| 2033D15.3 | | |
| SLC16A6 | miR-23-3p, miR-204-5p/211-5p, miR-219-5p, miR-26-5p, miR-26-5p, miR-221-3p/222- | 15 |
| | 3p, miR-302-3p/372-3p/373-3p/520-3p, miR-543, miR-17-5p/20-5p/93-5p/106-5p/519- | |
| | 3p, miR-135-5p, miR-181-5p, miR-193-3p, miR-325-3p, miR-148-3p/152-3p, miR-125- | |
| | 5p | |
| LRRC8C | miR-30-5p, miR-9-5p, miR-483-3p.1, miR-212-5p, miR-379-5p, miR-493-5p, miR-182- | 10 |
| | 5p, miR-499a-5p, miR-340-5p, miR-204-5p/211-5p | |
| NLRP6 | miR-331-3p | 1 |
| TLR8 | miR-378-3p | 1 |
| RGS18 | miR-205-5p | 1 |
| IRAK3 | miR-494-3p | 1 |
| LY96 | | 0 |
| LILRA5 | | 0 |
| AL009179.1 | | 0 |
| PSMB9 | miR-125-5p, miR-483-3p.2, miR-668-3p | 3 |
| KIAA0101 | miR-19-3p, miR-30-5p, miR-383-5p.1, miR-330-3p.2, miR-421, miR-505-3p.2, miR- | 13 |
| | 655-3p, miR-216a-5p, miR-200bc-3p/429, miR-495-3p, miR-371-5p, miR-330-3p, miR- | |
| | 183-5p.1 | |
| GCA | miR-323-3p, miR-137, miR-23-3p, miR-330-3p.2, miR-371-5p, miR-493-5p, miR-494- | 8 |
| | 3p, miR-27-3p | |
| CCDC109B | | 0 |
| CTSL | | 0 |
| CCR5 | | 0 |
| BTN3A2 | miR-199-3p, miR-665, miR-216b-5p, miR-1306-5p | 4 |
| STAMBPL1 | miR-103-3p/107, miR-19-3p, miR-494-3p | 3 |
| HLA-DPA1 | | 0 |
| CTD- | | 0 |
| 2313F11.1 | | |
| LAIR1 | | 0 |
| FES | miR-140-5p | 1 |
| RNASE1 | miR-325-3p | 1 |
| CXCL10 | miR-15-5p/16-5p/195-5p/424-5p/497-5p, miR-503-5p | 2 |
| TRIM22 | | 0 |
| HIST1H3H | miR-760 | 1 |
| CD52 | miR-325-3p | 1 |
| MYBL2 | miR-423-5p, miR-29-3p, miR-143-3p, miR-30-5p | 4 |
| RP11-403P17.6 | | 0 |
| RNU4-1 | | 0 |
| KLRC1 | | 0 |
| FEN1 | miR-325-3p, miR-1306-5p, miR-140-5p | 3 |
| MIR181A1HG | | 0 |
| HCG27 | | 0 |
| RP11-335I12.2 | | 0 |
| BIN2 | miR-125-5p | 1 |
| RP4-620F22.2 | | 0 |
| GBP5 | miR-219-5p | 1 |
| FYB | miR-142-3p.2, miR-23-3p, miR-494-3p, miR-139-5p | 4 |
| PCED1B | | 0 |
| AL031777.1 | | 0 |
| MMD | miR-494-3p, miR-205-5p, miR-1-3p/206, miR-1-3p/206, miR-1-3p/206, miR-25-3p/32- | 13 |
| | 5p/92-3p/363-3p/367-3p, miR-140-5p, miR-200bc-3p/429, miR-148-3p/152-3p, miR- | |
| | 128-3p, miR-182-5p, miR-30-5p, miR-27-3p | |
| DOK2 | | 0 |
| RP11-404F10.2 | | 0 |
| PPT1 | miR-421, miR-505-3p.2, miR-125-5p | 3 |
| FAM65B | miR-340-5p, miR-431-5p, miR-146-5p, miR-218-5p, miR-489-3p, miR-27-3p, miR-15- | 14 |
| | 5p/16-5p/195-5p/424-5p/497-5p, miR-369-3p, miR-128-3p, miR-410-3p, miR-29-3p, | |
| | miR-125-5p, miR-330-3p.2, miR-374-5p | |
| GVINP1 | | 0 |
| SERPINF1 | miR-325-3p | 1 |
| CARD16 | | 0 |
| CEP55 | miR-19-3p, miR-101-3p.2, miR-148-3p/152-3p, miR-15-5p/16-5p/195-5p/424-5p/497- | 6 |
| | 5p, miR-144-3p, miR-130-3p/301-3p/454-3p | |
| HIST1H2AL | miR-760 | 1 |
| CD86 | miR-496.1, let-7-5p/98-5p | 1 |
| CD2 | | 0 |
| JAK3 | miR-1193 | 1 |
| SELPLG | miR-325-3p | 1 |
| AC007278.3 | | 0 |
| FAM111B | | 0 |
| GYG1 | miR-194-5p | 1 |
| NLRP3 | miR-223-3p | 1 |
| CD74 | | 0 |
| ZWINT | | 0 |
| ITGA4 | miR-130-3p/301-3p/454-3p, miR-17-5p/20-5p/93-5p/106-5p/519-3p, miR-142-3p.2, | 5 |
| | miR-30-5p, miR-199-5p | |
| TRAC | | 0 |
| SELL | | 0 |
| GPR183 | miR-101-3p.2, miR-144-3p, miR-186-5p, miR-194-5p | 4 |
| TRG-AS1 | | 0 |
| ST8SIA4 | miR-19-3p, miR-203a-3p.2, miR-448, miR-218-5p, miR-183-5p.2, miR-103-3p/107, | 30 |
| | miR-30-5p, miR-374-5p, miR-505-3p.1, miR-30-5p, miR-27-3p, miR-433-3p, miR-26- | |
| | 5p, miR-377-3p, miR-653-5p, miR-365-3p, miR-181-5p, miR-125-5p, miR-124-3p.1, | |
| | miR-181-5p, miR-142-5p, miR-410-3p, miR-199-3p, miR-22-3p, miR-129-3p, miR- | |
| | 143-3p, miR-203a-3p.1, miR-9-5p, miR-128-3p, miR-383-5p.1 | |
| NCKAP1L | miR-653-5p, miR-204-5p/211-5p, miR-203a-3p.2, miR-15-5p/16-5p/195-5p/424- | 10 |
| | 5p/497-5p, miR-503-5p, miR-338-3p, miR-149-5p, miR-873-5p.2, miR-493-3p, miR- | |
| | 494-3p | |
| STK17B | miR-186-5p, miR-186-5p, miR-186-5p, miR-17-5p/20-5p/93-5p/106-5p/519-3p, miR- | 17 |
| | 17-5p/20-5p/93-5p/106-5p/519-3p, miR-17-5p/20-5p/93-5p/106-5p/519-3p, miR-186- | |
| | 5p, miR-183-5p.2, miR-203a-3p.2, miR-655-3p, miR-182-5p, miR-96-5p/1271-5p, miR- | |
| | 302c-3p.2/520-3p, miR-30-5p, miR-302-3p/372-3p/373-3p/520-3p, miR-203a-3p.1, | |
| | miR-455-3p.1 | |
| OLAH | | 0 |
| APOC1 | miR-325-3p | 1 |
| PCED1B-AS1 | | 0 |
| SLFN13 | miR-3064-5p | 1 |
| TIGIT | miR-455-3p.1, miR-23-3p, miR-219a-2-3p | 3 |
| ATHL1 | | 0 |
| HIST1H2BI | miR-760 | 1 |
| HLA-DMA | miR-325-3p | 1 |
| ENTPD1 | miR-205-5p, miR-9-5p, miR-136-5p, miR-150-5p, miR-125-5p, miR-483-3p.2, miR- | 15 |
| | 330-3p.2, miR-330-3p, miR-193-3p, miR-135-5p, miR-421, miR-376-3p, miR-346, | |
| | miR-340-5p, miR-142-5p | |
| SLCO2B1 | miR-224-5p, miR-490-3p, miR-125-5p | 3 |
| IFNG | miR-340-5p, miR-29-3p, miR-24-3p, miR-125-5p | 4 |
| CD300LF | miR-125-5p | 1 |
| F13A1 | miR-182-5p, miR-96-5p/1271-5p, miR-335-5p, miR-155-5p | 4 |
| NEIL3 | | 0 |
| TMEM14E | | 0 |
| ACSL4 | miR-130-3p/301-3p/454-3p, miR-144-3p, miR-15-5p/16-5p/195-5p/424-5p/497-5p, | 10 |
| | miR-101-3p.2, miR-145-5p, miR-19-3p, miR-34-5p/449-5p, miR-133a-3p.1, miR-204- | |
| | 5p/211-5p, miR-17-5p/20-5p/93-5p/106-5p/519-3p | |
| RN7SKP48 | | 0 |
| GZMH | | 0 |
| LYAR | | 0 |
| GPNMB | | 0 |
| IKZF1 | miR-218-5p, miR-153-3p, miR-27-3p, miR-27-3p, miR-448, miR-448, miR-182-5p, | 16 |
| | miR-218-5p, miR-493-5p, miR-34-5p/449-5p, miR-19-3p, miR-582-5p, miR-137, miR- | |
| | 320, miR-128-3p, miR-31-5p | |
| RP3-455J7.4 | | 0 |
| SCIMP | miR-3064-5p, miR-382-5p, miR-302c-3p.2/520-3p, miR-491-5p, miR-325-3p, miR- | 6 |
| | 302-3p/372-3p/373-3p/520-3p | |
| AMIGO2 | miR-185-5p | 1 |
| TLR5 | miR-150-5p | 1 |
| EXOC6 | | 0 |
| IFITM2 | miR-325-3p | 1 |
| HAVCR2 | | 0 |
| TLR2 | | 0 |
| CTB-111H14.1 | | 0 |
| FGL2 | miR-124-3p.2/506-3p, miR-199-3p, miR-302-3p/372-3p/373-3p/520-3p, miR-137 | 4 |
| A2M | miR-325-3p | 1 |
| LY9 | miR-154-5p | 1 |
| KIAA0825 | miR-320, miR-376-3p, miR-193-3p | 3 |
| NKG7 | | 0 |
| WDR76 | miR-199-5p, miR-182-5p | 2 |
| LILRB5 | | 0 |
| SLAMF6 | miR-186-5p, miR-665 | 2 |
| CTB-41I6.2 | | 0 |
| FGR | miR-340-5p | 1 |
| HELLS | miR-483-3p.1, miR-532-5p, miR-365-3p, miR-216b-5p, miR-103-3p/107, miR-140- | 17 |
| | 3p.1, miR-140-3p.1, miR-3064-5p, miR-205-5p, miR-335-5p, miR-483-3p.2, miR-203a- | |
| | 3p.2, miR-489-3p, miR-204-5p/211-5p, miR-325-3p, miR-455-3p.2, miR-145-5p | |
| CD274 | miR-155-5p, miR-382-3p, miR-653-5p, miR-377-3p, miR-17-5p/20-5p/93-5p/106- | 8 |
| | 5p/519-3p, miR-320, miR-142-5p, miR-140-3p.2 | |
| HIST1H2AB | miR-760 | 1 |
| MELK | miR-205-5p, miR-219a-2-3p, miR-181-5p, miR-505-3p.1, miR-542-3p, miR-802, miR- | 16 |
| | 873-5p.1, miR-140-3p.2, miR-142-5p, miR-382-3p, miR-376-3p, miR-325-3p, miR-23- | |
| | 3p, miR-543, miR-505-3p.2, miR-421 | |
| ASB2 | miR-874-3p | 1 |
| SIGLEC9 | | 0 |
| TPK1 | miR-140-3p.1, miR-1-3p/206, miR-224-5p, miR-29-3p | 4 |
| MAP2K6 | miR-1298-5p, miR-202-5p, miR-145-5p, miR-27-3p, miR-543, miR-543, miR-124-3p.1, | 33 |
| | miR-182-5p, miR-325-3p, miR-448, miR-153-3p, miR-329-3p/362-3p, miR-329- | |
| | 3p/362-3p, miR-493-5p, miR-483-3p.2, miR-30-5p, miR-140-3p.1, miR-150-5p, miR- | |
| | 140-3p.2, miR-142-3p.1, miR-505-3p.2, miR-425-5p, miR-374-5p, miR-223-3p, miR- | |
| | 101-3p.2, miR-494-3p, miR-15-5p/16-5p/195-5p/424-5p/497-5p, miR-142-5p, miR-29- | |
| | 3p, miR-369-3p, miR-154-3p/487-3p, miR-378-3p, miR-103-3p/107 | |
| RP11-347P5.1 | | 0 |
| HIST1H2AH | miR-760 | 1 |
| EVI2B | miR-203a-3p.2, miR-325-3p, miR-340-5p, miR-203a-3p.1 | 4 |
| CTA-373H7.7 | | 0 |
| FAM26F | | 0 |
| KLRB1 | | 0 |
| TRPV2 | miR-223-3p, miR-223-3p, miR-202-5p, miR-149-5p, miR-3064-5p, miR-202-5p | 6 |
| CHIT1 | miR-378-3p, miR-760 | 2 |
| HTATSF1P2 | | 0 |
| TSC22D3 | miR-221-3p/222-3p, miR-203a-3p.2, miR-760, miR-183-5p.2, miR-96-5p/1271-5p, | 12 |
| | miR-325-3p, miR-29-3p, miR-320, miR-495-3p, miR-216b-5p, miR-143-3p, miR-15- | |
| | 5p/16-5p/195-5p/424-5p/497-5p | |
| TLDC2 | miR-421 | 1 |
| MGAM | miR-330-3p | 1 |
| RP11-69L16.4 | | 0 |
| CXCR6 | | 0 |
| STAT4 | miR-320, miR-141-3p/200a-3p | 2 |
| TNFSF8 | miR-24-3p, miR-504-5p.1 | 2 |
| MB21D1 | | 0 |
| SLFN11 | miR-3064-5p | 1 |
| RP11-124N14.3 | | 0 |
| TYROBP | | 0 |
| ITGB2 | | 0 |
| LILRB4 | | 0 |
| CDK1 | miR-203a-3p.2, miR-31-5p, miR-143-3p, miR-96-5p/1271-5p, miR-182-5p, miR-330- | 9 |
| | 3p, miR-15-5p/16-5p/195-5p/424-5p/497-5p, miR-495-3p, miR-129-3p | |
| PSMB8 | miR-125-5p, miR-451 | 2 |
| HIST2H2AB | | 0 |
| DNA2 | let-7-5p/98-5p | 0 |
| PDCD1LG2 | miR-17-5p/20-5p/93-5p/106-5p/519-3p, miR-17-5p/20-5p/93-5p/106-5p/519-3p, miR- | 4 |
| | 302-3p/372-3p/373-3p/520-3p, miR-302c-3p.2/520-3p | |
| CCL18 | | 0 |
| RP11-25K21.6 | | 0 |
| HLA-DQA1 | miR-137, miR-325-3p | 2 |
| CELF2 | miR-129-5p, miR-129-5p, miR-129-5p, miR-129-5p, miR-129-5p, miR-129-5p, miR- | 84 |
| | 210-3p, miR-325-3p, miR-493-5p, miR-183-5p.2, miR-183-5p.2, miR-96-5p/1271-5p, | |
| | miR-182-5p, miR-182-5p, miR-33-5p, miR-146-5p, miR-143-3p, miR-326, miR-371- | |
| | 5p, miR-494-3p, miR-219-5p, miR-30-5p, miR-142-3p.1, miR-103-3p/107, miR-24-3p, | |
| | miR-140-5p, miR-183-5p.1, miR-873-5p.1, miR-25-3p/32-5p/92-3p/363-3p/367-3p, | |
| | miR-369-3p, miR-140-5p, miR-425-5p, miR-140-3p.1, miR-140-3p.1, miR-486-5p, | |
| | miR-199-5p, miR-140-3p.1, miR-199-3p, miR-199-3p, miR-802, miR-377-3p, miR-23- | |
| | 3p, miR-23-3p, miR-27-3p, miR-27-3p, miR-27-3p, miR-153-3p, miR-1224-5p, miR- | |
| | 653-5p, miR-489-3p, miR-1224-5p, miR-33-5p, miR-655-3p, miR-101-3p.2, miR-101- | |
| | 3p.2, miR-431-5p, miR-208-3p, miR-582-5p, miR-26-5p, miR-26-5p, miR-374-5p, | |
| | miR-137, miR-216a-5p, miR-200bc-3p/429, miR-101-3p.1, miR-216b-5p, miR-495-3p, | |
| | miR-495-3p, miR-17-5p/20-5p/93-5p/106-5p/519-3p, miR-543, miR-132-3p/212-3p, | |
| | miR-302-3p/372-3p/373-3p/520-3p, miR-129-3p, miR-29-3p, miR-1193, miR-302c- | |
| | 3p.2/520-3p, miR-196-5p, miR-452-5p/892-3p, miR-144-3p, miR-140-3p.2, miR-144- | |
| | 3p, miR-10-5p, miR-217, miR-452-5p/892-3p | |
| AIF1 | | 0 |
| LBR | miR-144-3p, miR-181-5p, miR-130-3p/301-3p/454-3p, miR-200bc-3p/429, miR-200bc- | 11 |
| | 3p/429, miR-101-3p.2, miR-24-3p, miR-340-5p, miR-148-3p/152-3p, let-7-5p/98-5p, | |
| | miR-23-3p, miR-202-5p | |
| AL021807.1 | | 0 |
| GMFG | miR-876-5p | 1 |
| PTGER2 | miR-421 | 1 |
| VIM | miR-124-3p.1, miR-320, miR-325-3p, miR-30-5p, miR-138-5p | 5 |
| CASP1 | | 0 |
| HCG4P11 | | 0 |
| RP4-671O14.5 | | 0 |
| HLA-DQB1 | miR-148-3p/152-3p | 1 |
| ZNF367 | miR-302c-3p.2/520-3p, miR-21-5p/590-5p, miR-21-5p/590-5p, miR-15-5p/16-5p/195- | 19 |
| | 5p/424-5p/497-5p, miR-19-3p, miR-142-3p.2, miR-101-3p.2, miR-146-5p, miR-139-5p, | |
| | miR-330-3p, miR-9-5p, miR-18-5p, miR-17-5p/20-5p/93-5p/106-5p/519-3p, miR-17- | |
| | 5p/20-5p/93-5p/106-5p/519-3p, miR-144-3p, miR-10-5p, miR-302-3p/372-3p/373- | |
| | 3p/520-3p, miR-302-3p/372-3p/373-3p/520-3p, miR-302-3p/372-3p/373-3p/520-3p | |
| VNN1 | | 0 |
| RP11-278C7.5 | | 0 |
| HCST | | 0 |
| SLC46A3 | miR-125-5p, miR-494-3p, miR-653-5p, miR-137, miR-302-3p/372-3p/373-3p/520-3p, | 8 |
| | miR-325-3p, miR-17-5p/20-5p/93-5p/106-5p/519-3p, miR-17-5p/20-5p/93-5p/106- | |
| | 5p/519-3p | |
| SLAMF8 | miR-199-5p | 1 |
| HLA-H | | 0 |
| CARD8-AS1 | | 0 |
| RP1-68D18.2 | | 0 |
| CTD- | | 0 |
| 2033D15.1 | | |
| NCAPG | miR-9-5p, miR-330-3p.2 | 2 |
| SMARCD3 | | 0 |
| MMP19 | miR-320, miR-30-5p, miR-193-3p, miR-411-5p.1, miR-148-3p/152-3p, miR-223-3p, | 9 |
| | miR-324-5p, miR-874-3p, miR-145-5p | |
| ARHGAP15 | miR-203a-3p.1 | 1 |
| FABP5P7 | | 0 |
| HLA-DQB1- | | 0 |
| AS1 | | |
| HIST1H2BO | miR-760, miR-542-3p | 2 |
| FCGR2A | | 0 |
| ITGAM | | 0 |
| MCM6 | | 0 |
| VCAN | miR-27-3p, miR-27-3p, miR-23-3p, miR-23-3p, miR-33-5p, miR-203a-3p.2, miR-539- | 28 |
| | 3p, miR-1298-5p, miR-653-5p, miR-101-3p.1, miR-144-3p, miR-141-3p/200a-3p, miR- | |
| | 543, miR-124-3p.1, miR-135-5p, miR-30-5p, miR-455-5p, miR-381-3p, miR-181-5p, | |
| | miR-142-3p.2, miR-103-3p/107, miR-203a-3p.1, miR-494-3p, miR-101-3p.2, miR-340- | |
| | 5p, miR-128-3p, miR-136-5p, miR-9-5p | |
| CD37 | | 0 |
| BTLA | miR-15-5p/16-5p/195-5p/424-5p/497-5p, miR-15-5p/16-5p/195-5p/424-5p/497-5p, | 10 |
| | miR-503-5p, miR-142-3p.1, miR-26-5p, miR-23-3p, miR-25-3p/32-5p/92-3p/363- | |
| | 3p/367-3p, miR-136-5p, miR-335-5p, miR-103-3p/107 | |
| Z98744.3 | | 0 |
| NMI | | 0 |
| RP11- | | 0 |
| 1049A21.2 | | |
| MARCH1 | | 0 |
| RP11-290C10.1 | | 0 |
| VNN2 | | 0 |
| HLA-DOA | miR-383-5p.2 | 1 |
| CD300E | | 0 |
| RGS2 | miR-96-5p/1271-5p, miR-183-5p.2, miR-22-3p | 3 |
| HIST1H3J | miR-760 | 1 |
| NCAPH | miR-493-5p | 1 |
| HIST1H2BE | miR-760 | 1 |
| HLA-B | | 0 |
| CD4 | miR-181-5p, miR-221-3p/222-3p | 2 |
| MSNP1 | | 0 |
| CTSC | | 0 |
| CXCL5 | miR-23-3p, miR-25-3p/32-5p/92-3p/363-3p/367-3p | 2 |
| AC008984.2 | | 0 |
| HIST1H2AE | miR-760, miR-9-5p | 2 |
| APMAP | miR-135-5p, miR-126-3p.2, miR-199-5p | 3 |
| CD84 | miR-188-5p, miR-203a-3p.1, miR-142-3p.1, miR-485-5p, miR-1197, miR-19-3p | 6 |
| SHC4 | miR-382-5p, miR-382-5p, miR-876-5p, miR-544a-5p, miR-330-3p, miR-124-3p.1, miR- | 14 |
| | 142-5p, miR-182-5p, miR-96-5p/1271-5p, miR-218-5p, miR-27-3p, miR-26-5p, miR- | |
| | 489-3p, miR-488-3p | |
| GLIPR2 | miR-423-5p, miR-378-3p, miR-136-5p, miR-665, miR-665, miR-1224-5p, miR-218-5p, | 12 |
| | miR-1306-5p, miR-22-3p, miR-489-3p, miR-193-3p, miR-200bc-3p/429 | |
| ZPLD1 | miR-130-3p/301-3p/454-3p, miR-155-5p, miR-19-3p, let-7-5p/98-5p | 3 |
| GPR171 | | 0 |
| FAM101B | miR-330-3p, miR-29-3p, miR-15-5p/16-5p/195-5p/424-5p/497-5p, miR-330-3p, miR- | 6 |
| | 325-3p, miR-1-3p/206 | |
| C5AR1 | | 0 |
| UBASH3B | miR-25-3p/32-5p/92-3p/363-3p/367-3p, miR-125-5p, miR-9-5p, miR-141-3p/200a-3p, | 15 |
| | miR-455-5p, miR-302c-3p.2/520-3p, miR-302-3p/372-3p/373-3p/520-3p, miR-377-3p, | |
| | miR-31-5p, miR-219-5p, miR-488-3p, miR-129-5p, miR-203a-3p.2, miR-17-5p/20- | |
| | 5p/93-5p/106-5p/519-3p, miR-182-5p | |
| FAM78A | miR-124-3p.1, miR-27-3p, miR-137, miR-218-5p, miR-96-5p/1271-5p, miR-182-5p, | 19 |
| | miR-182-5p, miR-302-3p/372-3p/373-3p/520-3p, miR-128-3p, miR-128-3p, miR-130- | |
| | 3p/301-3p/454-3p, miR-335-5p, miR-330-3p, miR-125-5p, miR-101-3p.1, miR-124- | |
| | 3p.2/506-3p, miR-124-3p.2/506-3p, miR-31-5p, miR-24-3p | |
| SLED1 | | 0 |
| CRTAM | miR-133a-3p.1, miR-33-5p, miR-142-3p.1, miR-25-3p/32-5p/92-3p/363-3p/367-3p, | 5 |
| | miR-133a-3p.2/133b | |
| PMP22 | miR-139-5p, miR-145-5p, miR-29-3p, miR-199-5p | 4 |
| ZEB2-AS1 | | 0 |
| CTC-301O7.4 | | 0 |
| PFN1P1 | | 0 |
| EPSTI1 | miR-223-3p, miR-505-3p.1, miR-421, miR-421 | 4 |
| HK3 | miR-137 | 1 |
| C18orf54 | miR-216b-5p, miR-135-5p, miR-223-3p, miR-23-3p | 4 |
| ATP8A1 | miR-148-3p/152-3p, miR-9-5p, miR-128-3p, miR-128-3p, miR-455-3p.2, miR-153-3p, | 22 |
| | miR-101-3p.2, miR-219a-2-3p, miR-31-5p, miR-30-5p, miR-33-5p, miR-144-3p, miR- | |
| | 495-3p, miR-141-3p/200a-3p, miR-18-5p, miR-181-5p, miR-302c-3p.2/520-3p, miR- | |
| | 135-5p, miR-217, miR-22-3p, miR-182-5p, miR-96-5p/1271-5p | |
| C11orf21 | | 0 |
| LCP1 | miR-124-3p.1, miR-96-5p/1271-5p, miR-183-5p.2, miR-543, miR-124-3p.2/ | 6 |
| | 506-3p, miR-802 | |
| FAM198B | | 0 |
| RP11-326C3.2 | | 0 |
| ETV7 | | 0 |
| WAS | | 0 |
| RP11-415J8.3 | | 0 |
| CYBB | miR-670-3p, miR-1298-5p, miR-1298-5p | 3 |
| CDCA7L | miR-199-5p, miR-378-3p, miR-28-5p/708-5p, miR-760, miR-218-5p, miR-874-3p, | 9 |
| | miR-325-3p, miR-137, miR-25-3p/32-5p/92-3p/363-3p/367-3p | |
| CMSS1 | | 0 |
| RBL1 | miR-302c-3p.2/520-3p, miR-302-3p/372-3p/373-3p/520-3p, miR-330-3p, miR-17- | 5 |
| | 5p/20-5p/93-5p/106-5p/519-3p, miR-124-3p.1 | |
| FLI1 | miR-137, miR-758-3p, miR-1-3p/206, miR-655-3p, miR-101-3p.2, miR-145-5p, miR- | 17 |
| | 145-5p, miR-145-5p, miR-101-3p.1, miR-144-3p, miR-365-3p, miR-141-3p/200a-3p, | |
| | miR-382-5p, miR-200bc-3p/429, miR-193-3p, miR-302c-3p.2/520-3p, miR-371-5p | |
| RNF175 | | 0 |
| IL27RA | | 0 |
| CSF1R | miR-22-3p, miR-34-5p/449-5p, miR-24-3p, miR-24-3p, miR-155-5p | 5 |
| TMX1 | miR-128-3p, miR-128-3p, miR-151-3p, miR-802, miR-411-3p, miR-323-3p, miR-224- | 20 |
| | 5p, miR-1-3p/206, miR-196-5p, miR-875-5p, miR-183-5p.2, miR-448, miR-153-3p, | |
| | miR-96-5p/1271-5p, miR-218-5p, miR-377-3p, miR-137, miR-377-3p, miR-653-5p, | |
| | miR-27-3p | |
| FAR2 | miR-539-3p, miR-371-5p | 2 |
| HAUS1 | miR-27-3p | 1 |
| AOAH | | 0 |
| AQP9 | miR-154-5p, miR-539-3p | 2 |
| ARHGEF6 | miR-135-5p, miR-142-5p, miR-496.1, miR-340-5p, miR-30-5p, miR-330-3p.2, miR- | 8 |
| | 448, miR-488-3p | |
| AC007278.2 | | 0 |
| CLEC2B | | 0 |
| CSGALNACT2 | miR-23-3p, miR-653-5p, miR-325-3p, miR-29-3p, miR-217, miR-302c-3p.2/520-3p | 6 |
| LILRB1 | | 0 |
| ITK | miR-873-5p.2, miR-155-5p, miR-140-3p.2 | 3 |
| LINC01093 | | 0 |
| P2RY14 | | 0 |
| FPR1 | | 0 |
| PLK4 | | 0 |
| RP11- | | 0 |
| 488L18.10 | | |
| MDM1 | miR-141-3p/200a-3p | 1 |
| PREX1 | miR-493-5p, miR-133a-3p.2/133b, miR-17-5p/20-5p/93-5p/106-5p/519-3p, miR-133a- | 16 |
| | 3p.1, miR-504-5p.1, miR-3064-5p, miR-142-5p, miR-411-5p.1, miR-410-3p, miR-148- | |
| | 3p/152-3p, miR-1-3p/206, miR-377-3p, miR-340-5p, miR-433-3p, miR-496.2, miR- | |
| | 411-5p.2 | |
| RP11-186N15.3 | | 0 |
| RP11-256L6.3 | | 0 |
| RP6-159A1.4 | | 0 |
| CD226 | miR-302-3p/372-3p/373-3p/520-3p, miR-17-5p/20-5p/93-5p/106-5p/519-3p, miR-378- | 7 |
| | 3p, miR-219-5p, miR-30-5p, miR-302c-3p.2/520-3p, miR-302c-3p.2/520-3p | |
| CD300LB | miR-133a-3p.1 | 1 |
| SNX10 | miR-30-5p | 1 |
| CEP85L | miR-218-5p, miR-133a-3p.1, miR-182-5p, miR-134-5p, miR-137, miR-27-3p, miR-23- | 28 |
| | 3p, miR-199-5p, miR-135-5p, miR-199-3p, miR-96-5p/1271-5p, miR-133a-3p.2/133b, | |
| | miR-369-3p, miR-200bc-3p/429, miR-10-5p, miR-29-3p, miR-224-5p, let-7-5p/98-5p, | |
| | miR-18-5p, miR-219-5p, miR-103-3p/107, miR-31-5p, miR-30-5p, miR-495-3p, miR- | |
| | 129-3p, miR-15-5p/16-5p/195-5p/424-5p/497-5p, miR-124-3p.1, miR-543, miR-135-5p | |
| CLEC10A | miR-3064-5p, miR-149-5p | 2 |
| LTB4R | miR-193a-5p, miR-873-5p.2, miR-22-3p, miR-320 | 4 |
| MYC | | 0 |
| CENPH | | 0 |
| FXYD4 | miR-383-5p.1 | 1 |
| TRAF5 | miR-29-3p | 1 |
| CHTA | miR-142-3p.2, miR-142-3p.1, miR-873-5p.1, miR-1197, miR-22-3p | 5 |
| SOAT1 | miR-653-5p, miR-382-5p, miR-192-5p/215-5p, miR-802, miR-144-3p, miR-200bc- | 9 |
| | 3p/429, miR-539-3p, miR-9-5p, miR-655-3p | |
| RP11-325F22.2 | | 0 |
| AGAP2 | miR-330-3p, miR-665, miR-302-3p/372-3p/373-3p/520-3p, miR-296-3p, miR-34- | 9 |
| | 5p/449-5p, miR-423-5p, miR-204-5p/211-5p, miR-136-5p, miR-3064-5p | |
| NAIP | | 0 |
| PLP2 | miR-7-5p, miR-150-5p, miR-124-3p.1 | 3 |
| PSMB8-AS1 | | 0 |
| ETS1 | miR-365-3p, miR-410-3p, miR-410-3p, miR-101-3p.1, miR-199-5p, miR-455-5p, miR- | 38 |
| | 338-3p, miR-338-3p, miR-338-3p, miR-1193, miR-193-3p, miR-181-5p, miR-410-3p, | |
| | miR-381-3p, miR-200bc-3p/429, miR-200bc-3p/429, miR-124-3p.1, miR-330-3p.2, | |
| | miR-495-3p, miR-144-3p, miR-760, miR-499a-5p, miR-124-3p.2/506-3p, miR-542-3p, | |
| | miR-208-3p, miR-129-5p, miR-145-5p, miR-155-5p, miR-155-5p, miR-101-3p.2, miR- | |
| | 125-5p, miR-9-5p, miR-203a-3p.1, miR-139-5p, miR-532-3p, miR-24-3p, miR-221- | |
| | 3p/222-3p, miR-1-3p/206 | |
| IRF4 | miR-125-5p, miR-142-3p.2, miR-27-3p, miR-203a-3p.1, miR-30-5p | 5 |
| NCF1 | miR-325-3p, miR-19-3p | 2 |
| E2F8 | miR-101-3p.2, miR-19-3p, miR-144-3p | 3 |
| CD33 | | 0 |
| BTNL8 | miR-216b-5p | 1 |
| CLEC4E | | 0 |
| POLR3GL | | 0 |
| IKZF3 | miR-873-5p.1, miR-543, miR-330-3p, miR-362-5p/500b-5p, miR-217, miR-140-5p, | 31 |
| | miR-193-3p, miR-382-5p, miR-140-3p.2, miR-653-5p, miR-455-3p.2, miR-145-5p, | |
| | miR-491-5p, miR-23-3p, miR-23-3p, miR-375, miR-129-5p, miR-96-5p/1271-5p, miR- | |
| | 182-5p, miR-182-5p, miR-96-5p/1271-5p, miR-371-5p, miR-203a-3p.1, miR-494-3p, | |
| | miR-146-5p, miR-140-3p.1, miR-125-5p, miR-346, miR-346, miR-760, miR-185-5p | |
| IL2RA | miR-30-5p, miR-302c-3p.2/520-3p | 2 |
| GFI1 | miR-142-3p.1, miR-194-5p, miR-142-3p.2, miR-377-3p, miR-26-5p, miR-543, miR- | 8 |
| | 495-3p, miR-200bc-3p/429 | |
| HHEX | miR-223-3p, miR-145-5p, miR-204-5p/211-5p | 3 |
| SNX20 | | 0 |
| APBB1IP | miR-493-5p | 1 |
| RP11-15A1.7 | | 0 |
| DCK | miR-411-3p, miR-192-5p/215-5p | 2 |
| SNORA12 | | 0 |
| EMR1 | miR-325-3p | 1 |
| APOBEC3G | | 0 |
| ANXA2R | | 0 |
| ANKRD22 | miR-325-3p, miR-876-5p | 2 |
| AC025048.1 | | 0 |
| MS4A7 | miR-496.1 | 1 |
| GNLY | | 0 |
| MSN | miR-338-3p, miR-133a-3p.2/133b, miR-200bc-3p/429, miR-200bc-3p/429, miR-369- | 15 |
| | 3p, miR-96-5p/1271-5p, miR-27-3p, miR-542-3p, let-7-5p/98-5p, miR-489-3p, miR- | |
| | 200bc-3p/429, miR-325-3p, miR-133a-3p.1, miR-192-5p/215-5p, miR-183-5p.2, miR- | |
| | 96-5p/1271-5p | |
| FABP5 | | 0 |
| PYHIN1 | | 0 |
| GPR141 | miR-216a-5p | 1 |
| RP11-455F5.5 | | 0 |
| HCG4P7 | | 0 |
| GAB3 | miR-155-5p, miR-26-5p, miR-330-3p, miR-365-3p | 4 |
| PARVG | | 0 |
| C17orf53 | miR-133a-3p.1 | 1 |
| RP11-297C4.2 | | 0 |
| SLC4A7 | miR-124-3p.1, miR-17-5p/20-5p/93-5p/106-5p/519-3p, miR-137, miR-182-5p, miR- | 10 |
| | 488-3p, miR-124-3p.2/506-3p, miR-15-5p/16-5p/195-5p/424-5p/497-5p, miR-200bc- | |
| | 3p/429, miR-34-5p/449-5p, miR-30-5p | |
| FLT3 | miR-204-5p/211-5p, miR-376c-3p, miR-15-5p/16-5p/195-5p/424-5p/497-5p, miR-411- | 4 |
| | 5p.2 | |
| TNFAIP6 | miR-23-3p | 1 |
| HJURP | | 0 |
| PATL2 | | 0 |
| TLR4 | miR-374-5p, miR-140-5p, miR-1197, miR-374-5p, miR-219a-2-3p, miR-326, miR-668- | 18 |
| | 3p, miR-124-3p.2/506-3p, miR-542-3p, miR-302-3p/372-3p/373-3p/520-3p, miR-216a- | |
| | 5p, miR-17-5p/20-5p/93-5p/106-5p/519-3p, miR-17-5p/20-5p/93-5p/106-5p/519-3p, | |
| | miR-421, miR-505-3p.2, miR-448, miR-489-3p, miR-145-5p | |
| LAPTM5 | miR-330-3p, miR-219-5p, miR-325-3p, miR-330-3p | 4 |
| RP11-876N24.3 | | 0 |
| RAB37 | miR-15-5p/16-5p/195-5p/424-5p/497-5p, miR-217, miR-200bc-3p/429, miR-24-3p, | 7 |
| | miR-296-5p, miR-488-3p, miR-127-3p | |
| LCK | miR-325-3p | 1 |
| MOB3C | miR-24-3p, miR-142-5p, miR-455-5p, miR-330-3p, miR-1-3p/206, miR-320 | 6 |
| GTF2H2C | | 0 |
| NGFR | miR-760, miR-1-3p/206, miR-423-5p, miR-296-5p, miR-27-3p, miR-299-3p, miR-128- | 10 |
| | 3p, miR-212-5p, miR-326, miR-326 | |
| KIF14 | miR-320, miR-144-3p | 2 |
| IGSF6 | miR-141-3p/200a-3p, miR-489-3p, miR-96-5p/1271-5p | 3 |
| TTC27 | | 0 |
| PPP2R2B | miR-26-5p, miR-141-3p/200a-3p, miR-494-3p | 3 |
| LGALS1 | miR-22-3p, miR-325-3p | 2 |
| RP5-1171I10.5 | | 0 |
| IKBIP | | 0 |
| XXbac- | | 0 |
| BPG299F13.14 | | |
| AC025171.1 | | 0 |
| CTSW | miR-140-3p.1 | 1 |
| SLC11A1 | miR-17-5p/20-5p/93-5p/106-5p/519-3p, miR-17-5p/20-5p/93-5p/106-5p/519-3p, miR- | 4 |
| | 874-3p, miR-302-3p/372-3p/373-3p/520-3p | |
| HGF | miR-181-5p, miR-369-3p, miR-142-5p, miR-381-3p, miR-140-3p.1, miR-199-5p, miR- | 27 |
| | 128-3p, miR-26-5p, miR-204-5p/211-5p, miR-27-3p, miR-543, miR-374-5p, miR-190- | |
| | 5p, miR-379-5p, miR-19-3p, miR-101-3p.2, miR-655-3p, miR-140-3p.2, miR-21- | |
| | 5p/590-5p, miR-144-3p, miR-144-3p, miR-101-3p.1, miR-653-5p, miR-1224-5p, miR- | |
| | 495-3p, miR-141-3p/200a-3p, miR-202-5p | |
| CNTRL | let-7-5p/98-5p | 0 |
| UGT1A6 | miR-141-3p/200a-3p | 1 |
| CD53 | miR-325-3p | 1 |
| RP11-701P16.2 | | 0 |
| DOCK11 | miR-182-5p, miR-376c-3p, miR-543, miR-381-3p, miR-182-5p, miR-155-5p, miR-136- | 14 |
| | 5p, miR-26-5p, miR-325-3p, miR-27-3p, miR-10-5p, miR-411-3p, miR-128-3p, miR- | |
| | 128-3p | |
| VIM-AS1 | | 0 |
| RP11-272L13.4 | | 0 |
| RP11-488L18.4 | | 0 |
| EMP3 | | 0 |
| CCL2 | miR-374-5p, miR-124-3p.2/506-3p | 2 |
| EXO5 | miR-361-5p, miR-150-5p, miR-125-5p, miR-532-3p, miR-873-5p.2 | 5 |
| PLA2G7 | | 0 |
| GPSM3 | miR-15-5p/16-5p/195-5p/424-5p/497-5p, miR-1224-5p | 2 |
| LINC01272 | | 0 |
| STAT1 | miR-338-3p, miR-150-5p, miR-802, miR-483-3p.1, miR-382-3p, miR-27-3p, miR-23- | 13 |
| | 3p, miR-129-5p, miR-3064-5p, miR-873-5p.2, miR-194-5p, miR-499a-5p, miR-128-3p | |
| GNG2 | miR-23-3p, miR-330-3p.2, miR-1298-5p, miR-194-5p, miR-212-5p, miR-493-3p, miR- | 16 |
| | 665, miR-138-5p, miR-411-3p, miR-29-3p, miR-124-3p.1, miR-181-5p, miR-299-3p, | |
| | miR-543, miR-203a-3p.2, miR-874-3p | |
| CR1 | miR-224-5p, miR-381-3p | 2 |
| SNRPF | miR-325-3p, miR-495-3p | 2 |
| TNNI2 | miR-325-3p | 1 |
| HCLS1 | | 0 |
| HIST1H3C | miR-760 | 1 |
| RP11-214O1.2 | | 0 |
| HENMT1 | | 0 |
| WIPF1 | miR-30-5p, miR-9-5p, miR-124-3p.2/506-3p, miR-153-3p, miR-153-3p, miR-655-3p, | 14 |
| | miR-141-3p/200a-3p, miR-124-3p.1, miR-200bc-3p/429, miR-200bc-3p/429, miR-382- | |
| | 3p, miR-135-5p, miR-200bc-3p/429, miR-182-5p | |
| TNFSF4 | miR-125-5p, miR-125-5p, miR-138-5p, miR-188-5p, miR-330-3p.2 | 5 |
| DOCK10 | miR-203a-3p.1, miR-203a-3p.2, miR-19-3p, miR-181-5p | 4 |
| ACSL1 | miR-205-5p, miR-142-3p.2, miR-202-5p, miR-124-3p.2/506-3p, miR-124-3p.2/506-3p, | 12 |
| | miR-203a-3p.2, miR-203a-3p.2, miR-124-3p.1, miR-130-3p/301-3p/454-3p, miR-376c- | |
| | 3p, miR-218-5p, miR-34-5p/449-5p | |
| IL10RA | miR-15-5p/16-5p/195-5p/424-5p/497-5p, miR-670-3p, miR-325-3p, miR-670-3p, miR- | 6 |
| | 665, miR-129-3p | |
| HIST1H2AK | miR-760 | 1 |
| CD97 | miR-505-3p.1, miR-21-5p/590-5p, miR-126-3p.2, miR-320 | 4 |
| ADAMTS2 | miR-330-3p.2, miR-29-3p, miR-29-3p, miR-29-3p, miR-330-3p, miR-145-5p, miR-340- | 10 |
| | 5p, miR-193a-5p, miR-873-5p.1, miR-29-3p | |
| MAD2L1 | miR-340-5p, miR-873-5p.1, miR-143-3p, miR-758-3p, miR-489-3p | 5 |
| SASH3 | miR-325-3p, miR-137 | 2 |
| SLC16A1 | miR-128-3p, miR-124-3p.1, miR-342-3p, miR-29-3p, miR-154-5p, miR-374-5p, miR- | 10 |
| | 539-3p, miR-582-5p, miR-124-3p.2/506-3p, miR-124-3p.2/506-3p | |
| RN7SKP203 | | 0 |
| RAC2 | | 0 |
| CD36 | | 0 |
| JAM3 | miR-101-3p.2, miR-582-5p, miR-544a-5p, miR-154-5p, miR-340-5p, miR-23-3p, miR- | 7 |
| | 340-5p | |
| BUB1B | | 0 |
| RRN3 | miR-25-3p/32-5p/92-3p/363-3p/367-3p, miR-340-5p, miR-381-3p | 3 |
| RAB20 | miR-128-3p, miR-27-3p | 2 |
| CTB-41I6.1 | | 0 |
| RNU4-2 | | 0 |
| CERKL | | 0 |
| TSPAN2 | miR-21-5p/590-5p, miR-30-5p, let-7-5p/98-5p, miR-219-5p, miR-299-5p | 4 |
| HLA-F | | 0 |
| CFP | miR-325-3p | 1 |
| ARL6IP6 | miR-200bc-3p/429, let-7-5p/98-5p, miR-30-5p, miR-26-5p | 3 |
| TNFRSF25 | | 0 |
| CPVL | miR-325-3p | 1 |
| CD6 | miR-216a-5p, miR-216b-5p | 2 |
| MYADM | miR-96-5p/1271-5p, miR-124-3p.2/506-3p, miR-182-5p, miR-15-5p/16-5p/195-5p/424- | 9 |
| | 5p/497-5p, miR-15-5p/16-5p/195-5p/424-5p/497-5p, miR-124-3p.1, miR-329-3p/362- | |
| | 3p, miR-873-5p.2, miR-34-5p/449-5p | |
| RP11-149I23.3 | | 0 |
| HLA-DRB5 | | 0 |
| RFC3 | | 0 |
| FBN1 | miR-144-3p, miR-144-3p, miR-425-5p, miR-140-5p, miR-302c-3p.2/520-3p, miR-101- | 29 |
| | 3p.1, miR-330-3p.2, miR-382-5p, miR-29-3p, miR-29-3p, miR-494-3p, miR-486-5p, | |
| | miR-9-5p, miR-9-5p, miR-101-3p.2, miR-101-3p.2, miR-582-5p, miR-340-5p, miR- | |
| | 542-3p, miR-190-5p, miR-182-5p, miR-133a-3p.2/133b, miR-329-3p/362-3p, miR-758- | |
| | 3p, miR-325-3p, miR-325-3p, miR-148-3p/152-3p, miR-23-3p, miR-25-3p/32-5p/92- | |
| | 3p/363-3p/367-3p | |
| TAP1 | miR-532-5p, miR-340-5p, miR-101-3p.1 | 3 |
| NUSAP1 | | 0 |
| PLXNC1 | miR-140-3p.2, miR-193-3p, miR-29-3p, miR-144-3p, miR-181-5p, miR-369-3p, miR- | 46 |
| | 410-3p, miR-15-5p/16-5p/195-5p/424-5p/497-5p, miR-129-3p, miR-302c-3p.2/520-3p, | |
| | miR-141-3p/200a-3p, miR-543, miR-18-5p, miR-330-3p.2, miR-320, miR-320, miR- | |
| | 200bc-3p/429, miR-409-3p, miR-200bc-3p/429, miR-302-3p/372-3p/373-3p/520-3p, | |
| | miR-382-5p, miR-194-5p, miR-101-3p.2, miR-142-3p.2, miR-485-5p, miR-340-5p, | |
| | miR-374-5p, miR-499a-5p, miR-134-5p, miR-19-3p, miR-218-5p, miR-23-3p, miR- | |
| | 505-3p.2, miR-421, miR-377-3p, miR-455-5p, miR-150-5p, let-7-5p/98-5p, miR-139- | |
| | 5p, miR-128-3p, miR-140-3p.1, miR-199-5p, miR-299-5p, miR-30-5p, miR-221- | |
| | 3p/222-3p, miR-219-5p, miR-221-3p/222-3p | |
| NLRC4 | | 0 |
| CTD- | | 0 |
| 2047H16.3 | | |
| GPR68 | miR-221-3p/222-3p | 1 |
| MAP7D3 | miR-219-5p, miR-194-5p | 2 |
| ARG1 | miR-340-5p | 1 |
| F2R | miR-181-5p | 1 |
| PIK3CD-AS1 | | 0 |
| GSG2 | miR-760, miR-204-5p/211-5p | 2 |
| HS3ST3B1 | miR-143-3p, miR-1-3p/206, miR-346, miR-330-3p, miR-9-5p, miR-494-3p, miR-218- | 24 |
| | 5p, miR-124-3p.2/506-3p, miR-340-5p, miR-539-3p, miR-375, miR-493-5p, miR-493- | |
| | 5p, miR-144-3p, miR-216b-5p, miR-216a-5p, miR-99-5p/100-5p, miR-29-3p, miR- | |
| | 376c-3p, miR-216a-5p, miR-15-5p/16-5p/195-5p/424-5p/497-5p, miR-1197, miR-382- | |
| | 5p, miR-129-5p | |
| IQGAP2 | miR-219a-2-3p, miR-199-3p, miR-299-5p, miR-133a-3p.2/133b, miR-411-5p.2, miR- | 11 |
| | 340-5p, miR-543, miR-124-3p.1, miR-181-5p, miR-25-3p/32-5p/92-3p/363-3p/367-3p, | |
| | miR-130-3p/301-3p/454-3p | |
| ALOX5AP | | 0 |
| AC007620.3 | | 0 |
| IDO1 | | 0 |
| BCL2A1 | | 0 |
| CALHM2 | | 0 |
| KIF11 | miR-101-3p.2, miR-381-3p, miR-655-3p, miR-381-3p | 4 |
| TTK | miR-455-3p.1, miR-140-5p, miR-338-3p, miR-101-3p.2, miR-582-5p, miR-132-3p/212- | 7 |
| | 3p, miR-142-3p.2 | |
| LTB | miR-493-3p, miR-15-5p/16-5p/195-5p/424-5p/497-5p, miR-134-5p, miR-146-5p | 4 |
| FCGR1A | | 0 |
| EEF1E1 | miR-204-5p/211-5p, miR-96-5p/1271-5p, miR-212-5p, miR-183-5p.2 | 4 |
| AC006460.2 | | 0 |
| RNASE2 | | 0 |
| KIF15 | | 0 |
| P2RY8 | | 0 |
| LPAR6 | miR-27-3p | 1 |

TABLE 2
AUC to evaluate performance of classifiers

| Disease State | AUC for lowest | AUC for highest |
|---|---|---|
| Comparisons | ML classifier | ML classifier |
| ABMR vs NR | 0.93 | 0.97 |
| TCMR vs NR | 0.93 | 0.99 |
| ABMR vs TCMR | 0.87 | 0.98 |
| mABMR vs NR | 0.93 | 0.96 |
| mTCMR vs NR | 0.96 | 0.99 |
| mABMR vs mTCMR | 0.82 | 0.89 |

TABLE 3
Genes Identified in Urine Effectively Differentiate Rejection States
in Kidney biopsy by t-Test, while those identified in blood do not

| | ABMR | TCMR | Mixed | ABMR |
|---|---|---|---|---|
| | vs NR | vs NR | vs NR | vs TCMR |
| All Genes | | | | |
| Count significant | 3538 | 8214 | 6120 | 4307 |
| (Bonferroni => a 0.01 / n comparisons) | | | | |
| Count total | 19416 | 19416 | 19416 | 19416 |
| Significance threshold | 2.03E−07 | 2.03E−07 | 2.03E−07 | 2.03E−07 |
| Percent genes significant | 18.22 | 42.31 | 31.52 | 22.18 |
| Count in top 10% by t-Test | 1941 | 1941 | 1941 | 1941 |
| Percent in top 10% t-Test | 10 | 10 | 10 | 10 |
| Urine Genes | | | | |
| Count significant | 34 | 38 | 37 | 29 |
| (Bonferroni => a 0.01 / n comparisons) | | | | |
| Count total | 49 | 49 | 49 | 49 |
| Significance threshold | 2.03E−07 | 2.03E−07 | 2.03E−07 | 2.03E−07 |
| Percent genes significant | 69.39 | 77.55 | 75.51 | 59.18 |
| Count in top 10% by t-Test | 31 | 30 | 31 | 23 |
| Percent in top 10% t-Test | 63.27 | 61.22 | 63.27 | 46.94 |
| PaxGene ® RNA Blood Genes | | | | |
| (Akalin et al. 2021) | | | | |
| Count significant | 3 | 6 | 4 | 3 |
| (Bonferroni => a 0.01 / n comparisons) | | | | |
| Count total | 11 | 11 | 11 | 11 |
| Significance threshold | 2.03E−07 | 2.03E−07 | 2.03E−07 | 2.03E−07 |
| Percent probes significant | 27.27 | 54.55 | 36.36 | 27.27 |
| Count in top 10% by t-Test | 3 | 2 | 3 | 2 |
| Percent in top 10% t-Test | 27.27 | 18.18 | 27.27 | 18.18 |

TABLE 4
Performance evaluation of target gene sets

| Logistic | ABMR | TCMR vs | ABMR vs | mABMR | mTCMR | mABMR vs |
|---|---|---|---|---|---|---|
| Regression | vs NR | NR | TCMR | vs NR | vs NR | mTCMR |
| Urine63p23g | 0.969 | 0.987 | 0.980 | 0.963 | 0.987 | 0.888 |
| Urine139p49g | 0.966 | 0.988 | 0.984 | 0.959 | 0.984 | 0.896 |
| MMDx39p39g | 0.988 | 0.995 | 0.994 | 0.984 | 0.994 | 0.944 |
| Akalin10p5g | 0.656 | 0.909 | 0.859 | 0.672 | 0.897 | 0.756 |
| Akalin30p11g | 0.862 | 0.979 | 0.924 | 0.867 | 0.974 | 0.803 |
| Akalin47p14g | 0.751 | 0.902 | 0.761 | 0.780 | 0.897 | 0.689 |

TABLE 5
evaluation of performance of target
genes by using the Banff samples

| | ABMR | TCMR | ABMR |
|---|---|---|---|
| LogisticRegression | vs NR | vs NR | vs TCMR |
| Urine63p23g | 0.895 | 0.896 | 0.903 |
| Urine139p49g | 0.889 | 0.872 | 0.901 |
| MMDx39p39g | 0.909 | 0.911 | 0.959 |
| Akalin10p5g | 0.625 | 0.82 | 0.736 |
| Akalin30p11g | 0.834 | 0.898 | 0.782 |
| Akalin47p14g | 0.748 | 0.834 | 0.663 |

TABLE 6
53 target genes that can differentiate
between kidney rejection states

| Urine saturated | | How |
|---|---|---|
| genes miRNA | | many |
| genes | miRNAs predicted to bind | found |
| ALB | | 0 |
| BASP1 | miR-96-5p/1271-5p, miR-493-5p, miR-183- | 8 |
| | 5p.2, miR-150-5p, miR-7-5p, miR-653-5p, | |
| | miR-200bc-3p/429, miR-212-5p | |
| BMP7 | miR-1298-5p, miR-137, miR-1298-5p, miR- | 11 |
| | 758-3p, miR-325-3p, miR-325-3p, miR-542-3p, | |
| | miR-25-3p/32-5p/92-3p/363-3p/367-3p, miR- | |
| | 495-3p, miR-30-5p, miR-873-5p.1 | |
| C11orf21 | | 0 |
| C1QB | | 0 |
| C3 | | 0 |
| CALHM6 | | 0 |
| CD14 | | 0 |
| CD3D | miR-146-5p, miR-325-3p, miR-505-3p.1 | 3 |
| CD3E | | 0 |
| CD46 | miR-539-3p | 1 |
| CD6 | miR-216a-5p, miR-216b-5p | 2 |
| CD74 | | 0 |
| CDH1 | miR-340-5p, miR-361-5p, miR-495-3p, miR- | 7 |
| | 338-3p, miR-217, miR-9-5p, miR-219a-2-3p | |
| CXCL10 | miR-15-5p/16-5p/195-5p/424-5p/497-5p, miR- | 2 |
| | 503-5p | |
| CXCL11 | miR-199-3p, miR-9-5p, miR-1-3p/206 | 3 |
| CXCL14 | miR-17-5p/20-5p/93-5p/106-5p/519-3p, miR- | 5 |
| | 302-3p/372-3p/373-3p/520-3p, miR-142-5p, | |
| | miR-302c-3p.2/520-3p, miR-340-5p | |
| CXCL9 | miR-325-3p | 1 |
| ENG | miR-326, miR-325-3p, miR-760, miR-138-5p | 4 |
| FN1 | miR-27-3p, miR-145-5p, miR-142-3p.2, miR-1- | 16 |
| | 3p/206, miR-101-3p.2, miR-96-5p/1271-5p, | |
| | miR-182-5p, miR-203a-3p.2, miR-183-5p.2, | |
| | miR-140-3p.1, miR-217, miR-183-5p.1, miR- | |
| | 199-3p, miR-200bc-3p/429, miR-144-3p, miR- | |
| | 101-3p.1 | |
| FOXP3 | miR-325-3p | 1 |
| GNLY | | 0 |
| GZMB | | 0 |
| HAVCR2 | | 0 |
| IFNGR1 | | 0 |
| IL18BP | miR-330-3p, miR-142-5p, miR-224-5p, miR- | 7 |
| | 330-3p.2, miR-148-3p/152-3p, miR-485-5p, | |
| | miR-122-5p | |
| IL2RA | miR-30-5p, miR-302c-3p.2/520-3p | 2 |
| IL32 | | 0 |
| INPP5D | miR-155-5p | 1 |
| IRAK2 | miR-15-5p/16-5p/195-5p/424-5p/497-5p, miR- | 3 |
| | 302-3p/372-3p/373-3p/520-3p, miR-320 | |
| ISG20 | | 0 |
| KLRC4- | | 0 |
| KLRK1 | | |
| LCK | miR-325-3p | 1 |
| MAP4K1 | | 0 |
| MRC1 | miR-23-3p | 1 |
| NAMPT | miR-1-3p/206, miR-124-3p.2/506-3p, miR-135- | 7 |
| | 5p, miR-381-3p, miR-26-5p, miR-182-5p, miR- | |
| | 96-5p/1271-5p | |
| NKG7 | | 0 |
| PRF1 | | 0 |
| PRPF31 | miR-1224-5p, miR-192-5p/215-5p, miR-1249-3p | 3 |
| PRPF32 | | 0 |
| PRPF33 | | 0 |
| PSMB10 | | 0 |
| PSMB9 | miR-125-5p, miR-483-3p.2, miR-668-3p | 3 |
| PYCARD | | 0 |
| RGS1 | miR-223-3p, miR-655-3p, miR-27-3p | 3 |
| RUNX3 | miR-382-5p, miR-330-3p, miR-130-3p/301- | 14 |
| | 3p/454-3p, miR-17-5p/20-5p/93-5p/106- | |
| | 5p/519-3p, miR-17-5p/20-5p/93-5p/106- | |
| | 5p/519-3p, miR-495-3p, miR-495-3p, miR-19- | |
| | 3p, miR-101-3p.2, miR-582-5p, miR-145-5p, | |
| | miR-873-5p.1, miR-302-3p/372-3p/373- | |
| | 3p/520-3p, miR-194-5p | |
| SERPINA1 | | 0 |
| SERPINB12 | | 0 |
| STAT1 | miR-338-3p, miR-150-5p, miR-802, miR-483- | 13 |
| | 3p.1, miR-382-3p, miR-27-3p, miR-23-3p, | |
| | miR-129-5p, miR-3064-5p, miR-873-5p.2, | |
| | miR-194-5p, miR-499a-5p, miR-128-3p | |
| TAP1 | miR-532-5p, miR-340-5p, miR-101-3p.1 | 3 |
| TBP | miR-200bc-3p/429, miR-15-5p/16-5p/195- | 4 |
| | 5p/424-5p/497-5p, miR-505-3p.1, miR-27-3p | |
| TGFB1 | miR-296-5p, miR-361-5p, miR-873-5p.1, miR- | 6 |
| | 873-5p.2, miR-744-5p, miR-425-5p | |
| TMEM156 | miR-218-5p, miR-135-5p, miR-496.1 | 3 |

TABLE 7
Gene Ontology Enrichment Analysis

| | Enrichment | | Pathway | Fold | |
|---|---|---|---|---|---|
| Gene set | FDR | Genes | Genes | Enrichment | Pathways |
| Akalin10p5g | No significant enrichment | | | | |
| Akalin30p11g | 7.70E−03 | 3 | 124 | 43.1 | Integrin-mediated signaling pathway |
| AlloMap 11 | 7.90E−03 | 5 | 965 | 10.3 | Cytokine-mediated signaling pathway |
| genes | 7.40E−03 | 6 | 1308 | 8.9 | Cellular response to cytokine stimulus |
| | 7.40E−03 | 6 | 1404 | 8.2 | Response to cytokine |
| | 7.90E−03 | 6 | 1658 | 6.9 | Cell activation |
| Urine139p49g | 8.40E−10 | 14 | 646 | 12.4 | Adaptive immune response |
| Urine 49 | 2.10E−10 | 18 | 965 | 8.7 | Cytokine-mediated signaling pathway |
| genes | 2.30E−10 | 19 | 1190 | 7.7 | Pos. reg. of immune system proc. |
| | 2.40E−10 | 20 | 1499 | 7 | Defense response to other organism |
| | 4.80E−13 | 25 | 1866 | 6.7 | Response to other organism |
| | 4.80E−13 | 25 | 1867 | 6.7 | Response to external biotic stimulus |
| | 2.50E−09 | 19 | 1308 | 6.6 | Cellular response to cytokine stimulus |
| | 8.30E−09 | 19 | 1404 | 6.1 | Response to cytokine |
| | 1.50E−08 | 20 | 1658 | 5.4 | Cell activation |

TABLE 8
Gene Ontology (GO) Enrichment Analysis

| | Akalin | Akalin | Akalin | MMDx | Urine | Urine |
|---|---|---|---|---|---|---|
| MSigDB Gene sets | 10p5g | 30p11g | 47p14g | 39p26g | 63p23g | 139p49g |
| hallmark_allograft | 1.0E+00 | 1.1E−01 | 1.0E+00 | 2.2E−03 | 8.9E−05 | 3.1E−18 |
| hallmark_inflammatory | 1.0E+00 | 1.0E+00 | 1.0E+00 | 2.8E−02 | 4.0E−06 | 8.2E−07 |
| kegg_allograft | 1.0E+00 | 1.0E+00 | 1.0E+00 | 4.9E−02 | 1.0E+00 | 4.6E−03 |
| kegg_T-cell | 2.8E−02 | 6.0E−02 | 7.6E−02 | 1.2E−05 | 7.4E−03 | 2.7E−03 |

TABLE 9
Apoptosis pathway genes

| Original | NCBI (Entrez) | Gene | Gene |
|---|---|---|---|
| Member | Gene Id | Symbol | Description |
| ADD1 | 118 | ADD1 | adducin 1 |
| AIFM3 | 150209 | AIFM3 | apoptosis inducing factor mitochondria |
| ANKH | 56172 | ANKH | ANKH inorganic pyrophosphate transport |
| ANXA1 | 301 | ANXA1 | annexin A1 |
| APP | 351 | APP | amyloid beta precursor protein |
| ATF3 | 467 | ATF3 | activating transcription factor 3 |
| AVPR1A | 552 | AVPR1A | arginine vasopressin receptor 1A |
| BAX | 581 | BAX | BCL2 associated X, apoptosis regulator |
| BCAP31 | 10134 | BCAP31 | B cell receptor associated protein 31 |
| BCL10 | 8915 | BCL10 | BCL10 immune signaling adaptor |
| BCL2L1 | 598 | BCL2L1 | BCL2 like 1 |
| BCL2L10 | 10017 | BCL2L10 | BCL2 like 10 |
| BCL2L11 | 10018 | BCL2L11 | BCL2 like 11 |
| BCL2L2 | 599 | BCL2L2 | BCL2 like 2 |
| BGN | 633 | BGN | biglycan |
| BID | 637 | BID | BH3 interacting domain death agonist |
| BIK | 638 | BIK | BCL2 interacting killer |
| BIRC3 | 330 | BIRC3 | baculoviral IAP repeat containing 3 |
| BMF | 90427 | BMF | Bcl2 modifying factor |
| BMP2 | 650 | BMP2 | bone morphogenetic protein 2 |
| BNIP3L | 665 | BNIP3L | BCL2 interacting protein 3 like |
| BRCA1 | 672 | BRCA1 | BRCA1 DNA repair associated |
| BTG2 | 7832 | BTG2 | BTG anti-proliferation factor 2 |
| BTG3 | 10950 | BTG3 | BTG anti-proliferation factor 3 |
| CASP1 | 834 | CASP1 | caspase 1 |
| CASP2 | 835 | CASP2 | caspase 2 |
| CASP3 | 836 | CASP3 | caspase 3 |
| CASP4 | 837 | CASP4 | caspase 4 |
| CASP6 | 839 | CASP6 | caspase 6 |
| CASP7 | 840 | CASP7 | caspase 7 |
| CASP8 | 841 | CASP8 | caspase 8 |
| CASP9 | 842 | CASP9 | caspase 9 |
| CAV1 | 857 | CAV1 | caveolin 1 |
| CCNA1 | 8900 | CCNA1 | cyclin A1 |
| CCND1 | 595 | CCND1 | cyclin D1 |
| CCND2 | 894 | CCND2 | cyclin D2 |
| CD14 | 929 | CD14 | CD14 molecule |
| CD2 | 914 | CD2 | CD2 molecule |
| CD38 | 952 | CD38 | CD38 molecule |
| CD44 | 960 | CD44 | CD44 molecule |
| CD69 | 969 | CD69 | CD69 molecule |
| CDC25B | 994 | CDC25B | cell division cycle 25B |
| CDK2 | 1017 | CDK2 | cyclin dependent kinase 2 |
| CDKN1A | 1026 | CDKN1A | cyclin dependent kinase inhibitor 1A |
| CDKN1B | 1027 | CDKN1B | cyclin dependent kinase inhibitor 1B |
| CFLAR | 8837 | CFLAR | CASP8 and FADD like apoptosis |
| CLU | 1191 | CLU | clusterin |
| CREBBP | 1387 | CREBBP | CREB binding protein |
| CTH | 1491 | CTH | cystathionine gamma-lyase |
| CTNNB1 | 1499 | CTNNB1 | catenin beta 1 |
| CYLD | 1540 | CYLD | CYLD lysine 63 deubiquitinase |
| DAP | 1611 | DAP | death associated protein |
| DAP3 | 7818 | DAP3 | death associated protein 3 |
| DCN | 1634 | DCN | decorin [Source: HGNC |
| DDIT3 | 1649 | DDIT3 | DNA damage inducible transcript 3 |
| DFFA | 1676 | DFFA | DNA fragmentation factor subunit |
| DIABLO | 56616 | DIABLO | diablo IAP-binding mitochondrial protein |
| DNAJA1 | 3301 | DNAJA1 | DnaJ heat shock protein family (Hsp40) |
| DNAJC3 | 5611 | DNAJC3 | DnaJ heat shock protein family (Hsp40) |
| DNM1L | 10059 | DNM1L | dynamin 1 like |
| DPYD | 1806 | DPYD | dihydropyrimidinc dehydrogenase |
| EBP | 10682 | EBP | EBP cholestenol delta-isomerase |
| EGR3 | 1960 | EGR3 | early growth response 3 |
| EMP1 | 2012 | EMP1 | epithelial membrane protein 1 |
| ENO2 | 2026 | ENO2 | enolase 2 |
| ERBB2 | 2064 | ERBB2 | erb-b2 receptor tyrosine kinase 2 |
| ERBB3 | 2065 | ERBB3 | erb-b2 receptor tyrosine kinase 3 |
| EREG | 2069 | EREG | epiregulin |
| ETF1 | 2107 | ETF1 | eukaryotic translation termination factor |
| F2 | 2147 | F2 | coagulation factor II, thrombin |
| F2R | 2149 | F2R | coagulation factor II thrombin receptor |
| FAS | 355 | FAS | Fas cell surface death receptor |
| FASLG | 356 | FASLG | Fas ligand |
| FDXR | 2232 | FDXR | ferredoxin reductase |
| FEZ1 | 9638 | FEZ1 | fasciculation and elongation protein |
| GADD45A | 1647 | GADD45A | growth arrest and DNA damage inducible |
| GADD45B | 4616 | GADD45B | growth arrest and DNA damage inducible |
| GCH1 | 2643 | GCH1 | GTP cyclohydrolase 1 |
| GNA15 | 2769 | GNA15 | G protein subunit alpha 15 |
| GPX1 | 2876 | GPX1 | glutathione peroxidase 1 |
| GPX3 | 2878 | GPX3 | glutathione peroxidase 3 |
| GPX4 | 2879 | GPX4 | glutathione peroxidase 4 |
| GSN | 2934 | GSN | gelsolin |
| GSR | 2936 | GSR | glutathione-disulfide reductase |
| GSTM1 | 2944 | GSTM1 | glutathione S-transferase mu 1 |
| GUCY2D | 3000 | GUCY2D | guanylate cyclase 2D, retinal |
| H1F0 | 3005 | H1-0 | H1.0 linker histone |
| HGF | 3082 | HGF | hepatocyte growth factor |
| HMGB2 | 3148 | HMGB2 | high mobility group box 2 |
| HMOX1 | 3162 | HMOX1 | heme oxygenase 1 |
| HSPB1 | 3315 | HSPB1 | heat shock protein family B (small) |
| IER3 | 8870 | IER3 | immediate early response 3 [Source: HGN . . . |
| IFITM3 | 10410 | IFITM3 | interferon induced transmembrane protein |
| IFNB1 | 3456 | IFNB1 | interferon beta 1 |
| IFNGR1 | 3459 | IFNGR1 | interferon gamma receptor 1 |
| IGF2R | 3482 | IGF2R | insulin like growth factor 2 receptor |
| IGFBP6 | 3489 | IGFBP6 | insulin like growth factor binding protein |
| IL18 | 3606 | IL18 | interleukin 18 |
| IL1A | 3552 | IL1A | interleukin 1 alpha |
| IL1B | 3553 | IL1B | interleukin 1 beta |
| IL6 | 3569 | IL6 | interleukin 6 |
| IRF1 | 3659 | IRF1 | interferon regulatory factor 1 |
| ISG20 | 3669 | ISG20 | interferon stimulated exonuclease gene |
| JUN | 3725 | JUN | Jun proto-oncogene, AP-1 |
| KRT18 | 3875 | KRT18 | keratin 18 |
| LEF1 | 51176 | LEF1 | lymphoid enhancer binding factor 1 |
| LGALS3 | 3958 | LGALS3 | galectin 3 |
| LMNA | 4000 | LMNA | lamin A/C |
| LPPR4 | 9890 | PLPPR4 | phospholipid phosphatase related 4 |
| LUM | 4060 | LUM | lumican |
| MADD | 8567 | MADD | MAP kinase activating death domain |
| MCL1 | 4170 | MCL1 | MCL1 apoptosis regulator, BCL2 family |
| MGMT | 4255 | MGMT | O-6-methylguanine-DNA methyltransferase |
| MMP2 | 4313 | MMP2 | matrix metallopeptidase 2 |
| NEDD9 | 4739 | NEDD9 | neural precursor cell expressed protein |
| NEFH | 4744 | NEFH | neurofilament heavy chain |
| PAK1 | 5058 | PAK1 | p21 (RAC1) activated kinase 1 |
| PDCD4 | 27250 | PDCD4 | programmed cell death 4 |
| PDGFRB | 5159 | PDGFRB | platelet derived growth factor receptor |
| PEA15 | 8682 | PEA15 | proliferation and apoptosis adaptor |
| PLAT | 5327 | PLAT | plasminogen activator, tissue type |
| PLCB2 | 5330 | PLCB2 | phospholipase C beta 2 |
| PMAIP1 | 5366 | PMAIP1 | phorbol-12-myristate-13-acetate-induceed protein |
| PPP2R5B | 5526 | PPP2R5B | protein phosphatase 2 regulatory subunit |
| PPP3R1 | 5534 | PPP3R1 | protein phosphatase 3 regulatory subunit |
| PPT1 | 5538 | PPT1 | palmitoyl-protein thioesterase 1 |
| PRF1 | 5551 | PRF1 | perforin 1 |
| PSEN1 | 5663 | PSEN1 | presenilin 1 |
| PSEN2 | 5664 | PSEN2 | presenilin 2 |
| PTK2 | 5747 | PTK2 | protein tyrosine kinase 2 |
| RARA | 5914 | RARA | retinoic acid receptor alpha |
| RELA | 5970 | RELA | RELA proto-oncogene, NF-kB subunit |
| RETSAT | 54884 | RETSAT | retinol saturase |
| RHOB | 388 | RHOB | ras homolog family member B |
| RHOT2 | 89941 | RHOT2 | ras homolog family member T2 |
| RNASEL | 6041 | RNASEL | ribonuclease L |
| ROCK1 | 6093 | ROCK1 | Rho associated coiled-coil containing |
| SAT1 | 6303 | SAT1 | spermidine/spermine N1-acetyltransferase |
| SATB1 | 6304 | SATB1 | SATB homeobox 1 |
| SC5DL | 6309 | SC5D | sterol-C5-desaturase |
| SLC20A1 | 6574 | SLC20A1 | solute carrier family 20 member 1 |
| SMAD7 | 4092 | SMAD7 | SMAD family member 7 |
| SOD1 | 6647 | SOD1 | superoxide dismutase 1 |
| SOD2 | 6648 | SOD2 | superoxide dismutase 2 |
| SPTAN1 | 6709 | SPTAN1 | spectrin alpha, non-erythrocytic 1 |
| SQSTM1 | 8878 | SQSTM1 | sequestosome 1 |
| TAP1 | 6890 | TAP1 | transporter 1 |
| TGFB2 | 7042 | TGFB2 | transforming growth factor beta 2 |
| TGFBR3 | 7049 | TGFBR3 | transforming growth factor beta receptor |
| TIMP1 | 7076 | TIMP1 | TIMP metallopeptidase inhibitor 1 |
| TIMP2 | 7077 | TIMP2 | TIMP metallopeptidase inhibitor 2 |
| TIMP3 | 7078 | TIMP3 | TIMP metallopeptidase inhibitor 3 |
| TNF | 7124 | TNF | tumor necrosis factor |
| TNFRSF12A | 51330 | TNFRSF12A | TNF receptor superfamily member 12A |
| TNFSF10 | 8743 | TNFSF10 | TNF superfamily member 10 |
| TOP2A | 7153 | TOP2A | DNA topoisomerase II alpha |
| TSPO | 706 | TSPO | translocator protein |
| TXNIP | 10628 | TXNIP | thioredoxin interacting protein |
| VDAC2 | 7417 | VDAC2 | voltage dependent anion channel 2 |
| WEE1 | 7465 | WEE1 | WEE1 G2 checkpoint kinase |
| XIAP | | | |

TABLE 10
The Urine enriched gene sets effectively differentiate rejection states

| | | 8 Classifiers of Kidney Disease | | | | | | | |
|---|---|---|---|---|---|---|---|---|---|
| | | lr | gnb | knn | dt | rf | gb | ab | bag |
| Urine | ABMR_NR | 0.969 | 0.945 | 0.958 | 0.933 | 0.969 | 0.968 | 0.950 | 0.966 |
| 63 | TCMR_NR | 0.987 | 0.985 | 0.988 | 0.927 | 0.990 | 0.990 | 0.990 | 0.982 |
| | ABMR_TCMR | 0.980 | 0.931 | 0.953 | 0.873 | 0.960 | 0.956 | 0.955 | 0.949 |
| | mABMR_NR | 0.963 | 0.934 | 0.951 | 0.929 | 0.963 | 0.962 | 0.956 | 0.957 |
| | mTCMR_NR | 0.987 | 0.981 | 0.986 | 0.959 | 0.988 | 0.988 | 0.988 | 0.984 |
| | mABMR_mTCMR | 0.888 | 0.848 | 0.854 | 0.824 | 0.872 | 0.876 | 0.875 | 0.868 |
| Urine | ABMR_NR | 0.966 | 0.928 | 0.954 | 0.933 | 0.972 | 0.973 | 0.969 | 0.966 |
| 139 | TCMR_NR | 0.988 | 0.979 | 0.983 | 0.939 | 0.988 | 0.990 | 0.989 | 0.984 |
| | ABMR_TCMR | 0.984 | 0.906 | 0.952 | 0.881 | 0.962 | 0.972 | 0.967 | 0.959 |
| | mABMR_NR | 0.959 | 0.918 | 0.949 | 0.920 | 0.965 | 0.964 | 0.958 | 0.959 |
| | mTCMR_NR | 0.984 | 0.977 | 0.982 | 0.957 | 0.987 | 0.987 | 0.986 | 0.987 |
| | mABMR_mTCMR | 0.896 | 0.827 | 0.848 | 0.824 | 0.873 | 0.870 | 0.867 | 0.860 |
| Mmdx | ABMR_NR | 0.988 | 0.972 | 0.980 | 0.959 | 0.987 | 0.987 | 0.985 | 0.984 |
| 39 | TCMR_NR | 0.995 | 0.993 | 0.995 | 0.934 | 0.994 | 0.994 | 0.994 | 0.994 |
| | ABMR_TCMR | 0.994 | 0.987 | 0.994 | 0.927 | 0.991 | 0.993 | 0.994 | 0.991 |
| | mABMR_NR | 0.984 | 0.961 | 0.976 | 0.941 | 0.982 | 0.983 | 0.979 | 0.980 |
| | mTCMR_NR | 0.994 | 0.992 | 0.991 | 0.931 | 0.994 | 0.993 | 0.992 | 0.993 |
| | mABMR_mTCMR | 0.944 | 0.917 | 0.932 | 0.865 | 0.931 | 0.944 | 0.958 | 0.937 |
| Akalin | ABMR_NR | 0.751 | 0.665 | 0.596 | 0.671 | 0.732 | 0.740 | 0.730 | 0.718 |
| 47 | TCMR_NR | 0.902 | 0.805 | 0.805 | 0.709 | 0.838 | 0.861 | 0.847 | 0.829 |
| | ABMR_TCMR | 0.761 | 0.682 | 0.685 | 0.633 | 0.715 | 0.713 | 0.697 | 0.672 |
| | mABMR_NR | 0.780 | 0.685 | 0.651 | 0.693 | 0.757 | 0.769 | 0.762 | 0.741 |
| | mTCMR_NR | 0.897 | 0.816 | 0.807 | 0.712 | 0.836 | 0.845 | 0.825 | 0.823 |
| | mABMR_mTCMR | 0.689 | 0.664 | 0.622 | 0.613 | 0.670 | 0.684 | 0.607 | 0.661 |

TABLE 11
MMDx30p39g set

| ABMR | | TCMR | |
|---|---|---|---|
| Symbol | Probe set ID | Symbol | Probe set ID |
| PLA1A | 11727116_a_at | IFNG | 11730947_at |
| LYPD5 | 11729803_a_at | SIRPG | 11754881_a_at |
| SH2D1B | 11740156_a_at | ADAMDEC1 | 11750826_x_at |
| SH2D1B | 11740157_at | LAG3 | 11730060_a_at |
| S1PR5 | 11752664_a_at | ADAMDEC1 | 11729110_s_at |
| ROBO4 | 11755778_a_at | CXCR6 | 11729977_a_at |
| LYPD5 | 11739775_a_at | ICOS | 11731360_a_at |
| FGFBP2 | 11722782_at | ADAMDEC1 | 11751267_a_at |
| GNLY | 11751857_a_at | ADAMDEC1 | 11729111_x_at |
| GNLY | 11763715_a_at | JAKMIP1 | 11750942_x_at |
| SH2D1B | 11749207_a_at | ADAMDEC1 | 11750825_a_at |
| GNLY | 11756632_a_at | ZBED2 | 11726909_at |
| KLRF1 | 11753219_a_at | CXCL13 | 11720162_at |
| TRDC | 11761790_x_at | CD8B | 11735474_a_at |
| CX3CR1 | 11723048_at | CXCL13 | 11720161_at |
| KLRF1 | 11740669_x_at | CTLA4 | 11730637_a_at |
| ROBO4 | 11755622_x_at | LAG3 | 11749522_a_at |
| TRDC | 11763447_x_at | BATF | 11724374_at |
| CXCL11 | 11732467_x_at | BTLA | 11735221_a_at |
| ADAM15 | 11719513_a_at | CD8A* | 11724729_a_* |

*Not included

TABLE 12

| | ABMR Vs | TCMR Vs |
|---|---|---|
| | NR | NR |
| Count Overexpressed | 17230 | 18848 |
| overall | | |
| Count Underexpressed | 32063 | 30445 |
| overall | | |
| Percent Overexpressed | 35 | 38.2 |
| Overall | | |
| Count Overexpressed | 3783 | 3667 |
| Top 10% ABMR | | |
| Count Underexpressed | 1146 | 1262 |
| Top 10% ABMR | | |
| Percent Overexpressed | 76.7 | 74.4 |
| Top 10% ABMR | | |
| Count Overexpressed | 3502 | 3503 |
| Top 10% TCMR | | |
| Count Underexpressed | 1427 | 1426 |
| Top 10% TCMR | | |
| Percent Overexpressed | 71 | 71.1 |
| Top 10% TCMR | | |
| Count Overexpressed | 115 | 119 |
| Urine Set | | |
| Count Underexpressed | 24 | 20 |
| Urine Set | | |
| Percent Overexpressed | 82.7 | 85.6 |
| Urine Set | | |
| Count Overexpressed | 15 | 23 |
| Akalin Set | | |
| Count Underexpressed | 15 | 7 |
| Akalin Set | | |
| Percent Overexpressed | 50 | 76.7 |
| Akalin Set | | |

What is claimed is:

1. A method of preparing a composition of nucleic acids derived from a urine sample of a kidney transplant recipient useful for determination of kidney transplant rejection or risk of kidney transplant rejection comprising:
(a) extracting nucleic acids from the urine sample of the kidney transplant recipient, wherein the extracted nucleic acids comprise one or more mRNAs of target genes and/or miRNAs associated with kidney transplant rejection;
(b) preparing a composition of nucleic acids from the extracted nucleic acids in step (a) by isolating mRNAs and/or miRNAs and removing contaminating molecules, optionally wherein preparing the composition comprises reverse transcribing complementary DNA (cDNA) from the nucleic acids extracted in step (a);
(c) measuring an amount of the one or more mRNAs and/or miRNAs, and generating one or more transplant rejection scores from the measured amount of the one or more mRNAs and/or miRNAs, wherein the one or more transplant rejection scores provide a quantitative value of kidney transplant rejection risk or the presence of kidney transplant rejection.

2. The method of claim 1, comprising generating two or more transplant rejection scores, wherein each transplant rejection score is based on a different subset of mRNAs and/or miRNAs.

3. The method of claim 1 or 2, wherein the one or more transplant rejection scores are generated using a predictive model, a machine learning based method, and/or an artificial intelligence method.

4. The method of claim 1 or 2, wherein the one or more transplant rejection scores are generated using logistic regression (Log Reg), t-test, violin plots, random forest (RE), a neural network, decision tree machine learning analysis, decision trees classification techniques, analysis of variants (ANOVA); Bayesian networks; boosting and Ada-boosting; bootstrap aggregating (or bagging) algorithms, Classification and Regression Trees (CART), boosted CART, Recursive Partitioning Trees (RPART), Curds and Whey (CW); Curds and Whey-Lasso; principal component analysis (PCA), factor rotation or factor analysis; discriminant analysis, Linear Discriminant Analysis (LDA), Eigengene Linear Discriminant Analysis (ELDA), quadratic discriminant analysis; Discriminant Function Analysis (DFA); factor rotation or factor analysis; genetic algorithms; Hidden Markov Models; kernel based machine algorithms such as kernel density estimation, kernel partial least squares algorithms, kernel matching pursuit algorithms, kernel Fisher's discriminate analysis algorithms, kernel principal components analysis algorithms; linear regression and generalized linear models, Forward Linear Stepwise Regression, Lasso (or LASSO) shrinkage and selection method, Elastic Net regularization and selection method; glmnet (Lasso and Elastic Net-regularized generalized linear model); meta-learner algorithms; nearest neighbor methods for classification or regression, Kth-nearest neighbor (KNN); non-linear regression or classification algorithms; neural networks; partial least square; rules based classifiers; shrunken centroids (SC); sliced inverse regression; Standard for the Exchange of Product model data, Application Interpreted Constructs (StepAIC); super principal component (SPC) regression; Support Vector Machines (SVM) and Recursive Support Vector Machines (RSVM), and/or combinations thereof.

5. The method of claim 1 or 2, wherein the one or more transplant rejection scores are generated using logistic regression (Log Reg), random forest (RE), a neural network, or decision tree machine learning analysis.

6. The method of claim 1 or 2, wherein the one or more mRNAs and/or miRNAs are examined by using 8 separate machine learning classifier methods based on 6 determined kidney disease states.

7. The method of any one of claims 1-6, wherein a performance of the determination of kidney transplant rejection or risk of kidney transplant rejection is measured and characterized by an AUC value from about 0.6 to about 0.99, from about 0.7 to about 0.99, from about 0.8 to about 0.99, from about 0.9 to about 0.99, from about 0.7 to about 0.79, from about 0.8 to about 0.89, from about 0.6 to about 0.89, or from about 0.6 to about 0.79.

8. The method of claim 7, wherein the AUC value is from about 0.8 to about 0.99.

9. The method of any one of claims 1-8, wherein the one or more transplant rejection scores provide a quantitative value of kidney transplant rejection risk that is predictive of a clinical assessment of a kidney disease state and can distinguish between two or more kidney disease states.

10. The method of claim 9, wherein the kidney disease state comprises non-rejection, T-cell mediated rejection (TCMR), antibody-mediated rejection (ABMR), or a mixed TCMR and ABMR disease state.

11. The method of claim 10, wherein the TCMR further comprises molecularly defined TCMR (mTCMR) and/or possible TCMR (pTCMR).

12. The method of claim 10 or 11, wherein the ABMR further comprises molecularly defined ABMR (mABMR) and/or possible ABMR (pABMR).

13. The method of any one of claims 10-12, wherein the one or more transplant rejection scores comprise a first transplant rejection score based on a set of mRNAs and/or miRNAs associated with TCMR, and a second transplant rejection score based on a set of mRNAs and/or miRNAs associated with ABMR.

14. The method of any one of claims 1-13, wherein the one or more transplant rejection scores comprise a transplant rejection score based on a set of mRNAs and/or miRNAs associated with inflammation, a transplant rejection score based on a set of mRNAs and/or miRNAs associated with allograft rejection, a transplant rejection score based on a set of mRNAs and/or miRNAs associated with T cell activation and/or differentiation, a transplant rejection score based on a set of mRNAs and/or miRNAs associated with B cell activation and/or differentiation, a transplant rejection score based on a set of mRNAs and/or miRNAs associated with a cytokine response, and/or a transplant rejection score based on a set of mRNAs and/or miRNAs associated with a chemokine response.

15. The method of any one of the preceding claims, comprising collecting the urine sample from the transplant recipient at different times and generating the one or more transplant rejection scores from each urine sample.

16. The method of claim 15, wherein the urine sample is collected from the transplant recipient prior to transplantation, simultaneous with transplantation, and/or after transplantation.

17. The method of claim 15 or 16, wherein the risk of transplant rejection is based on two or more transplant rejection scores generated at different time points, and wherein a change in two or more transplant rejection scores indicates a change in kidney disease state.

18. The method of any one of the preceding claims, wherein the kidney transplant recipient is administered a treatment for the determined kidney disease state or kidney transplant rejection.

19. The method of claim 18, wherein the treatment comprises an anti-rejection or an immunosuppressive agent.

20. The method of claim 18 or 19, wherein a change in one or more transplant rejection scores specifies a presence, absence, or extent of therapeutic response to the treatment.

21. The method of any one of claims 18 to 20, wherein the treatment is determined based on the one or more transplant rejection scores or a change in one or more transplant rejection scores.

22. The method of any one of claims 1-21, wherein the nucleic acids comprise cellular nucleic acids, extra-cellular nucleic acids, and/or nucleic acids obtained from extracellular vesicles.

23. The method of any one of claims 1-22, further comprising isolating cells from the urine samples, and extracting nucleic acids from the cells.

24. The method of any one of claims 1-23, further comprising isolating extracellular vesicles, and extracting nucleic acids from the extracellular vesicles.

25. The method of any one of claims 1-23, wherein the cDNA is amplified prior to measuring of the amount.

26. The method of any one of claims 1-24, wherein the extracted nucleic acids comprise one or more mRNAs.

27. The method of any one of claims 1-24, wherein the extracted nucleic acids comprise one or more miRNAs.

28. The method of any one of claims 1-24, wherein the extracted nucleic acids comprise one or more mRNAs and one or more miRNAs.

29. The method of any one of claims 1-28, wherein the step of preparing the composition of the nucleic acids extracted in step (a) or fractions thereof comprises amplification of cDNA derived from the nucleic acids.

30. The method of claim 29, wherein the amplification comprises performing multiplex targeted amplification of the cDNA at 10-50,000 target loci in a single reaction volume.

31. The method of claim 29 or 30, wherein the amplification comprises universal amplification.

32. The method of any one of claims 1-31, wherein the amount of one or more mRNAs and/or miRNAs is measured by using quantitative PCR, real-time PCR, digital PCR, or sequencing.

33. The method of any one of claims 1-32, the amount of one or more mRNAs and/or miRNAs is measured by using multiplex quantitative PCR, multiplex real-time PCR, and/or multiplex digital PCR.

34. The method of claim 32, wherein sequencing comprises next-generation whole genome sequencing.

35. The method of any one of claims 1-31, wherein the amount of one or more mRNAs and/or miRNAs is measured by using microarray.

36. The method of any one of claims 1-31, wherein the amount of one or more mRNAs and/or miRNAs is measured by using molecular barcodes and microscopic imaging (such as NanoString nCounter®).

37. The method of any one of claims 1-36, wherein the amount of one or more mRNAs and/or miRNAs is determined by measuring an absolute copy number of the one or more mRNAs and/or miRNAs per amount of total nucleic acids in the urine sample.

38. The method of any one of claims 1-37, wherein the one or more mRNAs and/or miRNAs are associated with antibody mediated transplant rejection (AMTR), T-cell mediated transplant rejection (TMTR), apoptosis pathways, cytokine, antimicrobial responses, and/or inflammatory cellular responses.

39. The method of claim 38, wherein the one or more mRNAs and/or miRNAs are associated with the antimicrobial responses are CXC motif chemokine ligand (CXCL) type genes.

40. The method of any one of claims 1-38, wherein the one or more mRNAs are expressed from a gene selected from the group consisting of:

| | | | | |
|---|---|---|---|---|
| ITM2A | SLAMF6 | IKZF3 | CCL5 | CXCL9 |
| IGHM | ITM2A | HIST1H2AJ | CXCL10 | CD3E |
| NKG7 | TRBC2 | HIST1H3I | TRGC2 | HIST1H1B |
| CTLA4 | PDCD1 | GZMA | IL2RB | IKZF3 |
| CD69 | SIT1 | CD96 | FAIM3 | ZAP70 |
| HIST1H1D | TRAC | RNASE6 | SLAMF6 | Z98744.2 |
| GZMB | IL18R1 | IL1RL1 | HIST1H4L | C1QB |
| CD8A | HIST1H2BM | HLA-DQB1 | LCK | ASB2 |
| HIST1H2AI | SLFN12L | CD28 | TRBV28 | RP4-620F22.2 |
| GIMAP1 | ICOS | ZNF831 | TRAT1 | GIMAP7 |
| P2RY10 | HCST | HIST1H2AK | GBP5 | EMB |
| CD2 | KLRC1 | CD6 | GPR174 | AL049822.1 |
| HLA-DQA1 | TIGIT | ITK | ST8SIA4 | GIMAP6 |
| GVINP1 | HLA-DQB1- | MRC1 | HLA-DPB1 | CCR2 |
| | AS1 | | | |
| CCND2-AS1 | AL009179.1 | EVI2B | FCGR3B | ETV7 |
| CTSW | HIST1H2AL | THEMIS | CH17- | BLM |
| | | | 373J23.1 | |
| PRF1 | DOK2 | CXCR6 | NGFR | KCNE1 |
| HIST1H3H | CTD- | HLA-DRA | CD8B | NCF1 |
| | 2313F11.1 | | | |
| GZMK | MLKL | HIST1H3B | CD3G | JAK3 |
| CD3D | C1QA | IFNG | CDCA7 | Z98744.3 |
| CXCL11 | RP11- | HLA-H | TOX | GIMAP4 |
| | 284N8.3 | | | |
| FAM78A | FAM65B | HCG4P7 | SMAP2 | EOMES |
| RASAL3 | CD163 | FCRL3 | IFIT2 | SH2D1A |
| HCG4P11 | RGS18 | MIR155HG | IFITM1 | HIST1H2BL |
| ITGA4 | RP11- | MATK | KCNJ2 | KLRB1 |
| | 463J10.3 | | | |
| HIST1H2AH | APOBEC3G | PSMB9 | PTPRC | RCSD1 |
| PYHIN1 | BTN3A3 | AC007278.2 | C11orf21 | RHOH |
| TMEM156 | HIST2H3D | NLRP3 | AF001548.5 | HIST1H2BI |
| CD74 | AGAP2 | CD52 | GBP1 | GMFG |
| LAX1 | SASH3 | GNLY | HIST2H2AB | MS4A6A |
| PDE3B | SLAMF8 | AGER | PREX1 | IRF8 |
| IKZF1 | TBC1D10C | FCGR3A | BCL11B | HLA-DRB1 |
| CD97 | CTB-186G2.1 | NLRC3 | TAP1 | CD247 |
| HIST1H3G | GBP4 | RP11-1049A21.2 | TMC8 | SELPLG |
| HIST1H2BO | CCND2 | CST7 | CCR5 | RP11- |
| | | | | 264B17.5 |
| GRAP2 | HLA-B | IKBIP | KRT1 | SERPINB12 |
| ALB | TGM3 | HS3ST6 | NRTN | FAM180A |
| FALEC | FGF22 | HCN2 | SLC24A3 | RP11- |
| | | | | 167H9.5 |
| C1QB | IL18R1 | KCNE1 | CCR2 | C1QA |
| CD163 | CST7 | SLC1A3 | RNASE6 | HIST1H1B |
| TRGC2 | MS4A6A | C1QC | RGS1 | MSR1 |
| HIST1H1D | HIST1H3I | CD28 | LSM11 | HIST1H3B |
| HIST1H2AJ | GIMAP6 | DAAM2 | CCL5 | CTLA4 |
| IGHG1 | C1orf162 | MIR155HG | ITM2A | GIMAP4 |
| HIST1H2BM | HIST1H3G | FCER1G | FKBP5 | IL1R2 |
| GZMA | CLEC12A | LINC01127 | Z98744.2 | GPR34 |
| HLA-DMB | HIST1H4L | RRM2 | TREM2 | GIMAP8 |
| CD3E | AL049822.1 | HLA-DPB1 | LMNB1 | CD300C |
| IFITM1 | BLM | SAMSN1 | IRF8 | CLSPN |
| IL1RL1 | SDS | RP11-274E7.2 | MS4A4A | PDCD1 |
| ICOS | RP11- | TNFSF13B | HIST1H3F | RP11- |
| | 848G14.5 | | | 777B9.5 |
| GIMAP2 | IL18RAP | VSIG4 | GIMAP7 | SRGN |
| TRBC2 | LIPA | SERPINE1 | CENPK | IL10 |
| GZMK | DTL | GLDN | SMAP2 | HIST1H2BB |
| SIT1 | IFITM3 | CTD-2033D15.2 | HLA-DRA | THBS1 |
| ADORA3 | STAB1 | AIM2 | EVI2A | TFPI2 |
| EMB | SLA | CD8A | RCSD1 | GIMAP1 |
| FAM105A | FPR2 | CD180 | RP11- | RP4- |
| | | | 463J10.3 | 737E23.5 |
| FCGR3A | HIST1H2BL | CD1D | UBE2C | PDK4 |
| MLKL | RP11-7F17.3 | TLR7 | PTPRC | KBTBD8 |
| CD96 | HIST1H2AI | FAIM3 | CD48 | IFI27L2 |
| HIST1H4F | CTD- | SLC16A6 | LRRC8C | NLRP6 |
| | 2033D15.3 | | | |
| TLR8 | IRAK3 | LY96 | LILRA5 | AL009179.1 |
| KIAA0101 | GCA | CCDC109B | CTSL | CCR5 |
| BTN3A2 | STAMBPL1 | HLA-DPA1 | CTD- | LAIR1 |
| | | | 2313F11.1 | |
| FES | RNASE1 | CXCL10 | TRIM22 | HIST1H3H |
| CD52 | MYBL2 | RP11-403P17.6 | RNU4-1 | KLRC1 |
| FEN1 | MIR181A1HG | HCG27 | RP11- | BIN2 |
| | | | 335I12.2 | |
| RP4- | GBP5 | FYB | PCED1B | AL031777.1 |
| 620F22.2 | | | | |
| MMD | DOK2 | RP11-404F10.2 | PPT1 | FAM65B |
| GVINP1 | SERPINF1 | CARD16 | CEP55 | HIST1H2AL |
| CD86 | CD2 | JAK3 | SELPLG | AC007278.3 |
| FAM111B | GYG1 | NLRP3 | ZWINT | ITGA4 |
| TRAC | SELL | GPR183 | TRG-AS1 | ST8SIA4 |
| NCKAP1L | STK17B | OLAH | APOC1 | PCED1B-AS1 |
| SLFN13 | TIGIT | ATHL1 | HIST1H2BI | HLA-DMA |
| ENTPD1 | SLCO2B1 | IFNG | CD300LF | F13A1 |
| NEIL3 | TMEM14E | ACSL4 | RN7SKP48 | GZMH |
| LYAR | GPNMB | IKZF1 | RP3-455J7.4 | SCIMP |
| AMIGO2 | TLR5 | EXOC6 | IFITM2 | HAVCR2 |
| TLR2 | CTB- | FGL2 | A2M | LY9 |
| | 111H14.1 | | | |
| KIAA0825 | WDR76 | LILRB5 | SLAMF6 | CTB-4116.2 |
| FGR | HELLS | CD274 | HIST1H2AB | MELK |
| ASB2 | SIGLEC9 | TPK1 | MAP2K6 | RP11- |
| | | | | 347P5.1 |
| HIST1H2AH | EVI2B | CTA-373H7.7 | FAM26F | KLRB1 |
| TRPV2 | CHIT1 | HTATSF1P2 | TSC22D3 | TLDC2 |
| MGAM | RP11-69L16.4 | CXCR6 | STAT4 | TNFSF8 |
| MB21D1 | SLFN11 | RP11-124N14.3 | TYROBP | ITGB2 |
| LILRB4 | CDK1 | PSMB8 | HIST2H2AB | DNA2 |
| PDCD1LG2 | CCL18 | RP11-25K21.6 | HLA-DQA1 | CELF2 |
| AIF1 | LBR | AL021807.1 | GMFG | PTGER2 |
| VIM | CASP1 | HCG4P11 | RP4- | HLA-DQB1 |
| | | | 671O14.5 | |
| ZNF367 | VNN1 | RP11-278C7.5 | HCST | SLC46A3 |
| SLAMF8 | HLA-H | CARD8-AS1 | RP1-68D18.2 | CTD- |
| | | | | 2033D15.1 |
| NCAPG | SMARCD3 | MMP19 | ARHGAP15 | FABP5P7 |
| HLA-DQB1- | HIST1H2BO | FCGR2A | ITGAM | MCM6 |
| AS1 | | | | |
| VCAN | CD37 | BTLA | Z98744.3 | NMI |
| RP11- | MARCH1 | RP11-290C10.1 | VNN2 | HLA-DOA |
| 1049A21.2 | | | | |
| CD300E | RGS2 | HIST1H3J | NCAPH | HIST1H2BE |
| HLA-B | CD4 | MSNP1 | CTSC | CXCL5 |
| AC008984.2 | HIST1H2AE | APMAP | CD84 | SHC4 |
| GLIPR2 | ZPLD1 | GPR171 | FAM101B | C5AR1 |
| UBASH3B | FAM78A | SLED1 | CRTAM | PMP22 |
| ZEB2-AS1 | CTC-301O7.4 | PFN1P1 | EPSTI1 | HK3 |
| C18orf54 | ATP8A1 | C11orf21 | LCP1 | FAM198B |
| RP11- | ETV7 | WAS | RP11-415J8.3 | CYBB |
| 326C3.2 | | | | |
| CDCA7L | CMSS1 | RBL1 | FLI1 | RNF175 |
| IL27RA | CSF1R | TMX1 | FAR2 | HAUS1 |
| AOAH | AQP9 | ARHGEF6 | AC007278.2 | CLEC2B |
| CSGALNACT2 | LILRB1 | ITK | LINC01093 | P2RY14 |
| FPR1 | PLK4 | RP11-488L18.10 | MDM1 | PREX1 |
| RP11- | RP11-256L6.3 | RP6-159A1.4 | CD226 | CD300LB |
| 186N15.3 | | | | |
| SNX10 | CEP85L | CLEC10A | LTB4R | MYC |
| CENPH | FXYD4 | TRAF5 | CIITA | SOAT1 |
| RP11- | AGAP2 | NAIP | PLP2 | PSMB8-AS1 |
| 325F22.2 | | | | |
| ETS1 | IRF4 | NCF1 | E2F8 | CD33 |
| BTNL8 | CLEC4E | POLR3GL | IKZF3 | IL2RA |
| GFI1 | HHEX | SNX20 | APBB1IP | RP11-15A1.7 |
| DCK | SNORA12 | EMR1 | APOBEC3G | ANXA2R |
| ANKRD22 | AC025048.1 | MS4A7 | MSN | FABP5 |
| PYHIN1 | GPR141 | RP11-455F5.5 | HCG4P7 | GAB3 |
| PARVG | C17orf53 | RP11-297C4.2 | SLC4A7 | FLT3 |
| TNFAIP6 | HJURP | PATL2 | TLR4 | LAPTM5 |
| RP11- | RAB37 | MOB3C | GTF2H2C | NGFR |
| 876N24.3 | | | | |
| KIF14 | IGSF6 | TTC27 | PPP2R2B | LGALS1 |
| RP5- | IKBIP | XXbac- | AC025171.1 | CTSW |
| 1171I10.5 | | BPG299F13.14 | | |
| SLC11A1 | HGF | CNTRL | UGT1A6 | CD53 |
| RP11- | DOCK11 | VIM-AS1 | RP11- | RP11- |
| 701P16.2 | | | 272L13.4 | 488L18.4 |
| EMP3 | CCL2 | EXO5 | PLA2G7 | GPSM3 |
| LINC01272 | STAT1 | GNG2 | CR1 | SNRPF |
| TNNI2 | HCLS1 | HIST1H3C | RP11- | HENMT1 |
| | | | 214O1.2 | |
| WIPF1 | TNFSF4 | DOCK10 | ACSL1 | IL10RA |
| HIST1H2AK | CD97 | ADAMTS2 | MAD2L1 | SASH3 |
| SLC16A1 | RN7SKP203 | RAC2 | CD36 | JAM3 |
| BUB1B | RRN3 | RAB20 | CTB-41I6.1 | RNU4-2 |
| CERKL | TSPAN2 | HLA-F | CFP | ARL6IP6 |
| TNFRSF25 | CPVL | CD6 | MYADM | RP11- |
| | | | | 149I23.3 |
| HLA-DRB5 | RFC3 | FBN1 | NUSAP1 | PLXNC1 |
| NLRC4 | CTD- | GPR68 | MAP7D3 | ARG1 |
| | 2047H16.3 | | | |
| F2R | PIK3CD-AS1 | GSG2 | HS3ST3B1 | IQGAP2 |
| ALOX5AP | AC007620.3 | IDO1 | BCL2A1 | CALHM2 |
| KIF11 | TTK | LTB | FCGR1A | EEF1E1 |
| AC006460.2 | RNASE2 | KIF15 | P2RY8 | LPAR6 |
| BASP1 | BMP7 | CALHM6 | CD14 | CD46 |
| CXCL14 | ENG | FN1 | FOXP3 | IFNGR1 |
| IL18BP | IL32 | INPP5D | IRAK2 | ISG20 |
| KLRC4- | MAP4K1 | NAMPT | PRPF31 | PRPF32 |
| KLRK1 | | | | |
| PRPF33 | PSMB10 | PYCARD | RUNX3 | SERPINA1 |
| TBP | TGFB1 | | | |
| ADD1 | AIFM3 | ANKH | ANXA1 | APP |
| ATF3 | AVPR1A | BAX | BCAP31 | BCL10 |
| BCL2L1 | BCL2L10 | BCL2L11 | BCL2L2 | BGN |
| BID | BIK | BIRC3 | BMF | BMP2 |
| BNIP3L | BRCA1 | BTG2 | BTG3 | CASP1 |
| CASP2 | CASP3 | CASP4 | CASP6 | CASP7 |
| CASP8 | CASP9 | CAV1 | CCNA1 | CCND1 |
| CCND2 | CD14 | CD2 | CD38 | CD44 |
| CD69 | CDC25B | CDK2 | CDKN1A | CDKN1B |
| CFLAR | CLU | CREBBP | CTH | CTNNB1 |
| CYLD | DAP | DAP3 | DCN | DDIT3 |
| DFFA | DIABLO | DNAJA1 | DNAJC3 | DNM1L |
| DPYD | EBP | EGR3 | EMP1 | ENO2 |
| ERBB2 | ERBB3 | EREG | ETF1 | F2 |
| F2R | FAS | FASLG | FDXR | FEZ1 |
| GADD45A | GADD45B | GCH1 | GNA15 | GPX1 |
| GPX3 | GPX4 | GSN | GSR | GSTM1 |
| GUCY2D | H1F0 | HGF | HMGB2 | HMOX1 |
| HSPB1 | IER3 | IFITM3 | IFNB1 | IFNGR1 |
| IGF2R | IGFBP6 | IL18 | IL1A | IL1B |
| IL6 | IRF1 | ISG20 | JUN | KRT18 |
| LEF1 | LGALS3 | LMNA | LPPR4 | LUM |
| MADD | MCL1 | MGMT | MMP2 | NEDD9 |
| NEFH | PAK1 | PDCD4 | PDGFRB | PEA15 |
| PLAT | PLCB2 | PMAIP1 | PPP2R5B | PPP3R1 |
| PPT1 | PRF1 | PSEN1 | PSEN2 | PTK2 |
| RARA | RELA | RETSAT | RHOB | RHOT2 |
| RNASEL | ROCK1 | SAT1 | SATB1 | SC5DL |
| SLC20A1 | SMAD7 | SOD1 | SOD2 | SPTAN1 |
| SQSTM1 | TAP1 | TGFB2 | TGFBR3 | TIMP1 |
| TIMP2 | TIMP3 | TNF | TNFRSF12A | TNFSF10 |
| TOP2A | TSPO | TXNIP | VDAC2 | WEE1 |
| XIAP | | | | |
| ALB | BASP1 | BMP7 | C11orf21 | C1QB |
| C3 | CALHM6 | CD14 | CD3D | CD3E |
| CD46 | CD6 | CD74 | CDH1 | CXCL10 |
| CXCL11 | CXCL14 | CXCL9 | ENG | FN1 |
| FOXP3 | GNLY | GZMB | HAVCR2 | IFNGR1 |
| IL18BP | IL2RA | IL32 | INPP5D | IRAK2 |
| ISG20 | KLRC4- | LCK | MAP4K1 | MRC1 |
| | KLRK1 | | | |
| NAMPT | NKG7 | PRF1 | PRPF31 | PRPF32 |
| PRPF33 | PSMB10 | PSMB9 | PYCARD | RGS1 |
| RUNX3 | SERPINA1 | SERPINB12 | STAT1 | TAP1 |
| TBP | TGFB1 | TMEM156 | | |
| RBM6 | PSMB1 | ITIH4 | TP53BP1 | DFNB31 |
| LGALS1 | AP4S1 | BRF2 | RAD23B | HNRNPA2B1 |
| ZNF436 | NECAB3 | HCST | AHDC1 | ARPP19 |
| C3orf20 | ACTR3B | UBAC2 | SETDB2 | STX17 |
| RAB30 | RPLP1 | SNRPF | CMTM3 | USP21 |
| LRIG1 | GTF2H2 | MPP7 | MERTK | ANGPT1 |
| AK9 | ITLN2 | CACHD1 | SYVN1 | ELF3 |
| UCN | ZMYM6 | BDH2 | DACT1 | COPS6 |
| 2-Sep | ZNF354A | BTD | SIGLEC17P | TADA3 |
| CTSF | LINC00469 | DMAP1 | DCTPP1 | MFSD6L |
| ADAMTSL5 | SUPT5H | S100A5 | ARMCX4 | ZNF502 |
| ZNF138 | ZNF841 | FICD | COL5A2 | RP11-15J10.1 |
| GNRHR2 | TRDC | AC016747.3 | AGGF1P1 | CHCHD3P3 |
| PRPF38AP1 | RP11- | AC010894.3 | RPL4P6 | AP000936.1 |
| | 242O24.3 | | | |
| LINC01278 | LINC00298 | AC159540.14 | LINC00299 | AMY2B |
| RPL34P18 | RP11- | RPP21 | RP11- | RP11- |
| | 1008M1.1 | | 20O24.4 | 392P7.6 |
| CDK11B | RP1-16A9.1 | RP11-585P4.5 | P2RX5- | RP11- |
| | | | TAX1BP3 | 644F5.11 |
| SPESP1 | RP11- | RP11-412D9.4 | RPL23AP97 | RP11- |
| | 603B24.1 | | | 529K1.2 |
| RP5- | RN7SL246P | PARD6G-AS1 | RP11- | RP11- |
| 1107A17.4 | | | 635N19.1 | 840I19.5 |
| RP11- | RP11- | LA16c-360A4.1 | F8A1 | uc_338 |
| 179B2.2 | 313P22.1 | | | |
| RP11- | RP11- | | | |
| 43N22.1 | 133M8.3, and | | | |
| | combinations thereof. | | | |

41. The method of any one of claims 1-38, wherein the one or more miRNAs are binding one or more expression products from a gene selected from the group consisting of:

| | | | | |
|---|---|---|---|---|
| ITM2A | SLAMF6 | IKZF3 | CCL5 | CXCL9 |
| IGHM | ITM2A | HIST1H2AJ | CXCL10 | CD3E |
| NKG7 | TRBC2 | HIST1H3I | TRGC2 | HIST1H1B |
| CTLA4 | PDCD1 | GZMA | IL2RB | IKZF3 |
| CD69 | SIT1 | CD96 | FAIM3 | ZAP70 |
| HIST1H1D | TRAC | RNASE6 | SLAMF6 | Z98744.2 |
| GZMB | IL18R1 | IL1RL1 | HIST1H4L | C1QB |
| CD8A | HIST1H2BM | HLA-DQB1 | LCK | ASB2 |
| HIST1H2AI | SLFN12L | CD28 | TRBV28 | RP4-620F22.2 |
| GIMAP1 | ICOS | ZNF831 | TRAT1 | GIMAP7 |
| P2RY10 | HCST | HIST1H2AK | GBP5 | EMB |
| CD2 | KLRC1 | CD6 | GPR174 | AL049822.1 |
| HLA-DQA1 | TIGIT | ITK | ST8SIA4 | GIMAP6 |
| GVINP1 | HLA-DQB1- | MRC1 | HLA-DPB1 | CCR2 |
| | AS1 | | | |
| CCND2-AS1 | AL009179.1 | EVI2B | FCGR3B | ETV7 |
| CTSW | HIST1H2AL | THEMIS | CH17- | BLM |
| | | | 373J23.1 | |
| PRF1 | DOK2 | CXCR6 | NGFR | KCNE1 |
| HIST1H3H | CTD- | HLA-DRA | CD8B | NCF1 |
| | 2313F11.1 | | | |
| GZMK | MLKL | HIST1H3B | CD3G | JAK3 |
| CD3D | C1QA | IFNG | CDCA7 | Z98744.3 |
| CXCL11 | RP11- | HLA-H | TOX | GIMAP4 |
| | 284N8.3 | | | |
| FAM78A | FAM65B | HCG4P7 | SMAP2 | EOMES |
| RASAL3 | CD163 | FCRL3 | IFIT2 | SH2D1A |
| HCG4P11 | RGS18 | MIR155HG | IFITM1 | HIST1H2BL |
| ITGA4 | RP11- | MATK | KCNJ2 | KLRB1 |
| | 463J10.3 | | | |
| HIST1H2AH | APOBEC3G | PSMB9 | PTPRC | RCSD1 |
| PYHIN1 | BTN3A3 | AC007278.2 | C11orf21 | RHOH |
| TMEM156 | HIST2H3D | NLRP3 | AF001548.5 | HIST1H2BI |
| CD74 | AGAP2 | CD52 | GBP1 | GMFG |
| LAX1 | SASH3 | GNLY | HIST2H2AB | MS4A6A |
| PDE3B | SLAMF8 | AGER | PREX1 | IRF8 |
| IKZF1 | TBC1D10C | FCGR3A | BCL11B | HLA-DRB1 |
| CD97 | CTB-186G2.1 | NLRC3 | TAP1 | CD247 |
| HIST1H3G | GBP4 | RP11-1049A21.2 | TMC8 | SELPLG |
| HIST1H2BO | CCND2 | CST7 | CCR5 | RP11- |
| | | | | 264B17.5 |
| GRAP2 | HLA-B | IKBIP | KRT1 | SERPINB12 |
| ALB | TGM3 | HS3ST6 | NRTN | FAM180A |
| FALEC | FGF22 | HCN2 | SLC24A3 | RP11- |
| | | | | 167H9.5 |
| C1QB | IL18R1 | KCNE1 | CCR2 | C1QA |
| CD163 | CST7 | SLC1A3 | RNASE6 | HIST1H1B |
| TRGC2 | MS4A6A | C1QC | RGS1 | MSR1 |
| HIST1H1D | HIST1H3I | CD28 | LSM11 | HIST1H3B |
| HIST1H2AJ | GIMAP6 | DAAM2 | CCL5 | CTLA4 |
| IGHG1 | C1orf162 | MIR155HG | ITM2A | GIMAP4 |
| HIST1H2BM | HIST1H3G | FCER1G | FKBP5 | IL1R2 |
| GZMA | CLEC12A | LINC01127 | Z98744.2 | GPR34 |
| HLA-DMB | HIST1H4L | RRM2 | TREM2 | GIMAP8 |
| CD3E | AL049822.1 | HLA-DPB1 | LMNB1 | CD300C |
| IFITM1 | BLM | SAMSN1 | IRF8 | CLSPN |
| IL1RL1 | SDS | RP11-274E7.2 | MS4A4A | PDCD1 |
| ICOS | RP11- | TNFSF13B | HIST1H3F | RP11- |
| | 848G14.5 | | | 777B9.5 |
| GIMAP2 | IL18RAP | VSIG4 | GIMAP7 | SRGN |
| TRBC2 | LIPA | SERPINE1 | CENPK | IL10 |
| GZMK | DTL | GLDN | SMAP2 | HIST1H2BB |
| SIT1 | IFITM3 | CTD-2033D15.2 | HLA-DRA | THBS1 |
| ADORA3 | STAB1 | AIM2 | EVI2A | TFPI2 |
| EMB | SLA | CD8A | RCSD1 | GIMAP1 |
| FAM105A | FPR2 | CD180 | RP11- | RP4- |
| | | | 463J10.3 | 737E23.5 |
| FCGR3A | HIST1H2BL | CD1D | UBE2C | PDK4 |
| MLKL | RP11-7F17.3 | TLR7 | PTPRC | KBTBD8 |
| CD96 | HIST1H2AI | FAIM3 | CD48 | IFI27L2 |
| HIST1H4F | CTD- | SLC16A6 | LRRC8C | NLRP6 |
| | 2033D15.3 | | | |
| TLR8 | IRAK3 | LY96 | LILRA5 | AL009179.1 |
| KIAA0101 | GCA | CCDC109B | CTSL | CCR5 |
| BTN3A2 | STAMBPL1 | HLA-DPA1 | CTD- | LAIR1 |
| | | | 2313F11.1 | |
| FES | RNASE1 | CXCL10 | TRIM22 | HIST1H3H |
| CD52 | MYBL2 | RP11-403P17.6 | RNU4-1 | KLRC1 |
| FEN1 | MIR181A1HG | HCG27 | RP11- | BIN2 |
| | | | 335I12.2 | |
| RP4- | GBP5 | FYB | PCED1B | AL031777.1 |
| 620F22.2 | | | | |
| MMD | DOK2 | RP11-404F10.2 | PPT1 | FAM65B |
| GVINP1 | SERPINF1 | CARD16 | CEP55 | HIST1H2AL |
| CD86 | CD2 | JAK3 | SELPLG | AC007278.3 |
| FAM111B | GYG1 | NLRP3 | ZWINT | ITGA4 |
| TRAC | SELL | GPR183 | TRG-AS1 | ST8SIA4 |
| NCKAP1L | STK17B | OLAH | APOC1 | PCED1B-AS1 |
| SLFN13 | TIGIT | ATHL1 | HIST1H2BI | HLA-DMA |
| ENTPD1 | SLCO2B1 | IFNG | CD300LF | F13A1 |
| NEIL3 | TMEM14E | ACSL4 | RN7SKP48 | GZMH |
| LYAR | GPNMB | IKZF1 | RP3-455J7.4 | SCIMP |
| AMIGO2 | TLR5 | EXOC6 | IFITM2 | HAVCR2 |
| TLR2 | CTB- | FGL2 | A2M | LY9 |
| | 111H14.1 | | | |
| KIAA0825 | WDR76 | LILRB5 | SLAMF6 | CTB-41I6.2 |
| FGR | HELLS | CD274 | HIST1H2AB | MELK |
| ASB2 | SIGLEC9 | TPK1 | MAP2K6 | RP11- |
| | | | | 347P5.1 |
| HIST1H2AH | EVI2B | CTA-373H7.7 | FAM26F | KLRB1 |
| TRPV2 | CHIT1 | HTATSF1P2 | TSC22D3 | TLDC2 |
| MGAM | RP11-69L16.4 | CXCR6 | STAT4 | TNFSF8 |
| MB21D1 | SLFN11 | RP11-124N14.3 | TYROBP | ITGB2 |
| LILRB4 | CDK1 | PSMB8 | HIST2H2AB | DNA2 |
| PDCD1LG2 | CCL18 | RP11-25K21.6 | HLA-DQA1 | CELF2 |
| AIF1 | LBR | AL021807.1 | GMFG | PTGER2 |
| VIM | CASP1 | HCG4P11 | RP4- | HLA-DQB1 |
| | | | 671O14.5 | |
| ZNF367 | VNN1 | RP11-278C7.5 | HCST | SLC46A3 |
| SLAMF8 | HLA-H | CARD8-AS1 | RP1-68D18.2 | CTD- |
| | | | | 2033D15.1 |
| NCAPG | SMARCD3 | MMP19 | ARHGAP15 | FABP5P7 |
| HLA-DQB1- | HIST1H2BO | FCGR2A | ITGAM | MCM6 |
| AS1 | | | | |
| VCAN | CD37 | BTLA | Z98744.3 | NMI |
| RP11- | MARCH1 | RP11-290C10.1 | VNN2 | HLA-DOA |
| 1049A21.2 | | | | |
| CD300E | RGS2 | HIST1H3J | NCAPH | HIST1H2BE |
| HLA-B | CD4 | MSNP1 | CTSC | CXCL5 |
| AC008984.2 | HIST1H2AE | APMAP | CD84 | SHC4 |
| GLIPR2 | ZPLD1 | GPR171 | FAM101B | C5AR1 |
| UBASH3B | FAM78A | SLED1 | CRTAM | PMP22 |
| ZEB2-AS1 | CTC-301O7.4 | PFN1P1 | EPSTI1 | HK3 |
| C18orf54 | ATP8A1 | C11orf21 | LCP1 | FAM198B |
| RP11- | ETV7 | WAS | RP11-415J8.3 | CYBB |
| 326C3.2 | | | | |
| CDCA7L | CMSS1 | RBL1 | FLI1 | RNF175 |
| IL27RA | CSF1R | TMX1 | FAR2 | HAUS1 |
| AOAH | AQP9 | ARHGEF6 | AC007278.2 | CLEC2B |
| CSGALNACT2 | LILRB1 | ITK | LINC01093 | P2RY14 |
| FPR1 | PLK4 | RP11-488L18.10 | MDM1 | PREX1 |
| RP11- | RP11-256L6.3 | RP6-159A1.4 | CD226 | CD300LB |
| 186N15.3 | | | | |
| SNX10 | CEP85L | CLEC10A | LTB4R | MYC |
| CENPH | FXYD4 | TRAF5 | CIITA | SOAT1 |
| RP11- | AGAP2 | NAIP | PLP2 | PSMB8-AS1 |
| 325F22.2 | | | | |
| ETS1 | IRF4 | NCF1 | E2F8 | CD33 |
| BTNL8 | CLEC4E | POLR3GL | IKZF3 | IL2RA |
| GFI1 | HHEX | SNX20 | APBB1IP | RP11-15A1.7 |
| DCK | SNORA12 | EMR1 | APOBEC3G | ANXA2R |
| ANKRD22 | AC025048.1 | MS4A7 | MSN | FABP5 |
| PYHIN1 | GPR141 | RP11-455F5.5 | HCG4P7 | GAB3 |
| PARVG | C17orf53 | RP11-297C4.2 | SLC4A7 | FLT3 |
| TNFAIP6 | HJURP | PATL2 | TLR4 | LAPTM5 |
| RP11- | RAB37 | MOB3C | GTF2H2C | NGFR |
| 876N24.3 | | | | |
| KIF14 | IGSF6 | TTC27 | PPP2R2B | LGALS1 |
| RP5- | IKBIP | XXbac- | AC025171.1 | CTSW |
| 1171I10.5 | | BPG299F13.14 | | |
| SLC11A1 | HGF | CNTRL | UGT1A6 | CD53 |
| RP11- | DOCK11 | VIM-AS1 | RP11- | RP11- |
| 701P16.2 | | | 272L13.4 | 488L18.4 |
| EMP3 | CCL2 | EXO5 | PLA2G7 | GPSM3 |
| LINC01272 | STAT1 | GNG2 | CR1 | SNRPF |
| TNNI2 | HCLS1 | HIST1H3C | RP11- | HENMT1 |
| | | | 214O1.2 | |
| WIPF1 | TNFSF4 | DOCK10 | ACSL1 | IL10RA |
| HIST1H2AK | CD97 | ADAMTS2 | MAD2L1 | SASH3 |
| SLC16A1 | RN7SKP203 | RAC2 | CD36 | JAM3 |
| BUB1B | RRN3 | RAB20 | CTB-41I6.1 | RNU4-2 |
| CERKL | TSPAN2 | HLA-F | CFP | ARL6IP6 |
| TNFRSF25 | CPVL | CD6 | MYADM | RP11- |
| | | | | 149I23.3 |
| HLA-DRB5 | RFC3 | FBN1 | NUSAP1 | PLXNC1 |
| NLRC4 | CTD- | GPR68 | MAP7D3 | ARG1 |
| | 2047H16.3 | | | |
| F2R | PIK3CD-AS1 | GSG2 | HS3ST3B1 | IQGAP2 |
| ALOX5AP | AC007620.3 | IDO1 | BCL2A1 | CALHM2 |
| KIF11 | TTK | LTB | FCGR1A | EEF1E1 |
| AC006460.2 | RNASE2 | KIF15 | P2RY8 | LPAR6 |
| BASP1 | BMP7 | CALHM6 | CD14 | CD46 |
| CXCL14 | ENG | FN1 | FOXP3 | IFNGR1 |
| IL18BP | IL32 | INPP5D | IRAK2 | ISG20 |
| KLRC4- | MAP4K1 | NAMPT | PRPF31 | PRPF32 |
| KLRK1 | | | | |
| PRPF33 | PSMB10 | PYCARD | RUNX3 | SERPINA1 |
| TBP | TGFB1 | | | |
| ADD1 | AIFM3 | ANKH | ANXA1 | APP |
| ATF3 | AVPR1A | BAX | BCAP31 | BCL10 |
| BCL2L1 | BCL2L10 | BCL2L11 | BCL2L2 | BGN |
| BID | BIK | BIRC3 | BMF | BMP2 |
| BNIP3L | BRCA1 | BTG2 | BTG3 | CASP1 |
| CASP2 | CASP3 | CASP4 | CASP6 | CASP7 |
| CASP8 | CASP9 | CAV1 | CCNA1 | CCND1 |
| CCND2 | CD14 | CD2 | CD38 | CD44 |
| CD69 | CDC25B | CDK2 | CDKN1A | CDKN1B |
| CFLAR | CLU | CREBBP | CTH | CTNNB1 |
| CYLD | DAP | DAP3 | DCN | DDIT3 |
| DFFA | DIABLO | DNAJA1 | DNAJC3 | DNM1L |
| DPYD | EBP | EGR3 | EMP1 | ENO2 |
| ERBB2 | ERBB3 | EREG | ETF1 | F2 |
| F2R | FAS | FASLG | FDXR | FEZ1 |
| GADD45A | GADD45B | GCH1 | GNA15 | GPX1 |
| GPX3 | GPX4 | GSN | GSR | GSTM1 |
| GUCY2D | H1F0 | HGF | HMGB2 | HMOX1 |
| HSPB1 | IER3 | IFITM3 | IFNB1 | IFNGR1 |
| IGF2R | IGFBP6 | IL18 | IL1A | IL1B |
| IL6 | IRF1 | ISG20 | JUN | KRT18 |
| LEF1 | LGALS3 | LMNA | LPPR4 | LUM |
| MADD | MCL1 | MGMT | MMP2 | NEDD9 |
| NEFH | PAK1 | PDCD4 | PDGFRB | PEA15 |
| PLAT | PLCB2 | PMAIP1 | PPP2R5B | PPP3R1 |
| PPT1 | PRF1 | PSEN1 | PSEN2 | PTK2 |
| RARA | RELA | RETSAT | RHOB | RHOT2 |
| RNASEL | ROCK1 | SAT1 | SATB1 | SC5DL |
| SLC20A1 | SMAD7 | SOD1 | SOD2 | SPTAN1 |
| SQSTM1 | TAP1 | TGFB2 | TGFBR3 | TIMP1 |
| TIMP2 | TIMP3 | TNF | TNFRSF12A | TNFSF10 |
| TOP2A | TSPO | TXNIP | VDAC2 | WEE1 |
| XIAP | | | | |
| ALB | BASP1 | BMP7 | C11orf21 | C1QB |
| C3 | CALHM6 | CD14 | CD3D | CD3E |
| CD46 | CD6 | CD74 | CDH1 | CXCL10 |
| CXCL11 | CXCL14 | CXCL9 | ENG | FN1 |
| FOXP3 | GNLY | GZMB | HAVCR2 | IFNGR1 |
| IL18BP | IL2RA | IL32 | INPP5D | IRAK2 |
| ISG20 | KLRC4- | LCK | MAP4K1 | MRC1 |
| | KLRK1 | | | |
| NAMPT | NKG7 | PRF1 | PRPF31 | PRPF32 |
| PRPF33 | PSMB10 | PSMB9 | PYCARD | RGS1 |
| RUNX3 | SERPINA1 | SERPINB12 | STAT1 | TAP1 |
| TBP | TGFB1 | TMEM156 | | |
| RBM6 | PSMB1 | ITIH4 | TP53BP1 | DFNB31 |
| LGALS1 | AP4S1 | BRF2 | RAD23B | HNRNPA2B1 |
| ZNF436 | NECAB3 | HCST | AHDC1 | ARPP19 |
| C3orf20 | ACTR3B | UBAC2 | SETDB2 | STX17 |
| RAB30 | RPLP1 | SNRPF | CMTM3 | USP21 |
| LRIG1 | GTF2H2 | MPP7 | MERTK | ANGPT1 |
| AK9 | ITLN2 | CACHD1 | SYVN1 | ELF3 |
| UCN | ZMYM6 | BDH2 | DACT1 | COPS6 |
| 2-Sep | ZNF354A | BTD | SIGLEC17P | TADA3 |
| CTSF | LINC00469 | DMAP1 | DCTPP1 | MFSD6L |
| ADAMTSL5 | SUPT5H | S100A5 | ARMCX4 | ZNF502 |
| ZNF138 | ZNF841 | FICD | COL5A2 | RP11-15J10.1 |
| GNRHR2 | TRDC | AC016747.3 | AGGF1P1 | CHCHD3P3 |
| PRPF38AP1 | RP11- | AC010894.3 | RPL4P6 | AP000936.1 |
| | 242O24.3 | | | |
| LINC01278 | LINC00298 | AC159540.14 | LINC00299 | AMY2B |
| RPL34P18 | RP11- | RPP21 | RP11- | RP11- |
| | 1008M1.1 | | 20O24.4 | 392P7.6 |
| CDK11B | RP1-16A9.1 | RP11-585P4.5 | P2RX5- | RP11- |
| | | | TAX1BP3 | 644F5.11 |
| SPESP1 | RP11- | RP11-412D9.4 | RPL23AP97 | RP11- |
| | 603B24.1 | | | 529K1.2 |
| RP5- | RN7SL246P | PARD6G-AS1 | RP11- | RP11- |
| 1107A17.4 | | | 635N19.1 | 840I19.5 |
| RP11- | RP11- | LA16c-360A4.1 | F8A1 | uc_338 |
| 179B2.2 | 313P22.1 | | | |
| RP11- | RP11- | | | |
| 43N22.1 | 133M8.3, and | | | |
| | combinations thereof. | | | |

42. The method of any one of claims 1-38, wherein the one or more mRNAs are expressed from a gene selected from the group consisting of:

| | | | | |
|---|---|---|---|---|
| ALB | BASP1 | BMP7 | C11orf21 | C1QB |
| C3 | CALHM6 | CD14 | CD3D | CD3E |
| CD46 | CD6 | CD74 | CDH1 | CXCL10 |
| CXCL11 | CXCL14 | CXCL9 | ENG | FN1 |
| FOXP3 | GNLY | GZMB | HAVCR2 | IFNGR1 |
| IL18BP | IL2RA | IL32 | INPP5D | IRAK2 |
| ISG20 | KLRC4-KLRK1 | LCK | MAP4K1 | MRC1 |
| NAMPT | NKG7 | PRF1 | PRPF31 | PRPF32 |
| PRPF33 | PSMB10 | PSMB9 | PYCARD | RGS1 |
| RUNX3 | SERPINA1 | SERPINB12 | STAT1 | TAP1 |
| TBP | TGFB1 | TMEM156, and | | |
| | | combinations | | |
| | | thereof. | | |

43. The method of any one of claims 1-38, wherein the one or more miRNAs are binding one or more mRNAs expressed from a gene selected from the group consisting of:

| | | | | |
|---|---|---|---|---|
| ALB | BASP1 | BMP7 | C11orf21 | C1QB |
| C3 | CALHM6 | CD14 | CD3D | CD3E |
| CD46 | CD6 | CD74 | CDH1 | CXCL10 |
| CXCL11 | CXCL14 | CXCL9 | ENG | FN1 |
| FOXP3 | GNLY | GZMB | HAVCR2 | IFNGR1 |
| IL18BP | IL2RA | IL32 | INPP5D | IRAK2 |
| ISG20 | KLRC4-KLRK1 | LCK | MAP4K1 | MRC1 |
| NAMPT | NKG7 | PRF1 | PRPF31 | PRPF32 |
| PRPF33 | PSMB10 | PSMB9 | PYCARD | RGS1 |
| RUNX3 | SERPINA1 | SERPINB12 | STAT1 | TAP1 |
| TBP | TGFB1 | TMEM156, and | | |
| | | combinations | | |
| | | thereof. | | |

44. The method of any one of claims 1-39, wherein the one or more mRNAs are expressed from a gene selected from the group consisting of:

| | | | | | | | | | |
|---|---|---|---|---|---|---|---|---|---|
| AD | AIF | AN | ANXA | APP | ATF | AVP | BA | BC | BC |
| BCL | BC | BC | BCL2 | BG | BID | BIK | BIR | BM | BM |
| BNI | BR | BT | BTG3 | CA | CAS | CAS | CA | CA | CA |
| CA | CA | CA | CCNA | CC | CCN | CD1 | CD | CD | CD |
| CD | CD | CD | CDKN | CD | CFL | CLU | CR | CT | CT |
| CYL | DA | DA | DCN | DDI | DFF | DIA | DN | DN | DN |
| DP | EB | EG | EMP1 | EN | ERB | ERB | ER | ETF | F2 |
| F2R | FA | FA | FDXR | FEZ | GAD | GAD | GC | GN | GP |
| GP | GP | GS | GSR | GS | GUC | H1F | HG | HM | HM |
| HS | IER | IFIT | IFNB1 | IFN | IGF2 | IGF | IL1 | IL1 | IL1 |
| IL6 | IRF | ISG | JUN | KRT | LEF | LGA | LM | LPP | LU |
| MA | MC | MG | MMP | NE | NEF | PAK | PD | PD | PE |
| PLA | PL | PM | PPP2 | PPP | PPT | PRF | PS | PS | PT |
| RA | RE | RE | RHO | RH | RNA | ROC | SA | SA | SC |
| SLC | SM | SO | SOD2 | SPT | SQS | TAP | TG | TG | TIM |
| TIM | TIM | TN | TNFR | TNF | TOP | TSP | TX | VD | WE |
| XIA | | | | | | | | | |

, and combinations thereof.

45. The method of any one of claims 1-38, wherein the one or more miRNAs are binding one or more mRNAs expressed from a gene selected from the group consisting of:

| | | | | | | | | | |
|---|---|---|---|---|---|---|---|---|---|
| AD | AIF | AN | ANXA | APP | ATF | AVP | BA | BC | BC |
| BCL | BCL | BCL | BCL2 | BG | BID | BIK | BIR | BM | BM |
| BNI | BR | BT | BTG3 | CAS | CAS | CAS | CA | CA | CA |
| CAS | CA | CA | CCNA | CC | CCN | CD1 | CD | CD | CD |
| CD6 | CD | CD | CDKN | CD | CFL | CLU | CR | CT | CT |
| CYL | DA | DA | DCN | DDI | DFF | DIA | DN | DN | DN |
| DPY | EB | EG | EMP1 | EN | ERB | ERB | ER | ETF | F2 |
| F2R | FAS | FAS | FDXR | FEZ | GAD | GAD | GC | GN | GP |
| GP | GP | GS | GSR | GST | GUC | H1F | HG | HM | HM |
| HSP | IER | IFIT | IFNB1 | IFN | IGF2 | IGF | IL18 | IL1 | IL1 |
| IL6 | IRF | ISG | JUN | KRT | LEF | LGA | LM | LPP | LU |
| MA | MC | MG | MMP2 | NE | NEF | PAK | PD | PD | PE |
| PLA | PLC | PM | PPP2 | PPP | PPT | PRF | PS | PS | PT |
| RA | REL | RE | RHOB | RH | RNA | ROC | SAT | SAT | SC |
| SLC | SM | SO | SOD2 | SPT | SQS | TAP | TG | TG | TIM |
| TIM | TIM | TNF | TNFR | TNF | TOP | TSP | TX | VD | WE |
| XIA | | | | | | | | | |

, and combinations thereof.

46. The method of any one of claims 1-38, wherein the one or more mRNAs are expressed from a gene selected from the group consisting of: CXCL9, CD3ϵ, IP-10, LCK, C1QB, PSMB9, Tim-3, Foxp3, FAM26F, ALB, CTB-186G2.1, CXCL11, GNLY, MRC1, RGS1, RP11-1049A21.2, TMEM156, Z98744.3, SERPINB12, C11orf21, AF001548, 18s ribosomal, Basp1, Cd6, Cxcl10, Cxcl9, Inpp5d, Isg20, Lck, Nkg7, Runx3, Tap1, GZMB, PRF1, CD74, IL32, STAT1, CXCL14, SERPINA1, B2M, C3, PYCARD, BMP7, TBP, NAMPT, IFNGR1, IRAK2, IL18BP, and combinations thereof.

47. The method of any one of claims 1-38, wherein the one or more mRNAs are expressed from a gene selected from the group consisting of: CXCL9, CD3ϵ, IP-10, LCK, C1QB, PSMB9, Tim-3, Foxp3, FAM26F, ALB, CTB-186G2.1, CXCL11, GNLY, MRC1, RGS1, RP11-1049A21.2, TMEM156, Z98744.3, SERPINB12, C11orf21, AF001548, 18s ribosomal, Basp1, Cd6, Cxcl10, Cxcl9, Inpp5d, Isg20, Lck, Nkg7, Runx3, Tap1, GZMB, PRF1, CD74, IL32, STAT1, CXCL14, SERPINA1, B2M, C3, PYCARD, BMP7, TBP, NAMPT, IFNGR1, IRAK2, Calhm6, Klrc4-Klrk1, Psmb10, CD3delta, Map4k1, IL2Ra, TGFB1, FN1, CDH1, PRPF31, HAVCR2, IL18BP, and combinations thereof.

48. The method of any one of claims 1-38, wherein the one or more mRNAs are expressed from a gene selected from the group consisting of: CXCL9, CD3ϵ, IP-10, LCK, C1QB, PSMB9, Tim-3, Foxp3, FAM26F, ALB, CTB-186G2.1, CXCL11, GNLY, MRC1, RGS1, RP11-1049A21.2, TMEM156, Z98744.3, SERPINB12, C11orf21, AF001548, 18s ribosomal, Basp1, Cd6, Cxcl10, Cxcl9, Inpp5d, Isg20, Lck, Nkg7, Runx3, Tap1, and combinations thereof.

49. The method of any one of claims 1-38, wherein the one or more mRNAs are expressed from a gene selected from the group consisting of: CXCL11, CXCL9, GNLY, PSMB9, TAP1, CXCL10, NKG7, ISG20, RUNX3, C11orf21, C1QB, LCK, INPP5D, CD6, CD3ε, MRC1, BASP1, TMEM156, ALB, FOXP3, RGS1, SERPINB12, and combinations thereof.

50. The method of any one of claims 1-38, wherein the one or more mRNAs are expressed from a gene selected from the group consisting of: CXCL11, CXCL9, GNLY, PSMB9, TAP1, CXCL10, NKG7, ISG20, RUNX3, C11orf21, C1QB, LCK, INPP5D, CD6, CD3ε, MRC1, BASP1, TMEM156, ALB, FOXP3, RGS1, SERPINB12, PRF1, CALHM6, KLRC4-KLRK1, CD74, GZMB, PSMB10, B2M, STAT1, CD3delta, PYCARD, IL18BP, MAP4K1, IL32, IL2RA, C3, IFNGR1, IRAK2, TGFB1, FN1, NAMPT, SERPINA1, TBP, CDH1, PRPF31, BMP7, CXCL14, HAVCR2, and combinations thereof.

51. The method of any one of claims 1-50, wherein the one or more mRNAs are not expressed from a gene selected from the group consisting of: PDCD1, MARCHF8, DCAF12, IL1R2, FLT3, ITGA4, ITGAM, PF4, C6orf25, SEMA7A, RHOU, and combinations thereof.

52. The method of any one of claims 1-50, wherein the one or more mRNAs are not expressed from a gene selected from the group consisting of: CCDC159, dcaf12, DECR1, ERCC5, EWSR1, FLT3, GABPB2, GPI, IL1R2, ITGA4, ITGAM, MAP3K3, MAPK9, MARCHF8, NONO, PDCD1, PF4, RHOU, SEMA7A, SRRM1, TBC1D10B, TOP2B, and combinations thereof.

53. The method of any one of claims 1-51, wherein the one or more miRNAs are selected from the group consisting of miR-186-5p, miR-665, miR-873-5p.1, miR-543, miR-330-3p, miR-362-5p/500b-5p, miR-217, miR-140-5p, miR-193-3p, miR-382-5p, miR-140-3p.2, miR-653-5p, miR-455-3p.2, miR-145-5p, miR-491-5p, miR-23-3p, miR-375, miR-129-5p, miR-96-5p/1271-5p, miR-182-5p, miR-371-5p, miR-203a-3p.1, miR-494-3p, miR-146-5p, miR-140-3p.1, miR-125-5p, miR-346, miR-760, miR-185-5p, miR-325-3p, miR-15-5p/16-5p/195-5p/424-5p/497-5p, miR-503-5p, miR-423-5p, miR-496.1, miR-155-5p, miR-142-3p.2, miR-24-3p, miR-874-3p, miR-25-3p/32-5p/92-3p/363-3p/367-3p, miR-17-5p/20-5p/93-5p/106-5p/519-3p, miR-181-5p, miR-142-5p, miR-130-3p/301-3p/454-3p, miR-21-5p/590-5p, miR-103-3p/107, miR-137, miR-340-5p, miR-490-3p, miR-143-3p, miR-409-3p, miR-27-3p, miR-138-5p, miR-485-5p, miR-328-3p, miR-326, miR-148-3p/152-3p, miR-9-5p, miR-31-5p, miR-452-5p/892-3p, miR-202-5p, miR-29-3p, miR-338-3p, miR-26-5p, let-7-5p/98-5p, miR-196-5p, miR-30-5p, miR-142-3p.1, miR-19-3p, miR-411-3p, miR-493-5p, miR-218-5p, miR-203a-3p.2, miR-495-3p, miR-425-5p, miR-135-5p, miR-154-3p/487-3p, miR-223-3p, miR-219-5p, miR-670-3p, miR-216b-5p, miR-200bc-3p/429, miR-320, miR-216a-5p, miR-141-3p/200a-3p, miR-144-3p, miR-128-3p, miR-455-3p.1, miR-219a-2-3p, miR-873-5p.2, miR-448, miR-183-5p.2, miR-374-5p, miR-505-3p.1, miR-433-3p, miR-377-3p, miR-365-3p, miR-124-3p.1, miR-410-3p, miR-199-3p, miR-22-3p, miR-129-3p, miR-383-5p.1, miR-1-3p/206, miR-296-5p, miR-299-3p, miR-212-5p, miR-331-3p, miR-378-3p, miR-136-5p, miR-1193, miR-505-3p.2, miR-302c-3p.2/520-3p, miR-421, miR-499a-5p, miR-302-3p/372-3p/373-3p/520-3p, miR-124-3p.2/506-3p, miR-34-5p/449-5p, miR-376c-3p, miR-139-5p, miR-221-3p/222-3p, miR-504-5p.1, miR-335-5p, miR-101-3p.1, miR-431-5p, miR-489-3p, miR-369-3p, miR-330-3p.2, miR-18-5p, miR-28-5p/708-5p, miR-133a-3p.2/133b, miR-205-5p, miR-199-5p, miR-455-5p, miR-126-3p.2, miR-7-5p, miR-483-3p.2, miR-668-3p, miR-1306-5p, miR-150-5p, miR-296-3p, miR-204-5p/211-5p, miR-3064-5p, miR-532-5p, miR-876-5p, miR-501-3p/502-3p, miR-33-5p, miR-153-3p, miR-214-5p, miR-655-3p, miR-342-3p, miR-133a-3p.1, miR-411-5p.1, miR-496.2, miR-411-5p.2, miR-582-5p, miR-381-3p, miR-188-5p, miR-383-5p.2, miR-486-5p, miR-183-5p.1, miR-208-3p, miR-193a-5p, miR-101-3p.2, miR-542-3p, miR-190-5p, miR-299-5p, miR-154-5p, miR-802, miR-323-3p, miR-532-3p, miR-224-5p, miR-339-5p, miR-194-5p, miR-149-5p, miR-493-3p, miR-382-3p, miR-132-3p/212-3p, miR-1197, miR-99-5p/100-5p, miR-877-5p, miR-483-3p.1, miR-10-5p, miR-361-5p, miR-539-3p, miR-191-5p, miR-329-3p/362-3p, miR-122-5p, miR-379-5p, miR-376-3p, miR-1298-5p, miR-451, miR-210-3p, miR-1224-5p, miR-324-5p, miR-544a-5p, miR-488-3p, miR-758-3p, miR-151-3p, miR-875-5p, miR-134-5p, miR-192-5p/215-5p, and miR-127-3p.

54. The method of any one of claims 1-51, wherein the one or more miRNAs are selected from the group consisting of miR-96-5p/1271-5p, miR-493-5p, miR-183-5p.2, miR-150-5p, miR-7-5p, miR-653-5p, miR-200bc-3p/429, miR-212-5p, miR-1298-5p, miR-137, miR-758-3p, miR-325-3p, miR-542-3p, miR-25-3p/32-5p/92-3p/363-3p/367-3p, miR-495-3p, miR-30-5p, miR-873-5p.1, miR-146-5p, miR-505-3p.1, miR-539-3p, miR-216a-5p, miR-216b-5p, miR-340-5p, miR-361-5p, miR-338-3p, miR-217, miR-9-5p, miR-219a-2-3p, miR-15-5p/16-5p/195-5p/424-5p/497-5p, miR-503-5p, miR-199-3p, miR-1-3p/206, miR-17-5p/20-5p/93-5p/106-5p/519-3p, miR-302-3p/372-3p/373-3p/520-3p, miR-142-5p, miR-302c-3p.2/520-3p, miR-326, miR-760, miR-138-5p, miR-27-3p, miR-145-5p, miR-142-3p.2, miR-101-3p.2, miR-182-5p, miR-203a-3p.2, miR-140-3p.1, miR-183-5p.1, miR-144-3p, miR-101-3p.1, miR-330-3p, miR-224-5p, miR-148-3p/152-3p, miR-485-5p, miR-122-5p, miR-155-5p, miR-320, miR-23-3p, miR-124-3p.2/506-3p, miR-135-5p, miR-381-3p, miR-26-5p, miR-1224-5p, miR-192-5p/215-5p, miR-1249-3p, miR-125-5p, miR-483-3p.2, miR-668-3p, miR-223-3p, miR-655-3p, miR-382-5p, miR-130-3p/301-3p/454-3p, miR-19-3p, miR-582-5p, miR-194-5p, miR-802, miR-483-3p.1, miR-382-3p, miR-129-5p, miR-3064-5p, miR-873-5p.2, miR-499a-5p, miR-128-3p, miR-532-5p, miR-296-5p, miR-744-5p, miR-425-5p, miR-218-5p, and miR-496.1.

55. The method of any one of the preceding claims, further comprising:
(i) measuring an amount of donor-derived cell-free DNA in a sample obtained from the transplant recipient, extracting cell-free DNA from the sample obtained from the transplant recipient, wherein the extracted cell-free DNA comprises donor-derived cell-free DNA and recipient-derived cell-free DNA;
(ii) performing targeted amplification of the extracted DNA at 50-50,000 target loci in a single reaction volume;
(iii) sequencing the amplified DNA by high-throughput sequencing to obtain sequencing reads and measuring the amount of donor-derived cell-free DNA based on the sequencing reads, generating a transplant rejection score indicating transplant rejection based on whether the measured amount of donor-derived cell-free DNA or a function thereof exceeds a cutoff threshold of cell-free DNA amount that indicates transplant rejection, wherein transplant rejection is determined based on both the one or more transplant rejection scores from the measured amount of one or more mRNAs and/or miRNAs and the transplant rejection score determined based on the measured amount of donor-derived cell-free DNA.

56. The method of any one of the preceding claims, wherein measuring the amount of mRNAs and/or miRNAs comprises amplification of at least 2, at least 5, at least 10, at least 20, at least 30, at least 50, at least 100 target nucleic acid molecules, from 2-10, 200-100, 50-500, or 50-2000 target nucleic acid molecules, using at least at least 2, at least 5, at least 10, at least 20, at least 30, at least 50, at least 100 target RNA molecules, from 2-10, 200-100, 50-500, or 50-2000 pairs of forward and reverse PCR primers.

57. The method of any one of the preceding claims, wherein the one or more mRNAs and/or miRNAs are determined by text mining databases.

58. The method of any one of the preceding claims, wherein the amount of one or more mRNAs and/or miRNAs is measured relative to a housekeeping gene.

59. A method of preparing a composition of nucleic acids derived from a urine sample of a kidney transplant recipient useful for determination of kidney transplant rejection or risk of kidney transplant rejection comprising:
(a) extracting nucleic acids from the urine sample of the kidney transplant recipient, wherein the extracted nucleic acids comprise one or more RNA molecules associated with a risk of kidney transplant rejection;
(b) preparing the composition of nucleic acids from the extracted nucleic acids from step (a) by isolating RNA molecules and removing contaminating molecules; optionally wherein preparing the composition comprises performing reverse transcription of the RNA molecules to synthesize cDNA;
(c) measuring an amount of RNA molecules associated with a risk of kidney transplant rejection in the composition of nucleic acids, and generating one or more transplant rejection scores from the measured amount of one or more RNA molecules, wherein the one or more transplant rejection scores provide a quantitative value of kidney transplant rejection risk or the presence of kidney transplant rejection.

60. The method of claim 59, wherein the one or more RNA molecules are mRNA or miRNA.

61. A method of preparing a composition of protein derived from a urine sample of a kidney transplant recipient useful for determination of kidney transplant rejection or risk of kidney transplant rejection comprising:
(a) extracting protein from the urine sample of the kidney transplant recipient, wherein the extracted proteins are associated with a risk of kidney transplant rejection;
(b) preparing the composition of protein from the protein extracted in step (a) by removing contaminating molecules;
(c) measuring an amount of proteins in the composition, and generating one or more transplant rejection scores from the measured amount, wherein the one or more transplant rejection scores provide a quantitative value of kidney transplant rejection risk or the presence of kidney transplant rejection.

62. The method of claim 61, wherein the measuring step is based on two or more transplant rejection scores, wherein each transplant rejection score is based on a different subset of proteins.

63. The method of claim 61 or 62, wherein the one or more transplant rejection scores are generated using a predictive model, a machine learning based method, and/or an artificial intelligence method.

64. The method of any one of claims 61-63, wherein the one or more transplant rejection scores provide a quantitative value of kidney transplant rejection risk that is predictive of a clinical assessment of a kidney disease state and can distinguish between two or more kidney disease states.

65. The method of claim 64, wherein the kidney disease state comprises non-rejection, T-cell mediated rejection (TCMR), antibody-mediated rejection (ABMR), or a mixed TCMR and ABMR disease state.

66. The method of claim 65, wherein the TCMR further comprises molecularly defined TCMR (mTCMR) and/or possible TCMR (pTCMR).

67. The method of claim 65 or 66, wherein the ABMR further comprises molecularly defined ABMR (mABMR) and/or possible ABMR (pABMR).

68. The method of any one of claims 65-67, wherein the one or more transplant rejection scores comprise a first transplant rejection score based on a set of proteins associated with TCMR, and a second transplant rejection score based on a set of proteins associated with ABMR.

69. The method of any one of claims 61-68, wherein the one or more transplant rejection scores comprise a transplant rejection score based on a set of proteins associated with inflammation, a transplant rejection score based on a set of proteins associated with allograft rejection, a transplant rejection score based on a set of proteins associated with T cell activation and/or differentiation, a transplant rejection score based on a set of proteins associated with B cell activation and/or differentiation, a transplant rejection score based on a set of proteins associated with a cytokine response, and/or a transplant rejection score based on a set of proteins associated with a chemokine response.

70. The method of any one of claims 61-69, comprising collecting the urine sample from the transplant recipient at different times and generating the one or more transplant rejection scores from each urine sample.

71. The method of claim 70, wherein the urine sample is collected prior to transplantation, simultaneous with transplantation, and/or after transplantation.

72. The method of claim 71, wherein the risk of transplant rejection is based on multiple transplant rejection scores generated at different time points, and wherein a change in one or more transplant rejection scores indicates a change in kidney disease state.

73. The method of any one of claims 61-72, wherein the kidney transplant recipient is administered a treatment for the determined kidney disease state or kidney transplant rejection.

74. The method of claim 73, wherein a change in one or more transplant rejection scores specifies a presence, absence, or extent of therapeutic response to the treatment.

75. The method of claim 74, wherein the treatment is determined based on the one or more transplant rejection scores or a change in one or more transplant rejection scores.

76. The method of claim 75, wherein the treatment comprises an anti-rejection or an immunosuppressive agent.

77. The method of any one of claims 61-76, further comprising isolating cells from the urine samples, and extracting protein from the cells.

78. The method of any one of claims 61-77, further comprising isolating extracellular vesicles, and extracting protein from the extracellular vesicles.

79. The method of any one of claims 61-78, wherein the one or more proteins are expressed from or regulated by a gene selected from the group consisting of:

| | | | | |
|---|---|---|---|---|
| ITM2A | SLAMF6 | IKZF3 | CCL5 | CXCL9 |
| IGHM | ITM2A | HIST1H2AJ | CXCL10 | CD3E |
| NKG7 | TRBC2 | HIST1H3I | TRGC2 | HIST1H1B |
| CTLA4 | PDCD1 | GZMA | IL2RB | IKZF3 |
| CD69 | SIT1 | CD96 | FAIM3 | ZAP70 |
| HIST1H1D | TRAC | RNASE6 | SLAMF6 | Z98744.2 |
| GZMB | IL18R1 | IL1RL1 | HIST1H4L | C1QB |
| CD8A | HIST1H2BM | HLA-DQB1 | LCK | ASB2 |
| HIST1H2AI | SLFN12L | CD28 | TRBV28 | RP4-620F22.2 |
| GIMAP1 | ICOS | ZNF831 | TRAT1 | GIMAP7 |
| P2RY10 | HCST | HIST1H2AK | GBP5 | EMB |
| CD2 | KLRC1 | CD6 | GPR174 | AL049822.1 |
| HLA-DQA1 | TIGIT | ITK | ST8SIA4 | GIMAP6 |
| GVINP1 | HLA-DQB1- | MRC1 | HLA-DPB1 | CCR2 |
| | AS1 | | | |
| CCND2-AS1 | AL009179.1 | EVI2B | FCGR3B | ETV7 |
| CTSW | HIST1H2AL | THEMIS | CH17- | BLM |
| | | | 373J23.1 | |
| PRF1 | DOK2 | CXCR6 | NGFR | KCNE1 |
| HIST1H3H | CTD- | HLA-DRA | CD8B | NCF1 |
| | 2313F11.1 | | | |
| GZMK | MLKL | HIST1H3B | CD3G | JAK3 |
| CD3D | C1QA | IFNG | CDCA7 | Z98744.3 |
| CXCL11 | RP11- | HLA-H | TOX | GIMAP4 |
| | 284N8.3 | | | |
| FAM78A | FAM65B | HCG4P7 | SMAP2 | EOMES |
| RASAL3 | CD163 | FCRL3 | IFIT2 | SH2D1A |
| HCG4P11 | RGS18 | MIR155HG | IFITM1 | HIST1H2BL |
| ITGA4 | RP11- | MATK | KCNJ2 | KLRB1 |
| | 463J10.3 | | | |
| HIST1H2AH | APOBEC3G | PSMB9 | PTPRC | RCSD1 |
| PYHIN1 | BTN3A3 | AC007278.2 | C11orf21 | RHOH |
| TMEM156 | HIST2H3D | NLRP3 | AF001548.5 | HIST1H2BI |
| CD74 | AGAP2 | CD52 | GBP1 | GMFG |
| LAX1 | SASH3 | GNLY | HIST2H2AB | MS4A6A |
| PDE3B | SLAMF8 | AGER | PREX1 | IRF8 |
| IKZF1 | TBC1D10C | FCGR3A | BCL11B | HLA-DRB1 |
| CD97 | CTB-186G2.1 | NLRC3 | TAP1 | CD247 |
| HIST1H3G | GBP4 | RP11-1049A21.2 | TMC8 | SELPLG |
| HIST1H2BO | CCND2 | CST7 | CCR5 | RP11- |
| | | | | 264B17.5 |
| GRAP2 | HLA-B | IKBIP | KRT1 | SERPINB12 |
| ALB | TGM3 | HS3ST6 | NRTN | FAM180A |
| FALEC | FGF22 | HCN2 | SLC24A3 | RP11- |
| | | | | 167H9.5 |
| C1QB | IL18R1 | KCNE1 | CCR2 | C1QA |
| CD163 | CST7 | SLC1A3 | RNASE6 | HIST1H1B |
| TRGC2 | MS4A6A | C1QC | RGS1 | MSR1 |
| HIST1H1D | HIST1H3I | CD28 | LSM11 | HIST1H3B |
| HIST1H2AJ | GIMAP6 | DAAM2 | CCL5 | CTLA4 |
| IGHG1 | C1orf162 | MIR155HG | ITM2A | GIMAP4 |
| HIST1H2BM | HIST1H3G | FCER1G | FKBP5 | IL1R2 |
| GZMA | CLEC12A | LINC01127 | Z98744.2 | GPR34 |
| HLA-DMB | HIST1H4L | RRM2 | TREM2 | GIMAP8 |
| CD3E | AL049822.1 | HLA-DPB1 | LMNB1 | CD300C |
| IFITM1 | BLM | SAMSN1 | IRF8 | CLSPN |
| IL1RL1 | SDS | RP11-274E7.2 | MS4A4A | PDCD1 |
| ICOS | RP11- | TNFSF13B | HIST1H3F | RP11- |
| | 848G14.5 | | | 777B9.5 |
| GIMAP2 | IL18RAP | VSIG4 | GIMAP7 | SRGN |
| TRBC2 | LIPA | SERPINE1 | CENPK | IL10 |
| GZMK | DTL | GLDN | SMAP2 | HIST1H2BB |
| SIT1 | IFITM3 | CTD-2033D15.2 | HLA-DRA | THBS1 |
| ADORA3 | STAB1 | AIM2 | EVI2A | TFPI2 |
| EMB | SLA | CD8A | RCSD1 | GIMAP1 |
| FAM105A | FPR2 | CD180 | RP11- | RP4- |
| | | | 463J10.3 | 737E23.5 |
| FCGR3A | HIST1H2BL | CD1D | UBE2C | PDK4 |
| MLKL | RP11-7F17.3 | TLR7 | PTPRC | KBTBD8 |
| CD96 | HIST1H2AI | FAIM3 | CD48 | IFI27L2 |
| HIST1H4F | CTD- | SLC16A6 | LRRC8C | NLRP6 |
| | 2033D15.3 | | | |
| TLR8 | IRAK3 | LY96 | LILRA5 | AL009179.1 |
| KIAA0101 | GCA | CCDC109B | CTSL | CCR5 |
| BTN3A2 | STAMBPL1 | HLA-DPA1 | CTD- | LAIR1 |
| | | | 2313F11.1 | |
| FES | RNASE1 | CXCL10 | TRIM22 | HIST1H3H |
| CD52 | MYBL2 | RP11-403P17.6 | RNU4-1 | KLRC1 |
| FEN1 | MIR181A1HG | HCG27 | RP11- | BIN2 |
| | | | 335I12.2 | |
| RP4- | GBP5 | FYB | PCED1B | AL031777.1 |
| 620F22.2 | | | | |
| MMD | DOK2 | RP11-404F10.2 | PPT1 | FAM65B |
| GVINP1 | SERPINF1 | CARD16 | CEP55 | HIST1H2AL |
| CD86 | CD2 | JAK3 | SELPLG | AC007278.3 |
| FAM111B | GYG1 | NLRP3 | ZWINT | ITGA4 |
| TRAC | SELL | GPR183 | TRG-AS1 | ST8SIA4 |
| NCKAP1L | STK17B | OLAH | APOC1 | PCED1B-AS1 |
| SLFN13 | TIGIT | ATHL1 | HIST1H2BI | HLA-DMA |
| ENTPD1 | SLCO2B1 | IFNG | CD300LF | F13A1 |
| NEIL3 | TMEM14E | ACSL4 | RN7SKP48 | GZMH |
| LYAR | GPNMB | IKZF1 | RP3-455J7.4 | SCIMP |
| AMIGO2 | TLR5 | EXOC6 | IFITM2 | HAVCR2 |
| TLR2 | CTB- | FGL2 | A2M | LY9 |
| | 111H14.1 | | | |
| KIAA0825 | WDR76 | LILRB5 | SLAMF6 | CTB-41I6.2 |
| FGR | HELLS | CD274 | HIST1H2AB | MELK |
| ASB2 | SIGLEC9 | TPK1 | MAP2K6 | RP11- |
| | | | | 347P5.1 |
| HIST1H2AH | EVI2B | CTA-373H7.7 | FAM26F | KLRB1 |
| TRPV2 | CHIT1 | HTATSF1P2 | TSC22D3 | TLDC2 |
| MGAM | RP11-69L16.4 | CXCR6 | STAT4 | TNFSF8 |
| MB21D1 | SLFN11 | RP11-124N14.3 | TYROBP | ITGB2 |
| LILRB4 | CDK1 | PSMB8 | HIST2H2AB | DNA2 |
| PDCD1LG2 | CCL18 | RP11-25K21.6 | HLA-DQA1 | CELF2 |
| AIF1 | LBR | AL021807.1 | GMFG | PTGER2 |
| VIM | CASP1 | HCG4P11 | RP4- | HLA-DQB1 |
| | | | 671O14.5 | |
| ZNF367 | VNN1 | RP11-278C7.5 | HCST | SLC46A3 |
| SLAMF8 | HLA-H | CARD8-AS1 | RP1-68D18.2 | CTD- |
| | | | | 2033D15.1 |
| NCAPG | SMARCD3 | MMP19 | ARHGAP15 | FABP5P7 |
| HLA-DQB1- | HIST1H2BO | FCGR2A | ITGAM | MCM6 |
| AS1 | | | | |
| VCAN | CD37 | BTLA | Z98744.3 | NMI |
| RP11- | MARCH1 | RP11-290C10.1 | VNN2 | HLA-DOA |
| 1049A21.2 | | | | |
| CD300E | RGS2 | HIST1H3J | NCAPH | HIST1H2BE |
| HLA-B | CD4 | MSNP1 | CTSC | CXCL5 |
| AC008984.2 | HIST1H2AE | APMAP | CD84 | SHC4 |
| GLIPR2 | ZPLD1 | GPR171 | FAM101B | C5AR1 |
| UBASH3B | FAM78A | SLED1 | CRTAM | PMP22 |
| ZEB2-AS1 | CTC-301O7.4 | PFN1P1 | EPSTI1 | HK3 |
| C18orf54 | ATP8A1 | C11orf21 | LCP1 | FAM198B |
| RP11- | ETV7 | WAS | RP11-415J8.3 | CYBB |
| 326C3.2 | | | | |
| CDCA7L | CMSS1 | RBL1 | FLI1 | RNF175 |
| IL27RA | CSF1R | TMX1 | FAR2 | HAUS1 |
| AOAH | AQP9 | ARHGEF6 | AC007278.2 | CLEC2B |
| CSGALNACT2 | LILRB1 | ITK | LINC01093 | P2RY14 |
| FPR1 | PLK4 | RP11-488L18.10 | MDM1 | PREX1 |
| RP11- | RP11-256L6.3 | RP6-159A1.4 | CD226 | CD300LB |
| 186N15.3 | | | | |
| SNX10 | CEP85L | CLEC10A | LTB4R | MYC |
| CENPH | FXYD4 | TRAF5 | CIITA | SOAT1 |
| RP11- | AGAP2 | NAIP | PLP2 | PSMB8-AS1 |
| 325F22.2 | | | | |
| ETS1 | IRF4 | NCF1 | E2F8 | CD33 |
| BTNL8 | CLEC4E | POLR3GL | IKZF3 | IL2RA |
| GFI1 | HHEX | SNX20 | APBB1IP | RP11-15A1.7 |
| DCK | SNORA12 | EMR1 | APOBEC3G | ANXA2R |
| ANKRD22 | AC025048.1 | MS4A7 | MSN | FABP5 |
| PYHIN1 | GPR141 | RP11-455F5.5 | HCG4P7 | GAB3 |
| PARVG | C17orf53 | RP11-297C4.2 | SLC4A7 | FLT3 |
| TNFAIP6 | HJURP | PATL2 | TLR4 | LAPTM5 |
| RP11- | RAB37 | MOB3C | GTF2H2C | NGFR |
| 876N24.3 | | | | |
| KIF14 | IGSF6 | TTC27 | PPP2R2B | LGALS1 |
| RP5- | IKBIP | XXbac- | AC025171.1 | CTSW |
| 1171I10.5 | | BPG299F13.14 | | |
| SLC11A1 | HGF | CNTRL | UGT1A6 | CD53 |
| RP11- | DOCK11 | VIM-AS1 | RP11- | RP11- |
| 701P16.2 | | | 272L13.4 | 488L18.4 |
| EMP3 | CCL2 | EXO5 | PLA2G7 | GPSM3 |
| LINC01272 | STAT1 | GNG2 | CR1 | SNRPF |
| TNNI2 | HCLS1 | HIST1H3C | RP11- | HENMT1 |
| | | | 214O1.2 | |
| WIPF1 | TNFSF4 | DOCK10 | ACSL1 | IL10RA |
| HIST1H2AK | CD97 | ADAMTS2 | MAD2L1 | SASH3 |
| SLC16A1 | RN7SKP203 | RAC2 | CD36 | JAM3 |
| BUB1B | RRN3 | RAB20 | CTB-41I6.1 | RNU4-2 |
| CERKL | TSPAN2 | HLA-F | CFP | ARL6IP6 |
| TNFRSF25 | CPVL | CD6 | MYADM | RP11- |
| | | | | 149I23.3 |
| HLA-DRB5 | RFC3 | FBN1 | NUSAP1 | PLXNC1 |
| NLRC4 | CTD- | GPR68 | MAP7D3 | ARG1 |
| | 2047H16.3 | | | |
| F2R | PIK3CD-AS1 | GSG2 | HS3ST3B1 | IQGAP2 |
| ALOX5AP | AC007620.3 | IDO1 | BCL2A1 | CALHM2 |
| KIF11 | TTK | LTB | FCGR1A | EEF1E1 |
| AC006460.2 | RNASE2 | KIF15 | P2RY8 | LPAR6 |
| BASP1 | BMP7 | CALHM6 | CD14 | CD46 |
| CXCL14 | ENG | FN1 | FOXP3 | IFNGR1 |
| IL18BP | IL32 | INPP5D | IRAK2 | ISG20 |
| KLRC4- | MAP4K1 | NAMPT | PRPF31 | PRPF32 |
| KLRK1 | | | | |
| PRPF33 | PSMB10 | PYCARD | RUNX3 | SERPINA1 |
| TBP | TGFB1 | | | |
| ADD1 | AIFM3 | ANKH | ANXA1 | APP |
| ATF3 | AVPR1A | BAX | BCAP31 | BCL10 |
| BCL2L1 | BCL2L10 | BCL2L11 | BCL2L2 | BGN |
| BID | BIK | BIRC3 | BMF | BMP2 |
| BNIP3L | BRCA1 | BTG2 | BTG3 | CASP1 |
| CASP2 | CASP3 | CASP4 | CASP6 | CASP7 |
| CASP8 | CASP9 | CAV1 | CCNA1 | CCND1 |
| CCND2 | CD14 | CD2 | CD38 | CD44 |
| CD69 | CDC25B | CDK2 | CDKN1A | CDKN1B |
| CFLAR | CLU | CREBBP | CTH | CTNNB1 |
| CYLD | DAP | DAP3 | DCN | DDIT3 |
| DFFA | DIABLO | DNAJA1 | DNAJC3 | DNM1L |
| DPYD | EBP | EGR3 | EMP1 | ENO2 |
| ERBB2 | ERBB3 | EREG | ETF1 | F2 |
| F2R | FAS | FASLG | FDXR | FEZ1 |
| GADD45A | GADD45B | GCH1 | GNA15 | GPX1 |
| GPX3 | GPX4 | GSN | GSR | GSTM1 |
| GUCY2D | H1F0 | HGF | HMGB2 | HMOX1 |
| HSPB1 | IER3 | IFITM3 | IFNB1 | IFNGR1 |
| IGF2R | IGFBP6 | IL18 | IL1A | IL1B |
| IL6 | IRF1 | ISG20 | JUN | KRT18 |
| LEF1 | LGALS3 | LMNA | LPPR4 | LUM |
| MADD | MCL1 | MGMT | MMP2 | NEDD9 |
| NEFH | PAK1 | PDCD4 | PDGFRB | PEA15 |
| PLAT | PLCB2 | PMAIP1 | PPP2R5B | PPP3R1 |
| PPT1 | PRF1 | PSEN1 | PSEN2 | PTK2 |
| RARA | RELA | RETSAT | RHOB | RHOT2 |
| RNASEL | ROCK1 | SAT1 | SATB1 | SC5DL |
| SLC20A1 | SMAD7 | SOD1 | SOD2 | SPTAN1 |
| SQSTM1 | TAP1 | TGFB2 | TGFBR3 | TIMP1 |
| TIMP2 | TIMP3 | TNF | TNFRSF12A | TNFSF10 |
| TOP2A | TSPO | TXNIP | VDAC2 | WEE1 |
| XIAP | | | | |
| ALB | BASP1 | BMP7 | C11orf21 | C1QB |
| C3 | CALHM6 | CD14 | CD3D | CD3E |
| CD46 | CD6 | CD74 | CDH1 | CXCL10 |
| CXCL11 | CXCL14 | CXCL9 | ENG | FN1 |
| FOXP3 | GNLY | GZMB | HAVCR2 | IFNGR1 |
| IL18BP | IL2RA | IL32 | INPP5D | IRAK2 |
| ISG20 | KLRC4- | LCK | MAP4K1 | MRC1 |
| | KLRK1 | | | |
| NAMPT | NKG7 | PRF1 | PRPF31 | PRPF32 |
| PRPF33 | PSMB10 | PSMB9 | PYCARD | RGS1 |
| RUNX3 | SERPINA1 | SERPINB12 | STAT1 | TAP1 |
| TBP | TGFB1 | TMEM156 | | |
| RBM6 | PSMB1 | ITIH4 | TP53BP1 | DFNB31 |
| LGALS1 | AP4S1 | BRF2 | RAD23B | HNRNPA2B1 |
| ZNF436 | NECAB3 | HCST | AHDC1 | ARPP19 |
| C3orf20 | ACTR3B | UBAC2 | SETDB2 | STX17 |
| RAB30 | RPLP1 | SNRPF | CMTM3 | USP21 |
| LRIG1 | GTF2H2 | MPP7 | MERTK | ANGPT1 |
| AK9 | ITLN2 | CACHD1 | SYVN1 | ELF3 |
| UCN | ZMYM6 | BDH2 | DACT1 | COPS6 |
| 2-Sep | ZNF354A | BTD | SIGLEC17P | TADA3 |
| CTSF | LINC00469 | DMAP1 | DCTPP1 | MFSD6L |
| ADAMTSL5 | SUPT5H | S100A5 | ARMCX4 | ZNF502 |
| ZNF138 | ZNF841 | FICD | COL5A2 | RP11-15J10.1 |
| GNRHR2 | TRDC | AC016747.3 | AGGF1P1 | CHCHD3P3 |
| PRPF38AP1 | RP11- | AC010894.3 | RPL4P6 | AP000936.1 |
| | 242O24.3 | | | |
| LINC01278 | LINC00298 | AC159540.14 | LINC00299 | AMY2B |
| RPL34P18 | RP11- | RPP21 | RP11- | RP11- |
| | 1008M1.1 | | 20O24.4 | 392P7.6 |
| CDK11B | RP1-16A9.1 | RP11-585P4.5 | P2RX5- | RP11- |
| | | | TAX1BP3 | 644F5.11 |
| SPESP1 | RP11- | RP11-412D9.4 | RPL23AP97 | RP11- |
| | 603B24.1 | | | 529K1.2 |
| RP5- | RN7SL246P | PARD6G-AS1 | RP11- | RP11- |
| 1107A17.4 | | | 635N19.1 | 840I19.5 |
| RP11- | RP11- | LA16c-360A4.1 | F8A1 | uc_338 |
| 179B2.2 | 313P22.1 | | | |
| RP11- | RP11-133M8.3, and | | | |
| 43N22.1 | combinations thereof. | | | |

80. The method of any one of claims 61-78, wherein the one or more proteins are expressed from a gene selected from the group consisting of:

| | | | | |
|---|---|---|---|---|
| ALB | BASP1 | BMP7 | C11orf21 | C1QB |
| C3 | CALHM6 | CD14 | CD3D | CD3E |
| CD46 | CD6 | CD74 | CDH1 | CXCL10 |
| CXCL11 | CXCL14 | CXCL9 | ENG | FN1 |
| FOXP3 | GNLY | GZMB | HAVCR2 | IFNGR1 |
| IL18BP | IL2RA | IL32 | INPP5D | IRAK2 |
| ISG20 | KLRC4-KLRK1 | LCK | MAP4K1 | MRC1 |
| NAMPT | NKG7 | PRF1 | PRPF31 | PRPF32 |
| PRPF33 | PSMB10 | PSMB9 | PYCARD | RGS1 |
| RUNX3 | SERPINA1 | SERPINB12 | STAT1 | TAP1 |
| TBP | TGFB1 | TMEM156, and | | |
| | | combinations | | |
| | | thereof. | | |

81. The method of any one of claims 61-78, wherein the one or more proteins are expressed from a gene selected from the group consisting of:

| | | | | | | | | | |
|---|---|---|---|---|---|---|---|---|---|
| AD | AIF | AN | ANXA | APP | ATF | AVP | BA | BC | BC |
| BCL | BCL | BCL | BCL2 | BG | BID | BIK | BIR | BM | BM |
| BNI | BR | BT | BTG3 | CAS | CAS | CAS | CA | CA | CA |
| CAS | CA | CA | CCNA | CC | CCN | CD1 | CD | CD | CD |
| CD6 | CD | CD | CDKN | CD | CFL | CLU | CR | CT | CT |
| CYL | DA | DA | DCN | DDI | DFF | DIA | DN | DN | DN |
| DPY | EB | EG | EMP1 | EN | ERB | ERB | ER | ETF | F2 |
| F2R | FAS | FAS | FDXR | FEZ | GAD | GAD | GO | GN | GP |
| GP | GP | GS | GSR | GST | GUC | H1F | HG | HM | HM |
| HSP | IER | IFIT | IFNB1 | IFN | IGF2 | IGF | IL18 | IL1 | IL1 |
| IL6 | IRF | ISG | JUN | KRT | LEF | LGA | LM | LPP | LU |
| MA | MC | MG | MMP2 | NE | NEF | PAK | PD | PD | PE |
| PLA | PLC | PM | PPP2 | PPP | PPT | PRF | PS | PS | PT |
| RA | REL | RE | RHOB | RH | RNA | ROC | SAT | SAT | SC |
| SLC | SM | SO | SOD2 | SPT | SQS | TAP | TG | TG | TIM |
| TIM | TIM | TNF | TNFR | TNF | TOP | TSP | TX | VD | WE |
| XIA | | | | | | | | | |

, and combinations thereof.

82. The method of any one of claims 61-78, wherein the one or more proteins are expressed from a gene selected from the group consisting of: CXCL9, CD3ϵ, IP-10, LCK, C1QB, PSMB9, Tim-3, Foxp3, FAM26F, ALB, CTB-186G2.1, CXCL11, GNLY, MRC1, RGS1, RP11-1049A21.2, TMEM156, Z98744.3, SERPINB12, C11orf21, AF001548, 18s ribosomal, Basp1, Cd6, Cxcl10, Cxcl9, Inpp5d, Isg20, Lck, Nkg7, Runx3, Tap1, GZMB, PRF1, CD74, IL32, STAT1, CXCL14, SERPINA1, B2M, C3, PYCARD, BMP7, TBP, NAMPT, IFNGR1, IRAK2, IL18BP, and combinations thereof.

83. The method of any one of claims 61-78, wherein the one or more proteins are expressed from a gene selected from the group consisting of: CXCL9, CD3ϵ, IP-10, LCK, C1QB, PSMB9, Tim-3, Foxp3, FAM26F, ALB, CTB-186G2.1, CXCL11, GNLY, MRC1, RGS1, RP11-1049A21.2, TMEM156, Z98744.3, SERPINB12, C11orf21, AF001548, 18s ribosomal, Basp1, Cd6, Cxcl10, Cxcl9, Inpp5d, Isg20, Lck, Nkg7, Runx3, Tap1, GZMB, PRF1, CD74, IL32, STAT1, CXCL14, SERPINA1, B2M, C3, PYCARD, BMP7, TBP, NAMPT, IFNGR1, IRAK2, Calhm6, Klrc4-Klrk1, Psmb10, CD3delta, Map4k1, IL2Ra, TGFB1, FN1, CDH1, PRPF31, HAVCR2, IL18BP, and combinations thereof.

84. The method of any one of claims 61-78, wherein the one or more proteins are expressed from a gene selected from the group consisting of: CXCL9, CD3ϵ, IP-10, LCK, C1QB, PSMB9, Tim-3, Foxp3, FAM26F, ALB, CTB-186G2.1, CXCL11, GNLY, MRC1, RGS1, RP11-1049A21.2, TMEM156, Z98744.3, SERPINB12, C11orf21, AF001548, 18s ribosomal, Basp1, Cd6, Cxcl10, Cxcl9, Inpp5d, Isg20, Lck, Nkg7, Runx3, Tap1, and combinations thereof.

85. The method of any one of claims 61-78, wherein the one or more proteins are expressed from a gene selected from the group consisting of: CXCL11, CXCL9, GNLY, PSMB9, TAP1, CXCL10, NKG7, ISG20, RUNX3, C11orf21, C1QB, LCK, INPP5D, CD6, CD3ε, MRC1, BASP1, TMEM156, ALB, FOXP3, RGS1, SERPINB12, and combinations thereof.

86. The method of any one of claims 61-78, wherein the one or more proteins are expressed from a gene selected from the group consisting of: CXCL11, CXCL9, GNLY, PSMB9, TAP1, CXCL10, NKG7, ISG20, RUNX3, C11orf21, C1QB, LCK, INPP5D, CD6, CD3ε, MRC1, BASP1, TMEM156, ALB, FOXP3, RGS1, SERPINB12, PRF1, CALHM6, KLRC4-KLRK1, CD74, GZMB, PSMB10, B2M, STAT1, CD3delta, PYCARD, IL18BP, MAP4K1, IL32, IL2RA, C3, IFNGR1, IRAK2, TGFB1, FN1, NAMPT, SERPINA1, TBP, CDH1, PRPF31, BMP7, CXCL14, HAVCR2 and combinations thereof.

87. The method of any one of claims 61-86, wherein the one or more proteins are not expressed from a gene selected from the group consisting of: PDCD1, MARCHF8, DCAF12, IL1R2, FLT3, ITGA4, ITGAM, PF4, C6orf25, SEMA7A, RHOU, and combinations thereof.

88. The method of any one of claims 61-86, wherein the one or more proteins are not expressed from a gene selected from the group consisting of: CCDC159, dcaf12, DECR1, ERCC5, EWSR1, FLT3, GABPB2, GPI, IL1R2, ITGA4, ITGAM, MAP3K3, MAPK9, MARCHF8, NONO, PDCD1, PF4, RHOU, SEMA7A, SRRM1, TBC1D10B, TOP2B, and combinations thereof.

89. The method of any one of claims 61-88, further comprising:
(i) measuring an amount of donor-derived cell-free DNA in a sample obtained from the transplant recipient, extracting cell-free DNA from the sample obtained from the transplant recipient, wherein the extracted cell-free DNA comprises donor-derived cell-free DNA and recipient-derived cell-free DNA;
(ii) performing targeted amplification of the extracted DNA at 50-50,000 target loci in a single reaction volume;
(iii) sequencing the amplified DNA by high-throughput sequencing to obtain sequencing reads and measuring the amount of donor-derived cell-free DNA based on the sequencing reads, generating a score indicating transplant rejection based on whether the measured amount of donor-derived cell-free DNA or a function thereof exceeds a cutoff threshold of cell-free DNA amount that indicates transplant rejection, wherein transplant rejection is determined based on both the one or more scores based on the measured amount of proteins and the score determined based on the measured amount of donor-derived cell-free DNA.
